(19) United States
(12) Patent Application Publication
(10) Pub. No.: US 2013/0189690 A1
(43) Pub. Date: Jul. 25, 2013

(54) SCN5A Splice Variants for Use in Methods Relating to Sudden Cardiac Death and Need for Implanted Cardiac Defibrillators

(71) Applicants: THE BOARD TRUSTEES OF THE UNIVERSITY OF ILLINOIS, Urbana, IL (US); The United States of Government as represented by the Department of Veterans Affairs, Washington, DC (US)

(72) Inventor: Samuel Dudley, Chicago, IL (US)

(73) Assignees: The United States Government as represented by the Department of Veterans Affairs, Washington, DC (US); THE BOARD OF TRUSTEES OF THE UNIVERSITY OF ILLINOIS, Urbana, IL (US)

(21) Appl. No.: 13/841,283

(22) Filed: Mar. 15, 2013

Related U.S. Application Data
(63) Continuation-in-part of application No. PCT/US2012/020564, filed on Jun. 1, 2012.
(60) Provisional application No. 61/557,203, filed on Nov. 8, 2011.
(60) Provisional application No. 61/527,890, filed on Aug. 26, 2011.
(60) Provisional application No. 61/527,916, filed on Aug. 26, 2011.

(51) Int. Cl. G06F 19/12; C12Q 1/68

(52) U.S. Cl. 435/6.11; 702/20

(57) ABSTRACT

Provided herein are methods of determining a subject's need for an implanted cardiac defibrillator, methods of determining a subject's risk for sudden cardiac death (SCD), arrhythmias, or heart failure, methods of determining a subject's need for an anti-arrhythmic agent, e.g., a sodium channel blocker, and methods of reducing risk of SCD in a subject. In exemplary embodiments, each of the methods comprise the step of determining a ratio, R_{S}, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts. Further provided herein are systems, computer-readable storage media, having stored thereon machine-readable instructions executable by a processor, and related methods implemented by a processor in a computer. Kits are additionally provided herein.

### CROSS-REFERENCE TO RELATED APPLICATIONS

This application claims priority to International Patent Application No. PCT/US2012/20564, filed on Jan. 6, 2012, which claims priority to U.S. Provisional Patent Application No. 61/430,462, filed Jan. 6, 2011, U.S. Provisional Application No. 61/527,890, filed Aug. 26, 2011, U.S. Provisional Application No. 61/527,916, filed Aug. 26, 2011, and U.S. Provisional Application No. 61/557,203, filed Nov. 8, 2011. The disclosures of each of these applications are incorporated herein by reference in their entirety.

### STATEMENT OF U.S. GOVERNMENTAL INTEREST

This invention was made with U.S. government support under National Institutes of Health (NIH) National Heart, Lung and Blood Institute (NHLBI) Grant No. R01 HL1024025-01A1 and NIH Small Business Technology Transfer (STTR) Grant No. 1R41HL112355-01A1. The government has certain rights in this invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 2 megabytes ASCII (Text) file named “46463B_SeqListing.txt,” created on Mar. 13, 2013.

### BACKGROUND

Heart failure (HF) represents a major health care concern in the United States. It has been estimated that approximately 5 million patients in the U.S. have HF, and, annually, another 550,000 people are diagnosed with this disease (Hunt et al., Circulation 112:e154-E235 (2005)). Not surprisingly, HF is the single most common diagnosis upon hospital admission.

HF patients have a risk for sudden cardiac death (SCD) which is 6 to 9 times greater than that of non-HF patients, and cardiac arrhythmias are the leading cause of death in HF patients (Roger et al., Circulation 121(7):e46-e215 (2010); Kannel et al., Am Heart J 115: 869-875 (1988)). The “gold standard” for preventing SCD is the implantation of an implanted cardiac defibrillator (ICD). Extensive clinical trials support that implantation of an ICD prolongs life.

Currently, ICDs are indicated for all patients with a chronic cardiac left ventricular ejection fraction (EF) of less than 35% (Hunt et al., Circulation 119:e391-E479 (2009)). Both the American College of Cardiology and the American Heart Association endorse the placement of an implanted cardiac defibrillator (ICD) for primary prevention of sudden cardiac death to reduce total mortality in patients with nonischemic dilated cardiomyopathy or ischemic heart disease at least 40 days post-myocardial infarction, a EF less than or equal to 35%, New York Heart Association (NYHA) functional class II or Ill symptoms while receiving chronic optimal medical therapy, and who have reasonable expectation of survival with a good functional status for more than 1 year. (Level of Evidence: A; Hunt et al., 2009, supra).

Despite the wide acceptance and practice of these guidelines, more than 60% of patients who have an ICD never experience and a shock from the ICD due to a lack of need therefor (Bardy et al., N Eng J Med 352:225-237 (2005)). Thus, there are many patients that unnecessarily receive an ICD.

On average, an ICD, costs between $20,000 and $50,000, excluding operative, follow-up, and complication costs. Also, the implantation surgery itself poses additional patient risk for complications, including major bleeding, pneumothorax, perforation of the heart, arrhythmia induction, stroke, heart attack, need for emergency heart surgery, and death.

Current methods for SCD risk stratification and determination of a patient's need for ICD placement fail to provide a simple, cost effective, method for achieving this end. Either the method essentially assesses only ejection fraction (EF), and oversimplifies the complexities of SCD risk stratification, or the method is overly complicated, invasive, or costly. While the methods that look at EF do not distinguish well between low risk patients and high risk patients, likely because they do not directly reflect an arrhythmogenic pathophysiological process, other FDA-approved techniques, e.g., signal averaged electrocardiogram (SAECG) and T-wave alternans, are not widely employed, because the costs of the equipment and personnel to implement them are too high. Also, some studies have demonstrated their lack of utility (8-10). Additionally, invasive electrophysiological testing also has been largely abandoned for similar reasons. Because risk can change with time, these more demanding techniques, if used at all, are often restricted to a single assessment per patient.

In view of the foregoing, there is a need in the art for a simple, inexpensive blood test for determining SCD risk and for determining patient need for ICD implants.

### SUMMARY

The invention provided herein is based in part on data demonstrating that patients with an implanted cardiac defibrillators (ICD), which provides shock to the patient, exhibit increased levels of truncated SCN5A Exon 28 splice variant transcripts and exhibit reduced levels of full length SCN5A Exon 28 transcripts.

The invention accordingly provides a method of determining a subject's need for an implanted cardiac defibrillator (ICD). In exemplary embodiments, the method comprises the step of determining a level of a full length SCN5A Exon 28 transcript and a level of a truncated SCN5A Exon 28 transcript, of a biological sample obtained from the subject. In exemplary embodiments, the method comprises the step of determining a level of all SCN5A Exon 28 transcripts, including both wild-type (WT) Exon 28 transcripts, E28A transcripts, E28B transcripts, E28C transcripts, and E28D transcripts.

In exemplary embodiments, the method of determining a subject's need for an ICD comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

The invention also provides a method of determining a subject's need for an ICD, wherein the method comprises the steps of (A) determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample and (B) comparing Rₛ as determined in (A) to a threshold ratio, R_{T}, wherein the R_{T} is determined by a system. In exemplary aspects, the system comprises a processor and a memory device coupled to the processor, wherein the memory device stores machine readable instructions that, when executed by the processor, cause the processor to:
(i) receive a plurality of data values, each data value is a ratio determined from a biological sample obtained from a subject of a first population, wherein the ratio compares a level of a truncated SCN5A Exon 28 transcript to (a) a level of a full length SCN5A Exon 28 transcript or (b) a level of all SCN5A Exon 28 transcripts, wherein each subject of the first population is a subject known as having an ICD that has given a shock;
(ii) fit the plurality of data values to a first Gaussian distribution;
(iii) determine a mean value, μ, and a standard deviation, σ, of the first Gaussian distribution;
(iv) set a first threshold ratio, R_{T}, at μ−Xσ, wherein X is a number between 0.7 and 4.0.

This system, as well other related systems, related computer-readable storage media having stored thereon machine-readable instructions executable by a processor, and related methods implemented by a processor in a computer, are also provided herein.

Patients who receive shocks from ICDs are at high risk for sudden cardiac death. Therefore, the invention further provides a method of determining a subject's risk for sudden cardiac death. In exemplary aspects, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, R, compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, R, compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

Patients at risk for sudden cardiac death are prescribed a form of an anti-arrhythmic therapy. Thus, the invention additionally provides a method of determining a subject's need for anti-arrhythmic therapy. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

The invention further provides a method of reducing risk of sudden cardiac death (SCD) in a subject. In exemplary embodiments, the method comprises the steps of (A) determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample and (B) administering to the subject an ICD or an anti-arrhythmic agent, when R_{S} is greater than or equal to a threshold ratio, R_{T}. In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

The invention provides a method of determining whether administration of an anti-arrhythmic therapy to a subject will be safe. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

Also provided is a method of determining a subject's risk for arrhythmias. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

The invention furthermore provides a method of determining a subject's risk for heart failure. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample In exemplary aspects, Rₛ compares a level of SCN5A Exon 28 Splice Variant C (E28C) of a biological sample obtained from the subject to a sum level of WT SCN5A Exon 28 transcript and SCN5A Exon 28 Splice Variant A (E28A). In exemplary aspects, Rₛ compares a level of E28C of a biological sample obtained from the subject to a sum level of all SCN5A Exon 28 transcripts: WT, E28A, E28B, E28C, E28D. In exemplary aspects, Rₛ compares a level of SCN5A Exon 28 Splice Variant D (E28D) of a biological sample obtained from the subject to a sum level of WT SCN5A Exon 28 transcript and SCN5A Exon 28 Splice Variant A (E28A). In exemplary aspects, Rₛ compares a level of E28D of a biological sample obtained from the subject to a sum level of all SCN5A Exon 28 transcripts: WT, E28A, E28B, E28C, E28D.

Furthermore provided herein is a kit useful for determining a subject's need for an ICD or for an anti-arrhythmic agent, a subject's risk for SCD, heart failure, or arrhythmias, or for determining whether administration of an anti-arrhythmic agent to a subject will be safe. In exemplary aspects, the kit comprises (i) reagents for measuring a level of full length SCN5A Exon 28 transcript of a biological sample and (ii) reagents for measuring a level of a truncated SCN5A Exon 28 transcript of a biological sample. In exemplary aspects, the kit comprises instructions for use, or access thereto. In exemplary aspects, the kit comprises a system or a computer-readable storage media having stored thereon machine-readable instructions executable by a processor, as further described herein.

### BRIEF DESCRIPTION OF THE DRAWINGS

FIG. 1 is a schematic representation of the splice variants identified in the 5′ end of the human SCN5A gene. The map shows the genomic structure of SCN5A with untranslated (open bars) or translated (closed bars) transcribed sequences and nontranscribed sequences (lines). Splicing patterns for each of the three exon 1 isoforms are identified.

FIG. 2 provides cDNA sequences for E1A, E1B1, E1B2, E1B3, E1B4, E2A, E2B1 and E2B2.

FIG. 3 is a schematic representation of the splice variants identified in the 3′ end of the human SCN5A gene. Above the map shows the genomic structure of SCN5A with untranslated (open bars) or translated (closed bars) transcribed sequences and nontranscribed sequences (lines). Splicing patterns for each of the four exon 28 isoforms are identified.

FIG. 4 provides cDNA sequences for E28A (FIGS. 4A and 4B), E28B (FIG. 4C), E28C (FIG. 4D), and E28D (FIG. 4E).

FIG. 5 is a photograph of a gel showing PCR results for detecting the transcription start site (TSS), exon 1 variants of the human cardiac SCN5A gene. Total RNA from fetal and adult heart was used to determinate the TSS and exon 1 isoforms with SCN5A specific primers. RACE-PCR result shows total three hands (380, 200 and 100 bp) in both fetal heart (FH) and adult heart (AH) contained cardiac specific Na+ channel sequences. The first band, 380 bp, corresponds to exon 1A, which is reported previously. A second band, 200 bp, is new exon 1 isoform, referred as to exon 1B (E1B) with multiple TSS, whereas the third 100 bp band is an exon 1 isoform referred as to exon 2B (E2B) with multiple TSS.

FIG. 6 is a photograph of a gel showing RACE-PCR results for detecting the 3′ UTR isoforms of the human cardiac SCN5A gene. Total RNA from human fetal and adult heart was used to determinate the 3′UTR with SCN5A gene specific primers GSP3′. The first two lines are first PCR of RACE product and the last two bands are 2nd RACE-PCR result showing the fetal heart lane (FH) demonstrates three bands, the larger visible band is corresponds to 834 bp short 3′UTR of human cardiac Na+ channel sequences. A second band (.about.1.4 kb) is noted in fetal heart (FH) only, whereas the third band is noted in both fetal and adult heart as 0.25 and 0.3 kb bands. The last band is 0.2 kb, which were detected only in adult heart. The presence of four exon 28 isoforms was confirmed by sequencing.

FIG. 7 is a schematic showing alignment of the nucleotide and amino acid sequences (single letter) of the four SCN5A transcriptional isoforms. The isoform name and nucleotide base pairs numbering starting at the initial AUG codon are indicated at the left. The sequences start from exon 27 (shaded) and continue to the poly-A tail. Introns are shown as dashed lines. Splicing of exons B, C, and D result in frame shifts and premature stop codons. Methionine at amino acid 1652 is bolded to indicate the site of introduction of a stop codon in the gene-targeted mouse.

FIG. 8 is a drawing showing the putative secondary structure of the cardiac Na+ channel.

FIG. 9 is a set of graphs showing developmental regulation of the human SCN5A exon 28 isoforms. The relative abundances of the four isoforms in fetal (FH, open bars) and adult heart (AH, closed bars) are shown in FIG. 9A. FIG. 9B shows the abundance of each isoform as a percentage of the total SCN5A mRNA. In each case, the full length transcript (E28A) was most prevalent. Exon 28D (E28D) was the second most abundant. Exon 28B (E28B) was least abundant in fetal heart, and exon 28C (E28C) was least abundant in adult heart. E28B and E28D were increased in adult heart when compared to fetal heart. *p<0.05 when comparing FH to AH.

FIG. 10 is a set of graphs showing that C-terminal isoform mRNA abundances vary between control and diseased hearts. Real-time PCR results show that the isoform abundances with respect to the total mRNA abundance in each sample. The changes were determined for the four exon 28 variants in control (black bars) and HF (open bars) patients for total ventricle (FIG. 10A), left ventricle (FIG. 10B), and right ventricle (FIG. 10C). Comparing left and right ventricle in FIG. 10B and FIG. 10C, respectively, shows that the isoform abundance changes are more prominent in the left ventricle. .beta.-Actin was used as a reference in all cases. All mRNA abundances were normalized to one of the normal human ventricular exon 28D mRNA abundances. *p<0.05 when comparing controls to HF hearts.

FIG. 11 is a set of photographs of gels showing tissue-specific expression of human SCN5A exon 28 isoforms. R_{T}-PCR results shows that isoform E28A and D were expressed in skeletal muscle (SKM), as well as in human heart. All four isoforms were found in lymphoblasts. The .beta.-actin was used as internal reference.

FIG. 12 is a set of graphs showing a model truncation that reduces cardiac Na+ channel current. In order to test the physiological role of the splice variations, a model truncation was introduced into a single allele of mouse ES cells by gene targeting. FIG. 12A shows cardiomyocytes with and without the altered allele were studied electrophysiologically. Na+ channel current-voltage curves are shown for wild-type (black squares) and the truncation (open squares). FIG. 12B shows peak current between the two cell types is compared. Introduction of a truncation in the region of the mRNA splice variations resulted in a substantial reduction in peak Na+ current. FIG. 12C shows action potentials (AP) recorded in the current clamp mode from a spontaneously beating wild-type (gray line) and mutant CMs (black line). FIG. 12D compares beating rate, AP amplitude, and AP upstroke velocity between the two types of CMs. The truncation mutant shows a reduced beating rate (beats per second), AP amplitude (mV/10), and maximum AP upstroke velocity (mV/ms), consistent with reduced Na+ current. *, p<0.05.

FIG. 13 is a set of graphs showing multi-electrode array (MEA) analysis of differentiated CMs with and without the introduction of the model truncation. FIG. 13A illustrates representative field potentials (FPs) from syncytial wild-type and truncation containing CMs recorded from a single MEA electrode. FIG. 13B compares field potential minima (FPmin) recorded at day 19. The FPmin was significantly decreased by introduction of the truncation. FIG. 13C shows that the time to FPₘᵢₙ (i.e. the field potential rise time, FPᵣᵢₛₑ) was slowed in the truncation CMs as compared to wild-type. Conduction velocity was decreased substantially in the truncation cardiomyocytes (FIG. 13D). All changes are consistent with a reduced Na+ current. *p<0.05 between wild-type and the truncation.

FIG. 14 is a set of graphs showing cell viability testing. FIG. 14A: H9c2 cardiomyocyte viability at 48 h as a function of angiotensin II concentration. FIG. 14B: H9c2 cardiomyocyte viability at 48 h as a function of H₂O₂. *, P<0.05 when compared with 10 .mu.mol/L H₂O₂.

FIG. 15 is a set of graphs showing downregulation of cardiac Na+ channel mRNA by AngII and H₂O₂. H9c2 or acutely isolated neonatal cardiomyocytes cultured in serum free media (SFM) or exposed to AngII (100 nM or 2 nM), H₂O₂ (20 .mu.M or 40 nM), or AngII (100 nM or 2 nM) and PEG-catalase (250 U/mL) for 48 h. AngII and H₂O₂ resulted in reduced Na+ channel mRNA abundance in both H9c2 (FIG. 15A) and neonatal cardiomyocytes (FIG. 15B). In both cases, the AngII effect could be prevented by PEG-catalase (AngII+CAT). *, P<0.05.

FIG. 16 is a set of graphs showing that cardiac Na+ channel current is downregulated by H₂O₂. FIG. 16A shows representative Na+ currents recorded from H9c2 CMs with voltage steps to −10 mV before and after 48 h of H₂O₂ exposure. FIG. 16B shows that H₂O₂ exposure (20 .mu.M for 48 h) resulted in a 46% (.+−.2.9%, p=0.01) reduction in peak Na+ current. *, P<0.05.

FIG. 17 shows that mutation of the NF-.kappa.B binding site in the Na+ channel promoter eliminates the effects of AngII and H₂O₂. FIG. 17A shows the relationship of the promoter-reporter fragment used to the mouse scn5a promoter (GenBank AY769981). The top line shows the structural organization of this region of the scn5a promoter (3.0 kb). Note the presence of untranslated exon 1C and part of exon 2, which contains the translation start site. Nucleotide numbering starts with +1 corresponding to the protein translation start site. The construct, APS3, containing the NF-.kappa.B site (.diamond-solid.) showed reduced activity after 48 h of exposure to 100 nM AngII or 20 .mu.M H₂O₂. Mutating the NF-.kappa.B binding site prevented the AngII or H₂O₂ effects (FIG. 17B). Data are presented as mean.+−.S.E.M and are based on 4 separate experiments in both groups.

FIG. 18 is a set of photographs of gels showing that AngII and H₂O₂ induce NF-.kappa.B binding to the scn5a promoter. FIG. 18A shows an electrophoretic mobility shift assays with nuclear extracts from H9c2 cardiomyocytes showed that under basal conditions (SFM), there was little binding of NF-.kappa.B to the scn5a promoter. H₂O₂ or AngII exposure resulted in the NF-.kappa.B binding to the promoter. TNF-.alpha. activated H9c2 cell nuclear extract (5 .mu.g) was used as positive control. CAPE (caffeic acid phenethyl ester), a NF-.kappa.B inhibitor, prevented binding in response to H₂O₂. AngII-induced NF-.kappa.B binding was inhibited by unlabeled probe but not mutant unlabeled probe. FIG. 18B shows that AngII and H₂O₂ promote binding of the p50 subunit of NF-.kappa.B to the cardiac Na+ channel promoter. Chromosomal immunoprecipitation assay using primers specific for scn5a promoter shows that the p65 subunit of NF-.kappa.B appears to be constitutively bound to the channel promoter but that the p50 subunit binds in response to AngII or H₂O₂. CAPE, and inhibitor of NF-.kappa.B, could prevent biding of both subunits to the channel promoter. Lanes 1 and 2 are positive and negative controls, respectively. The input DNA was diluted with 1:10.

FIG. 19 shows that overexpression of the p50 NF-.kappa.B subunit results in Na+ channel transcriptional downregulation. Panel A shows that the presence of the p50 or p65 NF-.kappa.B subunit RNA in H9c2 cells stably transfected with vectors encoding p50, p65, or both. Con represents control cells. Panel B: Quantitative real-time R_{T}-PCR result shows the relative scn5a mRNA abundance was decreased in cell lines expressing the p50 subunit in comparison with control (Con). * p<0.05 vs. control.

FIG. 20 is a graph showing a comparison of the abundances of SCN5A splice variants as a function of age. Comparing the relative mRNA abundances of the splice variants after dividing subjects into three age groups of 40-49 (40's), 50-59 (50's) and 60-69 (60's) shows that splice variant abundances did not appear to be a function of age.

FIG. 21 is a set of diagrams showing the targeting strategy to create a mouse SCN5A truncation model. FIG. 21A: Targeting vector pBSK.SCN5A^{1652stop} mapped to the native SCN5A exon 28 region (WT SCN5A allele). FIG. 21B: Map showing incorporation of the targeting vector into the WT allele. FIG. 21C: Map of the truncation mutation introduced into WT SCN5A allele after Cre-mediated excision of the neomycin resistance cassette to create SCN5A^{1652stop}. Restriction digests, PCR primers, and hybridization probes A (3.1-kb PvuII fragment) and B (3.71-kb PvuII fragment) for genotyping are indicated.

FIG. 22 is a set of photographs of gels showing the genotyping for homologous recombination of the SCN5A^{1652stop}. FIG. 22A. PCR analysis using upper and lower PCR amplicons (see methods and figure S2) demonstrates proper recombination in heterozygous (.+−.) and not in wild-type (WT) mice (+/+). FIGS. 22B and 22C: Southern blot analysis with external probes A and B showing proper incorporation of the truncation vector in a single allele of targeted ES cells (.+−.). FIG. 22D: PCR result of a properly targeted ES cell clone before (neo+) and after (neo−) successful excision of the neomycin resistance cassette. The targeting vector was used as a control. FIG. 22E: BspHI restriction digests to demonstrate incorporation of the targeting vector. Introduction of the coding mutation resulted in elimination of a BspHI restriction site. Therefore, the properly targeting allele displayed an additional 545 bp fragment representing the targeted allele (heterozygous, .+−.) as compared to the 395 bp and 150 bp fragments resulting from the native sequences (wild-type, WT) when performing a BspHI digest of a PCR amplicon spanning this region.

FIG. 23 demonstrates the effects of expressions of RBM25 and LUC7L3 in human HF tissue. (A) qPCR demonstrates the upregulation of RBM25 and LUC7L3 in human HF tissue. The relative expression changes of RBM25 and LUC7L3 in both normal control (white bars) and failing heart tissue (black bars) are shown. All mRNA abundances are normalized by β-actin. * P<0.05 when compared with control. (B) Western blots quantification confirms the upregulation of RBM25 and LUC7L3 in human HF tissue. Control represents the mixture of 4 normal human heart tissue samples. HF1, HF2, HF3 and HF4 represent the HF tissue sample 1, sample 2, sample 3 and sample 4, respectively. Quantification is based on three replications for each sample. All protein levels are normalized by GAPDH. * P<0.05 when compared with control.

FIG. 24 provides an illustration of the C-terminal structure of SCN5A and the variants E28C and E28D. (A)* indicates the RBM25 binding site CGGGCA in exon 28 (982 bp-987 bp). Gel mobility shift assays are performed using the biotinylated wild type (WT) probe (B) or the mutant (Mu) probe (C) and purified RBM25 protein. For loading samples from left to right, the amount of RBM25 in each binding reaction is increased by a fold. For the competition assay (D), 0, 1-, 5-, or 20-fold molar excesses of unlabeled WT or Mu probes are added in each binding reaction.

FIG. 25 demonstrates that. RBM25 and LUC7L3 are involved in SCN5A regulation in Jurkat cells. (A) qPCR demonstrates the upregulation of RBM25 and LUC7L3. The expression changes of RBM25 and LUC7L3 in hypoxia-treated (shaded bars) and Ang II-treated (black bars) vs. normal control Jurkat cells (white bars) are shown at 48 h. HIF-1α is an indicator of hypoxia. mRNA abundances are normalized by β-actin. * P<0.05 when compared with control (N=6). (B) Western blots quantification confirms the upregulation of RBM25 and LUC7L3 in Jurkat cells. Expressions of RBM25 and LUC7L3 are analyzed by time course. * P<0.05 when compared with control (N=6). (C) The expression changes of SCN5A and the variants E28C and E28D in hypoxia-treated (shaded bars) and Ang II-treated (black bars) vs. untreated Jurkat cells (white bars) are shown at 48 h. * P<0.05 when compared with control (N=6). Western blots indicate the downregulation of SCN5A in Jurkat cells with Ang II or hypoxia. (D) qPCR demonstrates that RBM25 and LUC7L3 siRNAs could block the induction of hypoxia on variants E28C and E28D. The representative results at 24 h are shown. * P<0.05 when compared with control (N=6). (E) qPCR demonstrates that RBM25 and LUC7L3 siRNAs could block the induction of variants E28C and E28D by Ang II. Representative results at 48 h are shown. * P<0.05 when compared with control (N=6). Scrambled siRNA had no effect on the induction by Ang II (data not shown). The knockdown efficiency of RBM25 and LUC7L3 siRNAs are evaluated by Western blots and compared to control and scrambled RNA. (F) qPCR demonstrates that RBM25 and LUC7L3 overexpressions decrease the full length SCN5A transcript and increase the variants E28C and E28D at 48 h. * P<0.05 when compared with control (N=6). Exogenously expressed RBM25-GFP and LUC7L3-GFP are detected by Western blot analysis with anti-GFP at 48 h after transfection in Jurkat cells. The representative Western blots show the downregulation of the full-length SCN5A transcript in Jurkat cells with exogenously expressed RBM25 and LUC7L3 as compared to the control group. Full-length SCN5A RNA is unchanged with GFP expression alone.

FIG. 26 demonstrates the effect of RBM25 shRNA on the expressions of SCN5A and the variants E28C and E28D in Ang II-treated hESC-CMs. (A) Ang II (200 nmol/L) treatment is given to all the experiment groups on infection day 3. R_{T}-PCR measurements are done at 24 h after Ang II treatment and normalized by β-actin. The expression changes of SCN5A and the variants E28C and E28D in Ang II-treated cardiomyocytes pre-infected by RBM25 pLKO.1 shRNA (shaded bars) and pre-infected by scrambled shRNA (black bars) vs. normal Ang II-treated cardiomyocytes (white bars) are shown at 24 h. * P<0.05 compared with normal Ang II-treated cardiomyocytes (N=6). (B) Confocal microscopy shows a hESC-CM. The GFP fluorescence indicates the infection by pGIPZ lentiviral shRNAmir. RBM25 knockdown efficiency is evaluated by qPCR and Western blot (N=6). Scrambled shRNA has no effect on RBM25.

FIG. 27 demonstrates the effect of Ang II (200 nmol/L) on Na+ currents in hESC-CMs at 24 h after treatment. The representative current traces are shown in the control (A), Ang II-treated (B), Ang II-treated with pre-infection of RBM25 shRNA (C), and TTX-treated groups (D). The peak current statistics of the three experiment groups are shown in I-V curves (E), where the control group (n=3) is represented by filled squares, the Ang II-treated group (n=3) by filled circles, and the Ang II-treated group with pre-infection of RBM25 shRNA (n=3) by open triangles. The data are represented as mean±SEM. The peak current is significantly reduced from −40 to +30 mV in the Ang II-treated group compared to the control group (P<0.05), the maximum reduction was 91.1±9.3% at −30 mV. No difference is observed between the Ang II-treated group with pre-infection of RBM25 shRNA and the control group (E). The specificity of sodium channel current is tested with TTX (a specific sodium channel blocker) in Group D. Scrambled shRNA had no effect on the I-V relationship compared to Ang II alone (data not shown).

FIG. 28 is a bar graph of relative ratios of variants E28C (“VC”) or E28D (“VD”) of control patients, heart failure (HF) patients, patients with ICDs that have not provided shock (ICD(−) Shock), and patients with ICDs that have provided shock (ICD(+) Shock).

FIG. 29 represent two graphs demonstrating the correlation of cardiac tissue and blood abundances of SCN5A splice variants normalized to total SCN5A expression. Left panel: Correlation for variant E28C (VC). Right panel: Correlation for variant E28D (VD).

FIG. 30 represent two graphs of Gaussian distributions of abundances of SCN5A splice variants in control patients, patients with an ICD that provides shocks to the patients, and patients with an ICD that do not provide shocks to the patients. Left panel: Distribution for variant E28C (VC). Right panel: Distribution for variant E28D (VD).

FIG. 31 is a receiver operator characteristics (ROC) curve for SCN5a variant D as a predictor of appropriate ICD discharges. The line of identity is shown.

FIG. 32 represents a graph of the univariate odds ratio of sudden death given the patient has elevated VC, elevated VD, NYHA class III/IV heart failure, is taking ACE inhibitor, is on an antiarrhythmic drug, or has an EF less than 30%, or has QRS duration widening greater than 120 ms, respectively.

FIG. 33 represent two graphs of Gaussian distributions of abundances of SCN5A splice variants in control patients, patients with an ICD that provides shocks to the patients, and patients with an ICD that do not provide shocks to the patients, wherein the abundances are neither normalized to a housekeeping gene nor relative to a level of a full length SCN5A transcript. Left panel: Distribution for variant E28C (VC). Right panel: Distribution for variant E28D (VD).

FIG. 34 is a schematic of an exemplary embodiment 101 of a system 100 for assessing a subject's need for an implanted cardiac defibrillator (ICD).

FIG. 35 Correlation of cardiac tissue and WBC mRNA abundances of SCN5A variants VC and VD. Panel A shows tissue levels of the variant VC as a function of WBC levels measured in the same patient. Panel B shows tissue levels of the variant VD as a function of WBC levels measured in the same patient. The best-fit linear regression is displayed as a solid black line. Grey lines represent the 95% confidence intervals.

FIG. 36. The WBC expression of SCN5A variants in the test groups. Panel A shows fold induction compared to control of SCN5A variant VC in the heart failure (HF), ICD(−)event, and ICD(+)event groups. Panel B shows fold induction compared to control of SCN5A variant VD in the heart failure (HF), ICD(−)event, and ICD(+)event groups. The fold induction values are displayed as median, interquartile ranges, minimum, and maximum. * p<0.05 as compared to control. ** p<0.05 comparing the ICD(+)Event group to the combined HF and ICD(−)Event groups.

FIG. 37. The effect of clinical characters on the WBC expression of SCN5A VC and VD. Panels A, C, E, G, I, and K as well as B, D, F, H, J, and L compare the effects of race, sex, origin of the cardiomyopathy, left ventricular ejection fraction (LVEF), New York Heart Association heart failure stage (NYHA), and QRS duration on WBC variant VC and VD levels, respectively.

FIG. 38. Univariate analysis of clinical characteristics on discrimination of ICD events. The data are presented as the odds ratio and 95% confidence intervals.

FIG. 39. Receiver operation characteristics curves for WBC SCN5A variant VC and VD discrimination of ICD events. Receiver operation characteristics (ROC) curves for normalized VC and VD are compared to LVEF<20%. The area under the ROC curve (95% CI) are 0.98 (0.95, 1.00), 0.97 (0.93, 1.00), and 0.56 (0.41, 0.71) for VC, VD and LVEF≦20%, respectively. The line of no discrimination is also shown.

### DETAILED DESCRIPTION

SCN5A Transcripts

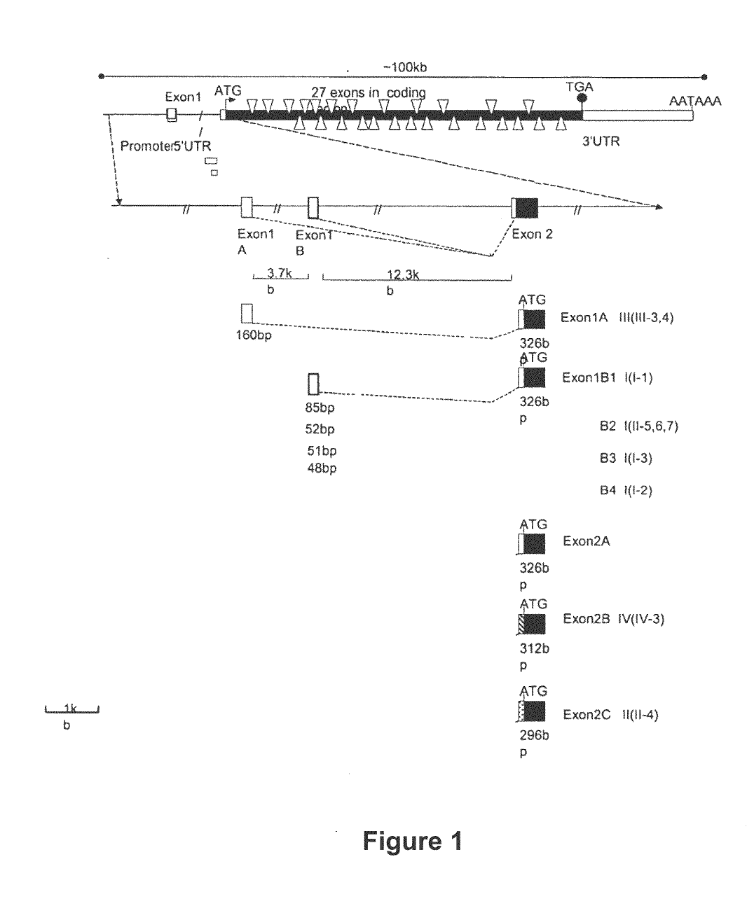

As described herein and in the art (e.g., U.S. Application Publication No. 2007/0212723 A1), analysis in or near the promoter and 5′ and 3′ untranslated regions (UTRs) of the SCN5A gene led to identification of multiple specific 5′ and 3′ mRNA splice variants. As shown in FIGS. 1 to 4, the splice variants include Exon 1 splice variants: E1B 1 (SEQ ID NO. 1), E1B2 (SEQ ID NO. 2), E1B3 (SEQ ID NO. 3), and E1B4 (SEQ ID NO. 4), and also include Exon 2 splice variants: E2B1 (SEQ ID NO. 5), E2B2 (SEQ ID NO. 6), and furthermore include Exon 28 splice variants E28B (SEQ ID NO. 7), E28C (SEQ ID NO. 8), or E28D (SEQ ID NO. 9).

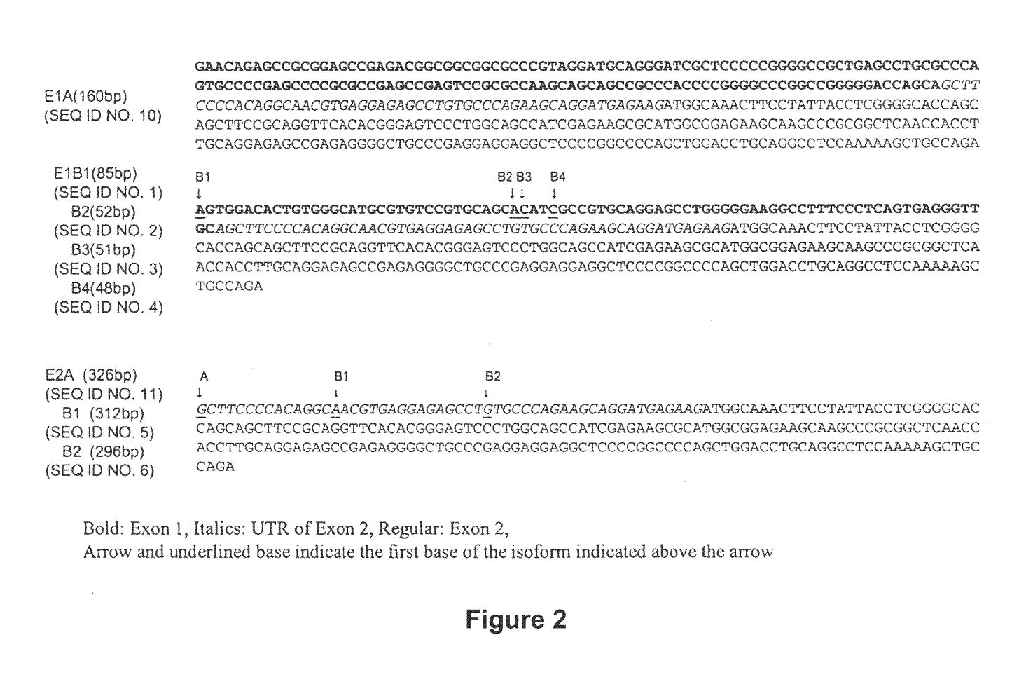

The E1B1, E1B2, E1B3, E1B4, E2B1, and E2B2 splice variants are from the 5′ region, the locations of which in the SCN5A gene and mRNA are depicted in FIG. 1. The nucleic acid sequences for E1B1, E1B2, E1B3, E1B4, E2B1, and E2B2 are shown in FIG. 2. E1A (SEQ ID NO. 10) is the wild-type (or full-length) isoform in and/or near the 5′UTR of exon 1, while E1B1, E1B2, E1B3, E1B4 are its various spliced (or truncated) variants. Similarly, E2A (SEQ ID NO. 11) is the wild-type (full-length) isoform in and/or near the 5′UTR of exon 2, while E2B1, and E2B2 are its various variants.

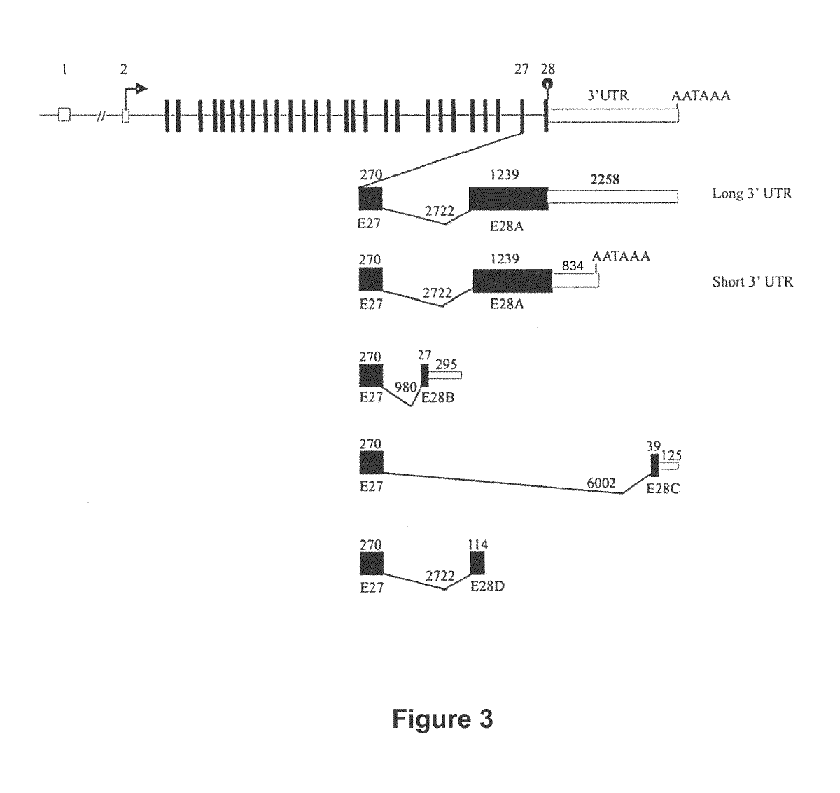

The E28B, E28C, or E28D splice variants are from or near the 3′ untranslated region, the locations of which in the SCN5A mRNA are depicted in FIG. 3. The nucleic acid sequences for E28B, E28C, and E28D are set forth in SEQ ID NOs: 7-9, respectively. Each of E28B, E28C, and E28D is a truncated splice variant encoding shortened, dysfunctional channels. Both E28B and E28C contains untranslated and translated regions while E28D contains only translated region of exon 28. The physiological significance of the E28B, E28C and E28D splice variants is supported by a premature stop codon in exon 28 of one of the two SCN5A alleles, resulting in an 86% reduction in the Na⁺ current (Shang et al., Circ. Res., 101:1146-1154, 2007).

The E28A splice variant is another isoform of the 3′ region of SCN5A Exon 28. There are two isoforms of the E28A: E28A-short (E28A-S) (SEQ ID NO. 12) and E28A-long (E28A-L) (SEQ ID NO. 13). Both isoforms of E28A contains 1239 base pairs in the translated region. The difference between E28-L and E28-S resides in the UTR where E28A-L contains 2295 base pairs of the 3′UTR, while E28A-S contains 834 base pairs of the 3′UTR. E28A-S contains only the first 834 base pairs of the 3′UTR. E28A-L represents the wild-type (WT) isoform. For purposes herein, outside of this paragraph, recitation of “E28A” refers to E28A-S, and E28A-L will be referred to as wild-type or WT, when referencing a SCN5A Exon 28 transcript.

As used herein, the term “truncated SCN5A Exon 28 transcript” refers to a transcript comprising a shortened or truncated translated region of Exon 28 of the SCN5A gene. In exemplary aspects, the truncated SCN5A Exon 28 transcript is a transcript comprising an E28B transcript, E28C transcript, or E28D transcript, which may be referred to herein as “E28B,” “E28C,” and “E28D,” respectively. In exemplary aspects, the truncated SCN5A Exon 28 transcript is a transcript comprising one of an E28C transcript or E28D transcript.

As used herein, the term “full length SCN5A Exon 28 transcript” refers to a transcript comprising the full length translated region of Exon 28 of the SCN5A gene. In exemplary aspects, the full length SCN5A Exon 28 transcript is a WT SCN5A Exon 28 transcript. In exemplary aspects, the full length SCN5A Exon 28 transcript is a spliced transcript which is shortened or truncated, as compared to WT SCN5A Exon 28 transcript, yet the spliced transcript still comprises the full length translated region of SCN5A Exon 28. In exemplary aspects, the full length SCN5A Exon 28 transcript is a transcript comprising an E28A transcript (E28A-S).

Implanted Cardiac Defibrillators

As used herein, the term “Implanted Cardiac Defibrillator” or “ICD” is synonymous with “implantable cardiac defibrillator” or “implanted cardiac device” or “implantable cardiac device” or “implantable cardioverter-defibrillator device” and refers to a small battery-powered electrical impulse generator that is programmed to deliver a jolt of electricity, when a cardiac arrhythmia is detected. ICDs are implanted into patients who are at risk of sudden cardiac death due to ventricular fibrillation and ventricular tachycardia. Under the current standards used to identify patients in need of an ICD (which are reviewed in Hunt et al., Circulation 119:e391-e479 (2009) and Epstein et al., J Am Coll Cardol 51:e1-e62 (2008)), approximately 60% of those patients that receive an ICD do not receive a shock from the ICD, presumably because the patient does not exhibit a cardiac arrythmia. Therefore, approximately 60% of those patients that receive an ICD do not actually need one.

The data provided herein demonstrate that patients with an ICD, which provides shocks to the patient, exhibit a profile of levels of SCN5A Exon 28 transcripts that is different from the profile of levels of SCN5A Exon 28 transcripts of patients with an ICD which do not provide shocks to the patient. The data demonstrate that patients with an ICD, which provides shocks to the patient exhibit increased levels of truncated SCN5A Exon 28 splice variant transcripts and exhibit reduced levels of full length SCN5A Exon 28 transcripts. Thus, these data suggest a basis by which the two patient populations (patients who truly need an ICD vs. patients who do not need an ICD) may be distinguished.

The invention accordingly provides a method of determining a subject's need for an ICD. In exemplary embodiments, the method comprises the step of determining a level of a level of a truncated SCN5A Exon 28 transcript and a level of a full length SCN5A Exon 28 transcript, of a biological sample obtained from the subject. In exemplary embodiments, the method comprises the step of determining a level of all SCN5A Exon 28 transcripts, including WT, E28A, E28B, E28C, E28D. In exemplary aspects, as further described herein, the levels of SCN5A Exon 28 transcripts referenced in the methods herein are normalized to a level of transcripts of a reference gene, e.g., a housekeeping gene.

In exemplary aspects, the method of determining a subject's need for an ICD comprises determining a ratio, R_{S}, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined as needing an ICD, when R_{S} is greater than or equal to a threshold ratio, R_{T}. Further descriptions of each of these ratios are found below.

Ratio, R_{S}

In exemplary aspects, the methods of the invention comprises the step of determining a ratio, R_{S}, which compares a level of a truncated SCN5A Exon 28 transcript of a biological sample obtained from the subject to which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

Accordingly, in some aspects, R_{S} is a ratio of [a level of a truncated SCN5A Exon 28 transcript of a biological sample obtained from the subject] to [a full length SCN5A Exon 28 transcript of the biological sample]. As described herein, a full length SCN5A Exon 28 transcript refers to either a WT SCN5A Exon 28 transcript or E28A-S. Accordingly, in specific aspects, R_{S} is a ratio of [a level of a truncated SCN5A Exon 28 transcript of a biological sample obtained from the subject] to [a level of WT SCN5A Exon 28 transcript of the biological sample] or R_{S} is a ratio of [a level of a truncated SCN5A Exon 28 transcript of a biological sample obtained from the subject] to [a level of E28A (E28A-S) of the biological sample].

In other aspects, R_{S} is [a level of a truncated SCN5A Exon 28 transcript of a biological sample obtained from the subject] to [a level of all SCN5A Exon 28 transcripts: WT, E28A, E28B, E28C, E28D, of the biological sample]. R_{S} therefore may be a ratio of [a level of a truncated SCN5A Exon 28 transcript of a biological sample obtained from the subject] to [a level of (WT SCN5A Exon 28+E28A (E28A-S)+E28B+E28C+E28D) of the biological sample]. In exemplary aspects, R_{S} is a ratio of the level of E28C transcripts to the sum of (the level of WT SCN5A Exon 28 transcript (E28A-L))+(the (the level of E28A-S)+(the level of E28B)+(the level of E28C)+(the level of E28D) of the biological sample. In exemplary aspects, R_{S} is a ratio of the level of E28D transcripts to the sum of (the level of WT SCN5A Exon 28 transcript (E28A-L))+(the (the level of E28A-S)+(the level of E28B)+(the level of E28C)+(the level of E28D) of the biological sample. In exemplary aspects, R_{S} is a ratio of the sum of (the level of E28C transcripts)+(the level of E28D transcripts) to the sum of (the level of WT SCN5A Exon 28 transcript (E28A-L))+(the (the level of E28A-S)+(the level of E28B)+(the level of E28C)+(the level of E28D) of the biological sample.

In other aspects, R_{S} is [a level of a truncated SCN5A Exon 28 transcript of a biological sample obtained from the subject] to [a level of SCN5A Exon 28 transcripts: E28A-S, E28B, E28C, E28D of the biological sample]. R_{S} therefore may be a ratio of [a level of a truncated SCN5A Exon 28 transcript of a biological sample obtained from the subject] to [a sum of (the level of E28A-S)+(the level of E28B)+(the level of E28C)+(the level of E28D) of the biological sample]. In exemplary aspects, R_{S} is a ratio of the level of E28C transcripts to the sum of (the level of E28A-S)+(the level of E28B)+(the level of E28C)+(the level of E28D) of the biological sample. In exemplary aspects, R_{S} is a ratio of the level of E28D transcripts to the sum of (the level of E28A-S)+(the level of E28B)+(the level of E28C)+(the level of E28D) of the biological sample. In exemplary aspects, R_{S} is a ratio of the sum of (the level of E28C transcripts)+(the level of E28D transcripts) to the sum of (the level of E28A-S)+(the level of E28B)+(the level of E28C)+(the level of E28D) of the biological sample.

In exemplary aspects, R_{S} is [a level of a truncated SCN5A Exon 28 transcript of a biological sample obtained from the subject] to [a level of a full length SCN5A Exon 28 transcript and a level or one or more truncated SCN5A Exon 28 transcripts of the biological sample]. In specific aspects, R_{S} is a ratio of [a level of a truncated SCN5A Exon 28 transcript of a biological sample obtained from the subject] to [a level of WT SCN5A Exon 28 and one, two, or all of E28B, E28C, E28D]. In specific aspects, R_{S} is a ratio of [a level of a truncated SCN5A Exon 28 transcript of a biological sample obtained from the subject] to [a level of WT SCN5A Exon 28 and all of E28B, E28C, E28D]. In specific aspects, R_{S} is a ratio of [a level of a truncated SCN5A Exon 28 transcript of a biological sample obtained from the subject] to [a level of WT SCN5A Exon 28 and (E28B and E28C). In specific aspects, R_{S} is a ratio of [a level of a truncated SCN5A Exon 28 transcript of a biological sample obtained from the subject] to [a level of WT SCN5A Exon 28 and (E28C and E28D). In specific aspects, R_{S} is a ratio of [a level of a truncated SCN5A Exon 28 transcript of a biological sample obtained from the subject] to [a level of WT SCN5A Exon 28 and (E28B and E28D). In specific aspects, R_{S} is a ratio of [a level of a truncated SCN5A Exon 28 transcript of a biological sample obtained from the subject] to [a level of E28A (E28A-S) and all of E28B, E28C, E28D]. In specific aspects, R_{S} is a ratio of [a level of a truncated SCN5A Exon 28 transcript of a biological sample obtained from the subject] to [a level of E28A (E28A-S) and (E28B and E28C). In specific aspects, R_{S} is a ratio of [a level of a truncated SCN5A Exon 28 transcript of a biological sample obtained from the subject] to [a level of E28A (E28A-S) and (E28C and E28D). In specific aspects, R_{S} is a ratio of [a level of a truncated SCN5A Exon 28 transcript of a biological sample obtained from the subject] to [a level of E28A (E28A-S) and (E28B and E28D).

In exemplary aspects, each level SCN5A Exon 28 transcript of R_{S} is a calibrated level (calibrated abundance) in which the level is normalized or calibrated to a level of a reference gene, e.g., a housekeeping gene. Suitable housekeeping genes are known in the art, some of which are further described herein.

In exemplary aspects, each level SCN5A Exon 28 transcript of R_{S} is a control-normalized level. The control-normalized level in exemplary aspects is a level which is the difference between the level of the subject and the level of a control subject. Control subjects are further described herein. In exemplary aspects, the control subject is a subject known as (i) not having an ICD, (ii) not having a cardiac disease, (iii) having normal left ventricular function, (iv) not having diastolic dysfunction, or (v) a combination thereof.

In exemplary aspects, the step of determining R_{S} comprises calculating values obtained from Real Time Polymerase Chain Reaction (real time PCR). In exemplary aspects, the ΔΔCt mathematical model of data analysis for real-time PCR is applied (Livak et al., Methods 25:402-408 (2001) and Yuan et al., BMC Bioinformatics 7:85 (2006)), such that the calibrated level (calibrated abundance) of a truncated SCN5A Exon 28 transcript=2 to the power of −ΔΔCt of a truncated SCN5A Exon 28 transcript (also, expressed as 2^{-ΔΔCttruncated SCN5A Exon 28 transcript}), wherein ΔΔCt_{truncated SCN5A Exon 28 transcript}=Δ_{Cttruncated SCN5A Exon 28 transcript}−ΔCt_{housekeeping gene}. In exemplary aspects, ΔCt_{truncated SCN5A Exon 28 transcript} is ([Ct of the truncated SCN5A Exon 28 transcript of the subject sample] minus [Ct of the truncated SCN5A Exon 28 transcript of a control sample] and ΔCt_{housekeeping gene} is ([Ct of the housekeeping gene of the subject sample] minus [Ct of the housekeeping gene a control sample]. In exemplary aspects, the control sample is a biological sample obtained from a control subject known as (i) not having an ICD, (ii) not having a cardiac disease, (iii) having normal left ventricular function, (iv) not having diastolic dysfunction, or (v) a combination thereof.

Accordingly, in exemplary aspects,

$$\begin{matrix}{R}_{S} = \frac{Truncated SCN 5 A Exon 28 transcript}{Full length SCN 5 A Exon 28 transcript} \\ = \frac{Calibrated abundance of truncated SCN 5 A Exon 28 transcript}{Calibrated abundance of full length SCN 5 A Exon 28 transcript}\end{matrix}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of subject sample]−[Ct of full length SCN5A Exon 28 transcript of control sample])

ΔCt_{truncated}=([Ct of truncated SCN5A Exon 28 transcript of subject sample]−[Ct of truncated SCN5A Exon 28 transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

Also, in exemplary aspects,

$$\begin{matrix}{R}_{S} = \frac{Truncated SCN 5 A Exon 28 transcript}{All SCN 5 A Exon 28 transcripts} \\ = \frac{Calibrated abundance of truncated SCN 5 A Exon 28 transcript}{Calibrated abundance of all SCN 5 A Exon 28 transcripts}\end{matrix}$$

wherein:

calibrated abundance of truncated SCN5A Exon 28 transcript=2^{-ΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of subject sample]−[Ct of all SCN5A Exon 28 transcripts of control sample])

ΔCt_{truncated}=([Ct of truncated SCN5A Exon 28 transcript of subject sample]−[Ct of truncated SCN5A Exon 28 transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, the level of the truncated SCN5A Exon 28 transcript comprises a level of SCN5A Exon 28 Splice Variant C (E28C) of a biological sample. In exemplary aspects, the level of the truncated SCN5A Exon 28 transcript comprises a level of SCN5A Exon 28 Splice Variant C (E28D). In exemplary aspects, the level of a truncated SCN5A Exon 28 transcript is a level of E28C of the biological sample and a level of E28D of the biological sample.

In exemplary aspects, the level of a full length SCN5A Exon 28 transcript is the level of WT SCN5A Exon 28 transcripts. In exemplary aspects, the level of a full length SCN5A Exon 28 transcript is a sum level of [a level of WT SCN5A Exon 28 transcripts]+[a level of SCN5A Exon 28 Splice Variant A (E28A)]. In exemplary aspects, the level of full length SCN5A Exon 28 transcript is a level of E28A (E28-S). In alternative aspects, the level of all SCN5A Exon 28 transcripts is a sum level of [a level of WT SCN5A Exon 28 transcripts]+[a level of E28A]+[a level of E28B]+[a level of E28C]+[a level of E28D].

In exemplary aspects, R_{S} compares a level of E28C of a biological sample obtained from the subject to (i) a sum level of [a level of WT SCN5A Exon 28 transcripts]+[a level of E28A-S] or (ii) to a level of E28A-S or (iii) to a level of WT. In exemplary aspects,

$$\begin{matrix}{R}_{S} = {R}_{E 28 C} \\ = \frac{Calibrated abundance of E 28 C transcript}{Calibrated abundance of a full length SCN 5 A Exon 28 transcript}\end{matrix}$$

wherein:

calibrated abundance of E28C transcript=2^{-ΔΔCtE28C transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{E28C transcript}=ΔCt_{E28C transcript}−ΔCt_{housekeeping gene}

ΔCt_{full length}=([Ct of full length SCN5A Exon 28 transcript of subject sample]−[Ct of full length SCN5A Exon 28 transcript of control sample])

ΔCt_{E28C}=([Ct of E28C transcript of subject sample]−[Ct of E28C transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, R_{S} compares a level of E28C of a biological sample obtained from the subject to a level of [all SCN5A Exon 28 transcripts: WT, E28A-S, E28B, E28C, and E28D. In exemplary aspects,

$$\begin{matrix}{R}_{S} = {R}_{E 28 C} \\ = \frac{Calibrated abundance of E 28 C transcript}{Calibrated abundance of all SCN 5 A Exon 28 transcripts}\end{matrix}$$

wherein:

calibrated abundance of E28C transcript=2^{-ΔΔCtE28C transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{E28C transcript}=ΔCt_{E28C transcript}−ΔCt_{housekeeping gene}

ΔCt_{all SCN5A Exon 28 transcripts}=([Ct of all SCN5A Exon 28 transcripts of subject sample]−[Ct of all SCN5A Exon 28 transcripts of control sample])

ΔCt_{E28C}=([Ct of E28C transcript of subject sample]−[Ct of E28C transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, R_{S} compares a level of E28D of a biological sample obtained from the subject to (i) a sum level of [a level of WT SCN5A Exon 28 transcripts]+[a level of E28A-S] or (ii) to a level of E28A-S or (iii) to a level of WT. In exemplary aspects,

$$\begin{matrix}{R}_{S} = {R}_{E 28 D} \\ = \frac{Calibrated abundance of E 28 D transcript}{Calibrated abundance of full length SCN 5 A Exon 28 transcript}\end{matrix}$$

wherein:

calibrated abundance of E28D transcript=2^{-ΔΔCtE28D transcript}

calibrated abundance of full length SCN5A Exon 28 transcript=2^{-ΔΔCtfull length SCN}5A Exon 28 transcript

ΔΔCt_{full length SCN5A Exon 28 transcript}=ΔCt_{full length}−ΔCt_{housekeeping gene}

ΔΔCt_{E28D transcript}=ΔCt_{E28D transcript}−ΔCt_{housekeeping gene}

ΔCt_{full length}=([Ct of full length SCN5A Exon 28 transcript of subject sample]−[Ct of full length SCN5A Exon 28 transcript of control sample])

ΔCt_{E28D}=([Ct of E28D transcript of subject sample]−[Ct of E28D transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, R_{S} compares a level of E28D of a biological sample obtained from the subject to a level of [all SCN5A Exon 28 transcripts: WT, E28A-S, E28B, E28C, and E28D. In exemplary aspects,

$$\begin{matrix}{R}_{S} = {R}_{E 28 D} \\ = \frac{Calibrated abundance of E 28 D transcript}{Calibrated abundance of all SCN 5 A Exon 28 transcripts}\end{matrix}$$

wherein:

calibrated abundance of E28D transcript=2^{-ΔΔCtE28D transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{E28D transcript}=ΔCt_{E28D transcript}−ΔCt_{housekeeping gene}

ΔCt_{all SCN5A Exon 28 transcripts}=([Ct of all SCN5A Exon 28 transcripts of subject sample]−[Ct of all SCN5A Exon 28 transcripts of control sample])

ΔCt_{E28D}=([Ct of E28D transcript of subject sample]−[Ct of E28D transcript of control sample])

ΔCt_{housekeeping gene}=([Ct of housekeeping gene of subject sample]−[Ct of housekeeping gene of control sample])

wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, the method comprises determining a first R_{S} and a second R_{S}, wherein the first R_{S} compares a level of E28C to the level of all SCN5A Exon 28 transcripts, and the second R_{S} compares a level of E28D to the level of all SCN5A Exon 28 transcripts. In exemplary aspects, the first R_{S} is an R_{E28c} and the second R_{S} is an R_{E28D}, as described above.

In exemplary aspects, R_{S} is as taught above but “all SCN5A Exon 28 transcripts” refers to the sum of the level of E28A-S, E28B, E28C, and E28D.

Threshold Ratio, R_{T}

As described herein, the methods of the invention involve a ratio R_{S}, and, in exemplary embodiments, an interpretation of R_{S} is made via its comparison to a threshold ratio, R_{T}. For example, as described further herein, when R_{S} is greater than or equal to R_{T}, the subject is in need of an ICD. Further interpretations of R_{S} relative to R_{T} are described herein.

In exemplary embodiments, R_{T} serves as a direct reference point for R_{S}, and R_{T} is matched to R_{S}. For example, if R_{S} is a ratio of [a level of a truncated SCN5A Exon 28 transcript of a biological sample obtained from the subject] to [a level of a full length SCN5A Exon 28 transcript of the biological sample obtained from the subject], then R_{T} is a matched ratio, e.g., a ratio of [a level of a truncated SCN5A Exon 28 transcript of a biological sample obtained from the control subject] to [a level of a full length SCN5A Exon 28 transcript of the biological sample obtained from the control subject]. Also, for example, if R_{S} is [a level of a truncated SCN5A Exon 28 transcript of a biological sample obtained from the subject] to [all SCN5A Exon 28 transcripts of the biological sample obtained from the subject], then R_{T} is [a level of a truncated SCN5A Exon 28 transcript of a biological sample obtained from the control subject] to [all SCN5A Exon 28 transcripts of the biological sample obtained from the control subject].

Also, like R_{S}, in exemplary aspects, each level of R_{T} is a calibrated level (calibrated abundance) in which the level is normalized or calibrated to a level of a reference gene, e.g., a housekeeping gene. Suitable housekeeping genes are known in the art, some of which are further described herein.

Also, like R_{S}, in exemplary aspects, each level of R_{T} is a control-normalized level. The control-normalized level in exemplary aspects is a level which is the difference between the level of the subject and the level of a control subject. Control subjects are further described herein. In exemplary aspects, the control subject is a subject known as (i) not having an ICD, (ii) not having a cardiac disease, (iii) having normal left ventricular function, (iv) not having diastolic dysfunction, or (v) a combination thereof.

R_{T} may be defined in one of a variety of manners, depending on factors, including but not limited to, the application of the comparison between R_{S} and R_{T} (e.g., use in determining subject's need for an ICD vs. determining subject's risk for SCD vs. determining need for anti-arrhythmic agent), guidelines, requirements, or policies set by the regulatory agency (e.g., Food and Drug Administration, or like agency) of the region in which the methods of the invention are practiced, current standard health care practices of the region in which the methods of the invention are practiced, and the like.

In exemplary aspects, R_{T} is a ratio that compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein the biological sample is obtained from a control subject known as (i) not having an ICD, (ii) not having a cardiac disease, (iii) having normal left ventricular function, (iv) not having diastolic dysfunction, or (v) a combination thereof.

In alternative aspects, R_{T}=μ+4.0σ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as (i) not having an ICD, (ii) not having a cardiac disease, (iii) having normal left ventricular function, (iv) not having diastolic dysfunction, or (v) a combination thereof, and wherein σ is the standard deviation of the Gaussian distribution of the data values. In exemplary aspects, the ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample obtained from the control subject is matched to the R_{S} to which it is being compared.

In other alternative aspects, R_{T} is a ratio that compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample obtained from a control subject known as having an ICD that has not given a shock.

In exemplary aspects, R_{T} is determined via Real Time PCR. In exemplary aspects, R_{T} is as follows:

$$\frac{Truncated SCN 5 A Exon 28 transcript}{Full length SCN 5 A Exon 28 transcript} = \frac{\begin{matrix}Calibrated abundance of truncated \\ SCN 5 A Exon 28 transcript\end{matrix}}{\begin{matrix}Calibrated abundance of full length \\ SCN 5 A Exon 28 transcript\end{matrix}}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of subject sample]−[Ct of full length SCN5A Exon 28 transcript of control sample])

ΔCt_{truncated}=([Ct of truncated SCN5A Exon 28 transcript of subject sample]−[Ct of truncated SCN5A Exon 28 transcript of control sample])

ΔCt_{housekeeping gene}=([Ct of housekeeping gene of subject sample]−[Ct of housekeeping gene of control sample])

wherein “subject sample” is a biological sample obtained from a subject known as having an ICD that has not given a shock to the subject and “control sample” is a biological sample obtained from a subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

Also, in exemplary aspects,

$$\begin{matrix}{R}_{T} = \frac{Truncated SCN 5 A Exon 28 transcript}{All SCN 5 A Exon 28 transcripts} \\ = \frac{Calibrated abundance of truncated SCN 5 A Exon 28 transcript}{Calibrated abundance of all SCN 5 A Exon 28 transcripts}\end{matrix}$$

wherein:

calibrated abundance of truncated SCN5A Exon 28 transcript=2^{-ΔΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of subject sample]−[Ct of all SCN5A Exon 28 transcripts of control sample])

ΔCt_{truncated}=([Ct of truncated SCN5A Exon 28 transcript of subject sample]−[Ct of truncated SCN5A Exon 28 transcript of control sample])

ΔCt_{housekeeping gene}=([Ct of housekeeping gene of subject sample]−[Ct of housekeeping gene of control sample])

wherein “subject sample” is a biological sample obtained from a subject known as having an ICD that has not given a shock to the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In yet other aspects, R_{T}=μ+Xσ, wherein μ=is the mean value of a Gaussian distribution of a set of data values, wherein each data value of the set represents a ratio that which compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein the control subject is a subject known as having an ICD that has not given a shock, wherein σ is the standard deviation of the Gaussian distribution of the data values, and X is a number between about 0.7 and about 4.0 (e.g., 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0).

In exemplary aspects, when R_{T}=μ+Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts, of the biological sample, and the

$$\begin{matrix}ratio = \frac{Truncated SCN 5 A Exon 28 transcript}{Full length SCN 5 A Exon 28 transcript} \\ = \frac{Calibrated abundance of truncated SCN 5 A Exon 28 transcript}{Calibrated abundance of full length SCN 5 A Exon 28 transcript}\end{matrix}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of ICD patient (−) shock]−[Ct of full length SCN5A Exon 28 transcript of control sample])

ΔCt_{truncated}=([Ct of truncated SCN5A Exon 28 transcript of ICD patient (−) shock]−[Ct of truncated SCN5A Exon 28 transcript of control sample])

ΔCt_{housekeeping gene}=([Ct of housekeeping gene of ICD patient (−) shock]−[Ct of housekeeping gene of control sample]).

wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, when R_{T}=μ+Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts, of the biological sample, and the

$$\begin{matrix}ratio = \frac{Truncated SCN 5 A Exon 28 transcript}{All SCN 5 A Exon 28 transcripts} \\ = \frac{Calibrated abundance of truncated SCN 5 A Exon 28 transcript}{Calibrated abundance of all SCN 5 A Exon 28 transcripts}\end{matrix}$$

wherein:

calibrated abundance of truncated SCN5A Exon 28 transcript=2^{-ΔΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of ICD patient (−) shock]−[Ct of all SCN5A Exon 28 transcripts of control sample])

ΔCt_{truncated}=([Ct of truncated SCN5A Exon 28 transcript of ICD patient (−) shock]−[Ct of truncated SCN5A Exon 28 transcript of control sample])

ΔCt_{housekeeping gene}=([Ct of housekeeping gene of ICD patient (−) shock]−[Ct of housekeeping gene of control sample])

wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

Alternatively, R_{T}=μ−Xσ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as having an ICD that has given a shock to the control subject, wherein σ is the standard deviation of the Gaussian distribution of the data values, and X is a number between about 0.7 and about 4.0 (e.g., 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0). In exemplary aspects, X is a number between about 0.7 and about 1.0 or a number between about 2.0 and about 4.0 or a number between about 2.3 and about 4.0. In exemplary aspect, X is 2.326.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{Full length SCN 5 A Exon 28 transcript} = \frac{\begin{matrix}Calibrated abundance of truncated \\ SCN 5 A Exon 28 transcript\end{matrix}}{\begin{matrix}Calibrated abundance of full length \\ SCN 5 A Exon 28 transcript\end{matrix}}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of ICD patient (+) shock]−[Ct of full length SCN5A Exon 28 transcript of control sample])

ΔCt_{truncated}=([Ct of truncated SCN5A Exon 28 transcript of ICD patient (+) shock]−[Ct of truncated SCN5A Exon 28 transcript of control sample])

ΔCt_{housekeeping gene}=([Ct of housekeeping gene of ICD patient (+) shock]−[Ct of housekeeping gene of control sample]).

wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{All SCN 5 A Exon 28 transcripts} = \frac{Calibrated abundance of truncated SCN 5 A Exon 28 transcript}{Calibrated abundance of all SCN 5 A Exon 28 transcripts}$$

wherein:

calibrated abundance of truncated SCN5A Exon 28 transcript=2^{-ΔΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of ICD patient (+) shock]−[Ct of all SCN5A Exon 28 transcripts of control sample])

ΔCt_{truncated}=([Ct of truncated SCN5A Exon 28 transcript of ICD patient (+) shock]−[Ct of truncated SCN5A Exon 28 transcript of control sample])

ΔCt_{housekeeping gene}=([Ct of housekeeping gene of ICD patient (+) shock]−[Ct of housekeeping gene of control sample])

wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, R_{T} is determined by a system. In exemplary aspects, the system comprises a processor and a memory device coupled to the processor, wherein the memory device stores machine readable instructions that, when executed by the processor, cause the processor to:
(i) receive a plurality of data values, each data value is a ratio determined from a biological sample obtained from a subject of a first population, wherein the ratio compares a level of a truncated SCN5A Exon 28 transcript to (a) a level of a full length SCN5A Exon 28 transcript of the biological sample or (b) a level of all SCN5A Exon 28 transcripts of the biological sample or (c) a level of a full length SCN5A Exon 28 transcript and a level or one or more truncated SCN5A Exon 28 transcripts of the biological sample, wherein each subject of the first population is a subject known as having an ICD that has given a shock;
(ii) fit the plurality of data values to a first Gaussian distribution;

(iii) determine a mean value, μ, and a standard deviation, σ, of the first Gaussian distribution;
(iv) set a first threshold ratio, R_{T}, at μ−Xσ, wherein X is a number between 0.7 and 4.0.

Further descriptions of suitable systems that may be used to determine R_{T} is provided herein below.

Sudden Cardiac Death and Methods Relating Thereto

Sudden cardiac death (SCD) accounts for approximately 325,000 deaths per year in the United States, which number is higher than the number of deaths attributed to lung cancer, breast cancer, or acquired immune deficiency syndrome (AIDS). SCD is responsible for about 50% of deaths from heart failure and often is the first expression of coronary disease. See, Sovari et al., “Sudden Cardiac Death,” e-medicine Cardiology, article 151907, updated Nov. 4, 2010; and Zheng et al., Circulation 104: 2158-2163 (2001). A common cause of SCD is ventricular arrhythmia, including, for example, ventricular tachycardia (VT), in which the resting heart rate is faster than normal, ventricular fibrillation (VF), in which there is uncoordinated contraction of the cardiac muscle of the ventricles in the heart, making the muscles quiver rather than contract properly, or an arrhythmic condition in which both VT and VF are present. See, Wedro, B., “Sudden Cardiac Arrest (Sudden Cardiac Death),” medicine.net, Kulick and Soppler, eds. Current methods of treating ventricular fibrillation include defibrillation via an electrical defibrillator or a precordial thump. Anti-arrhythmic therapy aims to treat or prevent ventricular arrhythmias, thereby, preventing SCD. A type of anti-arrhythmic therapy includes implantation of an ICD into a patient. ICDs are further described herein.

As discussed, data provided herein support a means by which patients who receive shocks from ICDs may be identified based on levels of SCN5A Exon 28 transcript levels. Because patients who receive shocks from ICDs are at high risk for sudden cardiac death (SCD), the data provided herein also supports, in part, a means by which patients at risk for SCD may be identified. Accordingly, the invention provides a method of identifying patients at risk for sudden cardiac death. Stated in an alternative way, the invention also provides methods of determining a subject's risk for sudden cardiac death (SCD). In exemplary embodiments, such methods comprise the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined to be at risk for SCD, when R_{S} is greater than or equal to a threshold ratio, R_{T}.

With regard to the methods of determining a subject's risk for sudden cardiac death provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein (e.g., those described above in the section entitled Threshold Ratio, R_{T}).

Methods of Monitoring Risk for Sudden Cardiac Death

In exemplary aspects, the step of determining a ratio, Rₛ, is repeated at least one, if not, two, or more times. In exemplary aspects, ratio, Rₛ is determined 2, 3, 4, 5, 6, 7, 8, 9 10, or more times. In such cases, the method may be considered as a method of monitoring a subject's risk for SCD. In exemplary aspects, ratio, Rₛ is determined every 6 to 12 months (e.g., every 6, every 7 months, every 8 months, every 9 months, every 10 months, every 11 months, every 12 months months) and each time, Rₛ is based on a different biological sample obtained from the same subject.

Methods of Determining Need for Anti-Arrhythmic Therapy

In some cases, patients who have been determined to be at risk for SCD are provided anti-arrhythmic therapy. Because the methods of the invention provide a means by which a subject's risk for SCD is determined, the methods of the invention also provide a means by which a subject's need for anti-arrhythmic therapy is determined. Accordingly, the invention provides a method of determining a subject's need for anti-arrhythmic therapy. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined to need anti-arrhythmic therapy, when R_{S} is greater than or equal to a threshold ratio, R_{T}.

With regard to the methods of determining a subject's need for anti-arrhythmic therapy provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein (e.g., those described above in the section entitled Threshold Ratio, R_{T}).

In exemplary aspects, the anti-arrhythmic therapy is implantation of an ICD, or related device. In exemplary aspects, the anti-arrhythmic therapy is administration of an anti-arrhythmic agent.

Anti-Arrhythmic Agents

For purposes herein, the anti-arrhythmic agent is any one of a group of pharmaceuticals that are used to suppress abnormal rhythms of the heart (cardiac arrhythmias), such as atrial fibrillation, atrial flutter, ventricular tachycardia, and ventricular fibrillation. In exemplary aspects, the anti-arrhythmic agent is a Singh Vaughan Williams (SVW) Class I, II, III, IV, or V anti-arrhythmic agent. In exemplary aspects, the anti-arrhythmic agent is a SVW Class IA, IB, IC, or III anti-arrhythmic agent. The anti-arrhythmic agent may be a fast-channel blocker, a beta blocker, a slow channel blocker, a sodium channel blocking agent, a potassium channel blocking agent, or a calcium channel blocking agent. The anti-arrhythmic agent in some aspects is one of Quinidine, Procainamide, Disopyramide, Lidocaine, Phenyloin, Mexiletine, Tocamide, Flecamide, Propafenone, Moricizine, Propranolol, Esmolol, Timolol, Metoprolol, Atenolol, Bisoprolol, Amiodarone, Sotalol, Ibutilide, Dofetilide, Dronedarone, E-4031, Verapamil, Diltiazem, Adenosine, Digoxin, or Magnesium Sulfate. In some aspects, the SVW Class IA is Quinidine, Procainamide, or Disopyramide. In some aspects, the SVW Class IB anti-arrhythmic agent is Lidocaine, Phenyloin, Mexiletine, or Tocamide. In some aspects, the SVW Class IC anti-arrhythmic agent is Flecamide, Propafenone, Moricizine, or Encamide. In some aspects, the SVW Class III anti-arrhythmic agent is Dronedarone, Amiodarone, or Ibutilide. In some aspects, the anti-arrhythmic agent is NAD+ or mitoTEMPO.

Methods Of Reducing Risk of Sudden Cardiac Death

The invention additionally provides methods of reducing risk of SCD in a subject. The method comprises determining a subject's risk for SCD and treating the subject, when the subject has been determined to be at risk for SCD. In exemplary embodiments, the methods of reducing risk of SCD in a subject provided herein comprises the steps of (A) determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample; and (B) providing therapy to the subject, when R_{S} is greater than or equal to a threshold ratio, R_{T}.

With regard to the methods of reducing risk of SCD provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein (e.g., those described above in the section entitled Threshold Ratio, R_{T}).

In exemplary aspects, the therapy provided to the subject, is an anti-arrhythmic therapy. In exemplary aspects, the therapy is implantation of an ICD. Accordingly, the methods of reducing risk of SCD provided herein may comprise the step of implanting an ICD, when R_{S} is greater than or equal to a threshold ratio, R_{T}. In exemplary aspects, the therapy is administration of an anti-arrhythmic agent. Suitable anti-arrhythmic agents are known in the art and described herein in the section called Anti-Arrhythmic agents. In exemplary aspects, the therapy is an angiotensin blocker, an ACE inhibitor, a sodium channel inhibitor, NAD+, a mitochondrial targeted anti-oxidant, mitoQ, midTEMPO, an activator of Protein Kinase A, forskolin, BH4, or a Src inhibitor.

Heart Failure and Methods Relating Thereto

Heart failure (HF) is defined as the ability of the heart to supply sufficient blood flow to meet the body's needs. In some embodiments, the signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. In some embodiments, the symptom of heart failure is one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) and |
| | slight limitation during ordinary activity. |
| III | Marked limitation in activity due to symptoms, even during less- |
| | than-ordinary activity, e.g. walking short distances (20-100 m). |
| | Comfortable only at rest. |
| IV | Severe limitations. Experiences symptoms even while at rest. |
| | Mostly bedbound patients. |

In exemplary aspects, the heart failure is a systolic heart failure, which is heart failure caused or characterized by a systolic dysfunction. In simple terms, systolic dysfunction is a condition in which the pump function or contraction of the heart (i.e., systole), fails. Systolic dysfunction may be characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output. In some aspects, the systolic heart failure is an ischemic heart failure. In alternative aspects, the systolic heart failure is a nonischemic heart failure.

As discussed herein, heart failure patients exhibited increased levels of truncated SCN5A Exon 28 transcripts and increased levels of full length SCN5A Exon 28 transcripts. Such findings support a means by which heart failure patients may be identified. Accordingly, the invention provides a method of identifying patients at risk for heart failure. Stated in an alternative way, the invention also provides methods of determining a subject's risk for heart failure. In exemplary embodiments, such methods comprise the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined to be at risk for HF, when R_{S} is greater than or equal to a threshold ratio, R_{T}.

With regard to the methods of determining a subject's risk for HF provided herein, R_{S}, in exemplary aspects, is the same as any one of those described herein (e.g., those described above in the subsection entitled: Ratio, R_{S}). The threshold ratio R_{T} is essentially the same as those described herein (e.g., those described above in the section entitled Threshold Ratio, R_{T}), except that, in some instances, the control sample, or control subject, or subject is different from those described in that section. For example, in respect to the methods of determining a subject's risk for HF provided herein, R_{T} in exemplary aspects is a ratio that compares a level of a truncated SCN5A Exon 28 transcript of a biological sample to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein the biological sample is obtained from a control subject known as (i) not having an ICD, (ii) not having a cardiac disease, e.g., heart failure, (iii) having normal left ventricular function, (iv) not having diastolic dysfunction, or (v) a combination thereof.

In alternative aspects, R_{T}=μ+4.0σ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as (i) not having an ICD, (ii) not having a cardiac disease, e.g., heart failure, (iii) having normal left ventricular function, (iv) not having diastolic dysfunction, or (v) a combination thereof, and wherein σ is the standard deviation of the Gaussian distribution of the data values. In exemplary aspects, the ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample obtained from the control subject is matched to the R_{S} to which it is being compared.

Alternatively, R_{T}=μ−Xσ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as having heart failure, wherein σ is the standard deviation of the Gaussian distribution of the data values, and X is a number between about 0.7 and about 4.0 (e.g., 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0). In exemplary aspects, X is a number between about 0.7 and about 1.0 or a number between about 2.0 and about 4.0 or a number between about 2.3 and about 4.0. In exemplary aspect, X is 2.326.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{Full length SCN 5 A Exon 28 transcript} = \frac{\begin{matrix}Calibrated abundance of truncated \\ SCN 5 A Exon 28 transcript\end{matrix}}{\begin{matrix}Calibrated abundance of full length \\ SCN 5 A Exon 28 transcript\end{matrix}}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of heart failure patient sample]−[Ct of full length SCN5A Exon 28 transcript of control sample])

ΔCt_{truncated}=([Ct of truncated SCN5A Exon 28 transcript of heart failure patient sample]−[Ct of truncated SCN5A Exon 28 transcript of control sample])

ΔCt_{housekeeping gene}=([Ct of housekeeping gene of heart failure patient sample]−[Ct of housekeeping gene of control sample]).

wherein “heart failure patient sample” is a biological sample obtained from a subject known as having heart failure and “control sample” is a biological sample obtained from a control subject known as (i) not having an ICD, (ii) not having a cardiac disease, e.g., heart failure, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{All SCN 5 A Exon 28 transcripts} = \frac{Calibrated abundance of truncated SCN 5 A Exon 28 transcript}{Calibrated abundance of all SCN 5 A Exon 28 transcripts}$$

wherein:

calibrated abundance of truncated SCN5A Exon 28 transcript=2^{-ΔΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of heart failure patient sample]−[Ct of all SCN5A Exon 28 transcripts of control sample])

ΔCt_{truncated}=([Ct of truncated SCN5A Exon 28 transcript of heart failure patient sample]−[Ct of truncated SCN5A Exon 28 transcript of control sample])

ΔCt_{housekeeping gene}=([Ct of housekeeping gene of heart failure patient sample]−[Ct of housekeeping gene of control sample])

wherein “heart failure patient sample” is a biological sample obtained from a subject known as having heart failure and “control sample” is a biological sample obtained from a control subject known as (i) not having an ICD, (ii) not having a cardiac disease, e.g., heart failure, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, R_{T} is determined by a system. In exemplary aspects, the system comprises a processor and a memory device coupled to the processor, wherein the memory device stores machine readable instructions that, when executed by the processor, cause the processor to:
(i) receive a plurality of data values, each data value is a ratio determined from a biological sample obtained from a subject of a first population, wherein the ratio compares a level of a truncated SCN5A Exon 28 transcript to (a) a level of a full length SCN5A Exon 28 transcript of the biological sample or (b) a level of all SCN5A Exon 28 transcripts of the biological sample or (c) a level of a full length SCN5A Exon 28 transcript and a level or one or more truncated SCN5A Exon 28 transcripts of the biological sample, wherein each subject of the first population is a subject known as having heart failure;
(ii) fit the plurality of data values to a first Gaussian distribution;
(iii) determine a mean value, μ, and a standard deviation, σ, of the first Gaussian distribution;
(iv) set a first threshold ratio, R_{T}, at μ−Xσ, wherein X is a number between 0.7 and 4.0.

Further descriptions of suitable systems that may be used to determine R_{T} is provided herein below.

Methods Of Monitoring Risk for HF

In exemplary aspects, the step of determining a ratio, Rₛ, is repeated at least one, if not, two, or more times. In exemplary aspects, ratio, Rₛ is determined 2, 3, 4, 5, 6, 7, 8, 9 10, or more times. In such cases, the method may be considered as a method of monitoring a subject's risk for HF. In exemplary aspects, ratio, Rₛ is determined every 6 to 12 months (e.g., every 6, every 7 months, every 8 months, every 9 months, every 10 months, every 11 months, every 12 months months) and each time, Rₛ is based on a different biological sample obtained from the same subject.

Methods of Determining Need for Anti-HF Therapy

The invention also provides a method of determining a subject's need for therapy or prophylaxis for HF. In exemplary embodiments, the method comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined to need therapy, when R_{S} is greater than or equal to a threshold ratio, R_{T}.

With regard to the methods of determining a subject's need for anti-HF therapy provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein in the section entitled Heart Failure and Methods Relating Thereto).

Methods Of Reducing Risk of HF

The invention additionally provides methods of reducing risk of HF in a subject. The method comprises determining a subject's risk for HF and treating the subject, when the subject has been determined to be at risk for HF. In exemplary embodiments, the methods of reducing risk of HF in a subject provided herein comprises the steps of (A) determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample; and (B) providing therapy to the subject, when R_{S} is greater than or equal to a threshold ratio, R_{T}.

With regard to the methods of reducing a subject's risk for HF provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein in the section entitled Heart Failure and Methods Relating Thereto).

Therapy For Heart Failure

Anti-HF therapies are known in the art. See, e.g., Abraham and Krum, “Heart

Failure: A Practical Approach to Treatment,” McGraw Hill Professional, 2007. The anti-HF therapy in exemplary aspects includes administration of one of more of: Angiotensin converting enzyme (ACE) inhibitors, Angiotensin II receptor blockers (ARBs), Beta-blockers, Digoxin, Diuretics, Blood vessel dilators, Potassium or magnesium, Aldactone Inhibitors, Calcium channel blockers, and Heart pump medication. In exemplary aspects, the anti-HF therapy includes one or more surgical procedures, including, but not limited to: bypass surgery, left ventricular assist device, heart valve surgery, infarct exclusion surgery, heart transplant.

Arrhythmia and Methods Related Thereto

Arrhythmias

The invention moreover provides a method of determining a subject's risk for arrhythmia. As used herein, the term “arrhythmia” is synonymous with “cardiac dysrhythmia” or “cardiac arrhythmia” and refers to any condition in which there is abnormal electrical activity in the heart. In exemplary embodiments, the cardiac arrhythmia is a ventricular arrhythmia, such as ventricular fibrillation, ventricular tachycardia, or an arrhythmic condition in which both ventricular fibrillation and ventricular tachycardia are present. In exemplary embodiments, the cardiac arrhythmia is an atrial arrhythmia, e.g., an atrial fibrillation, atrial tachycardia, or an arrhythmic condition in which both atrial fibrillation and atrial tachycardia are present. Other types of cardiac arrhythmias are described below.

In exemplary aspects, the cardiac arrhythmia is characterized by an abnormal heart rate. In exemplary aspects, the cardiac arrhythmia is characterized by a bradycardia or a tachycardia.

Bradycardia

In exemplary aspects, the cardiac arrhythmia is a bradycardia in which the resting heart rate is slower than normal. In exemplary aspects, the bradycardia is characterized by a resting heart rate in an adult human which is slower than 60 beats per minute. In exemplary aspects, the bradycardia is a sinus bradycardia. In exemplary aspects, the bradycardia is caused by sinus arrest or AV block or heart block. In exemplary aspects, the bradycardia is caused by a slowed electrical conduction in the heart. In exemplary aspects, the bradycardia is not the bradycardia which is exhibited by the normally functioning heart of an athlete or athletic person.

Tachycardia

In exemplary aspects, the cardiac arrhythmia is a tachycardia in which the resting heart rate is faster than normal. In exemplary aspects, the tachycardia is characterized by a resting heart rate in an adult human which is faster than 100 beats per minute. In exemplary aspects, the tachycardia is a sinus tachycardia. In exemplary aspects, the sinus tachycardia is not caused by physical exercise, emotional stress, hyperthyroidism, ingestion or injection of substances, such as caffeine or amphetamines. In exemplary aspects, the tachycardia is not a sinus tachycardia, e.g., a tachycardia resulting from automaticity, reentry (e.g., fibrillation), or triggered activity. In exemplary aspects, the tachycardia is caused by a slowed electrical conduction in the heart. In exemplary aspects, the tachycardia is caused by an ectopic focus. In exemplary aspects, the tachycardia is combined with abnormal rhythm.

In exemplary aspects, the cardiac arrhythmia is characterized by the mechanism by which it occurs. In exemplary aspects, the cardiac arrhythmia is caused by automaticity, re-entry, or fibrillation.

Automaticity

In exemplary aspects, the cardiac arrhythmia is an abnormal rhythm or a tachycardia caused by automaticity, a condition in which a cardiac muscle cell other than a cardiac muscle of the conduction system fires an impulse of its own. In exemplary aspects, the cardiac arrhythmia is caused by a muscle cell, other than a cell of the sino-atrial (SA) node, atrial-ventricular (AV) node, Bundle of His, or Purkinje fibers, firing an impulse of its own.

Re-Entry

In exemplary aspects, the cardiac arrhythmia is a re-entry arrhythmia in which an electrical impulse recurrently travels in a circle within the heart, rather than moving from one end of the heart to the other and then stopping. In exemplary aspects, the cardiac arrhythmia is a cardiac flutter, a paroxysmal supraventricular tachycardia, or a ventricular tachycardia.

Fibrillation

In exemplary aspects, the cardiac arrhythmia is a fibrillation. In exemplary aspects, the fibrillation is an atrial fibrillation. In exemplary aspects, the fibrillation is a ventricular fibrillation.

Triggered Beats

In exemplary aspects, the cardiac arrhythmia is a triggered beat that occurs when ion channels in the heart cells malfunction, resulting in abnormal propagation of electrical activity and possibly leading to abnormal rhythm.

In exemplary embodiments, the cardiac arrhythmia is classified by site of origin. In exemplary aspects, the cardiac arrhythmia is an atrial arrhythmia (e.g., premature atrial contraction, wandering atrial pacemaker, multifocal atrial tachycardia, atrial flutter, atrial fibrillation). In exemplary aspects, the cardiac arrhythmia is a junction arrhythmia (e.g., supraventricular tachycardia, AV nodal reentral tachycardia, paroxysmal supraventricular tachycardia, junctional rhythm, junctional tachycardia, premature junctional complex). In exemplary aspects, the cardiac arrhythmia is an atrio-ventricular arrhythmia (e.g., AV reentrant tachycardia). In exemplary aspects, the cardiac arrhythmia is a ventricular arrhythmia (e.g., premature ventricular contraction or ventricular extra beat, accelerated idoventricular rhythm, monomorphic ventricular tachycardia, polymorphic ventricular tachycardia, ventricular fibrillation). In exemplary aspects, the cardiac arrhythmia is a heart block (e.g., first degree heart block, Type I second degree heart block, Type 2 second degree heart block, third degree heat block). In exemplary aspects, the cardiac arrhythmia is a premature contraction.

In exemplary aspects, the cardiac arrhythmia is a condition in which two or more types of cardiac arrhythmias are present. In exemplary aspects, the cardiac arrhythmia is a condition in which both ventricular tachycardia and ventricular fibrillation are present. In exemplary aspects, the cardiac arrhythmia is a condition in which a bradycardia is not present.

In exemplary aspects, the methods of determining a subject's risk for arrhythmia comprises the step of determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, the subject is determined to be at risk for arrhythmia, when R_{S} is greater than or equal to a threshold ratio, R_{T}.

With regard to the methods of determining a subject's risk for arrhythmia provided herein, R_{S}, in exemplary aspects, is the same as any one of those described herein (e.g., those described above in the subsection entitled: Ratio, R_{S}). The threshold ratio R_{T} is essentially the same as those described herein (e.g., those described above in the section entitled Threshold Ratio, R_{T}), except that, in some instances, the control sample, or control subject, or subject is different from those described in that section. For example, in respect to the methods of determining a subject's risk for HF provided herein, R_{T} in exemplary aspects is a ratio that compares a level of a truncated SCN5A Exon 28 transcript of a biological sample to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein the biological sample is obtained from a control subject known as (i) not having an ICD, (ii) not having a cardiac disease, e.g., heart failure, (iii) having normal left ventricular function, (iv) not having diastolic dysfunction, or (v) a combination thereof.

In alternative aspects, R_{T}=μ+4.0σ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as (i) not having an ICD, (ii) not having a cardiac disease, e.g., heart failure, (iii) having normal left ventricular function, (iv) not having diastolic dysfunction, or (v) a combination thereof, and wherein σ is the standard deviation of the Gaussian distribution of the data values. In exemplary aspects, the ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample obtained from the control subject is matched to the R_{S} to which it is being compared.

Alternatively, R_{T}=μ−Xσ, wherein μ=is the mean value of a Gaussian distribution of a set of data values, wherein each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, wherein the control subject is a subject known as having arrhythmia, wherein σ is the standard deviation of the Gaussian distribution of the data values, and X is a number between about 0.7 and about 4.0 (e.g., 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0). In exemplary aspects, X is a number between about 0.7 and about 1.0 or a number between about 2.0 and about 4.0 or a number between about 2.3 and about 4.0. In exemplary aspect, X is 2.326.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{Full length SCN 5 A Exon 28 transcript} = \frac{\begin{matrix}Calibrated abundance of truncated \\ SCN 5 A Exon 28 transcript\end{matrix}}{\begin{matrix}Calibrated abundance of full length \\ SCN 5 A Exon 28 transcript\end{matrix}}$$

wherein:

calibrated abundance of truncated SCN5A Exon 28 transcript=2^{-ΔΔCttruncated SCN5A Exon 28 transcript}

calibrated abundance of full length SCN5A Exon 28 transcript=2^{-ΔΔCtfull length SCN5A Exon 28 transcript}

ΔΔCt_{full length SCN5A Exon 28 transcript}=ΔCt_{full length}−ΔCt_{housekeeping gene}

ΔΔCt_{truncated SCN5A Exon 28 transcript}=ΔCt_{truncated}−ΔCt_{housekeeping gene}

ΔCt_{full length}=([Ct of full length SCN5A Exon 28 transcript of arrhythmia patient sample]−[Ct of full length SCN5A Exon 28 transcript of control sample])

ΔCt_{truncated}=([Ct of truncated SCN5A Exon 28 transcript of arrhythmia patient sample]−[Ct of truncated SCN5A Exon 28 transcript of control sample])

ΔCt_{housekeeping gene}=([Ct of housekeeping gene of arrhythmia patient sample]−[Ct of housekeeping gene of control sample]).

wherein “arrhythmia patient sample” is a biological sample obtained from a subject known as having arrhythmia and “control sample” is a biological sample obtained from a control subject known as (i) not having an ICD, (ii) not having a cardiac disease, e.g., arrhythmia, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts.

In exemplary aspects, when R_{T}=μ−Xσ, each data value of the set represents a ratio that compares a level of a truncated SCN5A Exon 28 transcript of a biological sample obtained from a control subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample of the control subject, and the ratio is

$$\frac{Truncated SCN 5 A Exon 28 transcript}{All SCN 5 A Exon 28 transcripts} = \frac{Calibrated abundance of truncated SCN 5 A Exon 28 transcript}{Calibrated abundance of all SCN 5 A Exon 28 transcripts}$$

wherein:

calibrated abundance of truncated SCN5A Exon 28 transcript=2^{-ΔΔCttruncated SCN5A Exon 28 transcript}

calibrated abundance of all SCN5A Exon 28 transcripts=2^{-ΔΔCtall SCN5A Exon 28 transcripts}

ΔΔCt_{all SCN5A Exon 28 transcripts}=ΔCt_{all SCN5A Exon 28 transcripts}−ΔCt_{housekeeping gene}

ΔΔCt_{truncated SCN5A Exon 28 transcript}=ΔCt_{truncated}−ΔCt_{housekeeping gene}

ΔCt_{all SCN5A Exon 28 transcripts}=([Ct of all SCN5A Exon 28 transcripts of arrhythmia patient sample]−[Ct of all SCN5A Exon 28 transcripts of control sample])

ΔCt_{truncated}=([Ct of truncated SCN5A Exon 28 transcript of arrhythmia patient sample]−[Ct of truncated SCN5A Exon 28 transcript of control sample])

ΔCt_{housekeeping gene}=([Ct of housekeeping gene of arrhythmia patient sample]−[Ct of housekeeping gene of control sample])

wherein “arrhythmia patient sample” is a biological sample obtained from a subject known as having heart failure and “control sample” is a biological sample obtained from a control subject known as (i) not having an ICD, (ii) not having a cardiac disease, e.g., arrhythmia, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

In exemplary aspects, R_{T} is determined by a system. In exemplary aspects, the system comprises a processor and a memory device coupled to the processor, wherein the memory device stores machine readable instructions that, when executed by the processor, cause the processor to:
(i) receive a plurality of data values, each data value is a ratio determined from a biological sample obtained from a subject of a first population, wherein the ratio compares a level of a truncated SCN5A Exon 28 transcript to (a) a level of a full length SCN5A Exon 28 transcript of the biological sample or (b) a level of all SCN5A Exon 28 transcripts of the biological sample or (c) a level of a full length SCN5A Exon 28 transcript and a level or one or more truncated SCN5A Exon 28 transcripts of the biological sample, wherein each subject of the first population is a subject known as having arrhythmia;
(ii) fit the plurality of data values to a first Gaussian distribution;
(iii) determine a mean value, μ, and a standard deviation, ∝, of the first Gaussian distribution;
(iv) set a first threshold ratio, R_{T}, at μ−Xσ, wherein X is a number between 0.7 and 4.0.

Further descriptions of suitable systems that may be used to determine R_{T} is provided herein below.

Methods Of Monitoring Risk for Arrhythmia

In exemplary aspects, the step of determining a ratio, Rₛ, is repeated at least one, if not, two, or more times. In exemplary aspects, ratio, Rₛ is determined 2, 3, 4, 5, 6, 7, 8, 9 10, or more times. In such cases, the method may be considered as a method of monitoring a subject's risk for arrhythmia. In exemplary aspects, ratio, Rₛ is determined every 6 to 12 months (e.g., every 6, every 7 months, every 8 months, every 9 months, every 10 months, every 11 months, every 12 months months) and each time, Rₛ is based on a different biological sample obtained from the same subject.

Methods of Reducing Risk of Arrhythmia

The invention additionally provides methods of reducing risk of arrhythmia in a subject. The method comprises determining a subject's risk for arrhythmia and treating the subject, when the subject has been determined to be at risk for arrhythmia. In exemplary embodiments, the methods of reducing risk of arrhythmia in a subject provided herein comprises the steps of (A) determining a ratio, Rₛ, which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample; and (B) providing anti-arrhythmic therapy to the subject, when R_{S} is greater than or equal to a threshold ratio, R_{T}.

With regard to the methods of reducing a subject's risk for arrhythmia provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein in the section entitled Arrhythmia and Methods Relating Thereto). The anti-arrhythmic therapy provided in the methods of reducing risk for arrhythmia may be any suitable anti-arrhythmic therapy known in the art, some of which are described herein.

Methods of Determining Safe Use of a Therapeutic

The invention furthermore provides methods of determining whether administration of an anti-arrhythmic agent to a subject is safe. The method comprises determining a ratio R_{S} which compares a level of a truncated SCN5A Exon 28 transcript of a biological sample obtained from the subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. In exemplary aspects, when R_{S} is greater than or equal to a threshold ratio R_{T}, the administration of the anti-arrhythmic agent to the subject is safe.

With regard to the methods of determining whether administration of an anti-arrhythmic agent to a subject is safe provided herein, R_{S}, in exemplary aspects, is the same as any one of those described herein (e.g., those described above in the subsection entitled: Ratio, R_{S}). Also, in exemplary aspects the threshold ratio R_{T} is the same as any one of those described herein in the section entitled Threshold Ratio R_{T}). The anti-arrhythmic agent provided in the methods of reducing risk for arrhythmia may be any suitable anti-arrhythmic agent known in the art, some of which are described herein. In exemplary aspects, the anti-arrhythmic agent is a sodium channel blocking agent, e.g., NAD+, mitoTEMPO.

Levels of SCN5A Exon 28 Transcripts

The methods described herein reference one or more levels of SCN5A Exon 28 transcripts. The methods include, for example, a step of determining a ratio relating a level of a truncated SCN5A Exon 28 transcript of a biological sample to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample. The levels of SCN5A Exon 28 transcripts may be determined or obtained by any suitable means. In exemplary aspects, the levels are determined by measuring the levels from a biological sample. For instance, any of a level of a full length transcript of the SCN5A gene and a level of a truncated SCN5A Exon 28 transcript may be measured using suitable techniques of measuring transcripts known in the art. The measurement of these levels may be a direct measurement of SCN5A Exon 28 transcripts. Such methods may be considered as involving the measurement of expression levels of the SCN5A Exon 28 transcripts. In exemplary aspects, the level that is measured is an mRNA transcript level, or a level of the product encoded by the mRNA transcript, e.g., a protein or peptide expression level. Suitable methods of determining expression levels of proteins are known in the art and include immunoassays (e.g., Western blotting, an enzyme-linked immunosorbent assay (ELISA), a radioimmunoassay (RIA), and immunohistochemical assay. Suitable methods of determining expression levels of nucleic acids (e.g., mRNA) are known in the art and include quantitative polymerase chain reaction (qPCR), including, but not limited to, real time PCR, Northern blotting and Southern blotting.

In alternative aspects, the measurement of SCN5A Exon 28 transcripts may be an indirect measurement, wherein something other than the SCN5A Exon 28 transcripts are measured. For example, the SCN5A Exon 28 transcript levels may determined by measuring an expression level or biological activity level of a related protein, e.g., a protein which acts upstream or downstream of the SCN5A Exon 28 transcript. For example, data provided herein evidence that splicing factors hLuc7A and RBM25, as well as the transducer of the unfolded protein response (UPR), PERK, are associated with abnormal splicing of the SCN5A gene. Accordingly, the levels may be determined by measuring expression levels of splicing factor hLuc7a, splicing factor RBM25 and/or PERK. The expression levels of splicing factor hLuc7a, splicing factor RBM25 and/or PERK may be measured by measurement of gene expression, or expression of the gene products (e.g., mRNA, protein). Accordingly, in exemplary aspects, the methods described herein may comprise measuring a level of splicing factor hLuc7a protein, splicing factor RBM25 protein and/or PERK protein or, a splicing factor hLuc7a mRNA, splicing factor RBM25 mRNA and/or PERK mRNA.

Luc7A (NCBI Gene ID No. 51747) is also known as LUC7L3; LUC7-like 3; CRA; CROP; LUCIA; hLuc7A; CREAP-1; and OA48-18. Exemplary mRNA sequences of hLuc7A are set forth herein as SEQ ID NOs: 34 and 35 but may also found in the NCBI's nucleotide database as Accession No. NM_{—}006107.3 and as Accession No. NM_{—}016424.4. Exemplary amino acid sequences of hLuc7A are set forth herein as SEQ ID NOs: 36 and 37 but may also be found in the NCBI's Protein database as Accession No. NP_{—}006098.2 and as Accesssion No. NP_{—}057508.2. RBM25 (NCBI Gene ID No. 58517) is also known RNA binding motif protein 25; 5164; NET52; RNPC7; Snu71; RED120; fSAP94; MGC105088; and MGC117168. An exemplary mRNA sequence of RBM25 is set forth herein as SEQ ID NO: 38 but may be found in the NCBI's nucleotide database as Accession No. NM_{—}021239.2. An exemplary amino acid sequence of RBM25 is set forth herein as SEQ ID NO: 39 but may be found in the NCBI's Protein database as Accession No. NP_{—}067062.1. PERK (NCBI Gene ID No. 9451) is also known as eukaryotic translation initiation factor 2-alpha kinase 3, EIF2AK3, protein kinase R-like endoplasmic reticulum kinase, PKR-like ER kinase; PEK; WRS; and DKFZp781H1925. An exemplary mRNA sequence of PERK is set forth herein as SEQ ID NO: 40 but may also be found on the NCBI's nucleotide database as Accession No. NM_{—}004836.5. An exemplary amino acid sequence of PERK is set forth herein as SEQ ID NO: 41 but may also be found on the NCBI's Protein database as Accession No. NP_{—}004827.4.

Also, for example, the SCN5A Exon 28 transcript levels may be determined by measuring an expression level or biological activity level of a chaperone protein (e.g., calnexin, CHOP), since these proteins are upregulated once the UPR is activated via PERK, which, in turn, is activated by the certain truncated SCN5A transcripts. Accordingly, in exemplary aspects, the methods described herein may comprise measuring a level of a chaperone protein in a biological sample. In exemplary embodiments, the chaperone protein is CHOP or calnexin.

In exemplary aspects, the chaperone protein is CHOP. CHOP (NCBI Gene ID No. 1649) is also known as DDIT3, DNA-damage-inducible transcript 3, CEBPZ; CHOP10; CHOP-10; GADD153; MGC4154. Exemplary amino acid sequences of CHOP are set forth herein as SEQ ID NOs: 42-47 but are also found in the NCBI's Protein database as Accession Nos. NP_{—}001181982.1, NP_{—}001181983.1, NP_{—}001181984.1, NP_{—}001181985.1, NP_{—}001181986.1, NP_{—}004074.2. Exemplary nucleotide sequences of CHOP are set forth herein as SEQ ID NO: 48-53 but are also found in the NCBI's Nucleotide database as Accession Nos. NM_{—}001195053.1, NM_{—}001195054.1, NM_{—}001195055.1, NM_{—}001195056.1, NM_{—}001195057.1, and NM_{—}004083.5.

In exemplary aspects, the chaperone protein is calnexin. Calnexin (NCBI Gene ID No. 821) is also known as CANX; CNX; P90; IP90; FLJ26570. Exemplary amino acid sequences of calnexin are set forth herein as SEQ ID NOs: 54 and 55 but are also found in the NCBI's Protein database as Accession Nos. NP_{—}001019820.1 and NP_{—}001737.1. Exemplary nucleotide sequences of Calnexin are set forth herein as SEQ ID NO: 56 and 57 but are also found in the NCBI's Nucleotide database as Accession Nos. NM_{—}001024649.1 and NM_{—}001746.3.

In the methods in which the level of splicing factor hLuc7a, splicing factor RBM25, PERK and/or a chaperone protein is measured, the level may be an expression level (e.g., a protein level, an mRNA level, gene expression level) of the splicing factor hLuc7a, splicing factor RBM25, PERK, or chaperone protein. Alternatively, the level may be a biological activity level, such as, an enzymatic activity level or a binding activity level. In exemplary aspects, the measurement may be made by measuring the amount of RBM25 bound to the SCN5A gene or gene transcript.

Medical Record

In exemplary aspects, the levels are determined by obtaining the levels from a medical record. For example, the levels may have been measured and recorded at a time prior to when the steps of the methods of the invention are carried out, e.g., prior to when R_{S} is determined. In exemplary aspects, the levels are obtained from a medical record of a subject containing levels that were measured and recorded no more than 6 months prior to the time at which the steps of the method of the invention are carried out. In exemplary aspects, the levels are obtained from a medical record of a subject containing levels that were measured and recorded no more than 5 months or 4 months or 3 months or 2 months prior to the time at which the steps of the method of the invention are carried out. In exemplary aspects, the levels are obtained from a medical record of a subject containing levels that were measured and recorded no more than 1 month or 3 weeks or 2 weeks or 1 week prior to the time at which the steps of the method of the invention are carried out.

In exemplary aspects, some of the levels are determined by obtaining the levels from a medical record and some are measured. In exemplary aspects, the method comprises the steps of measuring the level of a full length transcript of the SCN5A gene and obtaining from a medical record of the subject the level of a SNC5A splice variant produced from alternative splicing within Exon 28 of the SCN5A gene. Alternatively, the method comprises the steps of obtaining from a medical record of the subject the level of a full length transcript of the SCN5A gene and measuring the level of a SNC5A splice variant produced from alternative splicing within Exon 28 of the SCN5A gene.

Additional Steps

With regard to the methods of the invention, the methods may include additional steps. For example, the method may include repeating one or more of the recited step(s) of the method. Accordingly, in exemplary aspects, the method comprises re-determining a ratio R_{S}. In exemplary aspects, the method comprises re-determining a ratio R_{S} every 6 to 12 months, wherein the determination of each R_{S} is based on a different biological sample obtained from the same subject. In some aspects, the method comprises obtaining a biological sample from the subject every 6 to 12 months and determining the R_{S} of each biological sample obtained.

In exemplary aspects, the method comprises administering a therapeutic agent or device once the need therefor or a risk has been determined. For example, the methods described herein may optionally comprise a step of providing an appropriate therapy (administering a pharmaceutical agent or implementing a standard of care) to the subject determined to have a need therefor. In exemplary aspects, the methods of the invention comprise one or more steps related to providing the appropriate therapy. The methods may, for example, comprise a step of implanting an ICD into a subject, or administering to the subject an anti-arrhythmic agent. The anti-arrhythmic agent may be any one known in the art, some of which are described herein. The anti-arrhythmic agent may be administered to the subject by any suitable route of administration known in the art, some routes of which are described herein below.

In exemplary aspects, the method comprises determining the levels of SCN5A Exon 28 transcript in more than one way. In exemplary aspects, the method comprises the steps of (i) measuring the levels of SCN5A Exon 28 transcripts from a biological sample obtained from the subject and (ii) obtaining the levels of SCN5A Exon 28 transcripts from a medical record of the subject. In exemplary aspects, the method comprises measuring the levels of SCN5A Exon 28 transcript via measurement of SCN5A Exon 28 mRNA transcripts and via measurement of the protein products encoded by the SCN5A Exon 28 mRNA transcripts. In exemplary aspects, the method comprises measuring the levels of two, three, four, five, six, seven, eight, or all of the following: (i) SCN5A Exon 28 mRNA transcripts, (ii) SCN5A Exon 28 proteins, (iii) expression levels of hLuc7A, (iv) expression levels of RBM25, (v) binding levels of RBM25 to the SCN5A gene or gene transcript, (vi) expression levels of PERK (vii) biological activity levels of PERK, (viii) expression levels of a chaperone protein, (ix) biological activity levels of a chaperone protein. In exemplary aspects, the method comprises measuring the levels of more than one truncated SCN5A Exon 28 transcript, e.g., comprises measuring both E28C and E28D levels.

In exemplary aspects, the method comprises determining the levels of more than one type of truncated SCN5A Exon 28 transcript (e.g., E28C and E28D) and/or more than one type of full-length SCN5A Exon 28 transcript (e.g., E28A-S and WT SCN5A Exon 28).

Any and all possible combinations of the steps described herein are contemplated for purposes of the inventive methods.

Biological Samples

With regard to the methods disclosed herein, in some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In exemplary aspects, the sample comprises white blood cells obtained from the subject. In exemplary aspects, the sample comprises only white blood cells. In exemplary aspects, the sample is muscle tissue (e.g., skeletal muscle tissue). In exemplary aspects, the sample is cardiac tissue (e.g., cardiac muscle tissue).

Subjects

With regard to the methods disclosed herein, the subject in exemplary aspects is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

Controls

In the methods described herein, the level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, BMI, current therapeutic regimen status, medical history, or a combination thereof, but differs from the subject being diagnosed in that the control does not suffer from the disease in question or is not at risk for the disease.

Relative to a control level, the level that is determined may an increased level. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

Housekeeping Genes

For purposes herein, the levels of SCN5A transcripts are determined (either obtained or measured) and the levels are normalized or calibrated to a level of a housekeeping gene. The housekeeping gene in some aspects is β-actin or GAPDH. In exemplary aspects, the housekeeping gene is any one of those set forth in the sequence listing as SEQ ID NOs: 61-635 or any one of those set forth in Table A below.

TABLE A

| Accession | | SEQ |
|---|---|---|
| No. | Name | ID NO |
| NM_001101 | Actin, beta (ACTB) | 60 |
| NM_000034 | aldolase A, fructose-bisphosphate (ALDOA) | 61 |
| NM_002046 | Glyceraldehyde-3-phosphate dehydrogenase (GAPDH) | 62 |
| NM_000291 | Phosphoglycerate kinase 1 (PGK1) | 63 |
| NM_005566 | Lactate dehydrogenase A (LDHA), | 64 |
| NM_002954 | Ribosomal protein S27a (RPS27A) | 65 |
| NM_000981 | Ribosomal protein L19 (RPL19) | 66 |
| NM_000975 | Ribosomal protein L11 (RPL11) | 67 |
| NM_007363 | Non-POU domain containing, octamer-binding (NONO) | 68 |
| NM_004309 | Rho GDP dissociation inhibitor (GDI) alpha (ARHGDIA) | 69 |
| NM_000994 | Ribosomal protein L32 (RPL32) | 70 |
| NM_022551 | Ribosomal protein S18 (RPS18), | 71 |
| NM_007355 | heat shock protein 90 kDa alpha (cytosolic), class B member 1 (HSP90AB1) | 72 |
| NM_004515 | Interleukin enhancer binding factor 2, 45 kDa (ILF2) | 73 |
| NM_004651 | Ubiquitin specific peptidase 11 (USP11) | 74 |
| NM_004888 | ATPase, H+ transporting, lysosomal 13 kDa, V1 subunit G1 (ATP6V1G1) | 75 |
| NM_003334 | Ubiquitin-like modifier activating enzyme 1 (UBA1) | 76 |
| NM_001320 | Casein kinase 2, beta polypeptide (CSNK2B) | 77 |
| NM_003915 | Copine I (CPNE1) | 78 |
| NM_001250 | CD40 molecule, TNF receptor superfamily member 5 (CD40) | 79 |
| NM_001904 | Catenin (cadherin-associated protein), beta 1, 88 kDa (CTNNB1) | 80 |
| NM_003753 | Eukaryotic translation initiation factor 3, subunit D (EIF3D) | 81 |
| NM_004541 | NADH dehydrogenase (ubiquinone) 1 alpha subcomplex, 1, 7.5 kDa (NDUFA1) | 82 |
| NM_001654 | V-raf murine sarcoma 3611 viral oncogene homolog (ARAF) | 83 |
| NM_002967 | Scaffold attachment factor B (SAFB) | 84 |
| NM_001183 | H+ transporting, lysosomal accessory protein 1 (ATP6AP1) | 85 |
| NM_003526 | Histone cluster 1, H2bc (HIST1H2BC) | 86 |
| NM_004718 | Cytochrome c oxidase subunit VIIa polypeptide 2 like (COX7A2L) | 87 |
| NM_004436 | Endosulfine alpha (ENSA) | 88 |
| NM_001207 | Basic transcription factor 3 (BTF3) | 89 |
| NM_004907 | Immediate early response 2 (IER2) | 90 |
| NM_004889 | ATP synthase, H+ transporting, mitochondrial Fo complex, subunit F2 (ATP5J2) | 91 |
| NM_003769 | Serine/arginine-rich splicing factor 9 (SRSF9) | 92 |
| NM_003910 | BUD31 homolog (S. cerevisiae) (BUD31) | 93 |
| NM_000100 | Cystatin B (stefin B) (CSTB) | 94 |
| NM_004785 | Solute carrier family 9 (sodium/hydrogen exchanger), member 3 regulator 2 (SLC9A3R2) | 95 |
| NM_001120 | Major facilitator superfamily domain containing 10 (MFSD10) | 96 |
| NM_000182 | Hydroxyacyl-CoA dehydrogenase/3-ketoacyl-CoA thiolase/enoyl-CoA hydratase (HADHA) | 97 |
| NM_003377 | Vascular endothelial growth factor B (VEGFB) | 98 |
| NM_003576 | Serine/threonine kinase 24 (STK24) | 99 |
| NM_000918 | Prolyl 4-hydroxylase, beta polypeptide (P4HB), | 100 |
| NM_004584 | RAD9 homolog A (S. pombe) (RAD9A) | 101 |
| NM_004952 | Ephrin-A3 (EFNA3) | 102 |
| NM_004308 | Rho GTPase activating protein 1 (ARHGAP1) | 103 |
| NM_003190 | TAP binding protein (tapasin) (TAPBP) | 104 |
| NM_004640 | DEAD (Asp-Glu-Ala-Asp) box polypeptide 39B (DDX39B) | 105 |
| NM_001064 | Transketolase (TKT) | 106 |
| NM_002117 | Major histocompatibility complex, class I, C (HLA-C) | 107 |
| NM_004161 | RAB1A, member RAS oncogene family (RAB1A) | 108 |
| NM_003339 | Ubiquitin-conjugating enzyme E2D 2 (UBE2D2) | 109 |
| NM_003969 | Ubiquitin-conjugating enzyme E2M (UBE2M) | 110 |
| NM_000516 | GNAS complex locus (GNAS) | 111 |
| NM_002819 | Polypyrimidine tract binding protein 1 (PTBP1) | 112 |
| NM_001001 | Ribosomal protein L36a-like (RPL36AL) | 113 |
| NM_004649 | Chromosome 21 open reading frame 33 (C21orf33), nuclear gene encoding mitochondrial protein | 114 |
| NM_000175 | Glucose-6-phosphate isomerase (GPI) | 115 |
| NM_001867 | Cytochrome c oxidase subunit VIIc (COX7C) | 116 |
| NM_001967 | Eukaryotic translation initiation factor 4A2 (EIF4A2) | 117 |
| NM_001863 | Cytochrome c oxidase subunit VIb polypeptide 1 (ubiquitous) (COX6B1) | 118 |
| NM_001997 | Finkel-Biskis-Reilly murine sarcoma virus (FBR-MuSV) ubiquitously expressed (FAU) | 119 |
| NM_002088 | Glutamate receptor, ionotropic, kainate 5 (GRIK5) | 120 |
| NM_001862 | Cytochrome c oxidase subunit Vb (COX5B) | 121 |
| NM_004255 | Cytochrome c oxidase subunit Va (COX5A) | 122 |
| NM_001788 | Septin 7 (SEPT7) | 123 |
| NM_004781 | Vesicle-associated membrane protein 3 (cellubrevin) (VAMP3) | 124 |
| NM_003801 | Glycosylphosphatidylinositol anchor attachment protein 1 homolog (yeast) (GPAA1) | 125 |
| NM_004643 | Poly(A) binding protein, nuclear 1 (PABPN1) | 126 |
| NM_001537 | Heat shock factor binding protein 1 (HSBP1) | 127 |
| NM_003680 | Tyrosyl-tRNA synthetase (YARS) | 128 |
| NM_003345 | Ubiquitin-conjugating enzyme E2I (UBE2I) | 129 |
| NM_002568 | Poly(A) binding protein, cytoplasmic 1 (PABPC1) | 130 |
| NM_001487 | Biogenesis of lysosomal organelles complex-1, subunit 1 (BLOC1S1) | 131 |
| NM_001861 | Cytochrome c oxidase subunit IV isoform 1 (COX4I1) | 132 |
| NM_004890 | Sperm associated antigen 7 (SPAG7) | 133 |
| NM_002812 | Proteasome (prosome, macropain) 26S subunit, non-ATPase, 8 (PSMD8) | 134 |
| NM_004926 | Zinc finger protein 36, C3H type-like 1 (ZFP36L1) | 135 |
| NM_002539 | Ornithine decarboxylase 1 (ODC1) | 136 |
| NM_000979 | Ribosomal protein L18 (RPL18) | 137 |
| NM_000977 | Ribosomal protein L13 (RPL13) | 138 |
| NM_001015 | Ribosomal protein S11 (RPS11) | 139 |
| NM_001760 | Cyclin D3 (CCND3) | 140 |
| NM_003973 | Ribosomal protein L14 (RPL14) | 141 |
| NM_002815 | Proteasome (prosome, macropain) 26S subunit, non-ATPase, 11 (PSMD11) | 142 |
| NM_000367 | Thiopurine S-methyltransferase (TPMT | 143 |
| NM_000973 | Ribosomal protein L8 (RPL8) | 144 |
| NM_004689 | Metastasis associated 1 (MTA1), | 145 |
| NM_001848 | Collagen, type VI, alpha 1 (COL6A1) | 146 |
| NM_004068 | Adaptor-related protein complex 2, mu 1 subunit (AP2M1) | 147 |
| NM_001687 | ATP synthase, H+ transporting, mitochondrial F1 complex, delta subunit (ATP5D) | 148 |
| NM_004197 | Serine/threonine kinase 19 (STK19) | 149 |
| NM_001028 | Ribosomal protein S25 (RPS25) | 150 |
| NM_001022 | Ribosomal protein S19 (RPS19) | 151 |
| NM_004759 | Mitogen-activated protein kinase-activated protein kinase 2 (MAPKAPK2) | 152 |
| NM_001623 | Allograft inflammatory factor 1 (AIF1) | 153 |
| NM_004894 | Chromosome 14 open reading frame 2 (C14orf2) | 154 |
| NM_002375 | Microtubule-associated protein 4 (MAP4) | 155 |
| NM_001013 | Ribosomal protein S9 (RPS9) | 156 |
| NM_003779 | UDP-Gal:betaGlcNAc beta 1,4-galactosyltransferase, polypeptide 3 (B4GALT3) | 157 |
| NM_001296 | Chemokine binding protein 2 (CCBP2) | 158 |
| NM_001009 | Ribosomal protein S5 (RPS5) | 159 |
| NM_003021 | Small glutamine-rich tetratricopeptide repeat (TPR)-containing, alpha (SGTA) | 160 |
| NM_004285 | Hexose-6-phosphate dehydrogenase (glucose 1-dehydrogenase) (H6PD), | 161 |
| NM_004142 | Matrix metallopeptidase-like 1 (MMPL1) | 162 |
| NM_001950 | E2F transcription factor 4, p107/p130-binding (E2F4) | 163 |
| NM_003815 | ADAM metallopeptidase domain 15 (ADAM15) | 164 |
| NM_001119 | Adducin 1 (alpha) (ADD1) | 165 |
| NM_001111 | Adenosine deaminase, RNA-specific (ADAR) | 166 |
| NM_003466 | Paired box 8 (PAX8) | 167 |
| NM_001155 | Annexin A6 (ANXA6) | 168 |
| NM_003465 | Chitinase 1 (chitotriosidase) (CHIT1) | 169 |
| NM_003186 | Transgelin (TAGLN) | 170 |
| NM_000802 | Folate receptor 1 (adult) (FOLR1), | 171 |
| NM_004924 | Actinin, alpha 4 (ACTN4) | 172 |
| NM_002931 | Ring finger protein 1 (RING1) | 173 |
| NM_000020 | Activin A receptor type II-like 1 (ACVRL1), | 174 |
| NM_001785 | Cytidine deaminase (CDA) | 175 |
| NM_004339 | Pituitary tumor-transforming 1 interacting protein (PTTG1IP) | 176 |
| NM_003860 | Barrier to autointegration factor 1 (BANF1) | 177 |
| NM_000214 | Jagged 1 (JAG1) | 178 |
| NM_002167 | Inhibitor of DNA binding 3, dominant negative helix-loop-helix protein (ID3) | 179 |
| NM_001664 | Ras homolog gene family, member A (RHOA) | 180 |
| NM_003166 | Sulfotransferase family, cytosolic, 1A, phenol-preferring, member 3 (SULT1A3) | 181 |
| NM_001746 | Calnexin (CANX) | 182 |
| NM_001662 | ADP-ribosylation factor 5 (ARF5) | 183 |
| NM_001660 | ADP-ribosylation factor 4 (ARF4) | 184 |
| NM_001658 | ADP-ribosylation factor 1 (ARF1) | 185 |
| NM_003313 | Tissue specific transplantation antigen P35B (TSTA3) | 186 |
| NM_001494 | GDP dissociation inhibitor 2 (GDI2) | 187 |
| NM_003145 | Signal sequence receptor, beta (translocon-associated protein beta) (SSR2) | 188 |
| NM_001619 | Adrenergic, beta, receptor kinase 1 (ADRBK1) | 189 |
| NM_001420 | ELAV (embryonic lethal, abnormal vision, Drosophila)-like 3 (Hu antigen C) (ELAVL3) | 190 |
| NM_004930 | Capping protein (actin filament) muscle Z-line, beta (CAPZB) | 191 |
| NM_004596 | Small nuclear ribonucleoprotein polypeptide A (SNRPA) | 192 |
| NM_004168 | Succinate dehydrogenase complex, subunit A, flavoprotein (Fp) (SDHA) | 193 |
| NM_004156 | Protein phosphatase 2 (formerly 2A) | 194 |
| NM_004910 | Phosphatidylinositol transfer protein, membrane-associated 1 (PITPNM1) | 195 |
| NM_004517 | Integrin-linked kinase (ILK) | 196 |
| NM_004494 | Hepatoma-derived growth factor (HDGF) | 197 |
| NM_004121 | Gamma-glutamyltransferase 5 (GGT5) | 198 |
| NM_004404 | Septin 2 (SEPT2) | 199 |
| NM_004394 | Death-associated protein (DAP) | 200 |
| NM_004383 | c-src tyrosine kinase (CSK) | 201 |
| NM_004074 | Cytochrome c oxidase subunit VIIIA (ubiquitous) (COX8A) | 202 |
| NM_004039 | Annexin A2 (ANXA2) | 203 |
| NM_001053 | Somatostatin receptor 5 (SSTR5) | 204 |
| NM_001328 | C-terminal binding protein 1 (CTBP1) | 205 |
| NM_001273 | Chromodomain helicase DNA binding protein 4 (CHD4) | 206 |
| NM_003430 | Zinc finger protein 91 (ZNF91) | 207 |
| NM_003314 | Tetratricopeptide repeat domain 1 (TTC1) | 208 |
| NM_003217 | Transmembrane BAX inhibitor motif containing 6 (TMBIM6) | 209 |
| NM_003132 | Spermidine synthase (SRM) | 210 |
| NM_000199 | N-sulfoglucosamine sulfohydrolase (SGSH) | 211 |
| NM_002818 | Proteasome (prosome, macropain) activator subunit 2 (PA28 beta) (PSME2) | 212 |
| NM_002733 | Protein kinase, AMP-activated, gamma 1 non-catalytic subunit (PRKAG1) | 213 |
| NM_002631 | Phosphogluconate dehydrogenase (PGD) | 214 |
| NM_002574 | Peroxiredoxin 1 (PRDX1) | 215 |
| NM_002512 | Non-metastatic cells 2, protein (NM23B) expressed in (NME2) | 216 |
| NM_002455 | Metaxin 1 (MTX1) | 217 |
| NM_002444 | Moesin (MSN) | 218 |
| NM_000529 | Melanocortin 2 receptor (adrenocorticotropic hormone) (MC2R) | 219 |
| NM_003573 | latent transforming growth factor beta binding protein 4 (LTBP4) | 220 |
| NM_002315 | LIM domain only 1 (rhombotin 1) (LMO1) | 221 |
| NM_000884 | IMP (inosine 5′-monophosphate) dehydrogenase 2 (IMPDH2) | 222 |
| NM_003641 | Interferon induced transmembrane protein 1 (9-27) (IFITM1) | 223 |
| NM_000841 | Glutamate receptor, metabotropic 4 (GRM4) | 224 |
| NM_002070 | Guanine nucleotide binding protein (G protein), alpha inhibiting activity polypeptide 2 (GNAI2) | 225 |
| NM_001493 | GDP dissociation inhibitor 1 (GDI1) | 226 |
| NM_002048 | Growth arrest-specific 1 (GAS1) | 227 |
| NM_002032 | Ferritin, heavy polypeptide 1 (FTH1) | 228 |
| NM_001418 | Eukaryotic translation initiation factor 4 gamma, 2 (EIF4G2) | 229 |
| NM_001350 | Death-domain associated protein (DAXX) | 230 |
| NM_001843 | Contactin 1 (CNTN1) | 231 |
| NM_001728 | basigin (Ok blood group) (BSG) | 232 |
| NM_001667 | ADP-ribosylation factor-like 2 (ARL2) | 233 |
| NM_001659 | ADP-ribosylation factor 3 (ARF3) | 234 |
| NM_003746 | Dynein, light chain, LC8-type 1 (DYNLL1) | 235 |
| NM_002127 | Major histocompatibility complex, class I, G (HLA-G) | 236 |
| NM_004712 | Hepatocyte growth factor-regulated tyrosine kinase substrate (HGS) | 237 |
| NM_003475 | Ras association (RalGDS/AF-6) domain family (N-terminal) member 7 (RASSF7) | 238 |
| NM_004046 | ATP synthase, H+ transporting, mitochondrial F1 complex, alpha subunit 1, cardiac muscle (ATP5A1) | 239 |
| NM_001894 | Casein kinase 1, epsilon (CSNK1E) | 240 |
| NM_003795 | Sorting nexin 3 (SNX3) | 241 |
| NM_001909 | Cathepsin D (CTSD) | 242 |
| NM_002792 | Proteasome (prosome, macropain) subunit, alpha type, 7 (PSMA7) | 243 |
| NM_002799 | Proteasome (prosome, macropain) subunit, beta type, 7 (PSMB7) | 244 |
| NM_002300 | Lactate dehydrogenase B (LDHB) | 245 |
| NM_004176 | Sterol regulatory element binding transcription factor 1 (SREBF1) | 246 |
| NM_002796 | Proteasome (prosome, macropain) subunit, beta type, 4 (PSMB4) | 247 |
| NM_002794 | Proteasome (prosome, macropain) subunit, beta type, 2 (PSMB2) | 248 |
| NM_002793 | Proteasome (prosome, macropain) subunit, beta type, 1 (PSMB1) | 249 |
| NM_002473 | Myosin, heavy chain 9, non-muscle (MYH9) | 250 |
| NM_001810 | centromere protein B, 80 kDa (CENPB) | 251 |
| NM_002624 | Prefoldin subunit 5 (PFDN5) | 252 |
| NM_004710 | Synaptogyrin 2 (SYNGR2) | 253 |
| NM_001127 | Adaptor-related protein complex 1, beta 1 subunit (AP1B1) | 254 |
| NM_002107 | H3 histone, family 3A (H3F3A) | 255 |
| NM_003899 | Rho guanine nucleotide exchange factor (GEF) 7 (ARHGEF7) | 256 |
| NM_003406 | Tyrosine 3-monooxygenase/tryptophan 5-monooxygenase activation protein (YWHAZ) | 257 |
| NM_002419 | Mitogen-activated protein kinase kinase kinase 11 (MAP3K11) | 258 |
| NM_001130 | Amino-terminal enhancer of split (AES) | 259 |
| NM_003379 | Ezrin (EZR) | 260 |
| NM_002636 | PHD finger protein 1 (PHF1) | 261 |
| NM_002622 | Prefoldin subunit 1 (PFDN1) | 262 |
| NM_001823 | Creatine kinase, brain (CKB) | 263 |
| NM_003405 | Tyrosine 3-monooxygenase/tryptophan 5-monooxygenase activation protein (YWHAH) | 264 |
| NM_002939 | Ribonuclease/angiogenin inhibitor 1 (RNH1) | 265 |
| NM_003562 | Solute carrier family 25 (mitochondrial carrier; oxoglutarate carrier), member 11 (SLC25A11) | 266 |
| NM_001916 | Cytochrome c-1 (CYC1) | 267 |
| NM_002823 | Prothymosin, alpha (PTMA) | 268 |
| NM_003096 | Small nuclear ribonucleoprotein polypeptide G (SNRPG) | 269 |
| NM_003321 | Tu translation elongation factor, mitochondrial (TUFM) | 270 |
| NM_003404 | Tyrosine 3-monooxygenase/tryptophan 5-monooxygenase activation protein (YWHAB) | 271 |
| NM_002946 | Replication protein A2, 32 kDa (RPA2) | 272 |
| NM_004356 | CD81 molecule (CD81) | 273 |
| NM_001743 | Calmodulin 2 (phosphorylase kinase, delta) (CALM2) | 274 |
| NM_004231 | ATPase, H+ transporting, lysosomal 14 kDa, V1 subunit F (ATP6V1F) | 275 |
| NM_004893 | H2A histone family, member Y (H2AFY) | 276 |
| NM_004146 | NADH dehydrogenase (ubiquinone) 1 beta subcomplex, 7, 18 kDa (NDUFB7) | 277 |
| NM_002128 | High mobility group box 1 (HMGB1) | 278 |
| NM_002002 | Fc fragment of IgE, low affinity II, receptor for (CD23) (FCER2) | 279 |
| NM_000858 | Guanylate kinase 1 (GUK1) | 280 |
| NM_001469 | X-ray repair complementing defective repair in Chinese hamster cells 6 (XRCC6) | 281 |
| NM_003766 | Beclin 1, autophagy related (BECN1) | 282 |
| NM_003906 | Minichromosome maintenance complex component 3 associated protein (MCM3AP) | 283 |
| NM_000757 | Colony stimulating factor 1 (macrophage) (CSF1) | 284 |
| NM_002149 | Hippocalcin-like 1 (HPCAL1) | 285 |
| NM_001694 | H+ transporting, lysosomal 16 kDa, V0 subunit c (ATP6V0C) | 286 |
| NM_004047 | H+ transporting, lysosomal 21 kDa, V0 subunit b (ATP6V0B) | 287 |
| NM_001696 | ATPase, H+ transporting, lysosomal 31 kDa, V1 subunit E1 (ATP6V1E1) | 288 |
| NM_001865 | Cytochrome c oxidase subunit VIIa polypeptide 2 (liver) (COX7A2) | 289 |
| NM_004373 | Cytochrome c oxidase subunit VIa polypeptide 1 (COX6A1) | 290 |
| NM_000801 | FK506 binding protein 1A | 291 |
| NM_000992 | Ribosomal protein L29 (RPL29) | 292 |
| NM_000988 | Ribosomal protein L27 (RPL27) | 293 |
| NM_001004 | ribosomal protein, large, P2 (RPLP2) | 294 |
| NM_001003 | Ribosomal protein, large, P1 (RPLP1) | 295 |
| NM_000405 | GM2 ganglioside activator (GM2A) | 296 |
| NM_000967 | Ribosomal protein L3 (RPL3) | 297 |
| NM_001428 | Enolase 1, (alpha) (ENO1) | 298 |
| NM_000999 | Ribosomal protein L38 (RPL38) | 299 |
| NM_000997 | Ribosomal protein L37 (RPL37) | 300 |
| NM_000995 | Ribosomal protein L34 (RPL34) | 301 |
| NM_002948 | Ribosomal protein L15 (RPL15) | 302 |
| NM_002952 | Ribosomal protein S2 (RPS2) | 303 |
| NM_001026 | Ribosomal protein S24 (RPS24) | 304 |
| NM_001020 | Ribosomal protein S16 (RPS16) | 305 |
| NM_001018 | Ribosomal protein S15 (RPS15) | 306 |
| NM_001017 | Ribosomal protein S13 (RPS13) | 307 |
| NM_000969 | Ribosomal protein L5 (RPL5) | 308 |
| NM_000985 | Ribosomal protein L17 (RPL17) | 309 |
| NM_000937 | Polymerase (RNA) II (DNA directed) polypeptide A, 220 kDa (POLR2A) | 310 |
| NM_001016 | Ribosomal protein S12 (RPS12) | 311 |
| NM_002140 | Heterogeneous nuclear ribonucleoprotein K (HNRNPK) | 312 |
| NM_002138 | Heterogeneous nuclear ribonucleoprotein D (AU-rich element RNA binding protein 1, 37 kDa) | 313 |
| | (HNRNPD) | |
| NM_004499 | Heterogeneous nuclear ribonucleoprotein A/B (HNRNPAB) | 314 |
| NM_001014 | Ribosomal protein S10 (RPS10) | 315 |
| NM_002383 | MYC-associated zinc finger protein (purine-binding transcription factor) (MAZ) | 316 |
| NM_002467 | V-myc myelocytomatosis viral oncogene homolog (avian) (MYC) | 317 |
| NM_001436 | Fibrillarin (FBL) | 318 |
| NM_004069 | Adaptor-related protein complex 2, sigma 1 subunit (AP2S1) | 319 |
| NM_001614 | Actin, gamma 1 (ACTG1) | 320 |
| NM_002355 | Mannose-6-phosphate receptor (cation dependent) (M6PR) | 321 |
| NM_004597 | Small nuclear ribonucleoprotein D2 polypeptide 16.5 kDa (SNRPD2) | 322 |
| NM_002308 | Lectin, galactoside-binding, soluble, 9 (LGALS9) | 323 |
| NM_000398 | Cytochrome b5 reductase 3 (CYB5R3) | 324 |
| NM_000754 | Catechol-O-methyltransferase (COMT) | 325 |
| NM_002406 | Mannosyl (alpha-1,3-)-glycoprotein beta-1,2-N-acetylglucosaminyltransferase (MGAT1) | 326 |
| NM_003752 | Eukaryotic translation initiation factor 3, subunit C (EIF3C) | 327 |
| NM_001355 | D-dopachrome tautomerase (DDT) | 328 |
| NM_004960 | Fused in sarcoma (FUS) | 329 |
| NM_004729 | Zinc finger, BED-type containing 1 (ZBED1) | 330 |
| NM_004587 | Ribosome binding protein 1 homolog 180 kDa (dog) (RRBP1) | 331 |
| NM_004552 | NADH dehydrogenase (ubiquinone) Fe—S protein 5, 15 kDa (NADH-coenzyme Q reductase) | 332 |
| | (NDUFS5) | |
| NM_004450 | Enhancer of rudimentary homolog (Drosophila) (ERH) | 333 |
| NM_004048 | Beta-2-microglobulin (B2M) | 334 |
| NM_000239 | Lysozyme (LYZ) | 335 |
| NM_000269 | Non-metastatic cells 1, protein (NM23A) expressed in (NME1) | 336 |
| NM_000431 | Mevalonate kinase (MVK) | 337 |
| NM_001247 | Ectonucleoside triphosphate diphosphohydrolase 6 (putative) (ENTPD6) | 338 |
| NM_003365 | Ubiquinol-cytochrome c reductase core protein I (UQCRC1) | 339 |
| NM_003329 | Thioredoxin (TXN) | 340 |
| NM_001069 | Tubulin, beta 2A class IIa (TUBB2A) | 341 |
| NM_000356 | Treacher Collins-Franceschetti syndrome 1 (TCOF1) | 342 |
| NM_003134 | Signal recognition particle 14 kDa (homologous Alu RNA binding protein) (SRP14) | 343 |
| NM_003131 | Serum response factor (c-fos serum response element-binding transcription factor) (SRF) | 344 |
| NM_000454 | Superoxide dismutase 1 | 345 |
| NM_003091 | Small nuclear ribonucleoprotein polypeptides B and B1 (SNRPB) | 346 |
| NM_003089 | Small nuclear ribonucleoprotein 70 kDa (U1) (SNRNP70) | 347 |
| NM_003016 | Serine/arginine-rich splicing factor 2 (SRSF2) | 348 |
| NM_003952 | Ribosomal protein S6 kinase, 70 kDa, polypeptide 2 (RPS6KB2) | 349 |
| NM_002950 | Ribophorin I (RPN1) | 350 |
| NM_002743 | Protein kinase C substrate 80K-H (PRKCSH) | 351 |
| NM_002686 | Phenylethanolamine N-methyltransferase (PNMT) | 352 |
| NM_002654 | Pyruvate kinase, muscle (PKM2) | 353 |
| NM_002648 | Pim-1 oncogene (PIM1) | 354 |
| NM_002635 | solute carrier family 25 (mitochondrial carrier; phosphate carrier), member 3 (SLC25A3) | 355 |
| NM_002494 | NADH dehydrogenase (ubiquinone) 1, subcomplex unknown, 1, 6 kDa (NDUFC1) | 356 |
| NM_002488 | NADH dehydrogenase (ubiquinone) 1 alpha subcomplex, 2, 8 kDa (NDUFA2) | 357 |
| NM_002434 | N-methylpurine-DNA glycosylase (MPG) | 358 |
| NM_002415 | Macrophage migration inhibitory factor (glycosylation-inhibiting factor) (MIF) | 359 |
| NM_002227 | Janus kinase 1 (JAK1) | 360 |
| NM_001536 | Arginine methyltransferase 1 (PRMT1) | 361 |
| NM_000183 | Hydroxyacyl-CoA dehydrogenase/3-ketoacyl-CoA thiolase/enoyl-CoA hydratase (HADHB) | 362 |
| NM_002085 | Glutathione peroxidase 4 (phospholipid hydroperoxidase) (GPX4) | 363 |
| NM_001502 | Glycoprotein 2 (zymogen granule membrane) (GP2) | 364 |
| NM_002080 | Glutamic-oxaloacetic transaminase 2, mitochondrial (aspartate aminotransferase 2) (GOT2) | 365 |
| NM_001440 | Exostoses (multiple)-like 3 (EXTL3) | 366 |
| NM_003754 | Eukaryotic translation initiation factor 3, subunit F (EIF3F) | 367 |
| NM_003755 | Eukaryotic translation initiation factor 3, subunit G (EIF3G) | 368 |
| NM_003757 | Eukaryotic translation initiation factor 3, subunit I (EIF3I) | 369 |
| NM_001360 | 7-dehydrocholesterol reductase (DHCR7) | 370 |
| NM_001344 | Defender against cell death 1 (DAD1) | 371 |
| NM_001914 | Cytochrome b5 type A (microsomal) (CYB5A) | 372 |
| NM_001834 | Clathrin, light chain B (CLTB) | 373 |
| NM_001833 | Clathrin, light chain A (CLTA) | 374 |
| NM_001281 | Tubulin folding cofactor B (TBCB) | 375 |
| NM_001749 | Calpain, small subunit 1 (CAPNS1) | 376 |
| NM_001697 | ATP synthase, H+ transporting, mitochondrial F1 complex, O subunit (ATP5O) | 377 |
| NM_001689 | ATP synthase, H+ transporting, mitochondrial Fo complex, subunit C3 (subunit 9) (ATP5G3) | 378 |
| NM_001675 | Activating transcription factor 4 (tax-responsive enhancer element B67) (ATF4) | 379 |
| NM_001642 | Amyloid beta (A4) precursor-like protein 2 (APLP2) | 380 |
| NM_014724 | Zinc finger protein 96 (ZNF96) | 381 |
| NM_005494 | DnaJ (Hsp40) homolog, subfamily B, member 6 (DNAJB6) | 382 |
| NM_006597 | Heat shock 70 kDa protein 8 (HSPA8) | 383 |
| NM_006623 | Phosphoglycerate dehydrogenase (PHGDH) | 384 |
| NM_015646 | RAP1B, member of RAS oncogene family (RAP1B) | 385 |
| NM_016532 | Inositol polyphosphate-5-phosphatase K (INPP5K) | 386 |
| NM_015292 | extended synaptotagmin-like protein 1 (ESYT1) | 387 |
| NM_005870 | Sin3A-associated protein, 18 kDa (SAP18) | 388 |
| NM_006833 | COP9 constitutive photomorphogenic homolog subunit 6 (Arabidopsis) (COPS6) | 389 |
| NM_005718 | Actin related protein 2/3 complex, subunit 4, 20 kDa (ARPC4) | 390 |
| NM_005719 | Actin related protein 2/3 complex, subunit 3, 21 kDa (ARPC3) | 391 |
| NM_006372 | Synaptotagmin binding, cytoplasmic RNA interacting protein (SYNCRIP) | 392 |
| NM_005180 | BMI1 polycomb ring finger oncogene (BMI1) | 393 |
| NM_018975 | Telomeric repeat binding factor 2, interacting protein (TERF2IP) | 394 |
| NM_005731 | Actin related protein 2/3 complex, subunit 2, 34 kDa (ARPC2) | 395 |
| NM_020151 | StAR-related lipid transfer (START) domain containing 7 (STARD7) | 396 |
| NM_005103 | Fasciculation and elongation protein zeta 1 (zygin I) (FEZ1) | 397 |
| NM_012179 | F-box protein 7 (FBXO7) | 398 |
| NM_020360 | phospholipid scramblase 3 (PLSCR3) | 399 |
| NM_014891 | PDGFA associated protein 1 (PDAP1) | 400 |
| NM_005745 | B-cell receptor-associated protein 31 (BCAP31) | 401 |
| NM_005418 | suppression of tumorigenicity 5 (ST5) | 402 |
| NM_006262 | Peripherin (PRPH) | 403 |
| NM_133476 | Zinc finger protein 384 (ZNF384) | 404 |
| NM_006570 | Ras-related GTP binding A (RRAGA) | 405 |
| NM_006333 | C1D nuclear receptor corepressor (C1D) | 406 |
| NM_007285 | GABA(A) receptor-associated protein-like 2 (GABARAPL2) | 407 |
| NM_006354 | Transcriptional adaptor 3 (TADA3) | 408 |
| NM_014302 | Sec61 gamma subunit (SEC61G) | 409 |
| NM_006118 | HCLS1 associated protein X-1 (HAX1) | 410 |
| NM_012100 | Aspartyl aminopeptidase (DNPEP) | 411 |
| NM_015680 | Cyclin Pas1/PHO80 domain containing 1 (CNPPD1) | 412 |
| NM_030796 | Vesicular, overexpressed in cancer, prosurvival protein 1 (VOPP1) | 413 |
| NM_024069 | KxDL motif containing 1 (KXD1) | 414 |
| NM_013234 | Eukaryotic translation initiation factor 3, subunit K (EIF3K) | 415 |
| NM_013310 | Chromosome 2 open reading frame 27A (C2orf27A) | 416 |
| NM_018507 | Hypothetical protein PRO1843 (PRO1843) | 417 |
| NM_017670 | OTU domain, ubiquitin aldehyde binding 1 (OTUB1) | 418 |
| NM_016292 | TNF receptor-associated protein 1 (TRAP1) | 419 |
| NM_014916 | Lemur tyrosine kinase 2 (LMTK2) | 420 |
| NM_014696 | G protein regulated inducer of neurite outgrowth 2 (GPRIN2) | 421 |
| NM_014630 | Zinc finger protein 592 (ZNF592) | 422 |
| NM_014761 | Increased sodium tolerance 1 homolog (yeast) (IST1) | 423 |
| NM_007286 | Synaptopodin (SYNPO) | 424 |
| NM_007263 | Coatomer protein complex, subunit epsilon (COPE) | 425 |
| NM_006349 | Zinc finger, HIT-type containing 1 (ZNHIT1) | 426 |
| NM_006004 | Ubiquinol-cytochrome c reductase hinge protein (UQCRH) | 427 |
| NM_005787 | Asparagine-linked glycosylation 3, alpha-1,3-mannosyltransferase homolog (S. cerevisiae) (ALG3) | 428 |
| NM_012412 | H2A histone family, member V (H2AFV) | 429 |
| NM_012401 | Plexin B2 (PLXNB2) | 430 |
| NM_007262 | Parkinson protein 7 (PARK7) | 431 |
| NM_007273 | Prohibitin 2 (PHB2) | 432 |
| NM_021009 | Ubiquitin C (UBC) | 433 |
| NM_023009 | MARCKS-like 1 (MARCKSL1) | 434 |
| NM_015456 | Cofactor of BRCA1 (COBRA1) | 435 |
| NM_005053 | RAD23 homolog A (S. cerevisiae) (RAD23A) | 436 |
| NM_006830 | Ubiquinol-cytochrome c reductase, complex III subunit XI (UQCR11), | 437 |
| NM_005682 | G protein-coupled receptor 56 (GPR56) | 438 |
| NM_012102 | Arginine-glutamic acid dipeptide (RE) repeats (RERE) | 439 |
| NM_005550 | Kinesin family member C3 (KIFC3) | 440 |
| NM_021960 | Myeloid cell leukemia sequence 1 (BCL2-related) (MCL1) | 441 |
| NM_021959 | Protein phosphatase 1, regulatory (inhibitor) subunit 11 (PPP1R11) | 442 |
| NM_014730 | Malectin (MLEC) | 443 |
| NM_014402 | Ubiquinol-cytochrome c reductase, complex III subunit VII, 9.5 kDa (UQCRQ) | 444 |
| NM_007067 | K(lysine) acetyltransferase 7 (KAT7) | 445 |
| NM_006086 | Tubulin, beta 3 class III (TUBB3) | 446 |
| NM_014972 | Transcription factor 25 (basic helix-loop-helix) (TCF25) | 447 |
| NM_024092 | Transmembrane protein 109 (TMEM109) | 448 |
| NM_021983 | Major histocompatibility complex, class II, DR beta 4 (HLA-DRB4) | 449 |
| NM_006510 | Tripartite motif containing 27 (TRIM27) | 450 |
| NM_006711 | RNA binding protein S1, serine-rich domain (RNPS1) | 451 |
| NM_006145 | DnaJ (Hsp40) homolog, subfamily B, member 1 (DNAJB1) | 452 |
| NM_033142 | Chorionic gonadotropin, beta polypeptide 7 (CGB7) | 453 |
| NM_006351 | Translocase of inner mitochondrial membrane 44 homolog (yeast) (TIMM44) | 454 |
| NM_014281 | Poly-U binding splicing factor 60 KDa (PUF60) | 455 |
| NM_012106 | ADP-ribosylation factor-like 2 binding protein (ARL2BP) | 456 |
| NM_021975 | V-rel reticuloendotheliosis viral oncogene homolog A (avian) (RELA), | 457 |
| NM_014874 | Mitofusin 2 (MFN2) | 458 |
| NM_006796 | AFG3 ATPase family gene 3-like 2 (S. cerevisiae)(AFG3L2) | 459 |
| NM_006666 | RuvB-like 2 (E. coli) (RUVBL2) | 460 |
| NM_005219 | Diaphanous homolog 1 (Drosophila) (DIAPH1) | 461 |
| NM_033546 | Myosin, light chain 12B, regulatory (MYL12B) | 462 |
| NM_032348 | Matrix-remodelling associated 8 (MXRA8) | 463 |
| NM_024798 | Sorting nexin 22 (SNX22) | 464 |
| NM_021103 | Thymosin beta 10 (TMSB10) | 465 |
| NM_020195 | Short chain dehydrogenase/reductase family 39U, member 1 (SDR39U1) | 466 |
| NM_017432 | Prostate tumor overexpressed 1 (PTOV1) | 467 |
| NM_014901 | Ring finger protein 44 (RNF44) | 468 |
| NM_014694 | ADAMTS-like 2 (ADAMTSL2) | 469 |
| NM_007369 | G protein-coupled receptor 161 (GPR161) | 470 |
| NM_006156 | Neural precursor cell expressed, developmentally down-regulated 8 (NEDD8) | 471 |
| NM_006429 | chaperonin containing TCP1, subunit 7 (eta) (CCT7) | 472 |
| NM_006513 | Seryl-tRNA synthetase (SARS) | 473 |
| NM_005022 | Profilin 1 (PFN1) | 474 |
| NM_014654 | Syndecan 3 (SDC3) | 475 |
| NM_007209 | Ribosomal protein L35 (RPL35) | 476 |
| NM_006082 | Tubulin, alpha 1b (TUBA1B) | 477 |
| NM_006362 | Nuclear RNA export factor 1 (NXF1) | 478 |
| NM_014228 | Solute carrier family 6 (neurotransmitter transporter, L-proline), member 7 (SLC6A7) | 479 |
| NM_006411 | 1-acylglycerol-3-phosphate O-acyltransferase 1(AGPAT1) | 480 |
| NM_021134 | Mitochondrial ribosomal protein L23 (MRPL23) | 481 |
| NM_021974 | Polymerase (RNA) II (DNA directed) polypeptide F (POLR2F) | 482 |
| NM_006808 | Sec61 beta subunit (SEC61B) | 483 |
| NM_005617 | Ribosomal protein S14 (RPS14) | 484 |
| NM_005520 | Heterogeneous nuclear ribonucleoprotein H1 (H) (HNRNPH1) | 485 |
| NM_006755 | Transaldolase 1 (TALDO1) | 486 |
| NM_006010 | Mesencephalic astrocyte-derived neurotrophic factor (MANF) | 487 |
| NM_005088 | A kinase (PRKA) anchor protein 17A (AKAP17A) | 488 |
| NM_014754 | Phosphatidylserine synthase 1 (PTDSS1) | 489 |
| NM_021953 | Forkhead box M1 (FOXM1), | 490 |
| NM_006908 | RAS-related C3 botulinum toxin substrate 1 (rho family, small GTP binding protein Rac1) (RAC1) | 491 |
| NM_014231 | Vesicle-associated membrane protein 1 (synaptobrevin 1) (VAMP1) | 492 |
| NM_014833 | KIAA0618 gene product (KIAA0618) | 493 |
| NM_005157 | c-abl oncogene 1, non-receptor tyrosine kinase (ABL1) | 494 |
| NM_006325 | RAN, member RAS oncogene family (RAN) | 495 |
| NM_007245 | Ataxin 2-like (ATXN2L) | 496 |
| NM_007008 | Reticulon 4 (RTN4) | 497 |
| NM_006782 | Zinc finger protein-like 1 (ZFPL1) | 498 |
| NM_006694 | Jumping translocation breakpoint (JTB) | 499 |
| NM_006703 | Nudix (nucleoside diphosphate linked moiety X)-type motif 3 (NUDT3), | 500 |
| NM_006032 | Copine VI (neuronal) (CPNE6) | 501 |
| NM_012227 | GTP binding protein 6 (putative) (GTPBP6) | 502 |
| NM_014604 | Tax1 (human T-cell leukemia virus type I) binding protein 3 (TAX1BP3), | 503 |
| NM_021642 | Fc fragment of IgG, low affinity IIa, receptor (CD32) (FCGR2A) | 504 |
| NM_005354 | Jun D proto-oncogene (JUND), | 505 |
| NM_020529 | Nuclear factor of kappa light polypeptide gene enhancer in B-cells inhibitor, alpha (NFKBIA) | 506 |
| NM_005561 | Lysosomal-associated membrane protein 1 (LAMP1) | 507 |
| NM_014774 | KIAA0494 | 508 |
| NM_014390 | Staphylococcal nuclease and tudor domain containing 1 (SND1) | 509 |
| NM_014623 | Male-enhanced antigen 1 (MEA1) | 510 |
| NM_014453 | Charged multivesicular body protein 2A (CHMP2A) | 511 |
| NM_012127 | CDKN1A interacting zinc finger protein 1 (CIZ1) | 512 |
| NM_012099 | CD3e molecule, epsilon associated protein (CD3EAP) | 513 |
| NM_006888 | Calmodulin 1 (phosphorylase kinase, delta) (CALM1) | 514 |
| NM_006867 | RNA binding protein with multiple splicing (RBPMS) | 515 |
| NM_006743 | Complement component 1, q subcomponent-like 1 (C1QL1) | 516 |
| NM_006688 | Tubulin, gamma complex associated protein 2 (TUBGCP2) | 517 |
| NM_006295 | Valyl-tRNA synthetase (VARS) | 518 |
| NM_006283 | Transforming, acidic coiled-coil containing protein 1 (TACC1) | 519 |
| NM_006221 | Peptidylprolyl cis/trans isomerase, NIMA-interacting 1 (PIN1) | 520 |
| NM_006148 | LIM and SH3 protein 1 (LASP1) | 521 |
| NM_005954 | Metallothionein 3 (MT3) | 522 |
| NM_006003 | Ubiquinol-cytochrome c reductase, Rieske iron-sulfur polypeptide 1 (UQCRFS1) | 523 |
| NM_005998 | Chaperonin containing TCP1, subunit 3 (gamma) (CCT3) | 524 |
| NM_005997 | Vacuolar protein sorting 72 homolog (S. cerevisiae) (VPS72) | 525 |
| NM_005629 | Solute carrier family 6 (neurotransmitter transporter, creatine) | 526 |
| NM_005548 | Lysyl-tRNA synthetase (KARS) | 527 |
| NM_005545 | Immunoglobulin superfamily containing leucine-rich repeat (ISLR) | 528 |
| NM_005507 | Cofilin 1 (non-muscle) (CFL1) | 529 |
| NM_005381 | Nucleolin (NCL) | 530 |
| NM_005439 | Myeloid leukemia factor 2 (MLF2) | 541 |
| NM_006445 | PRP8 pre-mRNA processing factor 8 homolog (S. cerevisiae) (PRPF8) | 542 |
| NM_019059 | translocase of outer mitochondrial membrane 7 homolog (yeast) (TOMM7) | 543 |
| NM_006039 | Mannose receptor, C type 2 (MRC2) | 544 |
| NM_006066 | Aldo-keto reductase family 1, member A1 (aldehyde reductase) (AKR1A1) | 545 |
| NM_013318 | Proline-rich coiled-coil 2B (PRRC2B) | 546 |
| NM_006098 | Guanine nucleotide binding protein (G protein), beta polypeptide 2-like 1 (GNB2L1), | 547 |
| NM_007359 | Cancer susceptibility candidate 3 (CASC3) | 548 |
| NM_017510 | Transmembrane emp24 protein transport domain containing 9 (TMED9), | 549 |
| NM_015399 | Breast cancer metastasis suppressor 1 (BRMS1) | 550 |
| NM_014508 | Apolipoprotein B mRNA editing enzyme, catalytic polypeptide-like 3C (APOBEC3C) | 551 |
| NM_018955 | Ubiquitin B (UBB) | 552 |
| NM_006368 | cAMP responsive element binding protein 3 (CREB3) | 553 |
| NM_015024 | Exportin 7 (XPO7) | 554 |
| NM_031420 | Mitochondrial ribosomal protein L9 (MRPL9) | 555 |
| NM_013232 | Programmed cell death 6 (PDCD6) | 556 |
| NM_005917 | Malate dehydrogenase 1, NAD (soluble) (MDH1) | 557 |
| NM_032801 | Junctional adhesion molecule 3 (JAM3) | 558 |
| NM_030662 | Mitogen-activated protein kinase kinase 2 (MAP2K2) | 559 |
| NM_006268 | D4, zinc and double PHD fingers family 2 (DPF2) | 560 |
| NM_006826 | Tyrosine 3-monooxygenase/tryptophan 5-monooxygenase activation protein (YWHAQ) | 561 |
| NM_144582 | Testis expressed 261 (TEX261) | 562 |
| NM_144565 | Dual oxidase maturation factor 1 (DUOXA1) | 563 |
| NM_005984 | Solute carrier family 25 (mitochondrial carrier; citrate transporter), member 1 (SLC25A1) | 564 |
| NM_017828 | COMM domain containing 4 (COMMD4) | 565 |
| NM_020150 | SAR1 homolog A (S. cerevisiae) (SAR1A) | 566 |
| NM_014610 | Glucosidase, alpha; neutral AB (GANAB) | 567 |
| NM_014225 | Protein phosphatase 2, regulatory subunit A, alpha (PPP2R1A) | 568 |
| NM_006937 | SMT3 suppressor of mif two 3 homolog 2 (S. cerevisiae) (SUMO2) | 569 |
| NM_005726 | Ts translation elongation factor, mitochondrial (TSFM) | 570 |
| NM_005347 | Heat shock 70 kDa protein 5 (glucose-regulated protein, 78 kDa) (HSPA5) | 571 |
| NM_014255 | Canopy 2 homolog (zebrafish) (CNPY2) | 572 |
| NM_006815 | KDEL (Lys-Asp-Glu-Leu) endoplasmic reticulum protein retention receptor 1 (KDELR1) | 573 |
| NM_005456 | Mitogen-activated protein kinase 8 interacting protein | 574 |
| NM_005273 | Guanine nucleotide binding protein (G protein), beta polypeptide 2 (GNB2) | 575 |
| NM_007260 | Lysophospholipase II (LYPLA2) | 576 |
| NM_007103 | NADH dehydrogenase (ubiquinone) flavoprotein 1, 51 kDa (NDUFV1) | 577 |
| NM_017797 | BTB (POZ) domain containing 2 (BTBD2) | 578 |
| NM_016237 | Anaphase promoting complex subunit 5 (ANAPC5) | 579 |
| NM_005801 | Eukaryotic translation initiation factor 1 (EIF1) | 580 |
| NM_005216 | Dolichyl-diphosphooligosaccharide-protein glycosyltransferase (DDOST) | 581 |
| NM_016457 | Protein kinase D2 (PRKD2) | 582 |
| NM_006442 | DR1-associated protein 1 (negative cofactor 2 alpha) (DRAP1) | 583 |
| NM_021019 | Myosin, light chain 6, alkali, smooth muscle and non-muscle (MYL6) | 584 |
| NM_005112 | WD repeat domain 1 (WDR1) | 585 |
| NM_005370 | RAB8A, member RAS oncogene family (RAB8A) | 586 |
| NM_006289 | Talin 1 (TLN1) | 587 |
| NM_005698 | Secretory carrier membrane protein 3 (SCAMP3) | 588 |
| NM_024011 | Cyclin-dependent kinase 11A (CDK11A) | 589 |
| NM_005105 | RNA binding motif protein 8A (RBM8A) | 590 |
| NM_006013 | Ribosomal protein L10 (RPL10) | 591 |
| NM_005786 | Teashirt zinc finger homeobox 1 (TSHZ1) | 592 |
| NM_007104 | Ribosomal protein L10a (RPL10A) | 593 |
| NM_005762 | Tripartite motif containing 28 (TRIM28) | 594 |
| NM_012138 | Apoptosis antagonizing transcription factor (AATF) | 595 |
| NM_015318 | Rho/Rac guanine nucleotide exchange factor (GEF) 18 (ARHGEF18) | 596 |
| NM_012423 | Ribosomal protein L13a (RPL13A) | 597 |
| NM_021128 | Polymerase (RNA) II (DNA directed) polypeptide L, 7.6 kDa (POLR2L) | 598 |
| NM_032635 | Transmembrane protein 147 (TMEM147) | 599 |
| NM_005080 | X-box binding protein 1 (XBP1) | 600 |
| NM_006389 | Hypoxia up-regulated 1 (HYOU1) | 601 |
| NM_024112 | Chromosome 9 open reading frame 16 (C9orf16) | 602 |
| NM_006817 | Transmembrane emp24 domain trafficking protein 2 (TMED2) | 603 |
| NM_022830 | Terminal uridylyl transferase 1, U6 snRNA-specific (TUT1) | 604 |
| NM_019884 | Glycogen synthase kinase 3 alpha (GSK3A) | 605 |
| NM_021107 | Mitochondrial ribosomal protein S12 (MRPS12) | 606 |
| NM_021074 | NADH dehydrogenase (ubiquinone) flavoprotein 2, 24 kDa (NDUFV2) | 607 |
| NM_014944 | Calsyntenin 1 (CLSTN1) | 608 |
| NM_014764 | DAZ associated protein 2 (DAZAP2) | 609 |
| NM_015343 | CTD nuclear envelope phosphatase 1 (CTDNEP1) | 610 |
| NM_014420 | Dickkopf homolog 4 (Xenopus laevis) (DKK4) | 611 |
| NM_005884 | p21 protein (Cdc42/Rac)-activated kinase 4 (PAK4) | 612 |
| NM_012407 | Protein interacting with PRKCA 1 (PICK1) | 613 |
| NM_012111 | AHA1, activator of heat shock 90 kDa protein ATPase homolog 1 (yeast) (AHSA1) | 614 |
| NM_007144 | Polycomb group ring finger 2 (PCGF2) | 615 |
| NM_007108 | Transcription elongation factor B (SIII), polypeptide 2 (18 kDa, elongin B) (TCEB2) | 616 |
| NM_007278 | GABA(A) receptor-associated protein (GABARAP) | 617 |
| NM_007100 | ATP synthase, H+ transporting, mitochondrial Fo complex, subunit E (ATP5I) | 618 |
| NM_006936 | SMT3 suppressor of mif two 3 homolog 3 (S. cerevisiae) (SUMO3) | 619 |
| NM_006899 | Isocitrate dehydrogenase 3 (NAD+) beta (IDH3B), nuclear gene encoding mitochondrial protein | 620 |
| NM_006801 | RNA binding motif (RNP1, RRM) protein 3 (RBM3) | 621 |
| NM_006612 | Kinesin family member 1C (KIF1C) | 622 |
| NM_006659 | Tubulin, gamma complex associated protein 2 (TUBGCP2) | 623 |
| NM_006595 | Apoptosis inhibitor 5 (API5) | 624 |
| NM_006401 | Acidic (leucine-rich) nuclear phosphoprotein 32 family, member B (ANP32B), | 625 |
| NM_006423 | Rab acceptor 1 (prenylated) (RABAC1) | 626 |
| NM_006460 | Hexamethylene bis-acetamide inducible 1 (HEXIM1) | 627 |
| NM_006356 | ATP synthase, H+ transporting, mitochondrial Fo complex, subunit d (ATP5H) | 628 |
| NM_005891 | Acetyl-CoA acetyltransferase 2 (ACAT2) | 629 |
| NM_005839 | Serine/arginine repetitive matrix 1 (SRRM1) | 630 |
| NM_005594 | Nascent polypeptide-associated complex alpha subunit (NACA) | 631 |
| NM_005340 | Histidine triad nucleotide binding protein 1 (HINT1) | 632 |
| NM_005175 | ATP synthase, H+ transporting, mitochondrial Fo complex, subunit C1 (subunit 9) (ATP5G1) | 633 |
| NM_005165 | Aldolase C, fructose-bisphosphate (ALDOC) | 634 |
| NM_005001 | NADH dehydrogenase (ubiquinone) 1 alpha subcomplex, 7, 14.5 kDa (NDUFA7) | 635 |

Routes of Administration

The following discussion on routes of administration is merely provided to illustrate exemplary embodiments and should not be construed as limiting the scope in any way.

Formulations suitable for oral administration can consist of (a) liquid solutions, such as an effective amount of the analog of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the analog of the present disclosure in a flavor, usually sucrose and acacia or tragacanth, as well as pastilles comprising the analog of the present disclosure in an inert base, such as gelatin and glycerin, or sucrose and acacia, emulsions, gels, and the like containing, in addition to, such excipients as are known in the art.

The analogs of the disclosure, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the analog is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The analog of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the analog of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the analog of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the analog of the disclosure can be formulated as inclusion complexes, such as cyclodextrin inclusion complexes, or liposomes.

Treatment and Prevention

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention in a mammal. Furthermore, the treatment or prevention can include treatment or prevention of one or more conditions or symptoms of the disease being treated or prevented. Also, for purposes herein, “prevention” can encompass delaying the onset of the disease, or a symptom or condition thereof.

Systems, Computer-Readable Storage Media, and Methods Implemented by a Computer Processor

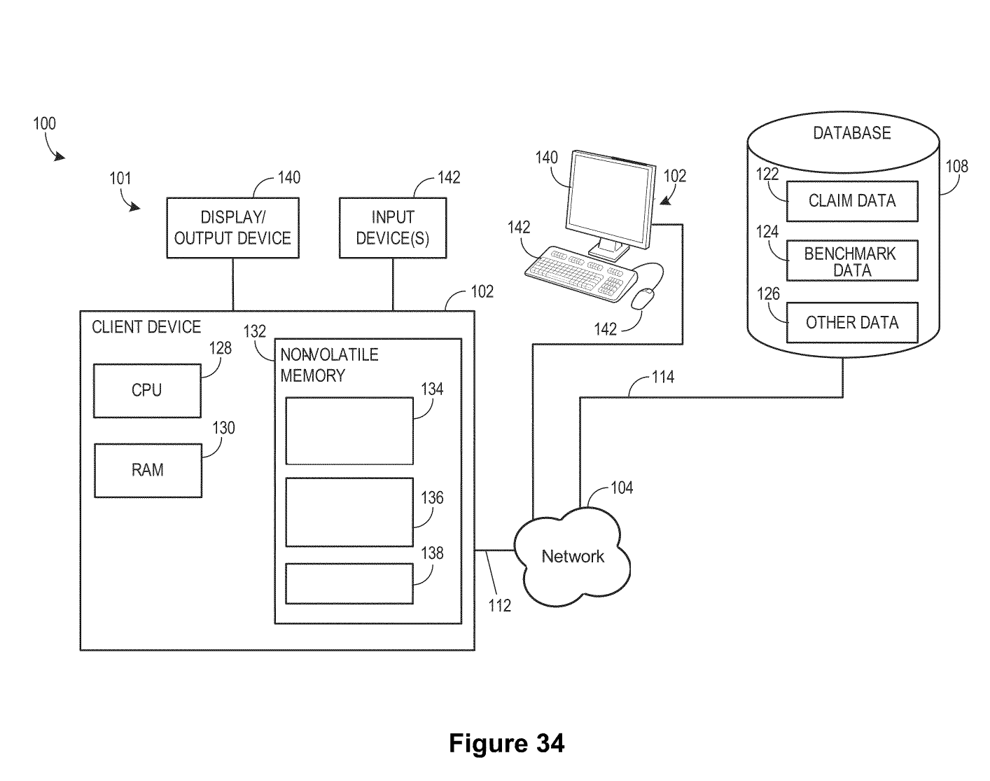

FIG. 34 illustrates an exemplary embodiment 101 of a system 100 for assessing a subject's need for an implanted cardiac defibrillator (ICD). Generally, the system 100 may include one or more client devices 102, a network 104, and a database 108. Each client device 102 may be communicatively coupled to the network 104 by one or more wired or wireless network connections 112, which may be, for example, a connection complying with a standard such as one of the IEEE 802.11 standards (“Wi-Fi”), the Ethernet standard, or any other appropriate network connection. Similarly, the database 108 may be communicatively coupled to the network 104 via one or more connections 114. (Of course, the database could alternatively be internal to one or more of the client devices 102.) The database 108 may store data related to the determination of a subject's need for an ICD including, but not limited to, data of a biological sample obtained from the subject, data of a biological sample obtained from a control or test population, data of a Gaussian distribution associated with the data of the biological samples, data of one or more threshold values associated with the data of the biological sample(s) and/or Gaussian distribution, etc. The data of the biological samples may be, for example, related to one or more of a level of a truncated SCN5A Exon 28 transcript, a level of a full length SCN5A Exon 28 transcript, a level of multiple SCN5A Exon 28 transcripts, etc., as described in greater detail below.

As will be understood, the network 104 may be a local area network (LAN) or a wide-area network (WAN). That is, network 104 may include only local (e.g., intra-organization) connections or, alternatively, the network 104 may include connections extending beyond the organization and onto one or more public networks (e.g., the Internet). In some embodiments, for example, the client device 102 and the database 108 may be within the network operated by a single company (Company A). In other embodiments, for example, the client device(s) 102 may be on a network operated by Company A, while the database 108 may be on a network operated by a second company (Company B), and the networks of Company A and Company B may be coupled by a third network such as, for example, the Internet.

Referring still to FIG. 34, the client device 102 includes a processor 128 (CPU), a RAM 130, and a non-volatile memory 132. The non-volatile memory 132 may be any appropriate memory device including, by way of example and not limitation, a magnetic disk (e.g., a hard disk drive), a solid state drive (e.g., a flash memory), etc. Additionally, it will be understood that, at least with regard to FIG. 34, the database 108 need not be separate from the client device 102. Instead, in some embodiments, the database 108 is part of the non-volatile memory 132 and the data 122, 124, 126 may be stored as data within the memory 132. For example, the data 122 may be included as data in a spreadsheet file stored in the memory 132, instead of as data in the database 108. In addition to storing the records of the database 108 (in some embodiments), the memory 132 stores program data and other data necessary to analyze data of one or more sample and/or control populations, determine a Gaussian fit for the data, determine a threshold against which data of the subject may be compared, and/or determine a subject's need for an ICD. For example, in an embodiment, the memory 132 stores a first routine 134, a second routine 136, and a third routine 138. The first routine 134 may receive data values related to one or more sample and/or control populations, and may fit the data values received by the routine 134 to a Gaussian distribution. The second routine 136 may computer one or more statistical parameters of the data collected by the first routine 134, such as determining a mean value, a standard deviation value, etc. of the Gaussian distribution. Additionally and/or alternatively, the second routine 136 may set a first threshold against which data from one or more subjects may be compared in order to determine whether each subject should receive an ICD. The third routine 138 may, for example, receive data for one or more subjects, compare the data of the one or more subjects to the threshold value(s) determined by the second routine 136, and/or determine whether each subject should receive an ICD according to the comparison of the subject's data to the threshold value. Regardless, each of the routines is executable by the processor 128 and comprises a series of compiled or compilable machine-readable instructions stored in the memory 132. Additionally, the memory 132 may store generated reports or records of data output by one of the routines 134 or 136. Alternatively, the reports or records may be output to the database 108. One or more display/output devices 140 (e.g., printer, display, etc.) and one or more input devices 142 (e.g., mouse, keyboard, tablet, touch-sensitive interface, etc.) may also be coupled to the client device 102, as is generally known.

As will be understood, although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

For example, the network 104 may include but is not limited to any combination of a LAN, a MAN, a WAN, a mobile, a wired or wireless network, a private network, or a virtual private network. Moreover, while only two clients 102 are illustrated in FIG. 34 to simplify and clarify the description, it is understood that any number of client computers are supported and can be in communication with one or more servers (not shown).

Additionally, certain embodiments are described herein as including logic or a number of routines. Routines may constitute either software routines (e.g., code embodied on a machine-readable medium or in a transmission signal) or hardware routines. A hardware routine is tangible unit capable of performing certain operations and may be configured or arranged in a certain manner. In example embodiments, one or more computer systems (e.g., a standalone, client or server computer system) or one or more hardware routines of a computer system (e.g., a processor or a group of processors) may be configured by software (e.g., an application or application portion) as a hardware routine that operates to perform certain operations as described herein.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Still further, the figures depict preferred embodiments of a map editor system for purposes of illustration only. One skilled in the art will readily recognize from the following discussion that alternative embodiments of the structures and methods illustrated herein may be employed without departing from the principles described herein

Upon reading this disclosure, those of skill in the art will appreciate still additional alternative structural and functional designs for a system and a process for identifying terminal road segments through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The invention provides systems comprising: a processor; a memory device coupled to the processor, and machine readable instructions stored on the memory device. In exemplary embodiments, the machine readable instructions that, when executed by the processor, cause the processor to
(i) receive a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) fit the plurality of data values to a first Gaussian distribution;
(iii) determine a mean value, μ, and a standard deviation, σ, of the first Gaussian distribution;
(iv) set a first threshold ratio, R_{T}, at μ−Xσ, wherein X is a number between 0.7 and 4.0.

In exemplary aspects, R_{T}=μ−Xσ, and X is a number between 0.7 and 1.0, a number between 2.0 and 4.0, or a number between 2.326 and 4.0. In exemplary aspects, the system comprises machine readable instructions that, when executed by the processor, cause the processor to receive as input an R_{S} and compare R_{S} to R_{T}. In exemplary aspects, the machine readable instructions that, when executed by the processor, cause the processor to provide an output indicating the relationship between R_{S} and R_{T}.

In alternative or additional aspects, the system of the invention comprises machine readable instructions that, when executed by the processor, cause the processor to:
(i) receive a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fit the second plurality of data values to a second Gaussian distribution;
(iii) determine a mean value, μ, and a standard deviation, σ, of the second Gaussian distribution;
(iv) set a second threshold ratio, R_{T2}, at μ+Xσ, wherein X is a number between 0.7 and 4.0.

In exemplary aspects, R_{T2}=μ+Xσ, and X is a number between 0.7 and 1.0, or a number between 2.0 and 4.0, or a number between 2.326 and 4.0.

In alternative or additional aspects, the system of the invention comprises machine readable instructions that, when executed by the processor, cause the processor to:
(i) receive a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fit the third plurality of data values to a third Gaussian distribution;
(iii) determine a mean value, μ, and a standard deviation, σ, of the third Gaussian distribution;
(iv) set a third threshold ratio, R_{T3}, at μ+4.0σ.

With respect to any of these systems, in some aspects, the system comprises machine readable instructions that, when executed by the processor, cause the processor to set a combined threshold ratio, R_{Tcombined}, at a point where the area under the curve of the first Gaussian distribution is maximized and the area under the curve of the second Gaussian distribution is minimized.

Also provided herein are computer-readable storage media having stored thereon machine-readable instructions executable by a processor. In exemplary embodiments, the instructions comprise:
(i) instructions for causing the processor to receive a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) instructions for causing the processor to fit the plurality of data values to a first Gaussian distribution;
(ii) instructions for causing the processor to determine a mean value, μ, and a standard deviation, σ, of the first Gaussian distribution;

(iii) instructions for causing the processor to set a first threshold ratio, R_{T}, at μ−Xσ, wherein X is a number between 0.7 and 4.0.

In exemplary aspects, R_{T}=μ−Xσ, and X is a number between 0.7 and 1.0, or between 2.0 and 4.0 or between 2.326 and 4.0. In exemplary aspects, the computer-readable storage medium comprises instructions for causing the processor to receive as input an Rₛ and compare Rₛ to R_{T}. In exemplary aspects, the computer-readable storage medium comprises instructions for causing the processor to provide an output indicating the relationship between Rₛ and R_{T}.

In alternative or additional embodiments, the computer-readable storage medium of the invention comprises instructions for causing the processor to:
(i) receive a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fit the second plurality of data values to a second Gaussian distribution;
(iii) determine a mean value, μ, and a standard deviation, σ, of the second Gaussian distribution;
(iv) set a second threshold ratio, R_{T2}, at μ+Xσ, wherein X is a number between 0.7 and 4.0.

In exemplary aspects, R_{T}=μ−Xσ, and X is a number between 0.7 and 1.0, or between 2.0 and 4.0 or between 2.326 and 4.0.

In alternative or additional embodiments, the computer-readable storage medium comprises instructions for causing the processor to:
(i) receive a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fit the third plurality of data values to a third Gaussian distribution;
(iii) determine a mean value, μ, and a standard deviation, σ, of the third Gaussian distribution;
(iv) set a third threshold ratio, R_{T3}, at μ+4.0σ.

The computer-readable storage medium in exemplary aspects comprises instructions for causing the processor to set a combined threshold ratio, R_{Tcombined}, at a point where the area under the curve of the first Gaussian distribution is maximized and the area under the curve of the second Gaussian distribution is minimized.

Further provided herein are methods implemented by a processor in a computer. In exemplary embodiments, the method comprises the steps of:
(i) receiving a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) fitting the plurality of data values to a first Gaussian distribution;
(ii) determining a mean value, μ, and a standard deviation, σ, of the first Gaussian distribution;
(iii) setting a first threshold ratio, R_{T}, at μ−Xσ, wherein X is a number between 0.7 and 4.0.

In exemplary aspects, R_{T}=μ−Xσ, and X is a number between 0.7 and 1.0, or between 2.0 and 4.0 or between 2.326 and 4.0. In exemplary aspects, the method comprises the step of receiving as input an Rₛ and compare Rₛ to R_{T}. In exemplary aspects, the method comprises the step of providing an output indicating the relationship between Rₛ and R_{T}.

In alternative or exemplary embodiments, the method comprises the steps of:
(i) receiving a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fitting the second plurality of data values to a second Gaussian distribution;
(iii) determining a mean value, μ, and a standard deviation, σ, of the second Gaussian distribution;
(iv) setting a second threshold ratio, R_{T2}, at μ+Xσ, wherein X is a number between 0.7 and 4.0.

In exemplary aspects, R_{T}=μ−Xσ, and X is a number between 0.7 and 1.0, or between 2.0 and 4.0 or between 2.326 and 4.0.

In alternative or exemplary aspects, the method comprises the steps of:
(i) receiving a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fitting the third plurality of data values to a third Gaussian distribution;
(iii) determining a mean value, μ, and a standard deviation, σ, of the third Gaussian distribution;
(iv) setting a third threshold ratio, R_{T3}, at μ+4.0σ.

In exemplary aspects, the method comprises the step of setting a combined threshold ratio, R_{Tcombined}, at a point where the area under the curve of the first Gaussian distribution is maximized and the area under the curve of the second Gaussian distribution is minimized.

In alternative embodiments of the systems, media, and methods described in this section, the invention further provides systems, media, and methods, wherein each subject of the first population is a subject known as having heart failure or arrhythmia. In exemplary aspects, the system, media, or method, further comprises instructions relating to receiving a third plurality of data values, each data value of the third plurality is a ratio determined from a biological sample obtained from a subject of a third population, wherein each subject of the third population is a subject known as (I) not having an ICD, (II) not having a cardiac disease, e.g., heart failure or arrhythmia, (III) having normal left ventricular function, (IV) not having diastolic dysfunction, or (V) a combination thereof.

Kits

Provided herein are kits, e.g., diagnostic kits, that may be used to diagnose or determine risk, in accordance with the methods set forth herein. In exemplary embodiments, the kit comprises: (a) an E28C binding agent and/or an E28D binding agent, (b) a WT SCN5A binding agent and/or an E28A-S binding agent and instructions for use. In exemplary aspects, the kit further comprises a binding agent to all SCN5A Exon 28 transcripts: WT, E28A-S, E28B, E28C, and E28D.

In additional or alternative embodiments, the kit comprises a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having an ICD that has given a shock; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values. In exemplary embodiments, the kit comprises access to the computer-readable storage medium. In exemplary embodiments, the kit comprises access to any one or more of (I) to (V).

In additional or alternative embodiments, the kit comprises a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having an ICD that has not given a shock; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values. In exemplary embodiments, the kit comprises access to the computer-readable storage medium. In exemplary embodiments, the kit comprises access to any one or more of (I) to (V).

In additional or alternative embodiments, the kit comprises a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as (I) not having an ICD, (II) not having a cardiac disease, (III) having normal left ventricular function, (IV) not having diastolic dysfunction, or (V) a combination thereof; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values. In exemplary embodiments, the kit comprises access to any one or more of (I) to (V).

In additional or alternative embodiments, the kit comprises a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having heart failure, or a risk therefor; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values. In exemplary embodiments, the kit comprises access to the computer-readable storage medium. In exemplary embodiments, the kit comprises access to any one or more of (I) to (V).

In additional or alternative embodiments, the kit comprises a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having arrhythmia, or a risk therefor; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values. In exemplary embodiments, the kit comprises access to the computer-readable storage medium. In exemplary embodiments, the kit comprises access to any one or more of (I) to (V).

In exemplary embodiments, the kit comprises a hLuc7a binding agent, a RBM25 binding agent, a PERK binding agent, and/or a chaperone protein binding agent, e.g., a CHOP binding agent, a calnexin binding agent.

As used herein, the term “binding agent” refers to any compound which specifically binds to the marker of interest (hLuc7A, RBM25, PERK, CHOP, calnexin, and the like).

With regard to the foregoing, the binding agent in some aspects is an antibody, antigen binding fragment, an aptamer, a protein or peptide substrate, or a nucleic acid probe. Such binding agents are known in the art. In some aspects, the kit comprises a collection of binding agents, e.g., a collection of antibodies, a collection of nucleic acid probes, each binding agent of which specifically binds to genes or nucleic acids encoding the marker. In some aspects, the collection of nucleic acid probes is formatted in an array on a solid support, e.g., a gene chip. In some aspects, the kit comprises a collection of antibodies which specifically bind to a marker. In some aspects, the kit comprises a multi-well microtiter plate, wherein each well comprises an antibody having a specificity which is unique to the antibodies of the other wells. In some aspects, the kit comprises a collection of substrates which specifically react with a marker. In some aspects, the kit comprises a multi-well microtiter plate, wherein each well comprises a substrate having a specificity which is unique to the substrates of the other wells.

In some aspects, the kits further comprises instructions for use. In some aspects, the instructions are provided as a paper copy of instructions, an electronic copy of instructions, e.g., a compact disc, a flash drive, or other electronic medium. In some aspects, the instructions are provided by way of providing directions to an internet site at which the instructions may be accessed by the user.

In some aspects, the kits further comprise a unit for a collecting a biological sample, e.g., any of the samples described herein, of the subject. In some aspects, the unit for collecting a sample is a vial, a beaker, a tube, a microtiter plate, a petri dish, and the like.

The following examples serve only to illustrate the invention or provide background information relating to the invention. The following examples are not intended to limit the scope of the invention in any way.

### EXAMPLES

### Example 1

This example demonstrates the detection of human SCN5A N-terminal and C-terminal mRNA splicing variants, as shown in U.S. Patent Application Publication No. 2007/0212723.

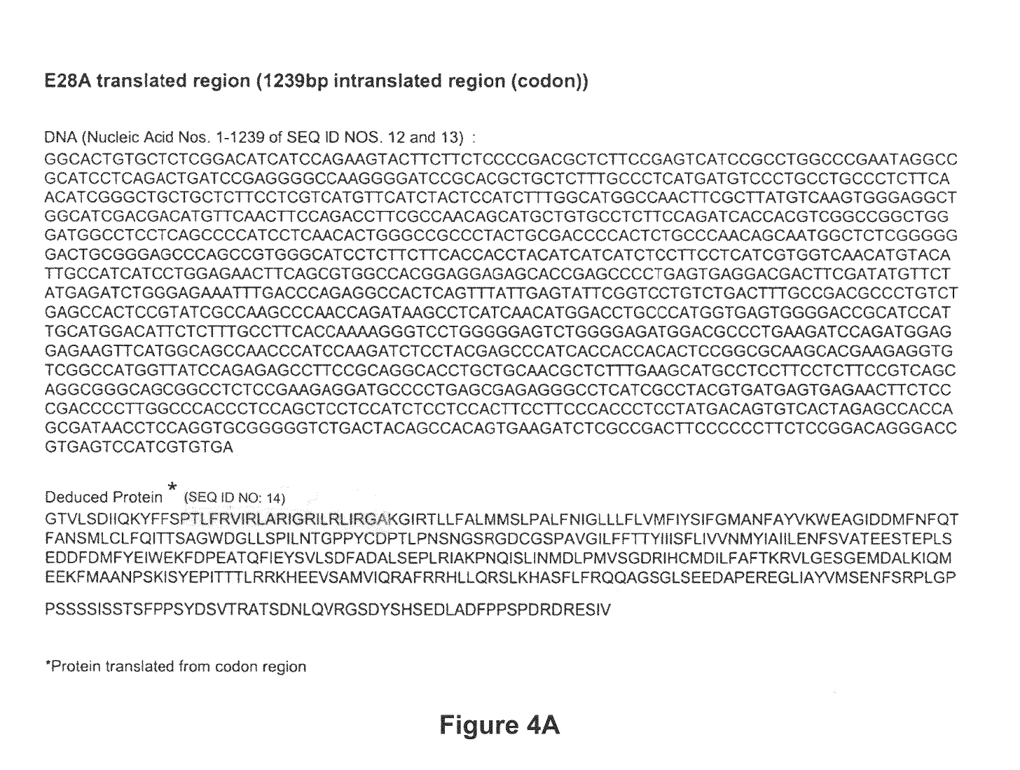

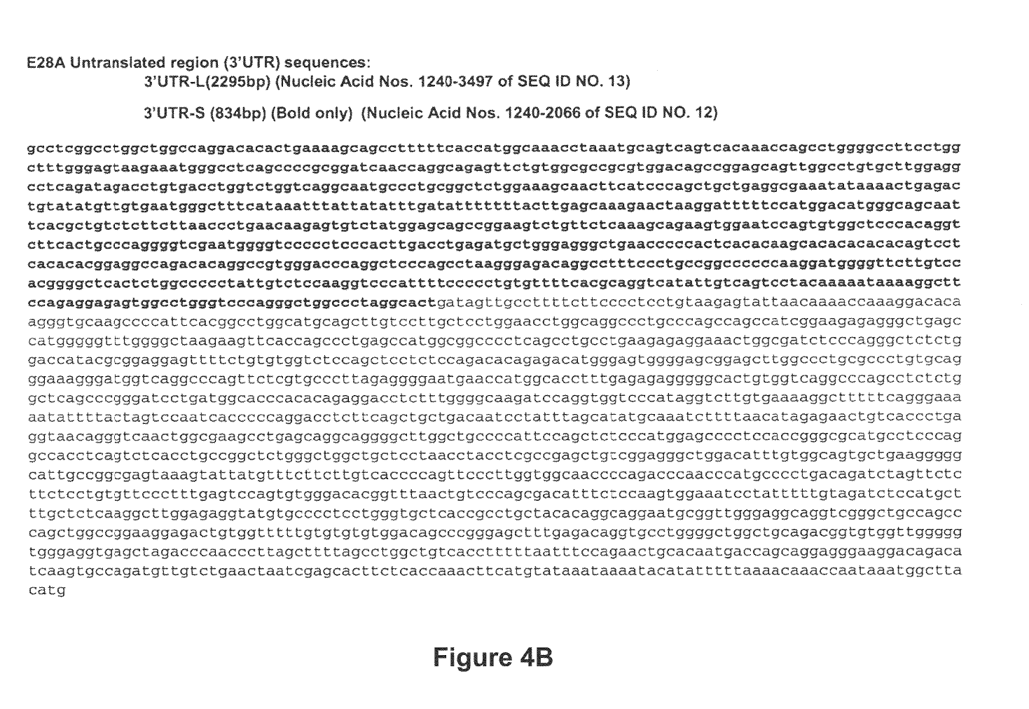

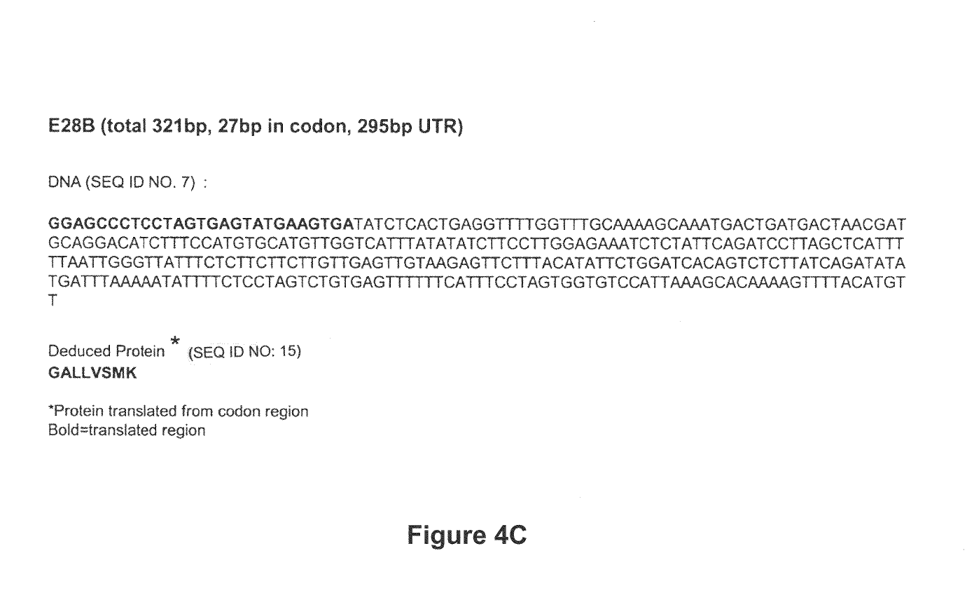

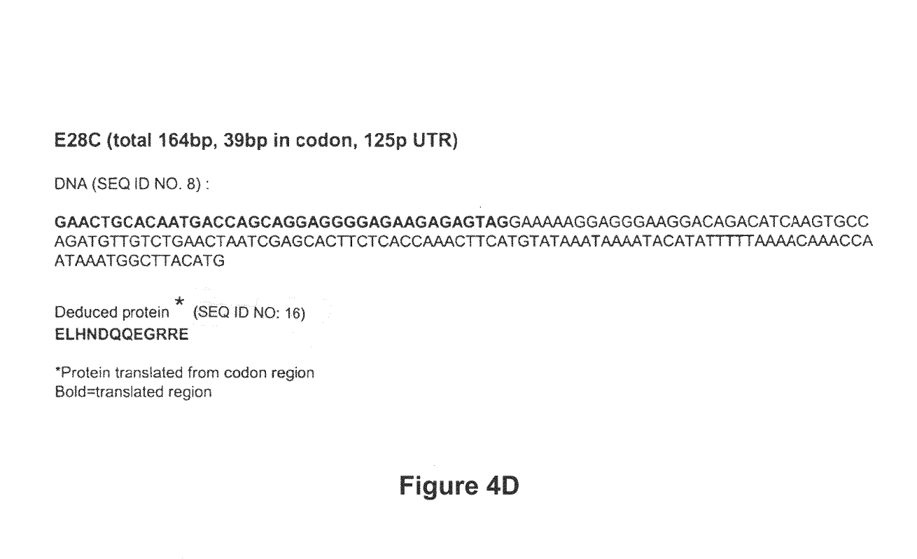

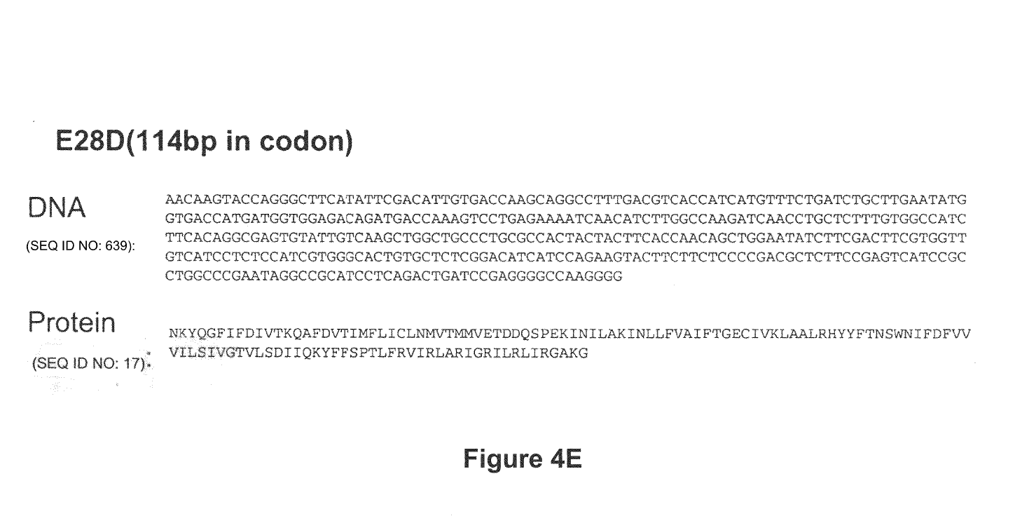

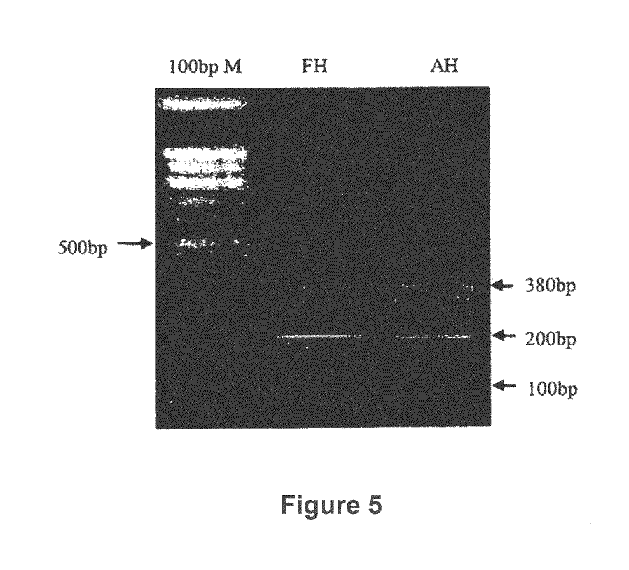

The first human SCN5A cDNA (reported by Sheng et al. (DNA and Cell Biology 13: 9-23 (1994), see, also, Zhang et al., Gene Expression 8: 85-103 (1999)) (accession # M77235) was 8.5-9.0 kb, with a 5′ end extending to 150 bp upstream of the ATG codon and a 2293 bp 3′ UTR, which contains neither polyadenylylation signal sequence nor a poly A region. The mouse scn5a gene has complex 5′ and 3′ UTR and 5′ and 3′ UTR splice variants are developmentally regulated (Shang, L. L. & Dudley, S. C., Jr, J. Biol. Chem. 280: 933-940 (2005)). Therefore, we sought to evaluate whether the previously reported human UTR sequences represented the full extent of the cDNA isoforms. The RACE procedure was employed to the 5′ and 3′ mRNA ends upstream of the start codon and downstream of the stop codon, respectively. PCR amplification of cDNA yielded several distinct bands on gel electrophoresis. Subsequent nested PCR amplification gave three bands (380 bp, 200 bp and 100 bp) in both fetal heart RNA and adult heart RNA (FIG. 5). Sequences of these bands showed that the all three bands were SCN5A gene specific. A comparison between the 5′RACE-PCR products and genomic sequences showed that there were two different exon 1 isoforms from reported data, one was 160 bp and located 16.0 kb upstream of exon 2, which is known. A novel isoform was 85 bp, which was separated by 12.3 kb from exon 2. Another new splicing exon 1 was found in exon 2, which means the there is no exon 1 splice. The RNA directly transcripted from exon 2, but the TSS was short 13 bp in comparison with the normal exon 2. From the RACE procedure, we were unable to obtain the exon 1 isoform reported previously (Shang, L. J. & Dudley, S. C., Biophysical Journal 86, 424A (2004)), suggesting that a total of three exon 1 splice variants existed. And the multiple TSS existed in all splicing isoforms. We named these untranslated cDNA fragments, exon 1 A (160 bp), exon 1B (85 bp), and exon 2B (313 bp), respectively. Exon 1A, identified by primer extension and RNase protection, has been reported previously in human and rat with a length ranging from 97-176 bp (Yang et al., Cardiovascular Research 61:, 56-65 (2004)). All isoforms of the SCN5A 5′UTR were summarized in FIGS. 1 and 3 and sequences were depicted in FIGS. 2 and 4.

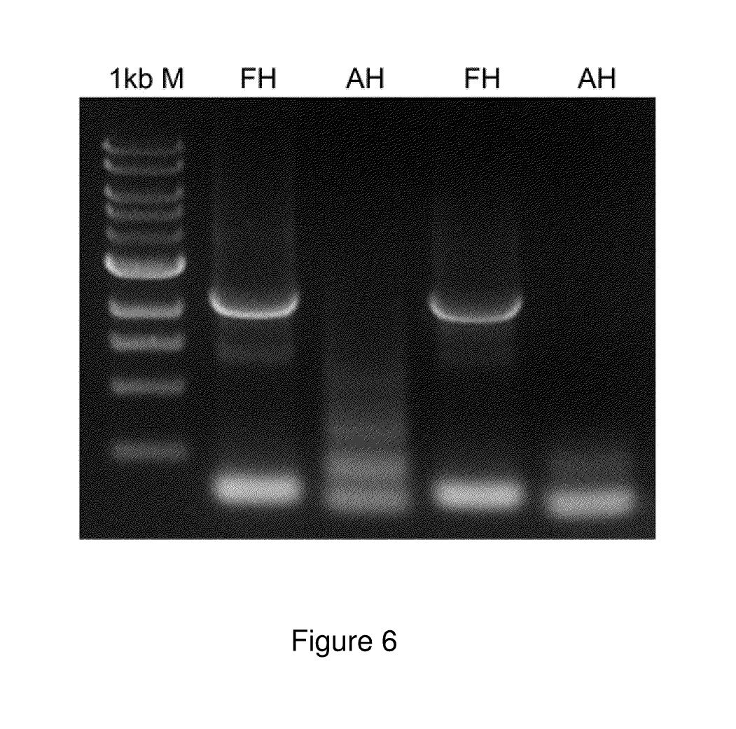

Analysis of the 3′UTR showed evidence of splice variations in fetal and adult heart. By RACE-PCR using GSP3′ and Oligo dT primers, we identified six alternative 3′UTRs (FIG. 6). Comparison of genomic and cDNA sequences showed that the 3′ UTRs had six different poly A splicing variants. One was long, 2295 bp after the stop codon, and was not found in Genebank, but it is consistent with the published mouse scn5a cDNA (accession # AJ271477). Additionally, there was a second, short variant of 824 bp, which corresponded to the human scn5a cDNA 5′UTR (accession # M77235). These two isoforms have the exon 28 bp the same splice referred to as E28A. Three Exon 28 splice variants were detected in the 3′UTR: E28B (27 bp), E28C (39 bp), and E28D (114 bp). The sequences of E28B, E28C, and E28D are set forth as Genbank accession numbers EF092292, EF092293 and EF092294, respectively, and here as SEQ ID NOs: 636-638, respectively). E28B is fetal specific, E28D is adult specific, whereas the E28C expressed in both fetal and human heart. In comparison with the full-length E28A variant, all three variants were shorter and result in prematurely truncated Na+ channel proteins missing the segments from domain IV, S3 or S4 to the C-terminus (George et al., Cytogenet Cell Genet. 68: 67-70 (1995)). A summary of the findings of the 3′UTR is presented in FIG. 3. The sequences of the all isoforms for the SCN5A 3′UTR were preformatted in FIG. 4.

The methods used in this example are as follows:

Human heart tissue from fetal and adult was homogenized, and total RNA isolated using the RNeasy Mini kit following the manufacturer's instructions (Qiagen, Valencia, Calif.). RNA ligase-mediated-rapid amplification cDNA ends (RLM-RACE) methods were used to characterize the 5′ and 3′ ends of the human SCN5A mRNA using GeneRacer kit (Invitrogen, Carlsbad, Calif.). Briefly, 1 .mu.g total RNA was treated with calf intestinal phosphatase to remove the 5′ phosphates of the truncated mRNA and non-mRNA forms of total RNA. Tobacco acid pyrophosphatase was used to remove the 5′ cap structure from intact, full-length mRNA, and T4 RNA ligase was use to add the GeneRacer RNA Oligo to the 5′ end of the mRNA. The first-strand cDNA was synthesized by SuperScript II reverse transcriptase using a reverse gene specific primer GSP5′ (5′CATCTTCCGGTTCAGTGCCACCA 3′ (SEQ ID NO: 640)) complementary to exon 3 of human SCN5A gene and the GeneRacer Oligo dT primer at the 5′ and 3′ ends, respectively. The 5′ and 3′ ends PCR reactions were performed with Platinum Pfx DNA polymerase using 10 .mu.M of GSP5′ and the GeneRacer 5′ primer for amplifying the 5′ end fragment and using 10 .mu.M of the forward GSP3′ (5′GCTGCCCTGCGCCACTACTACTTC3′ (SEQ ID NO: 641)) complementary to exon 27 of the human SCN5A and the GeneRacer Oligo dT primer to obtain 3′ end. An additional PCR reaction with nested primers was performed. The nested PCR products were cloned into pCRII-TOPO vector (Invitrogen, Carlsbad, Calif.) and sequenced to confirm the RACE-PCR products were from the human SCN5A cDNA.

Primary DNA sequence analysis was performed with Vector NTI 7 software (Informax, Frederick, Md.). The sequences were aligned to human genomic DNA and cDNA sequences in Genebank database to identify transcription start sites (TSSs) and 3′ polyadenylylation sites.

### Example 2

This example demonstrates data suggesting that human heart failure is associated with abnormal C-terminal splicing variants in the cardiac sodium channel, as shown in one or more of Shang et al., Circulation Research 101: 1146-1154 (2007) and U.S. Patent Application Publication No. 2007/0212723.

Detection of Three Novel Human SCN5A C-Terminal mRNA Splicing Variants

Two SCN5A mRNA variants that do not alter the coding sequence have been reported previously from mouse heart that differed in the length of the poly-A tail (Gellens et al., 1992, Proc. Natl. Acad. Sci. U.S.A. 89:554-558; and Shang, L. L., and Dudley, S. C., Jr., 2005, J. Biol. Chem. 280:933-940). Using RACE-PCR, we found analogous sodium channel mRNA isoforms in the human heart (FIG. 6). In addition to these bands, nested R_{T}-PCR revealed shorter bands in both fetal and adult human heart. Sequence analysis of the bands revealed three new mRNA splice variants, as described above and designated as exons E28B (27 bp), E28C (39 bp), and E28D (114 bp) (Genbank accession numbers EF092292, EF092293 and EF092294, respectively). The alternative splicing that produced these novel exons are summarized in FIG. 3. In comparison with the full-length E28A isoform, all three new isoforms were shorter and were predicted to result in prematurely truncated sodium channel proteins missing the segments from domain IV, S3 or S4 to the C-terminus.

### The Relative Abundances of the SCN5A Isoforms are Developmentally Regulated

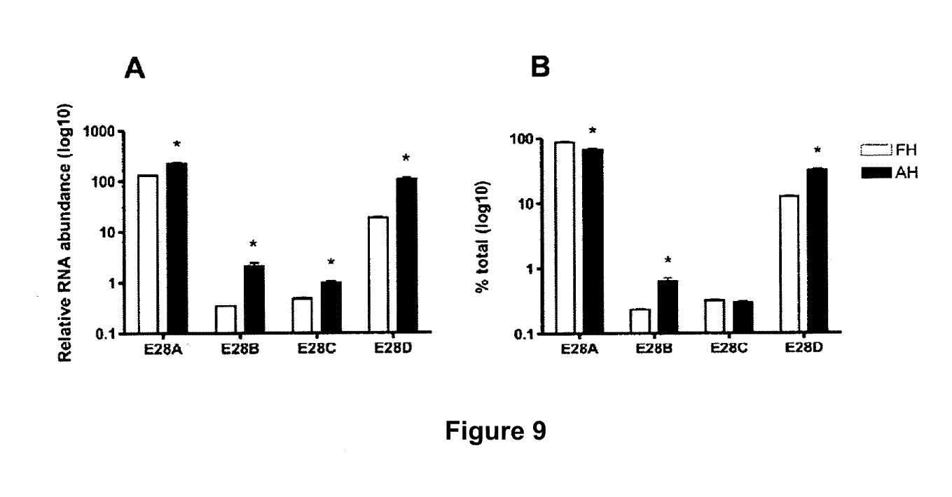

Splice variants of Naᵥ1.5 in C-terminus are known to vary during development. Therefore, we investigated whether our novel 3′ isoforms showed similar behavior. Quantitative real time R_{T}-PCR indicated that the relative abundances of each of the isoforms increased by 41.6% (p<0.001), 5.1 fold (p<0.01), 1.1 fold (p<0.01) and 4.8 fold (p<0.001) for E28A, E27B, E28C, and E28D from fetal to adult heart, respectively (FIG. 9A). FIG. 9B shows that as a percentage of the total transcripts, E28A was the most abundant in both fetal and adult heart. E28B was the least abundant in fetal heart but increased the most with development, changing 1.7.+−.0.2 fold. As a percentage of the total mRNA, the alternative splice variants E28B and E28D increased significantly, the full length E28A decreased, and the E28C abundance was unchanged during development.

### Heart Failure Increased Two of the Sodium Channel C-Terminal Splice Variants

The presence of splice variants was compared between the ventricles obtained from 12 patients whose hearts were removed during cardiac transplantation for HF (Table 1), and three control patients with no known cardiac disease.

| Patients # | Type of CM | Gender | Age |
|---|---|---|---|
| 1 | DCM | M | 60 |
| 2 | DCM | M | 54 |
| 3 | DCM | M | 62 |
| 4 | ICM | M | 43 |
| 5 | DCM | M | 45 |
| 6 | DCM | M | 51 |
| 7 | DCM | F | 59 |
| 8 | DCM | M | 63 |
| 9 | DCM | M | 60 |
| 10 | DCM | M | 45 |
| 11 | DCM | M | 63 |
| 12 | ICM | M | 47 |

DCM: dilated cardiomyopathy
ICM: ischemic cardiomyopathy
CM: cardiomyopathy
M: male
F: female

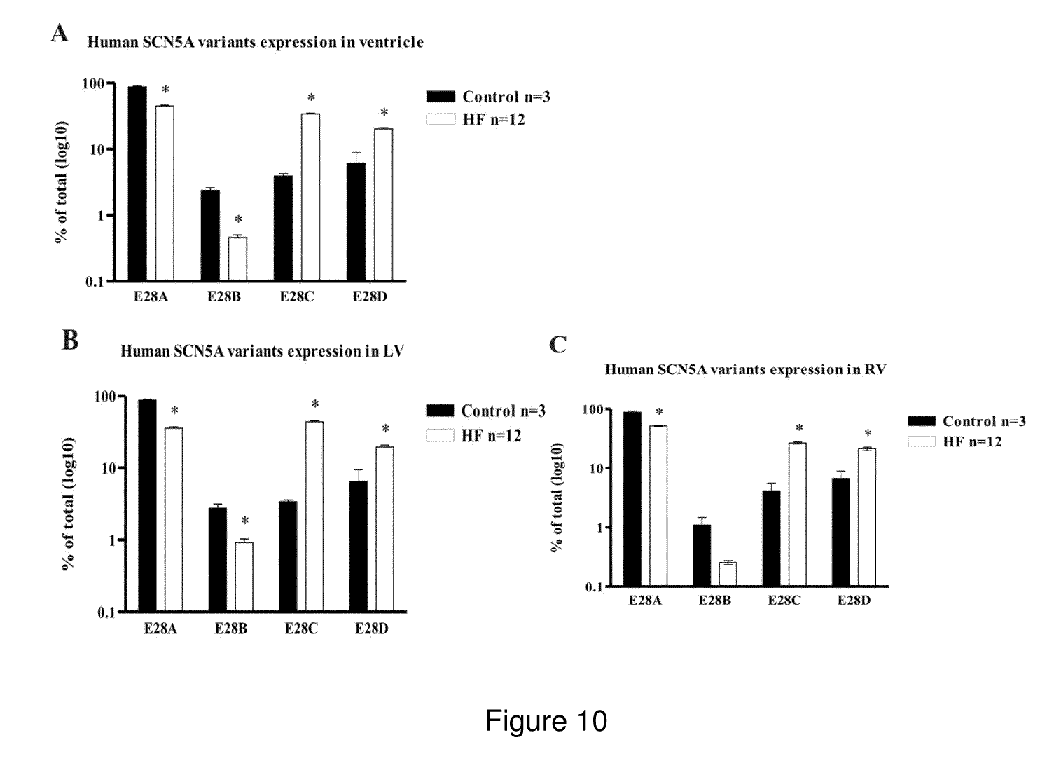

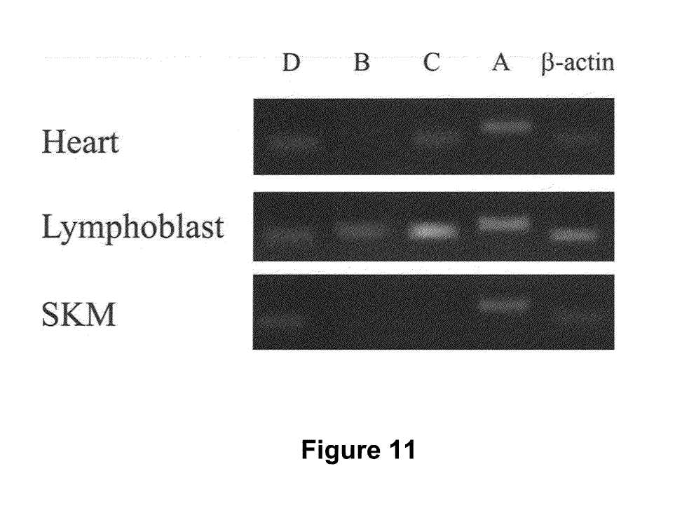

Real time R_{T}-PCR results indicated that the relative mRNA abundance of E28A full length isoform was decreased by 24.7% in HF patients compared to controls (p<0.001). Two of the truncated isoforms were increased in HF patients as compared to the controls (FIG. 10A), however. The E28C and E28D mRNA abundances were increased 14.2 fold (p<0.001) and 3.8 fold (p<0.001) respectively comparing controls to HF patients (FIG. 10A). On the other hand, the least abundant isoform, E28B, decreased 73.8% (p<0.01) in HF patients. As a percentage of the total transcript abundance, E28A and B decreased significantly from 87.5% (.+−.5.1) and 2.4% (.+−.0.4) in controls to 45.1% (.+−.4.5) and 0.5% (.+−.0.2) in HF patients. The E28C and D variants increased from 3.9% (.+−.0.6) and 6.2% (.+−.4.6) in controls to 34.3% (.+−.3.1) and 20.2% (.+−.3.3) in HF patients (FIG. 10A). The total percentage of short isoform variants went from 12.5% (.+−.5.1) of the total SCN5A mRNA in control subjects to 54.9% (.+−.4.5) in HF patients.

Because inhomogeneities of channel expression are thought to contribute to arrhythmic risk, the relative RNA abundance of the SCN5A isoforms were compared in the left (LV, FIG. 10B) and right ventricles (RV, FIG. 10C) of controls and HF patients. Normalized to the total SCN5A mRNA, E28A abundances were decreased in both the LV and RV. The pattern of changes for the truncation variants was similar in both ventricles with increases in E28C and E28D. The percentage of truncated mRNAs was increased more in the LV when compared to the RV (p=0.0003).

### Truncated Isoforms Reduce Sodium Channel Function

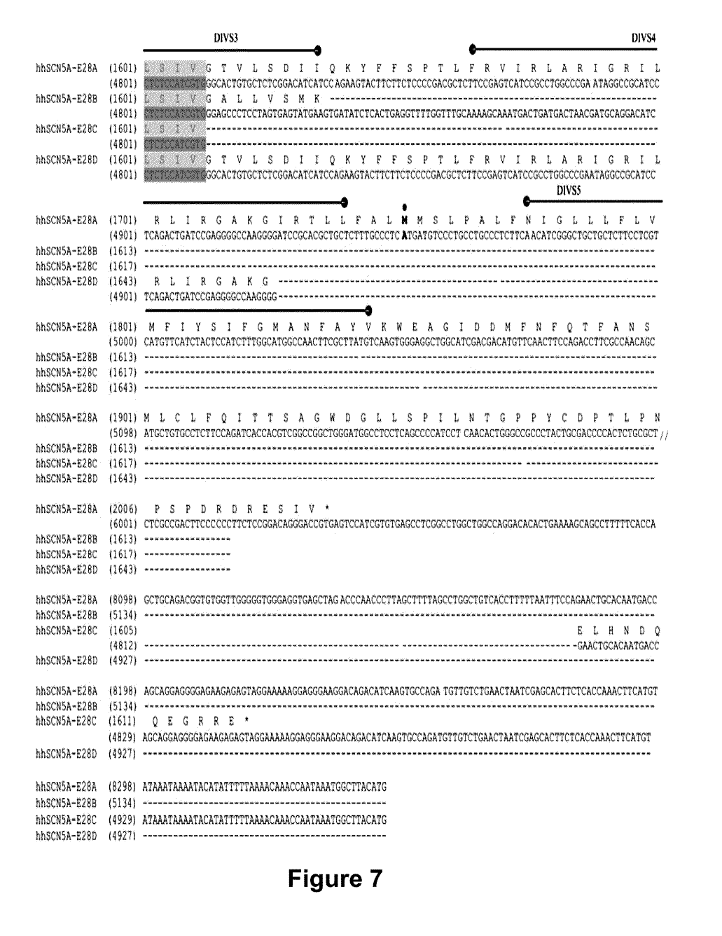

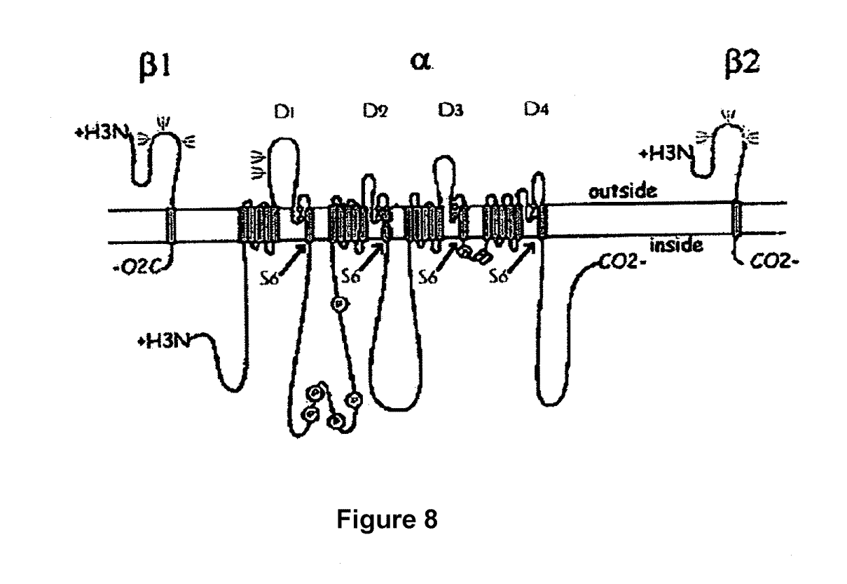

The three novel SCN5A splice variations identified were predicted to result in prematurely truncated, nonfunctional Sodium channels. We tested this idea by making a gene-targeted mouse model with a nonsense mutation in exon 28 (1652stop, FIG. 7). Proper homologous recombination in embryonic stems cells was confirmed by sequencing, restriction site analysis, and Southern blotting (see supplemental material). Embryonic stem (ES) cells were successfully injected into blastocyst and produced chimeric animals. Nevertheless, this mutation was lethal to embryos when attempting to breed these animals. Undifferentiated mouse ES cells heterozygous for the SCN5A^{1652stop} had normal growth characteristics and could be differentiated into spontaneously beating cardiomyocytes (CMs), however.

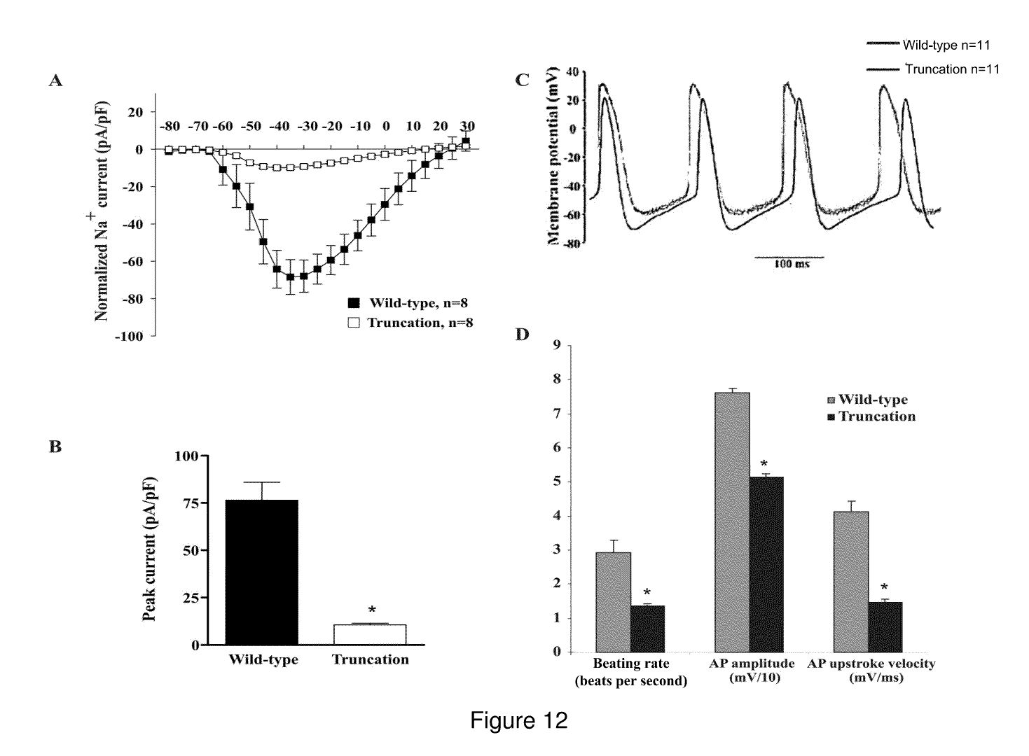

To assess the electrophysiological effect of the presence of the truncation, ES cells heterozygous for the mutation were differentiated in vitro to CMs and studied electrophysiologically. Current and action potentials (APs) were compared from single CMs enzymatically isolated at day 19. The Sodium channel current-voltage relationships from contracting CMs isolated from wild-type and truncation embryonic bodies derived from the respective ES cell lines are shown in FIG. 5A. The peak I_{Na} was decreased by 86.1% (.+−.5.2, n=8, p=0.0002) in differentiated CMs containing the truncation when compared to that of WT (76.4.+−.9.5 pA/pF to 10.6.+−.0.9 pA/pF; FIG. 5B). Action potentials recorded in the current clamp mode from spontaneously beating CMs showed significant slowing of the beating frequency from 2.9.+−.0.4 beats per second (bps) in the wild-type to 1.3 bps.+−.0.1 (p=0.02, n=11) in the truncation mutant. Action potentials also showed a significant reduction in the maximum rate of rise of the AP in the truncation mutation from 4.1.+−.0.3 mV/ms to 1.4.+−.0.1 mV/ms (p<0.01, n=11) and a reduced amplitude from 76.+−.1.4 mV to 52.+−.0.6 mV (p.ltoreq.0.01, n=11) in comparison with wild-type (WT) (FIGS. 12C and D). These changes are consistent with reduced sodium channel function.

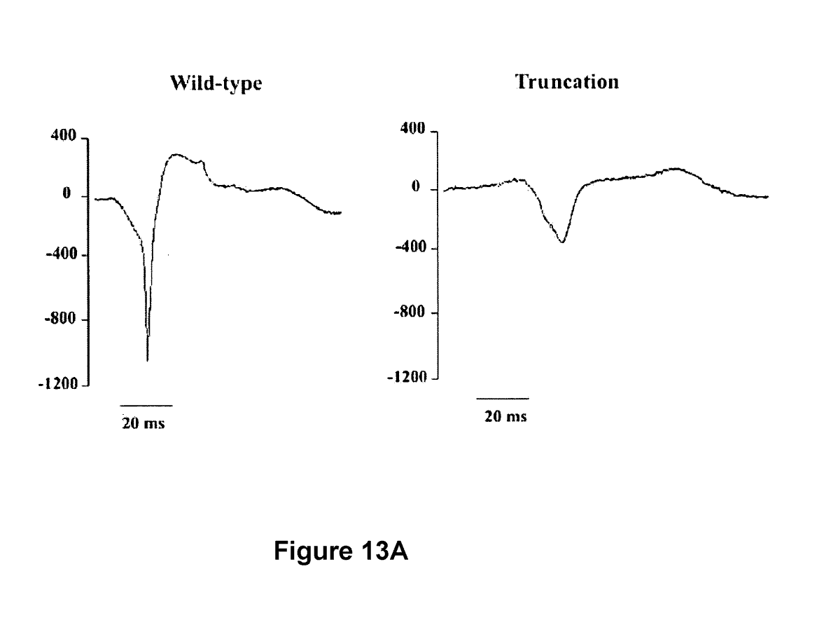

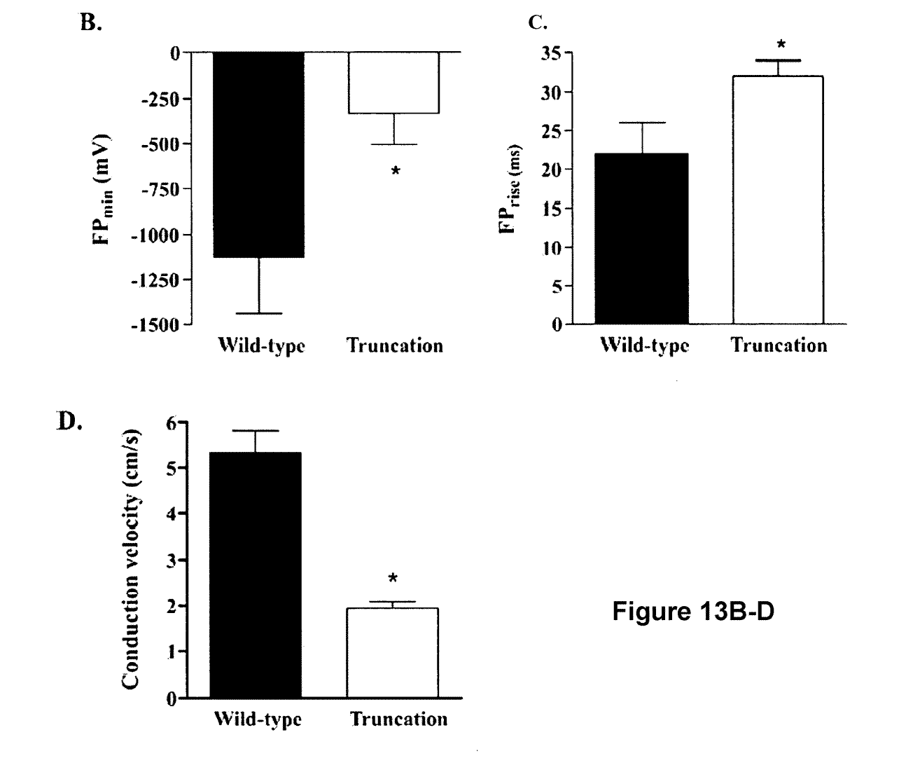

Syncytial properties of CMs containing the truncation mutation were studied by dissecting areas of CMs from embryoid bodies and placing them on top of planar multi-electrode arrays (MEAs) (Caspi, and Gepstein. 2004. Potential applications of human embryonic stem cell-derived cardiomyocytes. Ann. N.Y. Acad. Sci. 1015:285-298; and Kehat et al. 2002. High-resolution electrophysiological assessment of human embryonic stem cell-derived cardiomyocytes: a novel in vitro model for the study of conduction. Circ. Res. 91:659-661). Characteristics of bipolar extracellular electrograms from WT and mutated ES cells were compared. The time of the initial decline of the bipolar field potential (FP), the minimum FP amplitude, and conduction velocity are known to reflect sodium channel activity. Consistent with a physiologically significant reduction in sodium current as a result of the truncated mRNA, MEA recordings of CMs with the truncation mutation showed the minimum FP decreased by 70.5% (p<0.05) from −1126.+−.314 .mu.V (n=6) in WT to −332.+−. 174 .mu.V (n=7) in the truncation (FIGS. 13A and B). The FP rise slowed by 45.5% (p<0.05) from 22.+−.4 ms (n=6) to 32.+−.2 ms (n=7) in WT and the mutant (FIGS. 13A and C), respectively. The conduction velocity was decreased by 64.2% (p=0.03) from 5.3.+-.1.2 cm/s (n=6) in WT to 1.9.+−.4 cm/s (n=7) in the truncation (FIG. 13D).

The methods used in this example are as follows:

### Detection of Human SCN5A 3′UTR Isoforms by RACE-PCR

Total human RNA from normal fetal and adult whole hearts was purchased from Clontech (Mountain View, Calif.). The RNA ligase-mediated rapid amplification of cDNA ends (RLM-RACE) method was used to characterize the 3′ ends of the human SCN5A mRNA using the GeneRacer kit (Invitrogen, Carlsbad, Calif.). Briefly, 1 μg total RNA was used to synthesize first-strand cDNA by SuperScript II reverse transcriptase using the GeneRacer Oligo dT primer at the 3′ ends. The 3′ ends PCR reactions were performed with Platinum Pfx DNA polymerase using 10 .mu.M of a forward gene-specific primer (GSP) HE26F (5′ CATCCCACGGCCCCTGAACAAGTA 3′ (SEQ ID NO. 642)) complementary to exon 26 of human SCN5A gene and the GeneRacer 3′ primer for amplifying the 3′ end fragment. An additional PCR reaction with a nested GSP primer HE27F (5′ CTGCGCCACTACTACTTCACCAACA 3′ (SEQ ID NO. 643)) corresponding to exon 27 of human SCN5A gene and a nested GeneRacer 3′ primer was performed. The nested PCR products were cloned into pCR4-TOPO vector (Invitrogen, Carlsbad, Calif.) and sequenced. Sequences were compared to that of SCN5A using Vector NTI 7 software (Invitrogen).

### Isolation and Culture of Lymphoblasts

Human lymphoblast cell lines were developed from peripheral blood mononuclear cells of volunteers with normal cardiac function referred to the cardiac catheterization laboratory at the Atlanta Veterans Administration Medical Center (AVAMC). Procedures and consent forms were approved by the Emory Institutional Review Board and the AVAMC's Research & Development Committee. To initiate immortalized lymphoblast cell lines, peripheral blood was fractionated by Ficoll-Hypaque centrifugation and mononuclear cells were infected with the B95-8 strain of Epstein-Barr virus (EBV). After EBV-transformation, lymphoblasts were cultured in RPMI-1640 medium supplemented with 10% heat-inactivated fetal bovine serum, 2 mM L-glutamine, 100 U/mL penicillin, and 100 mg/mL streptomycin, at 37.degree. C. in a humid atmosphere with 5% CO₂. Medium was changed twice weekly. Cell counts and viability were assessed by trypan blue staining and fluorescence microscopy. Approximately 5.0.times.10⁷ lymphoblasts were collected by centrifugation and the RNA isolated using TRIzol reagent (Invitrogen) as described by manufacturer's manual.

### Real-Time SYBR Green PCR Quantification of SCN5A Transcript Isoforms

Ventricular tissue from hearts removed at the time of cardiac transplantation at Emory University Hospital under a protocol approved by the Emory Institutional Review Board was homogenized, and total RNA was isolated using the TRIzol reagent (Invitrogen, Carlsbad, Calif.) following the manufacturer's instructions. The total RNA from ventricles and skeletal muscle of the normal adults was bought from Ambion (Austin, Tex.) and Clontech, respectively. To determine the abundance of different cardiac mRNAs carrying variant exon 28 (E28) isoforms, total RNA from left and right ventricles of both normal and patients was used for synthesizing cDNA by reverse transcription using iScript cDNA synthesis Kit (Bio-Rad, Hercules, Calif.) following the manufacturer's instructions. The first strand cDNA was used as template for subsequent PCR. Each PCR reaction contained 12.5 .mu.L of QuantiTect SYBR Green PCR kit master mix (Qiagen, Valencia, Calif.) and 200 nM primer pairs in total 25 .mu.L reaction volume. The reversed primers for exon 28 variants were HSCN5AE28A/R (5′ GGAAGAGCGTCGGGGAGAAGAAGTA 3′ (SEQ ID NO. 21), E28A and 28D), HSCN5AE28B/R (5′ ATGCACATGGAAAGATGTCCTGC 3′ (SEQ ID NO. 644), E28B), HSCN5AE28C/R (5′ TCTTGGCACTTGATGTCTGTCCTTC 3′ (SEQ ID NO.645), E28C) and HSCN5AE28D/R (5′ TCATGAGGGCAAAGAGCAGCGT 3′ (SEQ ID NO. 646), E28A only), respectively. The forward primer, HE27F, was constant in each case. The reactions gave rise to 124 bp, 170 bp, and 143 bp and 211 bp PCR products, respectively. Amplification with primers HE27F and HSCN5AE28D/R produced the full length isoform, E28A. Amplification with HE27F and HSCN5AE28A/R produced a product comprised of both isoforms E28A and E28D. The amount of E28D was calculated by subtraction of the products of these two reactions. All amplifications were performed in triplicate and consisted of 40 cycles of 30 s at 94° C., 30 s at 65° C., and 1 min at 72° C. in a BioRed thermocycler iCycler (Hercules, Calif.). PCR products were analyzed by electrophoresis on 1.5% agarose gels.beta.-actin was used as an internal reference when making quantitative comparison.

### Generation of Truncated Scn5a Mouse Model

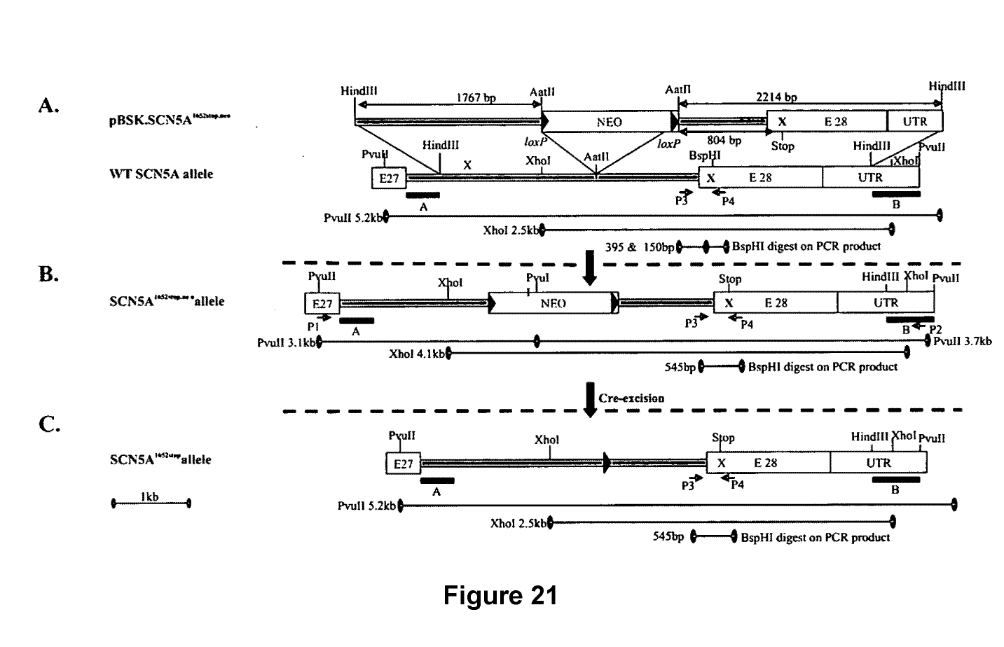

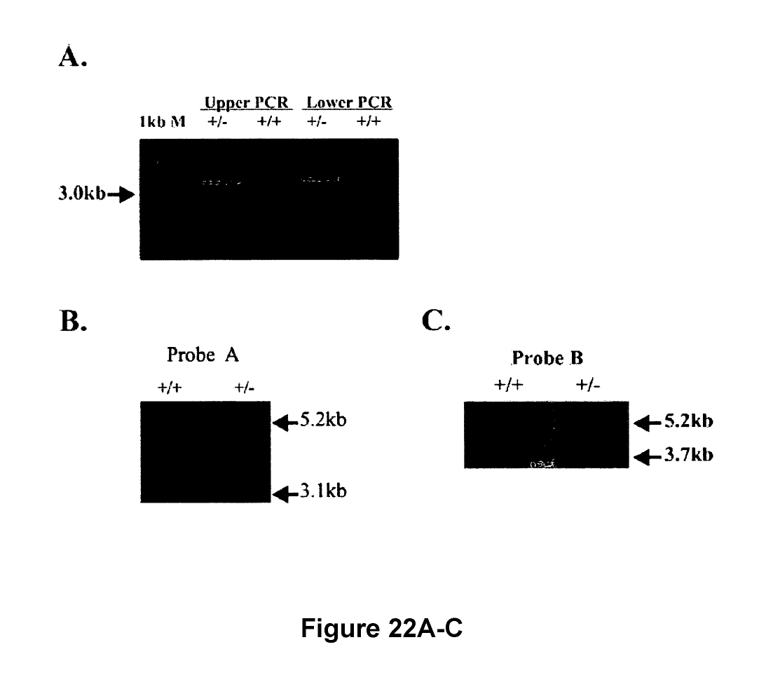

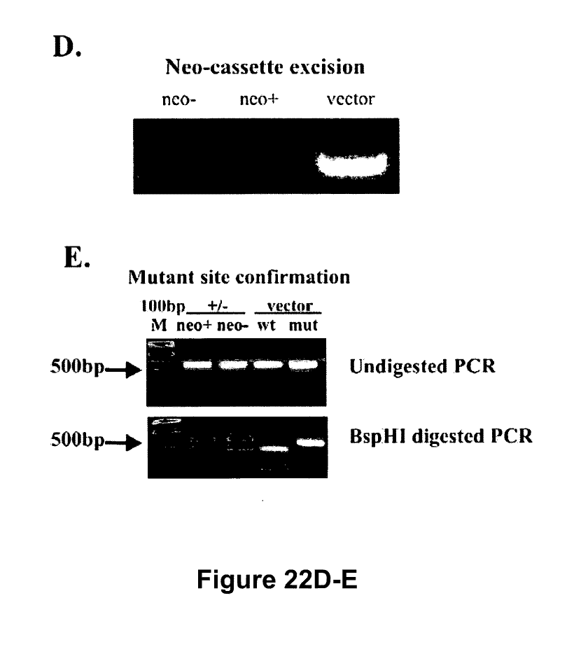

A 4.0-kb fragment of 129 Sv/J mouse genomic DNA was cloned from a mouse ES cell genomic library by PCR amplification using primers RHI28F/R (11) corresponding to the known mouse SCN5A exon 28 sequences (40). One PCR positive clone was used to construct the scn5a truncation targeting vector pBSK.SCN5A^{1652stop} by subcloning a 4.0-kb HindIII genomic fragment. The floxed PGK-neomycin cassette was inserted into AatII site (FIG. 52). An adenosine (A) was deleted in this codon. This deletion resulted in a frame shift turning the 1652 codon that formerly encoded for a methionine into a stop codon (TGA; NCBI ACCESSION NP₋₉₃₂₁₇₃; FIG. 7), predicted to cause premature Sodium channel truncation. The 5′ and 3′ arms of the targeting vector separated by neomycin resistance cassette were 1,767 and 2,214 bp, respectively, leaving a 942 bp homologous fragment on 5′ side of the mutation (FIG. 52). After electroporation with 20 .mu.g of the KpnI-linearized targeting construct into the mouse embryonic stem cells (R1), targeted clones were screened and identified by PCR using primers P1/neoR (P1:5′ GCTGCCCTGCGCCACTATTACTTC 3′ (SEQ ID NO. 647)); neoR: 5′ AAGAACTCGTCAAGAAGGCGATAGAAGGCG 3′ (SEQ ID NO. 648)) neoF/P2 (neorF: 5′ AGGATCTCCTGTCATCTCACCTTGCTCCTG 3′ (SEQ ID NO. 649); P2: 5′ AAGCAAGCTACGTGCCTGGCTG 3′ (SEQ ID NO. 650)) at the 5′ and 3′ ends and neo-cassette (FIGS. 21B and 22A), and using primers P3/P4 (P3: 5′ CAGAGCCCATGTATAGTTGATTTC 3′ (SEQ ID NO. 651); P4: 5′ GCTGTTGGCAAAGGTCTG 3′ (SEQ ID NO. 652)) surrounding the mutation site (FIGS. 21 and 22E). Southern blotting of the 5′ and 3′ ends with the external probes A and B was used to confirm the PCR results (FIGS. 21, 22B, and 22C). The floxed neomycin cassette was excised by expressing transfected Cre recombinase in correctly targeted clones confirmed by PCR using neorF/R (FIG. 21D). Heterozygosity was confirmed by P3-P4 PCR product with or without BspHI digestion (FIG. 21E). Experimental studies were performed on an ES cell line in which one allele of the Sodium channel was successfully targeted.

In Vitro Differentiation of ES Cell into Cardiomyocytes

R1 mouse ES cells with or without the mutation were maintained in the undifferentiated state using high glucose Dulbecco modified Eagle medium (DMEM, GibcoBRL, Life Technologies Inc., Rockville, Md.) with supplements and differentiated as described previously (Zhang, Y. M., Shang, L., Hartzell, C., Narlow, M., Cribbs, L., and Dudley, S. C., Jr. 2003. Characterization and regulation of T-type Ca²⁺ channels in embryonic stem cell-derived cardiomyocytes. Am. J. Physiol Heart Circ. Physiol 285:H2770-H2779). For patch clamp experiments, areas of beating CMs were mechanically dissected from 19 day old embryoid bodies, and single CMs were obtained by enzymatic digestion of the dissected areas (Shang et al. 2006. Analysis of arrhythmic potential of embryonic stem cell-derived cardiomyocytes. Methods Mol. Biol. 330:221-231). For the MEA experiments, areas of beating CMs were mechanically dissected from 17 day old embryoid bodies, placed on top of a MEA, and cultured for another 2 days prior to recording.

Recording of Sodium Current from ES-Derived Cardiomyocytes

Patch clamp experiments were performed 1 to 5 days after cell isolation. Cardiomyocytes with uniform contractions and beating rates were used in the study. Patch pipettes were pulled to resistance of 2 to 5 M.OMEGA. Current clamp experiments were conducted in a solution consisting of (in mM) NaCl 140, KCl 5.4, CaCl₂ 1.8, MgCl₂ 1, HEPES 10, and glucose 10 (pH 7.4 by NaOH). The intracellular solution contained (in mM) KCl 120, MgCl₂ 1, MGATP 3, HEPES 10, and EGTA 10 (pH 7.2 by KOH). For voltage clamp experiments, the glass pipettes were filled with a solution of (in mM) CsCl 60, Cesium aspartate 80, EGTA 11, HEPES 10, and Na₂ATP 5 (pH 7.2 with CsOH). The bath solution consisted of (in mM) NaCl 30, N-methyl-D-glucamine (NMDG) Cl 100, CsCl 5, CaCl₂ 2, MgCl₂ 1.2, HEPES 10, and Glucose 5 (pH 7.4 with HCl). Patch clamp data were collected with an Axopatch 200B amplifier and pCLAMP software (Axon Instruments). Data were digitized at 10 kHz and filtered for analysis at 1 kHz. Experiments were performed at 37° C.

### Functional Assessment of a Truncation Mutant by Multi-Electrode Array Recording

Extracellular recording from WT and truncation syncytial CMs derived from ES cells was performed using a MEA data acquisition system (Multi Channel System, Reutlingen, Germany). The MEA consists of a matrix of 60 titanium nitride coated gold electrodes (30 μm diameter) in an 8×8 layout grid with an inter-electrode distance of 200 μm. The MEA was inserted in the amplifier system. Simultaneous recordings of bipolar extracellular FPs from all electrodes were performed at a sampling frequency of 10 kHz and at 37° C. One electrode at the border of the array was grounded and used as a reference electrode. As described previously by Jiao et al. (2006. A possible mechanism of halocarbon-induced cardiac sensitization arrhythmias. J. Mol. Cell. Cardiol. 41: 698-705), the data were analyzed off-line with a customized toolbox programmed for MATLAB (Mathworks, Natick, Mass.). In order to measure conduction velocity (CV), we calculated the activation time at each point using a threshold-crossing algorithm to form an isochrone map. Three non co-linear points from an area with uniform, parallel isochrones were chosen to calculate CV in two orthogonal directions (e.g. vx and vy). The final CV was calculated as

v=((1/vx)²+(1/vy)²)^{-1/2}.

### Statistical Evaluations

All data are present as means.+−.S.E.M. Statistical analysis of mean values was carried out using unpaired t tests or one-way ANOVAs with post-hoc correction for multiple comparisons. A p value<0.05 was considered statistically significant.

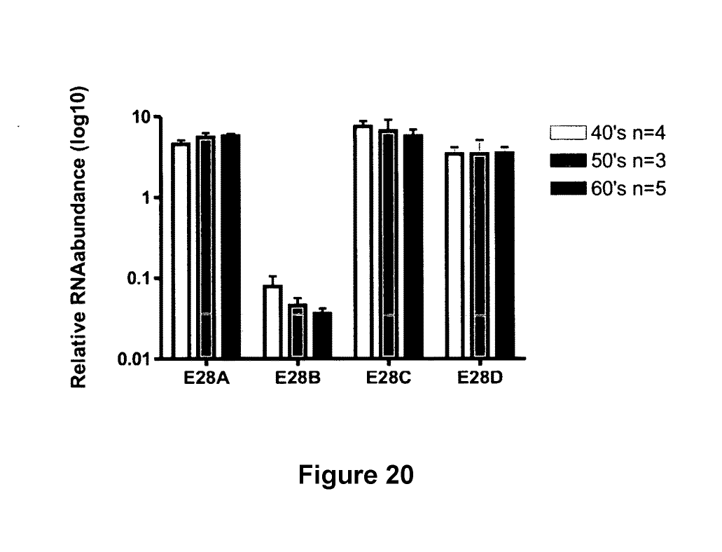

Finally, while the control subjects were younger than the HF patients, we could find no evidence of splice variation changes with age (FIG. 20).

In conclusion, we demonstrate that there are several alternatively truncated forms of SCN5A mRNA in human hearts. During HF, these alternatively spliced mRNA isoforms are likely to reduce sodium current to levels that might contribute to arrhythmic risk alone or in combination with other inciting causes.

### Example 3

This example provides data demonstrating NFκB dependent transcriptional regulation of the cardiac SCN5A sodium channel by angiotensin II, as shown in one or more of U.S. Patent Application Publication No. 2007/0212723 and Shang et al., Am J Physiol Cell Physiol 294: C372-C379.

### AngII and H₂O₂ Dose Ranging in H9c2 Cells

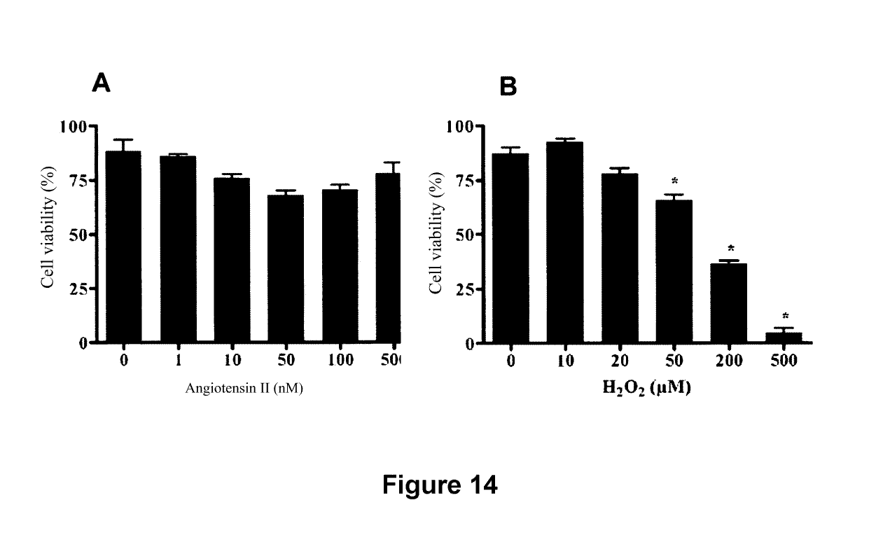

To determine appropriate concentrations of these agents in future experiments, rat H9c2 cardiomyocytes were treated with escalating concentrations of AngII and H₂O₂, and the dose-dependent cell viability was determined. H9c2 cardiomyocytes were tolerant of a wide range of AngII concentrations from 1-500 nM in serum free medium (FIG. 14A). On the other hand, higher doses of H₂O₂ induced cell death starting at concentrations of 50 μM (FIG. 14B). Therefore, we restricted further experiments to 20 μM H₂O₂, where there was no statistically significant increase in cell death over the time course of our experiments. Exposures of 48 h were used to allow sufficient time for transcriptional effects on the Na⁺ current.

Scn5a mRNA Abundance was Downregulated by AngII

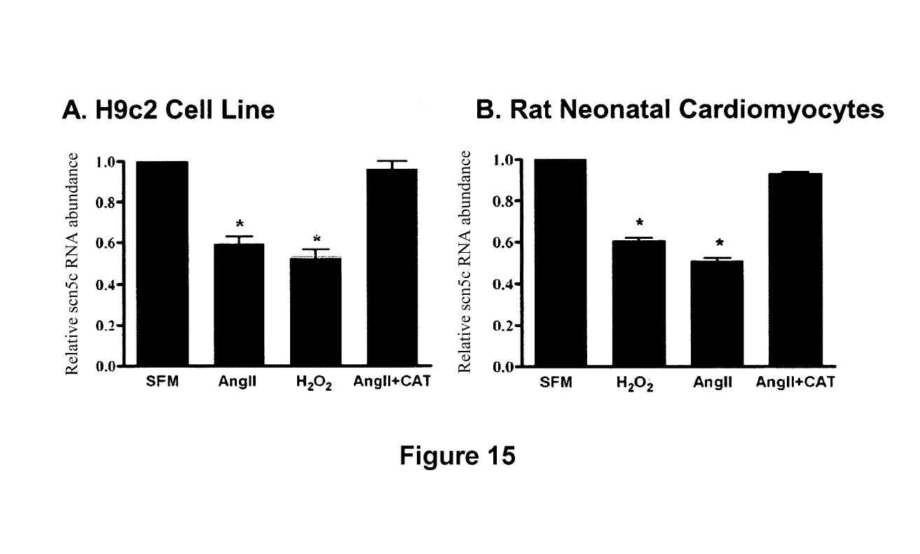

H9c2 cells treated with 100 nM AngII for 48 h showed a 47.3% (.+−.5.3%, n=7, P<0.01) reduction in scn5a mRNA abundance (FIG. 15A). Because H9c2 cells are generally responsive only to high doses of AngII (Tran et al. (1995) Biochim. Biophys. Acta 1259, 283-290; and Laufs et al. (2002) Cardiovasc. Res. 53, 911-920), experiments were repeated with acutely isolated rat neonatal cells and a more physiological concentration of AngII. In neonatal cardiomyocytes, a 48 h exposure to 2 nM AngII resulted in a 49.0% (.+−.5.2%, n=10, P<0.01) reduction in scn5a mRNA abundance in neonatal cardiomyocytes, suggesting that isolated myocytes were more sensitive and the effect was not limited to the H9c2 line (FIG. 15B).

Both Cardiac Na⁺ Channel mRNA and Current were Downregulated by H₂O₂

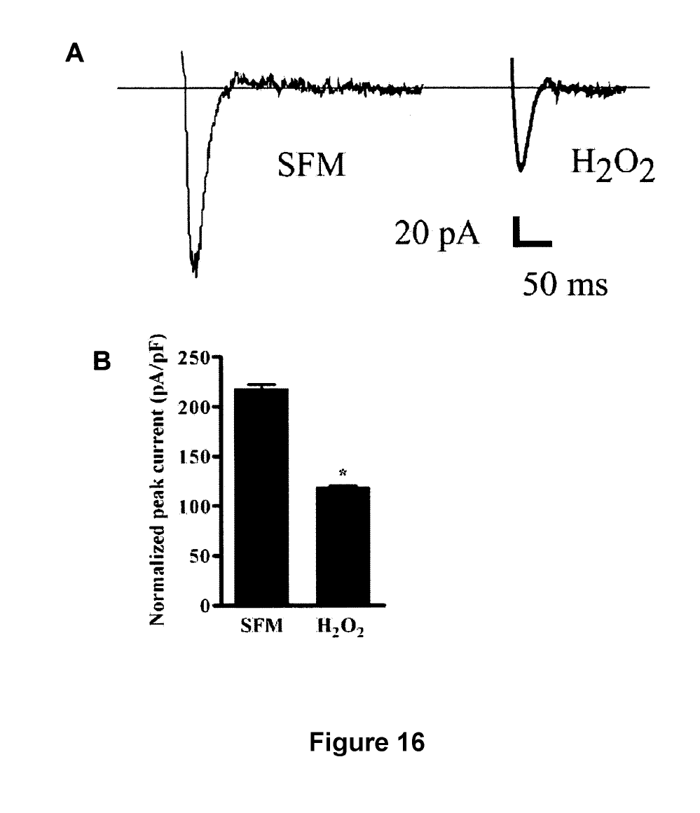

Because AngII is known to activate the NADPH oxidase generating superoxide that is dismutated to H₂O₂, we tested whether the H₂O₂ would recapitulate the AngII results. Exposure of H9c2 cells to 20 μmol/L H₂O₂ for 48 h caused a similar reduction in Na⁺ channel mRNA (46.9%.+−.3.6%, n=9, p<0.01; FIG. 15A). In isolated neonatal rat myocytes and consistent with their increased sensitivity to AngII, there was an analogous response to a reduced concentration of H₂O₂ for 48 h (40 nM; 45.4%.+−.7.5%, n=9, p<0.01; FIG. 15B). In both types of myocytes, the AngII-mediated downregulation of Na+ channel mRNA could be prevented by pretreatment of the cells with catalase, consistent with the idea that AngII was acting through H₂O₂ production. FIG. 16 shows that 20 won H₂O₂ exposure for 48 h resulted in a 46.0% (.+−.2.9%) decrease in Na⁺ current commensurate with the reduction in mRNA abundance (n=7 for control, n=9 in treated group, P=0.01).

### Evidence of NF-κB Regulation of the Cardiac Na⁺ Channel

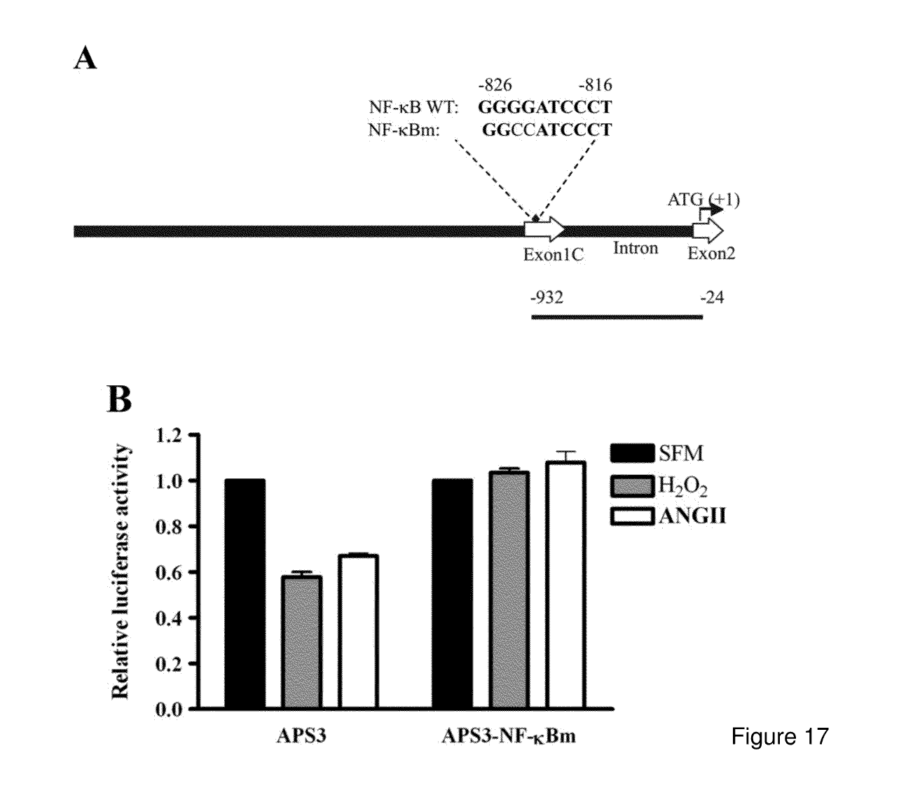

NF-κB is a known redox sensitive transcription factor (Zhou et al. (2001) Free Radic. Biol. Med. 31, 1405-1416). Previously, we have shown that the promoter region of the cardiac Na+ channel contains one NF-κB consensus binding sequence (Shang, L. L. & Dudley, S. C., Jr. (2005) J. Biol. Chem. 280, 933-940). Therefore, we investigated whether NF-κB might be involved in the AngII or H₂O₂-mediated Na⁺ channel transcriptional regulation. To test this idea, we constructed a mutated form of the scn5a promoter in which the NF-κB binding site had been altered, APS3-NF-κBm (FIG. 17A). Promoter-reporter constructs containing the intact NF-κB binding site showed reductions in activity in response to AngII or H₂O₂ (FIG. 17B). The scn5a Na+ channel promoter activity was depressed by 33.0% (.+−.2.6%, n=4, P<0.001) and 42.3% (.+−.4.5%, n=4, P<0.001), respectively, in H9c2 cells when cardiomyocytes transfected with the APS3 construct were compared with and without AngII or H₂O₂ exposures. On the other hand, the construct with a mutated NF-κB binding site showed no significant change in activity in the presence of AngII or H₂O₂.

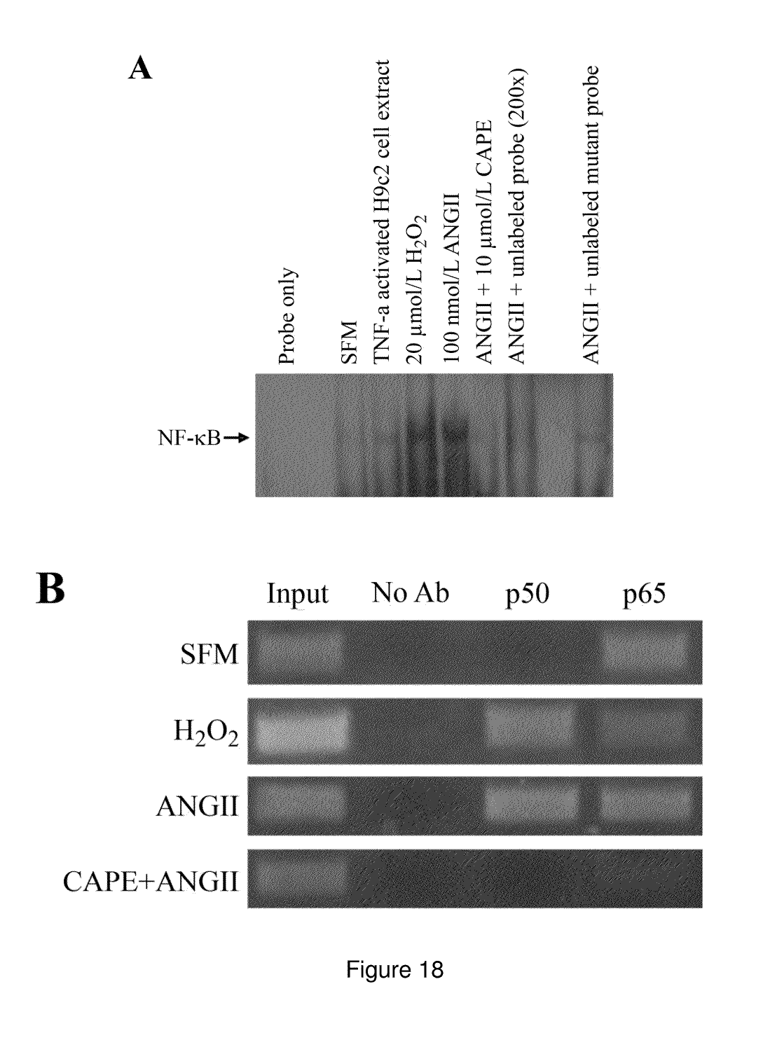

To confirm that NF-.kappa.B was binding to the scn5a promoter in response to AngII or H₂O₂ exposure, we employed electrophoretic mobility shift and chromatin immunoprecipitation (ChIP) assays. A 35 bp fragment of the scn5a promoter containing the NF-κB site was used as the probe (FIG. 18A). The NF-κB binding activity increased in the presence of AngII or H₂O₂ treatment. NF-κB B binding was blocked by caffeic acid phenethyl ester, an NF-κB inhibitor (Natarajan et al. (1996) Proc. Natl. Acad. Sci. U.S.A. 93, 9090-9095; Watabe et al. (2004) J. Biol. Chem. 279, 6017-6026). The ChIP assay showed that there was formation of the complete p50-p65 NF-κB heterodimer on the cardiac Na⁺ channel promoter in response to AngII or H₂O₂ treatment (FIG. 18B).

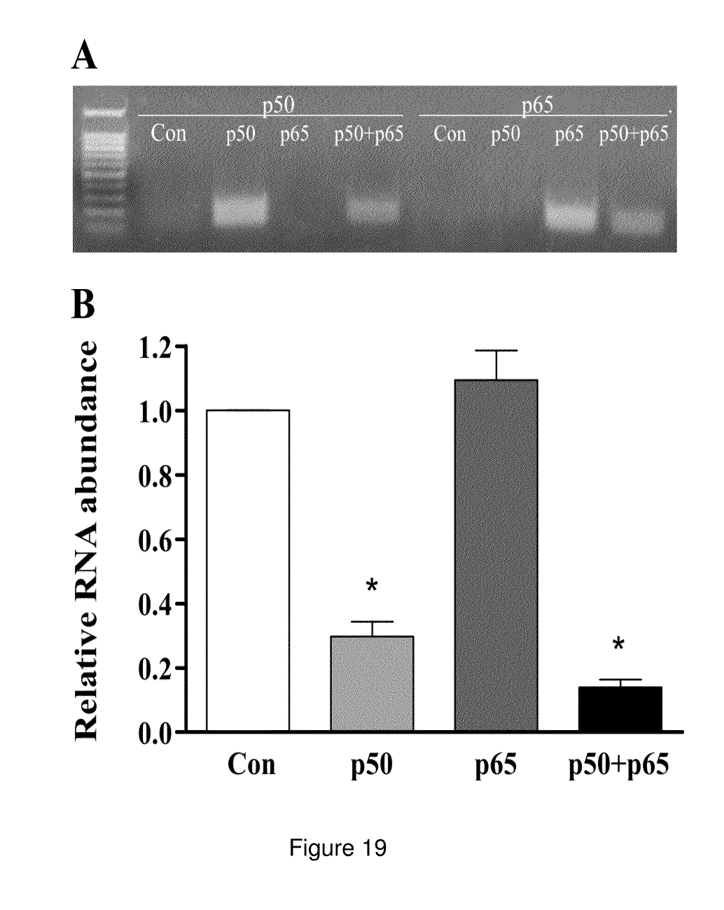

Overexpression of NF-κB subunits in H9c2 cells confirmed the necessity of the p50 subunit for Na⁺ channel downregulation. Quantitative real-time R_{T}-PCR result showed that the relative scn5a mRNA abundances were decreased in cell lines expressing p50 only or the combination of p50 and p65 by 77.3% (.+−.7.3, n=4) and 88.6% (.+−.4.8, n=4), respectively. There was no significantly change in Na⁺ channel mRNA in the presence of p65 overexpression alone, however (FIG. 19).

The following methods were used in this example.

### Cell Culture and Cell Viability Assay

The rat embryonic cardiomyocyte cell line, H9c2 (ATCC cat #CRL-1446), or acutely isolated neonatal rat heart cardiomyocytes were used. Rat neonatal ventricular cells were isolated from 3-day-old Sprague-Dawley rats (Charles River Laboratories Wilmington, Mass.) using a kit and following the manufacturer's instructions (Worthington Biochemical Corp. Lakewood, N.J.). Cells cultured in Dulbecco's modified Eagle's medium (DMEM; ATCC, Manassas, Va.) with 10% fetal calf serum (ATCC) under standard tissue culture conditions at 37° C. to 70-80% confluence were exposed to AngII (Sigma, St. Louis, Mo.) or H₂O₂ (Sigma) in serum free culture medium (SFM) for a total of 48 h in triplicate in 24 well plates. Experiments were repeated three times. After dissociation with 0.125% trypsin-EDTA, 20 μL of 0.4% Trypan-blue (Sigma, St. Louis, Mo.) was added to each well, and a Trypan-blue exclusion viability assay was performed. The use of rats conforms with the Guide for the Care and Use of Laboratory Animals published by the US National Institutes of Health (NIH Publication No. 85-23, revised 1996).

Quantification of scn5a Transcripts by Quantitative Real-Time R_{T}-PCR Assay

To determine the abundance of cardiac sodium channel (scn5a) mRNA under the various conditions, quantitative real-time R_{T}-PCR was used. Total RNA from untreated and treated cardiomyocytes was isolated using the RNeasy Mini Kit (Qiagen, Valencia, Calif.) with the addition of RNase-free DNase I. Reverse transcription was carried out at 42° C. for 30 min with iScript reverse transcriptase (Bio-Rad, Hercules, Calif.), 1 μg total RNA, and 4 .mu.L of 5.times. iScript reaction mix following the manufacturer's instructions. The first strand cDNA was used as Green Supermix (Bio-Rad) and 2.5 μM primer pairs in total 25 μL reaction volume. The forward primer rtPCRscn5aF (5′ GAAGAAGCTGGGCTCCAAGA 3′ (SEQ ID NO. 653)) recognized a sequence from exon 26. The reverse primer, rtPCRscn5aR (5′ CATCGAAGGCCTGCTTGGTC 3′ (SEQ ID NO. 654)), was complementary to exon 27 of scn5a cDNA. The reactions gave rise to a 101 bp PCR product. All amplifications were performed in triplicate and consisted of 40 cycles of 30 s at 95° C., 30 s at 60° C., and 30 s at 72° C. in a BioRad thermocycler icycler (Hercules, Calif.). PCR products were analyzed by relative standard curve methods β-Actin was used as a reference when making quantitative comparison.

### Electrophysiological Determination of Na+ Current

Three hours prior to the start of the patch-clamp experiments, H9c2 cells were trypsinized and plated on treated plastic coverslips. H9c2 cells were treated with or without H₂O₂ as indicated. Glass pipettes were pulled on a Sutter Model P-97 horizontal puller to a resistance of 0.5 to 1.5 MΩ. The glass pipettes were filled with a solution of (in mM) CsCl 60, Cesium Aspartate 80, EGTA sodium 11, HEPES 10, Na₂ATP 5 and pH 7.2 with CsOH. The bath solution consisted of (in mM) Na 30, N-methyl-D-glutamate chloride 100, CsCl 5, CaCl₂ 2, MgCl₂ 1.2, HEPES 10, Glucose 5 and pH 7.4 with NaOH. Once a seal was established, a small amount of suction was applied to obtain the whole cell configuration. From a holding potential of −100 mV, peak currents obtained at −10 mV were used for comparison. Cells were tested at 25° C. Data were sampled at 10 kHz and later filtered at 5 kHz for analysis. Currents were recorded and analyzed with an Axopatch 200B amplifier, Axon Digidata 1230A A/D converter and pClamp software (Molecular Devices Corporation, Sunnyvale, Calif.).

### Promoter-Reporter Constructs and Transient Transfection

The scn5a promoter region has previously been defined (Shang, L. L. & Dudley, S. C., Jr. (2005) J. Biol. Chem. 280, 933-940). For these experiments, a new promoter construct that contained the NF-κB consensus binding site was used to test the effect of treatments on scn5a transcription. This construct, pGL3-APS3, consisted of a 937 bp fragment starting from exon 1C to +32 base pairs relative to the start codon located on exon 2 of mouse scn5a gene.

H9c2 cardiomyocytes were plated in each well of 24-well plates at a density of 2.5×10⁴ cells in a final volume of 1 mL of culture medium, allowed to attach overnight, and expand to 70%-80% confluence. Transfection of 0.3 μg of the promoter-reporter construct and 0.013 μg of a plasmid containing the herpes simplex virus thymidine kinase (HSV-TK) promoter driving expression of a synthetic Renilla luciferase (phRL-TK; Promega, Madison, Calif.) was carried out with 0.9 μL of Fugene6 chemical transfection reagents (Roche, Indianapolis, Ind.) following the manufacturer's instructions. The serum free DMEM cultural media with or without AngII or H₂O₂ was changed every 24 h. After culture for 48 h, the cells were treated with passive lysis buffer (Promega, Madison, Calif.), and cell extracts were collected for analysis of firefly and Renilla luciferase activities using 100 .mu.L of luciferase assay substrate and 100 .mu.L of Stop & Glo reagent of the dual-luciferase reporter assay system (Promega, Madison, Calif.). Light emission was quantified in a Veritas microplate luminometer using Veritas-version 1.4.0 software (Tuener Biosystems, Sunnyvale, Calif.). Transfection efficiency of the reporter constructs was controlled by comparison to Renilla luciferase activity. The phRL-TK vector minimized any modulation of Renilla luciferase expression by the experimental conditions since it has been engineered to remove the majority of potential transcription factor binding sites. The luciferase activity of the all promoter-constructs was normalized to a pGL3-basic promoter-less control transfected simultaneously. Four separate transfection sessions were analyzed, and at each session, transfections were performed in triplicate. Three dual luciferase readings were taken for each transfection experiment.

### Site-Directed Mutagenesis of NF-κB Binding Site

Disruption of the NF-.kappa.B binding site was undertaken using the QuikChange II XL sitedirected mutagenesis kit according to the manufacturer's instructions (Stratagene, La Jolla, Calif.). Briefly, for PCR 10 ng of pGL3-APS3 was used as a template, and the nucleotide primers listed were used to mutate the NF-κB binding site (the bold as wild type, the underline as mutant) of pGL3-APS3: NFκB-mutCF: 5′GGTGCTGCACTCAGGCCATCCCTATGAGATCCTC 3′ (SEQ ID NO. 655) and NFκB-mutCR: 5′ GAGGATCTCATAGGGATGGCCTGAGTGCAGCACC 3′ (SEQ ID NO. 656). After digestion with DpnI, 2 μL of PCR product were used to transform XL10-Gold competent cells. Sequencing identified appropriate clones.

### Electrophoretic Mobility Shift Assay (EMSA/Gel-Shift)

The H9c2 cells were treated for 48 h with AngII or H₂O₂, with or without CAPE (caffeic acid phenethyl ester, an NF-κB inhibitor at 10 μM) starting 24 h after plating. Approximately 5×10⁶ cells were scraped for nuclear protein extraction by nuclear extract kit (Activemotif, Carlsbad, Calif.). A double-stranded oligonucleotide containing the consensus-binding sequence (bold) for NF-κB (5′GGTGCTGCACTCAGGGGATCCCTATGAGATCCTC 3′ (SEQ ID NO. 657)) and NF-κB mutant sequence (5′GGTGCTGCACTCAGGCCATCCCTATGAGATCCTC 3′ (SEQ ID NO. 658)) from scn5a promoter were used as probes to assay for binding activity of the nuclear extracts. Protein-DNA complexes were detected using biotin end-labeled double-stranded DNA probes prepared by annealing complementary oligonucleotides. Oligonucleotides were labeled in a reaction using terminal deoxynucleotide transferase and biotin-N4-CTP (Pierce, Rockford, Ill.) following the biotin 3′ end DNA labeling kit manual. The binding reaction was performed using the LightShift kit (Pierce). Briefly, 30 μg of nuclear extracts and binding buffer were incubated on ice for 5 min in a volume of 20 μL, then the labeled probe (20 fmol) was added, and the reaction was allowed to incubate for an additional 25 min. Following electrophoresis, the DNA-protein complexes were transferred onto nylon membranes and detected using chemiluminescence. TNF-α activated H9c2 cell nuclear extract (5 μg) was used as positive control. The reaction products were separated on a 6% retardation gel. Specificity was confirmed by addition of unlabelled probe in 200-fold excess.

### Chromatin Immunoprecipitation (ChIP) Assay

Formaldehyde cross-linking and chromatin immunoprecipitation was performed as described in manufactory's manual (ChIP-IT™ kit, Activemotif). Briefly, proteins were crosslinked with chromatin using 1% formaldehyde in H9c2 cells with or without treatment. The cells were subsequently sonicated in lysis buffer, and an aliquot of the lysate was used in a PCR reaction. The remaining lysate was cleared with protein G beads. One half of the cleared lysate was incubated with p50 or p65 antibody, while the other half was used as a negative control without the antibody. After reversing the cross-linking, the immuno-complex was digested with proteinase K, and the DNA was purified. DNA was analyzed by PCR with the PicoMax Polymerase (Stratagene, La Jolla, Calif.) and primers specific to the APS3 promoter region.

### Stable H9c2 Cell Lines Overexpressed NF-κB Subunits p50 and p65

The H9c2 cells were co-transfected with expression vectors carrying human NF-.kappa.B subunits p50 and/or p65 (Lindholm et al. (2003) J. Hypertens. 21, 1563-1574) and pDsRed-express-N1 vector carrying red fluorescent protein as marker (Clontech, Mountain View, Calif.) and selected with 400 .mu.g/mL geneticin (Invitrogen) for at least for four weeks. At which time, over 90% of the cells showed red fluorescence. Transfection was confirmed by R_{T}-PCR using human p50 or p65 specific primers. The SYBR quantitative real-time R_{T}-PCR was used to assay the Na+ channel expression.

### Statistical Evaluations

All data are present as means.+−.S.E.M. Statistical analysis of mean values was carried out using Student's paired or unpaired t tests. ANOVA was used for comparison of variance between multiple means. A p value<0.05 was considered statistically significant.

### Example 4

The following is related to data provided herein.

Heart Failure Increases Two of the Na+ Channel C-Terminal Splice Variants

The presence of splice variants was compared between explanted ventricles and control patients with no known cardiac disease. R_{T}-PCR results indicated that the relative mRNA abundance of E28A full-length variant was decreased by 24.7% in HF patients compared to controls (p<0.001). E28C and E28D mRNA abundances were increased 14.2 fold (p<0.001) and 3.8 fold (p<0.001) respectively comparing controls to HF patients. As a percentage of the total SCN5A transcript, E28A and B decreased significantly from 87.5% (±5.1) and 2.4% (±0.4) in controls to 45.1% (±4.5) and 0.5% (±0.2) in HF patients. The E28C and D variants increased from 3.9% (±0.6) and 6.2% (±4.6) in controls to 34.3% (±3.1) and 20.2% (±3.3) in HF patients. The total percentage of short variants went from 12.5% (±5.1) of the total SCN5A mRNA in control subjects to 54.9% (±4.5) in HF patients. Similar amounts of truncated channel variants are known to cause Brugada syndrome (Chen et al., Nature 392: 293-296 (1998); Makiyama et al., J Am Coll Cardiol 46:2100-2106 (2005); Priori et al., Circulation 105: 1342-1347 (2002); Schulze-Bahr et al., Hum Mutat 21: 651-652 (2003); Smits et al., J Am Coll Cardiol 40: 350-356 (2002)). The pattern of changes for the truncation variants was similar in both ventricles with increases in E28C and E28D. Corresponding to the RNA effects, Western analysis of human control and heart failure tissue revealed a 62.8% (±9.7, n=3, p<0.01) protein reduction in HF comparing to normal heart. No bands that might correspond to truncation variants were observed in normal and failing heart.

Truncation Variants Reduce Na+ Channel Protein and Current.

Variant cDNA was expressed in the human embryonic kidney (HEK)-SCN5A cell line stably expressing the full-length E28A channel. The expression of E28D reduced the E28A variant mRNA abundance. Using increasing ratios of vector encoding E28D resulted in progressive reductions in full-length transcript mRNA abundance. Neither E28C nor E28D variants generated current when transfected into HEK cells alone, and when transfected into the HEK-SCN5A cell line stably expressing the full-length Na+ channel, both variants reduced Na+ current. The presence of the C or D variants resulted in 54.6% (±8.5, p<0.01, n=14) and 56.0% (±8.9, p<0.01, n=10) reductions in peak current respectively when compared to native alone. The reduction of current was dependent on the ratio of variant to full-length vector used. Fluorescent microscopy of HEK cells transfected with Na+ channel C-terminal labeled variants demonstrated markedly reduced amounts C or D variant Na⁺ channel protein when compared to an equal amount of the full-length E28A variant.

Physiological Significance of SCN5A Truncation Variants.

The physiological significance of truncations in SCN5A Exon 28 was tested by making a gene-targeted mouse model with a nonsense mutation in Exon 28 between the truncations caused by the C and D variants. This mutation was lethal to embryos. Undifferentiated mouse embryonic stem cells heterozygous for the SCN5A1652stop had normal growth characteristics and could be differentiated into spontaneously beating cardiomyocytes (CMs). The peak I_{Na} was decreased by 86.1% (±5.2, n=8, p=0.0002) in differentiated CMs containing the truncation when compared to that of WT, again showing a dominant negative effect of the truncation on the wild-type channel. Action potentials recorded in the current clamp mode from spontaneously beating CMs showed significant slowing of the beating frequency (p=0.02, n=11), a significant reduction in the maximum rate of rise of the action potential in the truncation mutation (p<0.01, n=11), and a reduced amplitude (00.01, n=11) in comparison with wild-type. These changes are consistent with reduced Na+ channel function (Smits et al., J Mol Cell Cardiol 38: 969-981 (2005); Tan et al., Nature 409: 1043-1047 (2001); Lei et al., J Physiol 567: 387-400 (2005)). Syncytial properties of these CMs were studied using multielectrode arrays (MEAs) (Caspi et al., Ann NY Acad Sci 1015: 285-298 (2004); Kehat et al., Circ Res 91:659-661 (2002)). Consistent with a physiologically significant reduction in Na+ current as a result of the truncated mRNA, MEA recordings of CMs with the truncation mutation showed conduction velocity was decreased by 64.2% (p<0.03) as compared to the wild-type (Halbach et al., Cell Physiol Biochem 13:271-284 (2003)).

### Example 5

This example demonstrates that splicing factors hLuc7A and RBM25 are associated with abnormal splicing of SCN5A, as shown in one or both of Gao et al., Circulation, 124(10): 1124-31 (published online on Aug. 22, 2011) and in International Patent Application Publication No. WO/2010/129964.

The following paragraphs describe the methods used in Examples 5 and 6:

### Cell Culture

Jurkat T cell clones E6.1 (ATCC, Manassas, Va.) were cultured in RPMI 1640 medium supplemented with 10% heat inactivated fetal calf serum, 4 mM glutamine, 75 units/mL streptomycin and 100 units/mL penicillin.

Human embryonic stem (ES) cells were maintained on mouse embryonic fibroblasts (MEFs) as previously described. 14 Cardiomyocytes were differentiated from WA09 (H9) ES cells using a directed differentiation approach in defined media for efficient cardiogenesis. After 30 days of differentiation, the human embryonic stem cell-derived cardiomyocytes (hESC-CMs) were used in this study.

### Real-Time PCR Quantification

Total RNA was isolated from cultured cells and human ventricular tissue using the RNeasy Mini Kit and RNeasy Lipid Tissue Mini Kit respectively (Qiagen, Valencia, Calif.). Human heart tissue was obtained from a tissue bank maintained at Advocate Christ Cardiac Surgery Clinical Research Center.

### Transfection and Infection Assays

Fugene 6 reagents from Roche (Madison, Wis.) were used for transfection assays by following the manufacturer's instructions. Small inhibitory RNAs (siRNAs) for LUC7L3 and RBM25 were purchased from Santa Cruz Biotechnology (Santa Cruz, Calif.). Human pGIPZ lentiviral short hairpin RNAmir particles were purchased from Open Biosystems (Huntsville, Ala.). Human cardiomyocytes were placed in a 24-well plate at the density of 200,000/well. RBM25 short hairpin RNA (shRNA; 5 μL for each well based on pre-titer results) was pre-incubated with polybrene (Sigma, Milwaukee, Wis.) at final concentration 8 μg/mL for 1 h and aliquoted to each well. The scrambled shRNA group followed the same protocol. The media was replaced by regular culture media after 5 h. The infection rates and RBM25 knockdown rates were evaluated by confocal microscope (Carl Zeiss GmbH, Oberkochen, Germany) and qPCR respectively on day 2 and day 3. Green fluorescent protein (GFP)-tagged open reading frame clones of Homo sapiens LUC73 and RBM25 were purchased from ORIGENE (Rockville, Md.). The transfection assays followed the manufacturer's instructions.

### Electrophysiology

hESC-CMs were trypsinized (2.5%, Invitrogen) for 10 min and plated on 35 mm glass bottomed culture dish (MatTek, Ashland, Mass.) at cell density 40,000 cells/dish on the day before the experiments. Na+ channel currents were measured by using the whole-cell patch-clamp technique in the voltage-clamp configuration at room temperature. To measure Na+ channel currents, pipettes (3 to 4MΩ) were filled with a pipette solution containing (in mmol/L): CsCl 80, cesium aspartate 80, EGTA 11, MgCl₂ 1, CaCl₂ 1, HEPES 10, and Na₂ATP 5 (adjusted to pH 7.4 with CsOH). The bath solution consisted of (in mmol/L): NaCl 130, CsCl 5, CaCl₂ 2, MgCl₂ 1.2, HEPES 10, and glucose 5 (adjusted to pH 7.4 with CsOH).15 The holding potential was −100 mV. A voltage step protocol ranging from −80 to +70 mV with steps of 10 mV was applied to establish the presence of Na+ channel currents. The peak current density was used to plot current-voltage (1-V) curves. Nifedipine (10 μM, Sigma) was added in the bath solution to block L-type Ca+ channel currents.

### Gel Mobility Shift Assays

RNA gel mobility shift assays were performed by using the LightShift Chemiluminescent RNA electrophoretic mobility shift assay (EMSA) Kit (Pierce, Appleton, Wis.). In brief, biotinylated wild-type (CAGCAGGCGGGCAGCGGCCU) and mutant (CAGCAGGUUAGAGGCGGCCU) RNA substrates were synthesized by Invitrogen. Binding of biotinylated RNA to RBM25 was achieved by incubating 0.2 nmol/L of RNA and variable amounts of protein for 30 min at 4° C. in 20 μL of binding buffer. For the competition assays, a molar excess of unlabeled competitor RNAs at various fold levels was added to the pre-incubated reaction mixture. Samples were fractionated in a native 5% polyacrylamide gel and transferred to Hybond-N+nylon membranes (Pierce, Appleton, Wis.). The biotin-labeled RNA was detected using the streptavidin horseradish peroxidase conjugate and a chemiluminescent substrate.12

### Western Blots Assays

The Mini-PROTEAN® Tetra Electrophoresis System from BioRad (Hercules, Calif.) was used for Western blots analysis. Anti-RBM25 antibodies were provided by Dr Shu-Ching Huang (Dana-Farber Cancer Institute). Anti-LUC7L3 antibodies were purchased from Millipore (Billerica, Mass.). Anti-GFP was purchased from ORIGENE (Rockville, Md.).

### Statistics

Data are presented as means±standard error of the mean (SEM). Means were compared using unpaired Student's t test or one-way analysis of variance (ANOVA). A probability value P<0.05 was considered statistically significant.

### Clinical Specimens

The microarray study samples were composed of end-stage cardiomyopathy hearts (n=10) and nonfailing control hearts (n=6).1 End-stage cardiomyopathy heart samples were obtained at the time of left ventricular assist device (LVAD) placement or cardiac transplantation. Subjects with end-stage cardiomyopathy exhibited severely reduced ejection fraction, left ventricular dilation, elevated pulmonary arterial and wedge pressures, and a reduced cardiac index. The control subjects were younger (median age 42 years with an interquartile range of 24-50 years) and predominantly male.

### Data Analysis Methods

The GeneSifter gene expression microarray data analysis system was used to identify and compare significant differentially expressed genes from human (GEO accession: GSE1869)1 heart failure tissue-derived gene expression data. The data were uploaded to GeneSifter by Batch Upload with the option to use Affymetrix probe IDs. No data points were missing from any of the files. The z score was used as a measure of the significance of observed genes compared to a normal distribution. A z score was considered significant if it was >2 or <−2, implying the genes were significantly over-represented or under-represented. Statistically significant gene changes were identified using an unpaired Student's t test, p value<0.05 and a 5% Benjamini and Hochberg false discovery rate (FDR) correction. These settings are similar to those used by Kittleson et al.1 A range of fold change cutoffs was used. For functional analysis, gene ontology (GO) reports were generated according to the method of Doniger et al.2 Genes associated with RNA splicing were found under the biological process GO term “GO:0008380 RNA splicing”. The upregulated splicing factors are listed in Table 1. No significant downregulation of splicing factors was observed. Hierarchical clustering of these genes across samples was done using the average correlation approach through Bioconductor (Fred Hutchinson Cancer Research Center). The heatmap (FIG. 1) represents the relationship of genes and samples to one another with green representing downregulated and red representing upregulated genes relative to the mean expression value of each gene.

Altered mRNA Profiles of Splicing Factors in Human HF Tissue

A mRNA microarray analysis was used to identify and compare splicing factors in both normal and human HF tissues. Of the 181 known human splicing factors analyzed, 17 were upregulated in HF. These splicing factors were grouped according to known pathogenic regulators, such as hypoxia, inflammation, wall tension, or hormonal factors, involved in HF (Table 1).

TABLE 1
Comprehensive list for human HF-related splicing factors

| Pathogenic regulation | |
|---|---|
| involved | Gene symbol |
| Hypoxia | RBM25; LUC7L3; TARDBP; HNRPH3; PPIG |
| Inflammation | BAT1; RBM39; IVNS1ABP |
| Hormone level changing | BAT1 |
| Overloading pressure | RBM39 |
| Others | YTHDC1; SFPQ; QKI; HNRNPA1; TRA2A |

### Upregulation of RBM25 and LUC7L3 in Human HF Tissue

The cis-element, CGGGCA, of splicing factor RBM25 was found to be near the splicing sites of SCN5A variants E28C and E28D. RBM25 requires LUC7L3 to be active in splicing regulation, so both RBM25 and LUC7L3 were evaluated further for a role in SCN5A mRNA splicing regulation. Of the other 45 splicing factors upregulated, based on known cis-element sequence, none is known to bind to or has canonical binding sequences that are present in SCN5A.

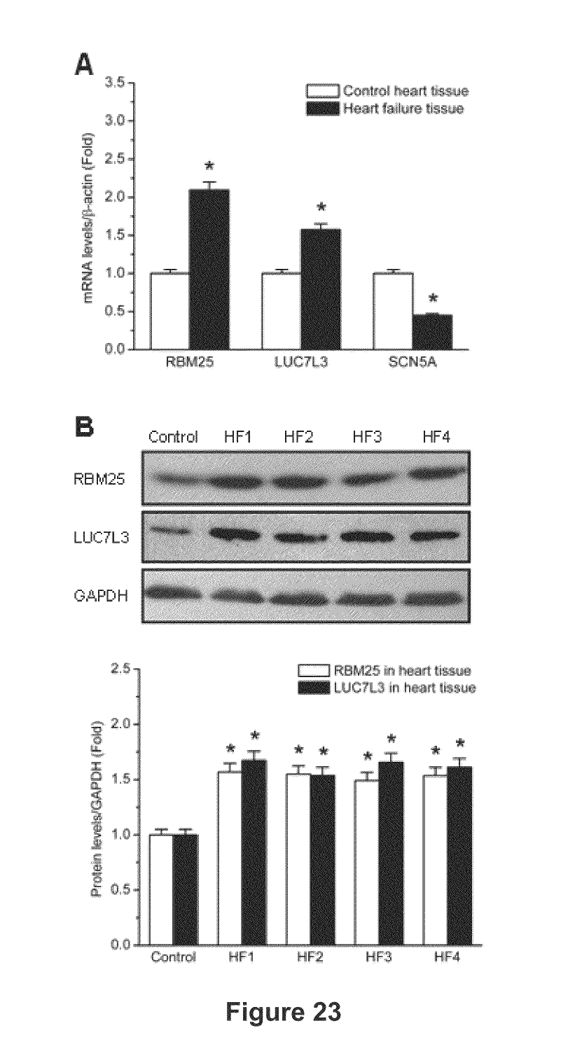

The upregulation of splicing factors RBM25 and LUC7L3 was confirmed in human HF tissue by qPCR. Compared to the normal human heart tissue, the results indicated that the relative abundances of RBM25 and LUC7L3 were increased by 1.1-fold and 0.6-fold in HF tissue respectively (P<0.05, FIG. 23A). Full length SCN5A mRNA was reduced by 0.6-fold in HF tissue (P<0.05, FIG. 23A), a result similar to our previous report.8 mRNA findings were correlated with protein expression by Western blots. The representative Western blots are shown in FIG. 23B. Compared to the control group (mixture of 4 normal human heart tissue samples), Western blot quantification showed that RBM25 was increased by 0.5- to 0.6-fold in HF tissue samples 1-4, respectively (P<0.05, three replications for each sample). The gel density of LUC7L3 was increased by 0.6- to 0.7-fold in the same HF tissue samples (P<0.05, three replications for each sample).

RBM25 Associates with SCN5A and Interacts with CGGGCA

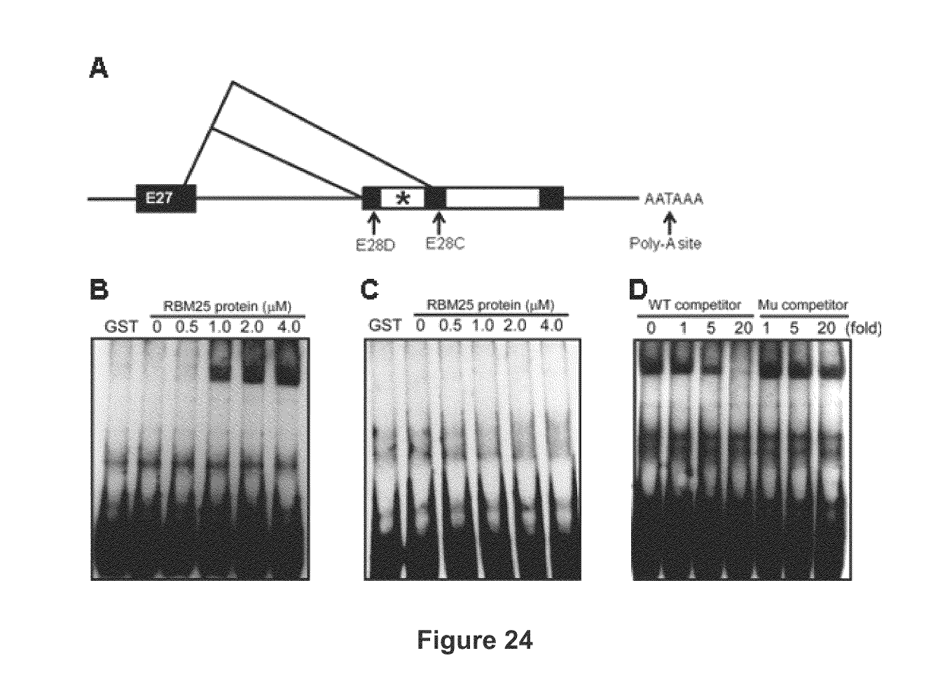

Gel mobility shift assays showed that RMB25 was bound to the canonical sequence, CGGGCA, in SCN5A exon 28 (FIG. 24). Scanning the entire SCN5A RNA sequence revealed only a single binding site for RBM25 at the place where SCN5A splicing variants were detected (FIG. 2A). Binding of biotinylated wild-type (CAGCAGGCGGGCAGCGGCCU) RNA to RBM25 was observed in FIGS. 2B-D. The results illustrated that RBM25 binding was specific to the sequence CGGGCA. RBM25 was bound to the wild-type SCN5A sequence in a concentration-dependent manner (FIG. 24B). For the competition assays, a molar excess of unlabeled competitor RNAs at various fold levels was added to the pre-incubated reaction mixture (FIG. 2D). Specificity was confirmed by showing a lack of this binding to a mutated canonical binding sequence (FIG. 24C) and the inability of unlabeled probe to compete with labeled probe for RBM25 binding (FIG. 24D).

### Example 6

This example demonstrates Ang-II and hypoxia regulate RBM25, hLuc7A, and SCN5A mRNA splicing, as shown in one or both of Gao et al., Circulation, 124(10):1124-31 (published online on Aug. 22, 2011) and in International Patent Application Publication No. WO/2010/129964.

Ang II and Hypoxia Regulated RBM25, LUC7L3 Expression as well as SCN5A mRNA Splicing

Ang II and hypoxia are common pathogenic factors in HF and were identified in the microarray analysis as possible upstream stimuli responsible for the changes in mRNA splicing factors (Table 1). SCN5A mRNA is known to be transcribed in skeletal muscle and leukocytes. We have reported that leukocytes have a similar mRNA splicing pattern to that in heart. Moreover, circulating leukocytes from HF patients showed a four-fold increase in the Ang II type 1 receptor (data not shown). Therefore, Jurkat cells, an immortalized line of T lymphocyte cells, which prominently express SCN5A, were chosen to be as an initial model to study the SCN5A regulation mechanism.

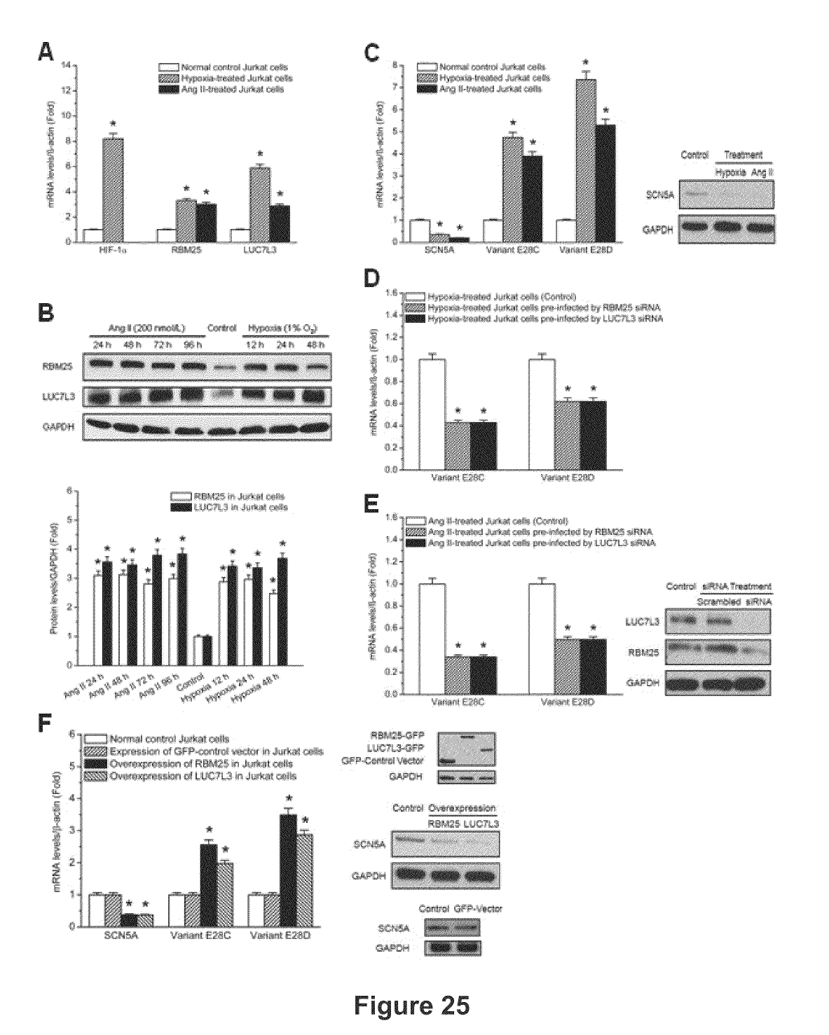

Jurkat cells were divided into three experiment groups: untreated control, hypoxia-treated (1% O₂), and Ang II-treated (200 nmol/L). The cells were harvested from each experiment group at four time points (30 min, 24 h, 48 h, and 72 h), and total mRNA was extracted. The expressions of RBM25 and LUC7L3 were examined by qPCR, and the results at 48 h are shown in FIG. 25A. HIF-1α was used as the indicator of cellular hypoxic stress. Under the hypoxia-treated condition, the expressions of RBM25 and LUC7L3 in Jurkat cells were increased by 2.4-fold and 4.9-fold, respectively (P<0.05). Under the Ang II-treated condition, the expressions of RBM25 and LUC7L3 in Jurkat cells were increased by 2.1-fold and 1.9-fold respectively (P<0.05). The expressions of RBM25 and LUC7L3 were analyzed by Western blots at three time points (12 h, 24 h, and 48 h) for the hypoxia-treated group and at four time points (24 h, 48 h, 72 h, and 96 h) for the Ang II-treated group. Western blot quantification showed that RBM25 was increased by 1.9-, 2.0- and 1.5-fold in the hypoxia-treated group at time points 12 h, 24 h and 48 h, respectively and was increased by 2.1-, 2.1-, 1.9- and 2.0-fold in the Ang II-treated group at 24 h, 48 h, 72 h and 96 h, respectively (P<0.05). The gel density of LUC7L3 was increased by 2.4-, 2.4- and 2.7-fold in the hypoxia-treated group at time points 12 h, 24 h and 48 h, respectively and was increased by 2.6-, 2.5-, 2.8- and 2.8-fold in the Ang II-treated group at time points 24 h, 48 h, 72 h and 96 h, respectively (P<0.05). The representative Western blots and quantification (based on three replications for each group) are shown in FIG. 25B.

The effect of hypoxia and Ang II on the SCN5A variants E28C and E28D in Jurkat cells was studied also to correlate SCN5A variants with RBM25 and LUC7L3 abundances. The expressions of the full length SCN5A transcript and SCN5A variants E28C and E28D at 48 h are shown in FIG. 25C. With hypoxia, the expressions of SCN5A variants E28C and E28D were increased by 3.7-fold and 6.4-fold, respectively (P<0.05), while the expression of the full length SCN5A transcript was decreased by 0.7-fold (P<0.05). With Ang II, the expressions of SCN5A variants E28C and E28D were increased by 2.9-fold and 4.3-fold, respectively (P<0.05), while the expression of the full length SCN5A transcript was decreased by 0.8-fold (P<0.05).

siRNAs for these two splicing factors were found to block partially the increases in the hypoxia or Ang II-induced SCN5A variants E28C and E28D at 48 h (FIG. 25D-E). The partial effect on reducing abnormal splicing may be due to the siRNA knockdown efficiencies were 60±5% and 70±5% estimated by qPCR and 63±7% and 69±6%. by Western blots for RBM25 and LUC7L3, respectively.

While downregulation of the two splicing factors in Jurkat cells reduced the SCN5A variants E28C and E28D, overexpression of RBM25 and LUC7L3 increased E28C and E28D and decreased the full length SCN5A mRNA abundances. The expressions of the full length SCN5A transcript and SCN5A variants E28C and E28D at 48 h are shown in FIG. 25F. With overexpression of RBM25, the expressions of SCN5A variants E28C and E28D were increased by 1.6-fold and 2.5-fold, respectively (P<0.05), while the expression of the full length SCN5A transcript was decreased by 0.6-fold (P<0.05). With overexpression of LUC7L3, the expressions of SCN5A variants E28C and E28D were increased by 1.1-fold and 1.9-fold, respectively (P<0.05), while the expression of the full length SCN5A transcript was decreased by 0.7-fold (P<0.05).

The Effect of Ang II on Na⁺ Channels in hESC-CMs

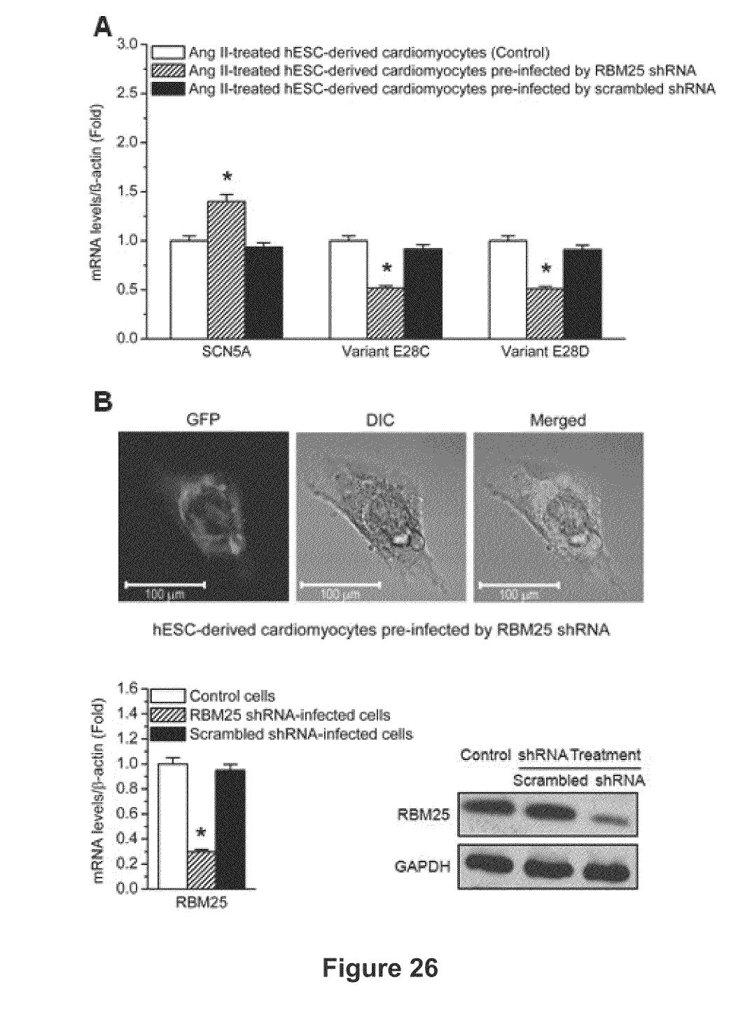

The effect of Ang II on the cardiac Na+ channel was investigated in hESC-CMs. hESC-CMs were plated on a 24-well culture plate on day 30 of differentiation. The cells were divided into three experiment groups: Ang II-treated (200 nmol/L), Ang II-treated (200 nmol/L) and pre-infected by pGIPZ lentiviral RBM25 shRNAmir, and Ang II-treated (200 nmol/L) and pre-infected by scrambled shRNA. Ang II (200 nmol/L) treatment was given to all the experiment groups on infection day 3. When cells were pre-infected by RBM25 shRNA, the expression of the full length SCN5A transcript was increased by 0.4-fold, while the expressions of SCN5A variants E28C and E28D were decreased by 0.4-fold and 0.5-fold, respectively (P<0.05). qPCR measurements were performed in each experiment group at 24 h after Ang II treatment and normalized by β-actin. No changes were observed when cells were pre-infected by scrambled shRNA, however. The results indicated that Ang II-mediated SCN5A downregulation was dependent on the splicing factor RBM25 (FIG. 26A). The infection rate was 90±6%, evaluated by the ratio of GFP positive cells (pGIPZ lentiviral infected cells) to total cells. RBM25 knockdown efficiency was 70±5%, evaluated by both qPCR and Western blots (FIG. 26B).

Abnormal Na⁺ Channel mRNA Processing Altered Na⁺ Channel Current

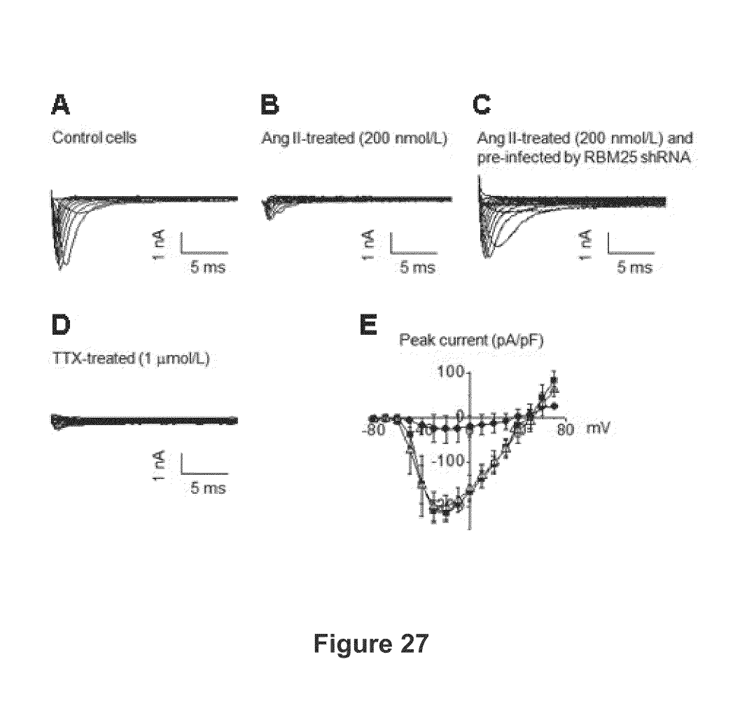

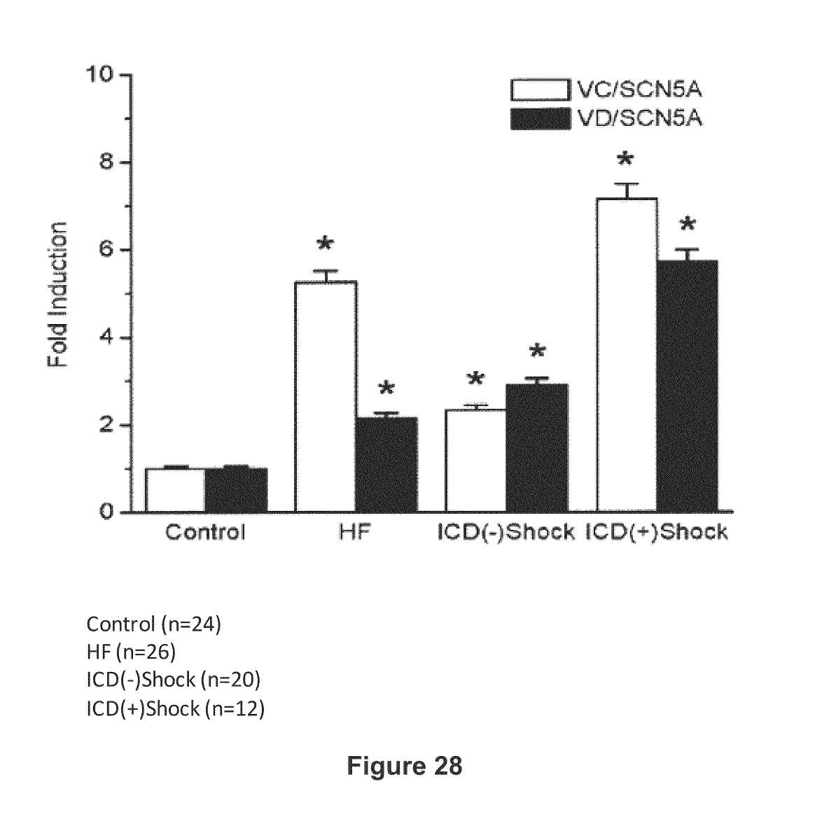

The implications of Na+ channel mRNA processing changes were tested by measuring Na+ current in hESC-CMs by the whole-cell voltage-clamp technique. hESC-CMs were used to most accurately mimic clinical conditions and because a suitable animal model has not been validated. The cells were divided into four experiment groups: Control, Ang II-treated (200 nmol/L), Ang II-treated (200 nmol/L) and pre-infected by RBM25 shRNA, and Ang II-treated (200 nmol/L) and pre-infected by scrambled shRNA. Given that RBM25 regulates pre-mRNA alternative splicing by recruiting LUC7L3,12 loss-of-function of RBM25 with lentiviral shRNA was used exclusively to suppress abnormal channel splicing. The macroscopic Na+ channel currents in each experiment group were measured. The results from the first three experiment groups at 24 h after Ang II treatment are shown in FIG. 27. There was a significant difference in peak currents between control cells and Ang II-treated cells at membrane potentials ranging from −40 to +30 mV (P<0.05). The effect of Ang II on peak current was not observed when the cells were pre-infected by RBM25 shRNA before Ang II treatment. Non-specific effects of the lentivirus were excluded by comparing with the cells pre-infected by scrambled shRNA, which had no effect on the I-V relationship compared to Ang II alone (data not shown). The results indicated that Ang II could downregulate Na+ channel currents in hESC-CMs and that this downregulation was dependent on the splicing factor RBM25.

### Example 7

This example demonstrates that SCN5A variants activate the unfolded protein response (UPR), as shown in International Patent Application Publication No. WO/2010/129964.

Previously, we have shown that SCN5A splicing variants have a dominant negative effect on Na⁺ current (Shang et al., Circ. Res., 101:1146-1154 (2007)). Since the Na⁺ channel is encoded by a single mRNA, it is unclear how truncated forms might have a dominant negative effect on full-length channel production. The following Example investigated whether truncated SCN5A variant activate the unfolded protein response (UPR) pathway.

Hypoxia and AngII were used to increase abnormal SCN5A splicing. The expressions of PERK and sXBP1 were measured by R_{T}-PCR. The expression of PERK was increased at 48 h by 18.6±0.8 fold (p<0.05) and 14.2±0.6 fold (p<0.05) under hypoxia-treated and Ang II-treated conditions respectively, while no expression upregulation of sXBP1 was observed. To test if this upregulation of one arm of the UPR was mediated by SCN5A mRNA variants, exogenous E28C and E28D were introduced. The Jurkat cells were divided into four experiment groups: normal control, empty vector control, variant E28C overexpressioned cells, and variant E28D overexpressioned cells. The expression of PERK was measured by R_{T}-PCR in each group at the time point 48 h. Results indicated that the expression of PERK was increased by 6.3±0.4 (p<0.05) fold and 7.9±0.5 fold (p<0.05) when the variants E28C or E28D were overexpressed. The upregulation of PERK in Jurkat cells was further confirmed by Western blot. Compared to the control group, Western blot analysis showed that the density of PERK was increased by 437.9±11.2%, 383.2±10.7% under hypoxia-treated and Ang II-treated conditions respectively (p<0.05), and was increased by 262.6±9.6% and 359.5±10.1% in cells overexpressing variants E28C or E28D respectively (p<0.05). Furthermore, siRNA against PERK partially reversed the downregulation of full-length SCN5A expression after hypoxia or Ang II treatment. siRNA knockdown efficiency not less than 50%.

### Example 8

This example demonstrates the role of PERK-mediated unfolded protein response pathway in the regulation of cardiac sodium channel during human heart failure, as previously described.

The unfolded protein response (UPR) is a series of interrelated signaling pathways that occur when the endoplasmic reticulum (ER) experiences excess secretory load, accumulates misfiled proteins, or is subject to other pathological conditions. UPR acts to attenuating general protein synthesis, induces the expression of ER chaperone proteins, and enhances the degradation of misfiled proteins. In published work, we have shown that heart failure (HF) increases alternative splicing of the SCN5A gene (encoding cardiac sodium channel), generating mRNA variants E28C and E28D encoding truncated, nonfunctional sodium channel. The presence of these variants causes a dominant negative downregulation of the wild-type SCN5A mRNA and sodium current to a sufficient extent to be arrthmogenic. We tested whether PERK-mediated UPR contributed to the dominant negative effect on Na⁺ current when truncated sodium channel mRNA variants are present in HF.

The correlation of expression changes among PERK, major human embryonic stem cell-derived cardiomyocytes (hESC-CMs). Hypoxia and Ang II were used as induces or abnormal splicing since they have been shown to mediate some of the pathological consequences of HF>hESC-CMs were divided into six experimental groups: normoxic, 1% O₂ hypoxia treated, 100 nmol/L Ang II-treated, E28C overexpressed, E28D overexpressed, and empty vector control. E28C and E28D constructs were transduced to overexpress the truncated proteins.

The expression of major UPR components (PERK, calnexin, CHOP) were increased in HF tissues and cardiac Na+ channels were downregulated. In hESC-CMs, induction of SCN5A variants E28C and E28D with Ang II or hypoxia as well as expression of exogenous variants could induce major UPR components (PERK, calnexin, CHOP). Finally, downregualtion of PERK prevented the loss of full-length SCN5A mRNA abundance with these stimuli.

SCN5A variants could induce the expression of major UPR components and could induce PERK-mediated Na+ channeled downregulation. The results indicate that the UPR contributes to Na+ channel downregulation during human HF.

### Example 9

We have reported that SCN5A, the gene encoding the α-subunit of the cardiac Na+ channel, has two mRNA alternative splicing variants that are upregulated in human heart failure (HF). These splicing variants do not form functional channels, and their presence reduces conduction velocity between cardiomyocytes. Therefore, abnormal Na+ channel splicing may contribute to arrhythmic risk in HF. Further studies indicated that splicing factors RBM25 and hLuc7A lead to the abnormal mRNA processing. Our data also show that immortalized B cells express cardiac Na+ channel variants identically to those in heart tissue and may serve as a surrogate for abnormal cardiac splicing.

We tested whether white blood cell (WBC) Na+ channel mRNA splicing varied as a function of the presence or absence of HF.

Methods:

One hundred eighty adult patients were recruited into this study, 45 controls without HF (Ejection Fraction (EF)>60%) and 135 with HF (EF<35%). Patients with congenital heart disease, infections, and inflammatory conditions were excluded. Total RNA was extracted from WBCs. The mRNA abundances of SCN5A, SCN5A variants, and the splicing factors RBM25 and hLuc7a were determined by real-time PCR.

Results:

The ratio of WBC SCN5A variants E28C or E28D to the full-length SCN5A transcript was increased in HF patients as compared to the control group. The average fold inductions were 5.0±2.7 and 7.0±3.5 for SCN5A variants E28C and E28D respectively (p<0.05). These changes were greater than those observed in cardiac tissue. The WBC mRNA abundances of RBM25 and hLuc7a also were increased in HF. The average increases for RBM25 and hLuc7A were 67.0±7.8% and 73.0±9.3% respectively (p<0.05). These changes were similar to those in heart tissue which we reported before. Conclusion: A distinct pattern of increased pathogenic splicing factors and abnormal Na+ channel splicing was present in circulating WBCs of HF patients, suggesting that WBC mRNA splicing may be affected by the same processes as occur in the heart and that WBC mRNA splicing may serve as a surrogate for the arrhythmic risk related to Na+ channel downregulation in the heart.

### Example 10

This example provides a description of a human clinical trial known as SOCS-HEFT (Sodium Channel Splicing in Heart Failure Trial, NCT01185587), which to date is an ongoing clinical trial.

We hypothesized that (1) patients with reduced left ventricular ejection fraction have increased abundances of truncated mRNA splice variants of the SCN5A gene, which portends to sodium channel dysfunction and an increased risk for sudden cardiac death and (2) patients with implantable cardioverter-defibrillator devices (ICDs) who have experienced shock therapy have increased abundances of truncated mRNA splice variants of the SCN5A gene compared to similar congestive heart failure patients who have not experienced shock therapy.

To test these hypotheses, we initiated the SOCS-HEFT trial as essentially described below.

The specific aims and objectives of this study was to (i) determine the abundances of SCN5A mRNA splice variants in patients with chronic heart failure (CHF) and baseline ejection fractions less than 35% versus normal controls of similar age groups and (ii) to compare the abundances of SCN5A mRNA splice variants in patients with ICD devices who have and have not experienced ICD shock therapy. The study was designed to correlate the amount of white cell Na+ channel splice variants with ejection fraction in patients with an without heart failure and to correlate the amount of white cell Na+ channel splice variants with the number of appropriate ICD shock in patients with ICDs in place.

The study participants were primarily adult patients with acquired heart failure (not secondary to congenital heart disease) from any cause both with and without ICD devices. Patients with normal left ventricular function and no evidence of diastolic dysfunction by echocardiographic assessment were also included in the study as control patients. These patients did not have cardiac disease or ICD devices.

The following eligibility criteria was used when selecting study participants:
1. All patients must be greater than 18 years of age
2. Patients with reduced left ventricular function (i.e., heart failure patients) must have acquired heart failure and an ejection fraction less than 35% documented in the last two years by any methodology
3. Control population patients must be free of heart failure symptoms, diastolic dysfunction, and left ventricular systolic dysfunction documented by any methodology within 1 year of study enrollment
4. Patients with an ICD in place for more than 1 year and evidence of ICD events
5. Patients with an ICD in place for more than 1 year and no evidence of ICD events
6. All patients must be able to give informed consent

The following ineligibility criteria was used when excluding study participants:
1. Patients less than 18 years of age.
2. History of congenital heart disease as cause of impaired left ventricular function.
3. Control patients with impaired left ventricular systolic function or the presence of diastolic dysfunction.
4. Control or Study group patients with a history of congenital electrophysiological disorders like the long-QT syndrome or Brugada disease will not be included.
5. Control or Study group patients who require antiarrhythmic drugs other than Vaughn-Williams Class II and IV agents.
6. Control patients with a history of significant illness that may otherwise impair cardiac function within 12 months of study enrollment. These conditions include: myocardial infarction, cardiac hospitalization, cardiac arrhythmia, infection, or cancer.
7. ICD patients suffering from any other terminal or chronic inflammatory illness.
8. Patients taking immunosuppressive medications, have chronic infection, or have an acute or chronic inflammatory illness that might alter white cell mRNA expression.
9. Patients with any illness expected to result in death within 18 months of enrollment.
10. Patients with white blood cell dyscrasia or cancers.
11. Current illicit drug use.
12. Inability to give informed consent.

The following pre-enrollment evaluation was performed on potential study participants:
1. History and physical exam
2. Record current medications, including but not limited to angiotensin converting enzyme inhibitors (ACE inhibitors), statins, antiarrhythmic drugs, and angiotensin receptor blockers (ARBs).
3. Patients with ICD devices will have their device interrogations reviewed for the presence of shocks.

The duration of the subject participation was determined as follows: A single blood draw was requested at the time of enrollment and analyzed for levels of SCN5A mRNA splice variants. Patients had their medical records examined retrospectively from the date of enrollment for cardiac specific information such as ICD interrogation records, ECG, Echo lab results, etc. This study did not require any change in the standard of care. All study participants were subjected to phlebotomy at the time of enrollment. There were no monitoring parameters in this study. A patient may voluntarily withdrawal from the study at any time.

The outcome assessment was as follows: During the initial evaluation, demographic and past medical history data were recorded. This information included: age, race, sex, body mass index, blood pressure, New York Heart Association class, history of myocardial infarction and hypertension, diabetes status, tobacco and alcohol use, presence and type of pacemaker device. Age at diagnosis of heart failure was recorded. Additionally, review of previous cardiac testing was recorded. The types of tests reviewed included electrocardiograms, echocardiograms, coronary angiograms, and results of cardiac nuclear studies. A sample of each study participant's blood was taken for assessing the levels of SCN5A mRNA splice variants. We will also looked at angiotensin converting enzyme (ACE) level and activity, angiotensin II (Ang II), and hypoxia-inducible factor (HIF-1α) mRNA because we have recently shown Ang II and hypoxia are upstream signals for abnormal SCN5A mRNA splicing.

The sample collection and processing of this study occurred as follows: About 15 ml of blood was drawn from study participants from UIC or JBVAMC who have given informed consent for phlebotomy and study participation. Samples were delivered immediately by study staff for processing within 2 hours of collection. Levels of mRNA were measured and some of the processed sample may have been stored in a −80° F. freezer in the same lab for up to 7 years. Samples were not stored or processed at JBVAMC or any other facility.

The statistical considerations of the study were as follows: The relationship of Na⁺ channel mRNA variant abundances were compared in subjects with and without heart failure and in subjects with and without ICD events. The primary endpoint was the comparison of mRNA variant abundances. Dependent variables included heart failure and number of shocks. The number of patients needed for goal of this study was determined by the variance of the test, the mean difference expected, and some consideration of the number of covariates that will need to analyzed in the regression analysis. Previously, we showed that the least sensitive measure was a reduction in E28A abundance by 24%. If we assume that the same percentage reduction will happen in goal 1 and 2, then we would need about 45 patients in each group to have a 90% power to detect this difference, assuming a 10% loss rate due to technical errors in the assays. Therefore, we would need a total of 180 patients for the total trial, 45 with heart failure, 45 controls, 45 ICD patients with events, and 45 patients without events.

Baseline data was expressed as mean±SD for continuous variables, and frequencies for categorical variables. Differences in baseline characteristics between the groups will be examined by use of Fisher exact and Mann-Whitney tests for categorical and continuous variables, respectively. Because the number of ICD events recorded is a function of the observation time, Poisson regression was used to model any relationship. In this model, the number of ICD events observed is assumed to be distributed following a Poisson distribution. That is, for a given period of time, the probability that a certain number of events has occurred is a function of the event rate multiplied by the duration of observation. In order to estimate the effects of mRNA variants on the rate of event occurrence, it was assumed that the event rate was log linear with respect to the predictors of interest. Solving this equation gave rate ratios comparing the rate of event occurrence in subjects with and without ICD events. Multiple expressions for the mRNA variant abundances were considered, such as the relative abundance of each variant individually, the abundance of the individual variant as a function of the total Na⁺ channel mRNA, and the ratio of the truncations to the full-length Na⁺ channel mRNA. The regression coefficient was estimated for the relationship between the dependent variable, ICD events, and the independent variables as the log of the rate ratio estimates. Statistical significance was determined by using the likelihood ratio test. A p-value of 0.05 or less was taken to be statistically significant. Results were reported as the risk ratio and its associated 95% confidence interval. In order to select variables to be included in the model, we considered, conservatively, those variables with a different distribution between the two groups at a p<0.20. The possibility of multicolinearity was evaluated. Linear and non-linear terms were considered. Normality of the variable distributions were tested by a normal probability plot and by a Shapiro-Wilk test. While regression is fairly tolerant of violations in this regard, transformations were investigated as necessary. Homoscedasticity was evaluated by plot of residuals versus predicted values. Discrimination of the model was evaluated by an overall C index and validated by bootstrap methods.

The safety monitoring and assessment of this study were as follows: As this was a cross-sectional cohort comparison trial with minimal risk to patients. There was no data safety monitoring board. All data was collected in compliance with existing law and regulations. Data was stored in a de-identified manner in a controlled access location. We do not anticipate any complications with acquiring blood samples, analyzing mRNA variants, or performing the statistics. Nevertheless, the major limitation to this trial is its retrospective nature. Nevertheless, this data will be useful in the design of future prospective trials.

The following references were considered in this example:
1. Hunt S, et al. ACC/AHA Guideline Update for the Diagnosis and Management of Chronic Heart Failure in the Adult. Circulation 2005; 112:e154.
2. Rosamond R et al. Heart Disease and Stroke Statistics 2008 Update. A Report From the American Heart Association Statistics Committee and Stroke Statisitics Subcommitte. Circulation Epub 2007 Dec. 13.
3. Kannel W B, Plehn J F and Cupples L A. Cardiac failure and sudden death in the Framingham Study. Am Heart J. 1988; 115:869-75.
4. Bardy G H et al. Amiodarone or an Implantable Cardioverter-Defibrillator for Congestive Heart Failure. N Engl J Med 2005; 352:225-37.
5. Meregalli P G, Wilde A A M, and Tan, H L. Pathophysiological mechanisms of Brugada syndrome: depoloarization disorder, repolarization disorder, or more? Cardiovasc Res. 2005; 64:367-378.
6. Wang Q, Shen J, Splawski I, et al. SCN5A Mutations Associated with an Inherited Cardiac Arrhythmia, Long Q T Syndrome. Cell; 1995:80:805-811.
7. Makita n, Horie M, Nakamura T, et al. Drug-induced long-WT Syndrome Associated with a Subclinical SCN5A Mutation. Circulation. 2002; 106:1269-1274.
8. Nguyen T P, Want D W, Rhodes T H, George A L. Divergent Biophysical Defects Caused by Mutant Sodium Channels in Dilated Cardiomyopathy with Arrhythmia. Circ Res. 2008; 102:0-0.
9. Chen Q, Kirsch G E, Zhang D, Brugada R et al. Genetic Basis and Molecular Mechanism for Idiopathic Ventricular Fibrillation. Nature. 1998; 392:293-295.
10. Valdivia C R, Chu W W, P J, Foell J D, et al. Increased Late Sodium Current in Myocytes from a Canine Heart Failure Model and from Failing Human Heart. J Moll Cell Cardiol. 2005; 38:475-483.
11. Smits J P, Koopmann T T, Wilders R, et al. A Mutation in the Human Cardiac Sodium Channel (E161K) Contributes to Sick Sinus Syndrome, Conduction Disease, and Brugada Syndrome in Two Families. J Mol Cell Cardiol. 2005; 38:969-81.
12. Janse M J. Electrophysiological Changes in Heart Failure and Their Relationship to Arrhythmogenesis. Cardiovasc Res. 2004; 208-17.
13. Armoundas A A, Wu R, Juang G, et al. Electrical and Structural Remodeling of the Failing Ventricle. Pharmacol Ther. 2001; 92:213-30.
14. Albert C M, Nam E G, Rimm E B, et al. Cardiac Sodium Channel Gene Variants and Sudden Cardiac Death in Women. Circulation. 2008:117; 00-00.
15. Hong K, Guerchicoff A, ollevick G D, et al. Crypitc 5′ splice site activation in SCN5A associated with Brugada Syndrome. J Mol Cell Cardiol. 2005:38; 555-560.
16. Shang L L, Pfahnl A E, Sanyal S, et al. 2007:101; 1146-54.
17. Halbach M, Egert U, Hescheler J, and Banach K. Estimation of Action Potential Changes from Field Potential Recordings in Multicellular Mouse Cardiac Myocyte Cultures. 2003:13; 271-284.

### Example 11

This example demonstrates white blood cell (WBC) SCN5A alternative splicing correlates with cardiac SCN5A splicing.

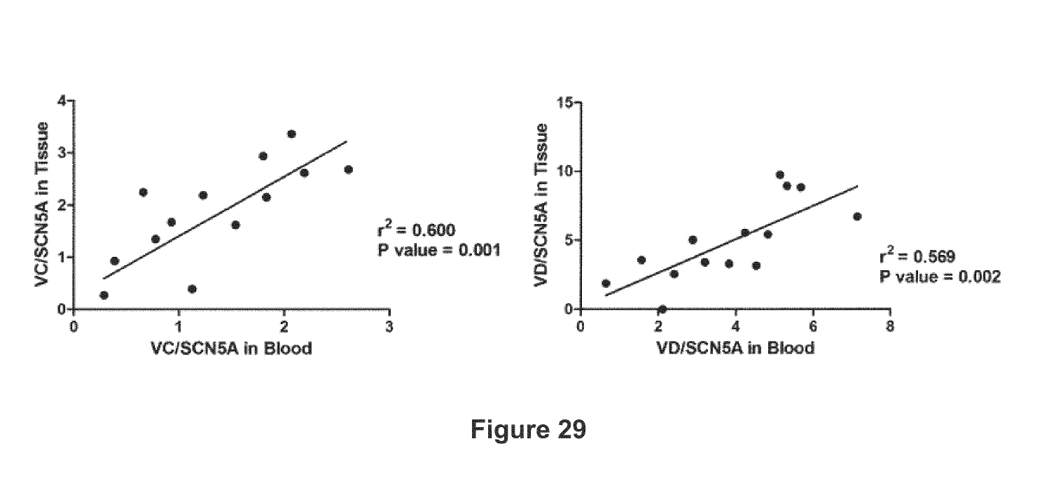

Based on data from SOCS-HEFT, we have established that WBC and left ventricular SCN5A splicing variant abundances are highly correlative. Using Left Ventricular Assist Device core samples and concurrently obtained blood samples, we have shown that there is a significant degree of correlation between normalized variant levels in the heart and blood. (FIG. 29) These data suggest that a blood test for variants is likely to reflect changes in the heart.

### Example 12

This example demonstrates that increased WBC splicing variants are predictive of appropriate ICD discharge.

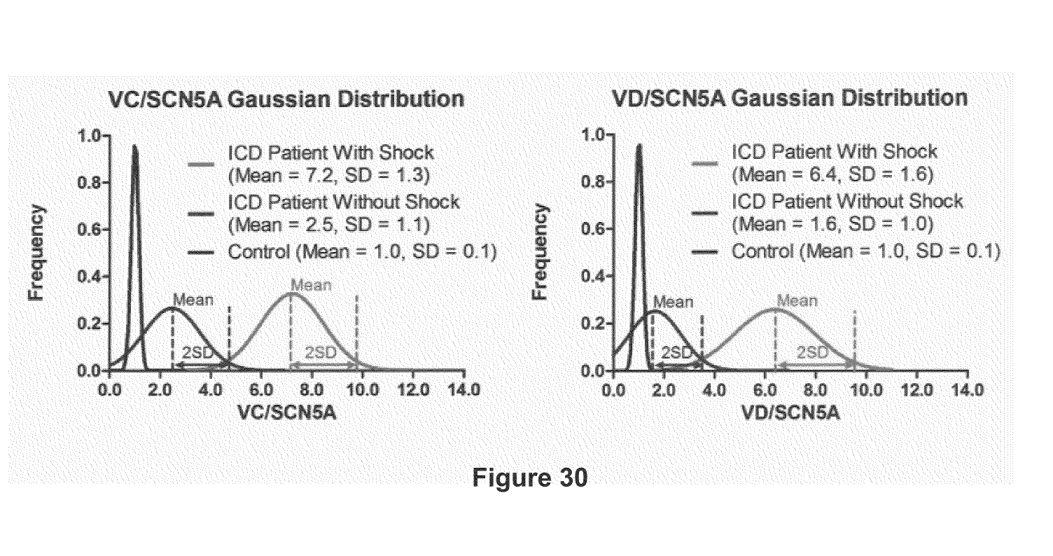

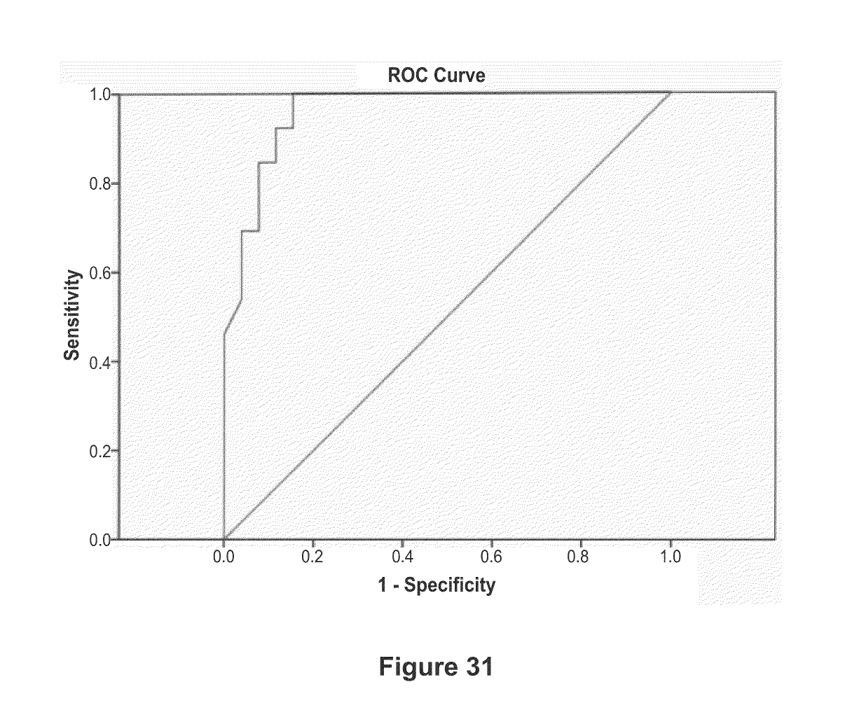

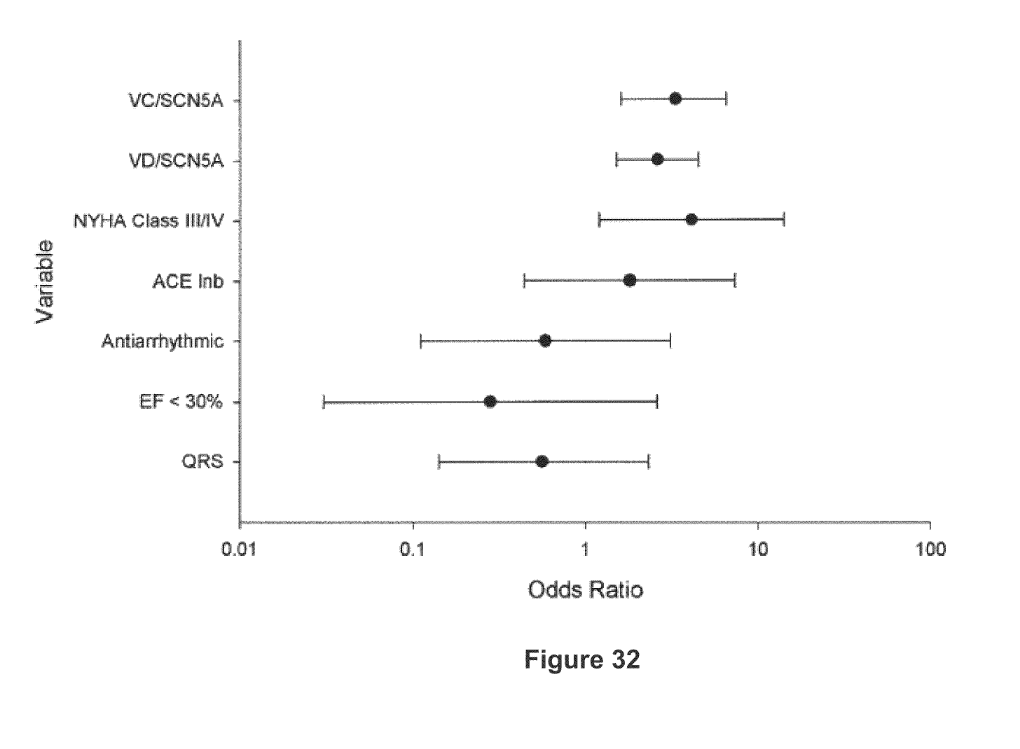

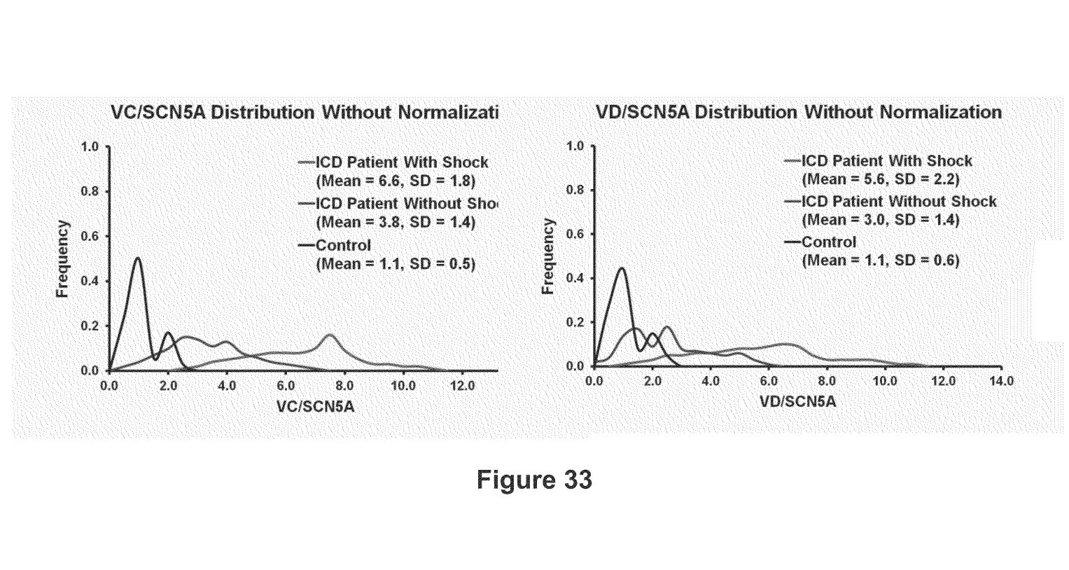

Based on data from the SOCS-HEFT trial, we were able to show that both variants E28C and E28D were increased in the blood of ICD patients as compared to controls. In addition, variant levels were considerably elevated in patients with an appropriate ICD discharge for ventricular tachycardia or fibrillation within a 12-month period preceding the sample acquisition, and there was little overlap in the distribution of variant abundances between groups (FIG. 30). Current trial enrollment is 28 controls, 34 ICD patients without a discharge, and 15 patients with an appropriate discharge (a reasonable surrogate for sudden death). Although retrospective and with small numbers, the trial results are already statistically significant for the use of SCN5A variants to predict shock risk in patients with an ICD.

Moreover, using a cut off of 4.0 for E28C or 2.8 for E28D, the sensitivity and specificity for prediction of shock risk is 100% and 85%, respectively with an area under the curve (AUC) of 0.96±0.03 for E28C and 0.95±0.03 for E28D (p<0.001, FIG. 30). This implies that the proposed blood test has the highest AUC of any test available for prediction of sudden death.

The following demonstrates how calculations were made based on the Gaussian distribution curves of FIG. 30:

In probability theory, Gaussian distribution is a continuous probability distribution that has a bell-shaped probability density function, known as the Gaussian function:

$$f ( x ) = \frac{1}{\sqrt{2 {πσ}^{2 }}} {e}^{- \frac{{( x - μ )}^{2}}{2 {σ}^{2}}}$$

where parameter μ is the mean or expectation (location of the peak) and σ is the standard deviation.

Distributions of E28C/SCN5a (abundance ratio of SCN5a splice variant E28C to SCN5a) and E28D/SCN5a (abundance ratio of SCN5a splice variant E28D to SCN5a) in control and ICD patients (with or without shock) follow Gaussian distribution. The control's Gaussian distribution has a smaller mean and a smaller standard deviation, corresponding to a very narrow bell-shaped probability distribution. The Gaussian distribution of ICD patients without shock has a larger mean and a larger standard deviation, corresponding to a wider bell-shaped probability distribution. The Gaussian distribution of ICD patients with shock has the largest mean and the largest standard deviation, corresponding to the widest bell-shaped probability distribution farthest away from the ordinate.

Normally the abundances of SCN5a and its splice variants E28C and E28D in ICD patients are calibrated by some kind of algorithm, prior to the calculation of VC/SCN5a and VD/SCN5a. The following is an example. First, the abundance difference ΔCt between ICD patients and control is calculated for β-actin, SCN5a and its splice variants E28C and E28D. Second, ΔΔCt_{SCN5a}=ΔCt_{SCN5a}−ΔCt_{β-actin}, ΔΔCt_{E28c}=ΔCt_{E28C}−ΔCt_{β-actin} and ΔΔCt_{E28D}=ΔCt_{E28D}−ΔCt_{β-actin} are calculated. Then the calibrated abundances of SCN5a and its splice variants E28C and E28D in ICD patients are obtained by the formula: 2 to the power of minus ΔΔCt_{SCN5a}, minus ΔΔCt_{E28c} and minus ΔΔCt_{E28D respectively. Last, the values of VC/SCN}5a (ratio of calibrated variant E28C abundance to calibrated SCN5a abundance) and VD/SCN5a (ratio of calibrated variant E28D abundance to calibrated SCN5a abundance) are used to fit Gaussian distributions in ICD patients.

To fit Gaussian distributions, the raw values of VC/SCN5a and VD/SCN5a can be binned to maximize the variance of the distribution of values within each bin. For example, when VC/SCN5a or VD/SCN5a is larger than or equal to 5.5 and smaller than 6.5, all the relevant values can be bucketized to the same bin center value 6.0, which has a bin width 1.0. The Gaussian distribution fitting results depend to some degree on the value of bin width.

Fitting a Gaussian distribution can be done by software, such as GraphPad Prism. The frequency distribution is specified to be plotted as an XY plot, wherein the frequencies are Y values, and VC/SCN5a or VD/SCN5a bin centers are X values. When software GraphPad Prism is used, Analyze function is clicked after the input of XY values. Then nonlinear regression, the Gaussian family of equations and the Gaussian model are chosen in turn to create a Gaussian-type frequency distribution.

VC/SCN5a value of 4.1 and VD/SCN5a value of 2.6 can be chosen as the criteria to predict the possibility with which ICD patients may have a shock. According to the existing clinical data, 99% of ICD patients with shock have VC/SCN5a values larger than 4.1 and VD/SCN5a values larger than 2.6, while 8% of ICD patients without shock have VC/SCN5a values larger than 4.1 and 16% of ICD patients without shock have VD/SCN5a values larger than 2.6.96% of ICD patients with shock have VC/SCN5a values larger than 4.7 and VD/SCN5a values larger than 3.5, while 3% of ICD patients without shock have VC/SCN5a values larger than 4.7 and VD/SCN5a values larger than 3.5.

The obtained values of VC/SCN5a and VD/SCN5a are then compared with the cut off values chosen as the criteria to predict the possibility with which ICD patients may have a shock. The cut off values may be specified in order to have a negative predictive value around 99%.

Based on the Gaussian distribution value table, 99% of data values are larger than the criterion value calculated by the following formula: μ−2.33σ, where μ is the mean or expectation (location of the peak) and σ is the standard deviation.

The mean value μ can be calculated by the following formula:

$$μ = \frac{1}{N} {\sum }_{i = 1}^{N} {x}_{i}$$

where N is the total number of data values, and each data value is denoted by x, (i=1, . . . , N).

The standard deviation σ can be calculated by the following formula:

$$σ = \sqrt{\frac{1}{N - 1} {\sum }_{i = 1}^{N} {( {x}_{i} - μ )}^{2}}$$

According to the existing clinical data, the Gaussian distribution of ICD patients with shock has a mean μ of 7.2 and a standard deviation σ of 1.3 for VC/SCN5a, and has a mean μ of 6.4 and a standard deviation σ of 1.6 for VD/SCN5a. Therefore, μ−2.33σ=4.1 for VC/SCN5a, and μ−2.33σ=2.6 for VD/SCN5a. As VC/SCN5a value of 4.1 and VD/SCN5a value of 2.6 are chosen as the criteria, 99% of ICD patients with shock can be identifies as positive, i.e. their VC/SCN5a>4.1 and VD/SCN5a>2.6. When only VC/SCN5a value of 4.1 is chosen as the criterion, the corresponding sensitivity and specificity is 91.7% and 91.1% respectively, and the corresponding positive and negative predictive value is 92.5% and 98.9% respectively. When only VD/SCN5a value of 2.6 is chosen as the criterion, the corresponding sensitivity and specificity is 85.3% and 83.2% respectively, and the corresponding positive and negative predictive value is 86.1% and 98.8% respectively.

The following are the formulas to calculate the sensitivity, specificity, positive predictive value and negative predictive value:

Sensitivity=TP/(TP+FP+FN)

Specificity=TN/(TN+FP+FN)

Positive predictive value=TP/(TP+FP)

Negative predictive value=TN/(TN+FN)

where TP denotes True Positive, TN denotes True Negative, FP denotes False Positive, and FN denotes False Negative. For example, once a threshold is determined, TN are those data values in the no shock group to the left of the cut off. FNs are the data values in the ICD shock group to the left of the cut off. TP and FP are the shock group and no shock group right of the cut off, respectively.

### Example 13

This example demonstrates that WBC splicing variants are increased in HF patients.

In the same SOCS-HEFT trial, we were able to show that both variants were increased in the blood of HF patients as compared to controls (FIG. 30). Current trial enrollment is 42 males and 14 females. This suggests that WBC SCN5A mRNA splicing varies as a function of HF, and since HF is associated with arrhythmic risk, it stands to reason that WBC mRNA variants may be able to predict sudden death risk.

### Example 14

This example demonstrates a cross-sectional, cohort study comparing patients with and without ICD therapies for ventricular arrhythmia.

Each patient of this study will have an ICD. Potential subjects will come from the cohort of several thousand patients followed by the three University of Illinois at Chicago teaching hospitals. Patients with adjudicated ICD therapies for malignant ventricular tachycardia (i.e. ICD events) along with an equal number of subjects without ICD events identified from the same database and chosen randomly will be asked to provide another blood sample for analysis of WBC Na+ channel mRNA splice variant abundances. Then, these abundances will be correlated with the presence of ICD events after correction for covariates discussed below. Eligibility criteria include: (1) greater than 18 years of age and (2) an ICD in place for more than 1 year so that we can evaluate retrospective risk over a reasonable period. Ineligibility criteria are listed in the Human Subjects section and will include: history of congenital heart disease, congenital arrhythmic disorders, patients taking immunosuppressive medications, having chronic infection that might alter white cell mRNA expression, patients with white blood cell dyscrasia or cancers. All patients enrolled will give written consent.

Data will be collected from subject interviews and review of hospital and clinic charts. Demographic data obtained will include: age, race, body mass index, New York Heart Association (NYHA) functional class, and a history of previous myocardial infarction, hypertension, diabetes, smoking, or alcohol use. Additionally, all medications being taken at the time of enrollment and the date and method of EF determination will be recorded.

A single blood draw will be performed at the time of enrollment. Total RNA will be isolated acutely from WBCs and will be analyzed for the various forms of Na+ channel splice variations using real-time R_{T}-PCR. Total RNA will be used for synthesizing cDNA by reverse transcription using iScript cDNA synthesis Kit (Bio-Rad, Hercules, Calif.) following the manufacturer's instructions. All amplifications will be performed in duplicate and consist of 40 cycles of 30 s at 94° C., 30 s at 65° C., and 1 min at 72° C. in a BioRad thermocycler iCycler (Hercules, Calif.). PCR products will be analyzed by electrophoresis on 1.5% agarose gels. Beta-actin and GAPDH will be used as internal references when making quantitative comparison.

The smallest change in mRNA isoform abundance noted above between HF and control patients was for the full-length transcript, which was reduced by 25%. Using this smallest change as the basis for a power analysis, enrolling 37 subjects in each group would give a 90% power to detect this difference using a two-tailed alpha level of 0.05. Based on this and the need to have enough subjects to correct for covariates, we will enroll 50 subjects in each group.

We anticipate abnormal SCN5A splicing patterns will be associated with ICD events. The relationship of Na+ channel mRNA variant abundances and patterns will be compared in subjects with and without ICD events. As above, various mRNA isoform levels will be expressed as the relative mRNA abundance and as the percent of the total Na4 channel mRNA. Baseline data will be expressed as mean±SD for continuous variables, and frequencies for categorical variables. Differences in baseline characteristics between the groups will be examined by use of Fisher exact and Mann-Whitney tests for categorical and continuous variables, respectively. Because the number of ICD events recorded is a function of the observation time, Poisson regression will be used to model any relationship. In this model, the number of ICD events observed is assumed to be distributed following a Poisson distribution. That is, for a given period of time, the probability that a certain number of events has occurred is a function of the event rate multiplied by the duration of observation. In order to estimate the effects of mRNA variants on the rate of event occurrence, it is assumed that the event rate is log linear with respect to the predictors of interest. Solving this equation gives rate ratios comparing the rate of event occurrence in subjects with and without ICD events. Multiple expressions for the mRNA variant abundances will be considered, such as the relative abundance of each variant individually, the abundance of the individual variant as a function of the total Na+ channel mRNA, and the ratio of the truncations to the full-length Na+ channel mRNA. The regression coefficient will be estimated for the relationship between the dependent variable, ICD events, and the independent variables as the log of the rate ratio estimates. Statistical significance will be determined by using the likelihood ratio test. A p-value of 0.05 or less will be taken to be statistically significant. Results will be reported as the risk ratio and its associated 95% confidence interval. In order to select variables to be included in the model, we will consider, conservatively, those variables with a different distribution between the two groups at a p<0.20. The possibility of multicolinearity will be evaluated. Linear and non-linear terms will be considered. Normality of the variable distributions will be tested by a normal probability plot and by a Shapiro-Wilk test. While regression is fairly tolerant of violations in this regard, transformations will be investigated as necessary. Homoscedasticity will be evaluated by plot of residuals versus predicted values. Discrimination of the model will be evaluated by an overall C index and validated by bootstrap methods. Data analysis will be done in collaboration with the UIC Center for Clinical Translational Science, recently funded by the National Center for Research Resources, NIH, Award Number UL 1RR029879, which maintains a biostatistical core.

We do not anticipate any complications with acquiring blood samples, analyzing mRNA variants, or performing the statistics, which are similar to that used in a recent interim GRADE analysis55 or our Statins for the Prevention of Atrial fibrillation trial (StoP-AF, NCT00252967) (56). Nevertheless, while we have shown that AngII and hypoxia affect splice variation abundance, one potential complication is other conditions may exist that influence Na+ channel splicing aside from those directed related to HF. The sample size should be large enough to allow for statistical correction of other factors, and identification of such factors may lead to risk mitigation strategies. For example, it is possible that splicing is influenced by the type of cardiomyopathy, race, gender, or EF, and this will be investigated. Our preliminary data indicates that age does not affect variation abundance in F-IF patients (data not shown). Additionally, it is recognized that even if this retrospective trial is positive, a prospective, multicenter trial will be necessary to firmly establish the usefulness of Na+ channel variants in the prediction of ICD events.

### Example 15

This example demonstrates a study for identifying patients to which administration of an anti-arrhythmic drug is safe.

Patients with an ICD and taking an anti-arrhythmic drug will be enrolled in this study. This protects them against any adverse proarrhythmic events of the drug. Levels of sodium channel variants, sodium channel splicing factors, and/or unfolded protein response (UPR) will be assessed prior to starting an anti-arrhythmic drug administration regimen. After starting the patients on the drug, patients will be monitored for appropriate shock risk therefor, in the case of ventricular arrhythmia safety and efficacy or onset/recurrence of atrial arrhythmia. The use of these drugs in atrial arrhythmias is much more common and represents a larger market.

Differences in time to relevant arrhythmia onset will be analyzed using a proportional hazards regression, estimates of the median time to the arrhythmia, and the proportion endpoint-free. Cox proportional hazards regression will be used to derive the hazards ratio for the study endpoint after adjustment for variables that may influence the outcome including age, sex, body mass index, blood pressure, NYHA classification, hypertension, smoking, number of previous CVs, LA dimension, estimated LV ejection fraction, and LV wall thickness. In the analysis, time to onset of the relevant arrhythmia will be the dependent variable and the baseline marker levels, demographic, and clinical variables will serve as independent variables. This approach will test whether treatment outcome is predicted by marker levels. The modeling will take into consideration that statins, ACE inhibitors/ARBs, nitrates and other drugs may affect our markers by including dummy indicator variables for these treatments. Discrimination of the model will be evaluated by an overall C index, validated by bootstrap methods, and fit by examining the relationship between predicted and observed recurrent arrhythmia over a range of predicted events. The proportional hazards assumption will be checked by various methods (examination of log-log plots, testing Schoenfeld residuals, inclusion of terms representing time-dependent interactions between each of the independent variables and time). Results will be reported as the hazard ratio and its associated 95% confidence interval. The significance of this hazards ratio will be assessed by the likelihood ratio test. If the hypothesis is true then the therapy must reduce the hazard ratio of AF or AFlut recurrence and also reduce markers of oxidative stress. The secondary endpoint of the ability of the intervention to decrease oxidative stress at 30 days will be analyzed by paired t tests and by multiple regression techniques to adjust for covariates.

The overall, adjusted R² will be taken as a measure of goodness of fit. The models will be refined using a stepwise backward procedure will be used, excluding variables above a value of p=0.05 for the null hypothesis that the coefficient of the independent variable in question is equal to zero. Assumptions in the analysis such as linearity of terms will be investigated examination of residuals and scatter plots of the independent variable with respect to the dependent variables. If nonlinearity is detected, transformations of the variables or fitting a nonlinear model will be attempted, and terms will be evaluated for their improvement of the model. Normality of the variable distributions will be tested by a normal probability plot and by a Shapiro-Wilk test. While regression is fairly tolerant of violations in this regard, transformations will be investigated as necessary. Homoscedasticity will be evaluated by plot of residuals versus predicted values. If the assumptions appear to be violated, we will consider non-parametric methods (e.g. Kruskall-Wallis test).

### Example 16

This example provides the results of the completed SOCS-HEFT trial originally described in Example 10.

The following is a description of the methods carried out in this example.

The clinical characteristics of study population (Table 2) and recruitment criteria. This was a cross-sectional, cohort, comparison trial, entitled “Sodium Channel Splicing in Heart Failure Trial,” (SOCS-HEFT, ClinicalTrials.gov Identifier NCT01185587) conducted at the University of Illinois at Chicago (UIC) and the Jesse Brown Veterans Administration Medical Center (JBVAMC) in Chicago, Ill. The study was approved by the Collaborative UIC/Northwestern/JBVAMC Institutional Review Board (IRB). Human heart tissue was obtained under UIC IRB approved protocol (2009-0881). This study focused on adult patients with acquired HF not secondary to congenital heart disease. All the subjects were recruited into four groups: control; HF; implantable cardioverter-defibrillator (ICD) devices without appropriate event therapy [ICD(−)Event)] and ICD with appropriate event therapy [ICD(+)Event]. An appropriate ICD event was adjudicated by an independent, blinded, clinical cardiac electrophysiologist as any device therapy delivered to interrupt ventricular fibrillation or ventricular tachycardia excluding anti-tachycardia pacing. Patients with normal left ventricular function and no evidence of diastolic dysfunction by echocardiographic assessment were included in the study as control patients. The pre-enrollment evaluation for all groups included: a history and physical examination and recording current medications including angiotensin converting enzyme inhibitors (ACE inhibitors), statins, antiarrhythmic drugs, and angiotensin receptor blockers (ARBs). All study subjects signed a written informed consent prior to enrollment. ICD programming was at the discretion of the attending physician. The ICD implant indication was predominantly primary prevention (77%). Data were collected from subject interviews and review of hospital and clinic charts. Demographic data obtained included: age, race, body mass index, and New York Heart Association (NYHA) functional class. Additionally, all medications were recorded at the time of enrollment, and the date and method of left ventricular ejection fraction (LVEF) determination were recorded. All LVEF determinations were made by echocardiography or cardiac magnetic resonance imaging. LVEF was determined in a 2-year window prior to enrollment.

TABLE 2
The clinical characteristics of the study population

| | Control | HF | ICD Without | ICD With | P Value |
|---|---|---|---|---|---|
| | (n = 28) | (n = 43) | Event (n = 42) | Event (n = 21) | (VC/VD) |
| Age-yr | 68.1 ± 12.1 | 59.9 ± 15.1 | 63.5 ± 11.1 | 62.1 ± 12.0 | 0.317/0.485 |
| | | | | | (Age≦60 yrs) |
| Male Sex-no. (%) | 26 (92.9) | 28 (65.1) | 29 (69.0) | 15 (71.4) | 0.777/0.722 |
| Race-no. (%) | | | | | |
| African American | 23 (82.1) | 29 (67.4) | 26 (61.9) | 16 (76.2) | 0.705/0.575 |
| Caucasian | 5 (17.9) | 3 (7.0) | 5 (11.9) | 1 (4.8) | 0.470/0.422 |
| Hispanic | 0 (0) | 9 (20.9) | 9 (21.4) | 4 (19.0) | 0.493/0.432 |
| Asian | 0 (0) | 1 (2.3) | 1 (2.4) | 0 (0) | 0.839/0.079 |
| Other | 0 (0) | 1 (2.3) | 1 (2.4) | 0 (0) | 0.532/0.646 |
| NYHA Class-no. (%) | | | | | 0.006/0.002 |
| | | | | | (I/II vs. III/IV) |
| I | 0 (0) | 2 (4.7) | 0 (0) | 0 (0) | |
| II | 0 (0) | 9 (20.9) | 9 (21.4) | 0 (0) | |
| III | 0 (0) | 11 (25.6) | 18 (42.9) | 4 (19.0) | |
| IV | 0 (0) | 21 (48.8) | 15 (35.7) | 17 (81.0) | |
| Ischemic Cardio- | 7 (25.0) | 19 (44.2) | 31 (73.8) | 12 (57.1) | 0.814/0.462 |
| myopathy-no. (%) | | | | | |
| Medications-no. (%) | | | | | |
| β Blocker | 8 (28.6) | 35 (81.4) | 37 (88.1) | 21 (100) | 0.005/0.046 |
| ACE Inhibitor | 12 (42.9) | 29 (67.4) | 29 (69.0) | 15 (71.4) | 0.661/0.607 |
| ARB | 3 (10.7) | 3 (7.0) | 7 (16.7) | 4 (19.0) | 0.450/0.709 |
| Aldosterone Antagonist | 0 (0) | 3 (7.0) | 14 (33.3) | 8 (38.1) | 0.301/0.904 |
| Antiarrhythmic Drug | 0 (0) | 2 (4.7) | 4 (9.5) | 8 (38.1) | 0.021/0.052 |
| QRS Duration >120 ms- | 2 (7.1) | 8 (20.0) | 18 (43.9) | 15 (71.4) | 0.014/0.150 |
| no. (%) | | | | | |
| LVEF % | 54.6 ± 1.9 | 26.0 ± 7.7 | 27.4 ± 7.0 | 26.1 ± 5.5 | 0.414/0.382 |
| | | | | | (LVEF≦20%) |

ACE, angiotensin converting enzyme;
ARB, angiotensin receptor blocker;
LVEF, left ventricular ejection factor;;
NYHA Class, New York Heart Association class

Patients in the three HF groups had to be at least 18 years of age and have a reduced LVEF<35% documented in the last two years. Patients with an ICD in place, both with and without appropriate event therapy, had to have the device implanted for more than one year. Patients taking immunosuppressive medications, having a chronic infection, having an acute or chronic inflammatory illness that might alter WBC mRNA expression, having any illness expected to result in death within 18 months of enrollment, or currently using illicit drugs were excluded from this study. Control patients had to be free of HF symptoms, diastolic dysfunction, and left ventricular systolic dysfunction documented by any methodology within one year of study enrollment. Other exclusion criteria for the control group included Long-QT Syndrome, Brugada Syndrome, or a history of significant illness (i.e. myocardial infarction, cardiac hospitalization, cardiac arrhythmia, infection, or cancer) within 12 months of study enrollment.

Laboratory methods. Blood samples were collected in PAX tubes (Fisher Scientific, Pittsburgh, Pa.) following the manufacturer's procedure. Samples were stored for up to three days at room temperature or five days at 2-8° C. Total RNA was isolated using the PAXgene Blood RNA isolation kit and then converted to cDNA using the High Capacity cDNA Reverse Transcription Kit (Qiagen, Valencia, Calif.).

The heart tissue samples were obtained from residual cores removed after left ventricular assist device (LVAD) placement at our affiliate, Christ Advocate Hospital. Eligible patients were over 18 years of age, had a LVEF of <35% documented in the last year, and had a need for LVAD implantation. Paired blood samples were collected simultaneously from patients undergoing LVAD placement. Total RNA was isolated from WBCs and human heart tissue with the RNeasy Mini and RNeasy Lipid Tissue Mini Kits, respectively (Qiagen) and then converted to cDNA using the High Capacity cDNA Reverse Transcription Kit (Qiagen). Quantitative RT-PCR was done using iQ™ SYBR® Green Supermix. The primer sequences used were SCN5A (5′-TTACGCACCTTCCGAGTCCTCC-3′; 5′-GATGAGGGCAAAGACGCTGAGG-3′); HSCN5A E28C/Reverse (5′-TCTCTTCTCCCCTCCTGCTGGTCA-3′); HSCN5A E28D/Reverse (5′-GGAAGAGCGTCGGGGAGAAGAAGTA-3′). Quantitative reverse transcription polymerase chain reaction (qRT-PCR) thermal cycling conditions were an initial uracil-N-glycosylase incubation at 50° C. for two minutes. iTaq™ DNA polymerase was activated with an initial denaturation step at 95° C. for five minutes, followed by 40 cycles of denaturation at 95° C. for 15 seconds, and annealing and extension at 60° C. for one minute. Each sample was measured for the target gene SCN5A, VC, VD, and β-actin. Variants levels were expressed as a percentage of the variant with respect to the total Na+ channel mRNA (as measured using primers for Exon 28 Variant A+Exon 28 Variant B+Exon 28 Variant C (VC)+Exon 28 Variant D (VD)) to correct for differences in WBC SCN5A expression between subjects.

Statistical analysis. Age, sex, race, ischemia, LEVF, medications, New York Heart Association (NYHA) Class, and QRS duration measurements were recorded. Clinical characteristics were reported as means±standard deviations for continuous variables and frequencies for categorical variables. Differences between the groups were examined by t-tests and chi-square tests for continuous and categorical variables, respectively. Results with p<0.05 were considered statistically significant in all analyses.

Linear regression, based on ordinary least squares (OLS), was used to determine the degree of correlation between normalized variant levels in the ventricle and blood. A probability value P<0.05 was taken to indicate statistical correlation. The diagnostic odds ratio (DOR) is an overall measure of diagnostic accuracy that combines both sensitivity and specificity: [sensitivity/(1−sensitivity)]/[(1−specificity)/specificity]. We compared the summary DORs and their corresponding 95% confidence intervals (CIs) across different diagnostic predictors: normalized variants VC and VD in the blood, New York Heart Association (NYHA) class III/IV, ACE inhibitors, antiarrhythmic drugs, LVEF≦20%, and QRS duration≧120 ms. Univariate analysis was performed to calculate DORs and their corresponding 95% CIs.

Receiver Operating Characteristic (ROC) curves were generated for both splicing variants and LVEF≦20%. Sensitivity (the proportion of true positive ICD patients with an event) and the specificity (the proportion of ICD patients without an event) were evaluated. A commonly used measure of overall diagnostic effectiveness is the Youden index, defined as: (sensitivity+specificity)−1. We determined the optimal cutoff value that maximized the Youden index. The sensitivities and specificities were calculated from the data across all possible cutoff values within the range of the test results, and we selected the cutoff value leading to the highest Youden index.

The following is a description of the results of this example.

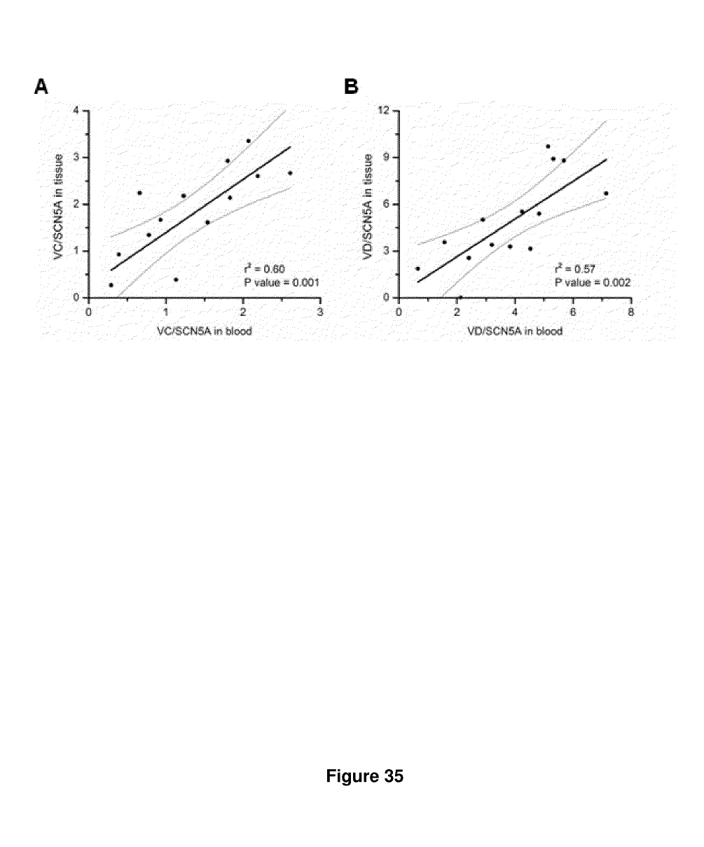

Correlation of cardiac tissue and blood abundances of VC and VD. In order to show that WBC SCN5A variants might be an acceptable surrogate for the physiologically relevant levels of variants in heart, we designed paired analysis of WBC and ventricular tissue variants from the same patient. A total 14 paired blood and heart tissue samples were collected. The correlation between blood and tissue variants levels is shown for VC and VD in FIGS. 35A and 35B, respectively. The coefficients of determination, r2, were 0.60 and 0.57 and the correlation coefficients, r, were 0.78 and 0.75 for variants VC and VD, respectively, demonstrating the high degree of correlation of tissue and blood SCN5A variants.

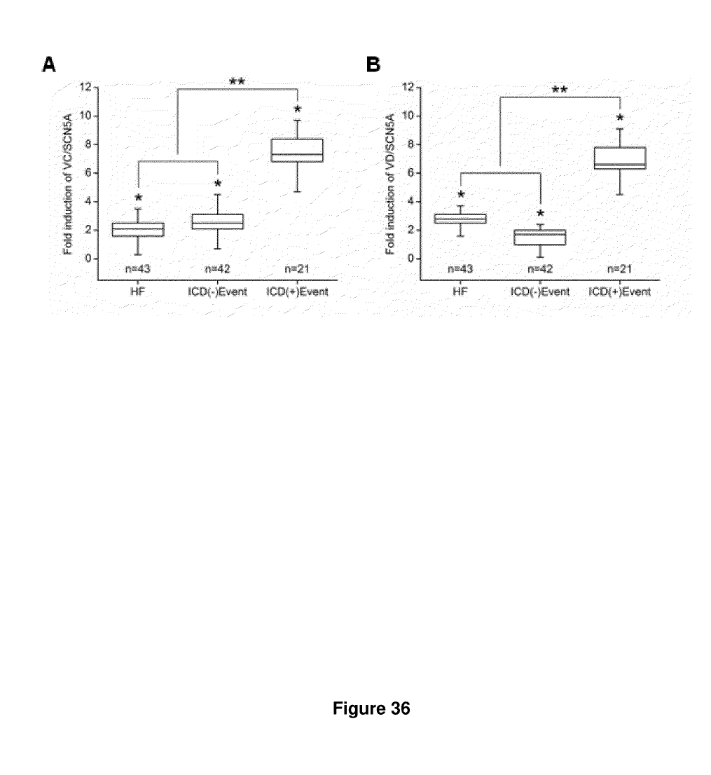

SCN5A variants were increased in participants with ICD events. Total WBCs were collected from control, HF, ICD(−)Event, and ICD(+)Event groups. The fold inductions of VC (FIG. 36A) and VD (FIG. 36B) in the HF, ICD(−)Event, and ICD(+)Event groups were compared to the control group. Medians and interquartile ranges (IQR) for VC in the three groups were HF 2.1 (IQR 1.7-2.5), ICD(−)Event 2.5 (IQR 2.1-3.1) and ICD(+)Event 7.3 (IQR 6.8-8.4) respectively. Medians and IQRs for VD were HF 2.8 (IQR 2.5-3.1), ICD(−)Event 1.7 (IQR 1.1-2.0) and ICD(+)Event 6.6 (IQR 6.3-7.8). The expressions of VC and VD were significantly increased in the HF, ICD(−)Event, ICD(+)Event groups when compared to the control group (p<0.05). Moreover, the expressions of VC and VD were significantly increased in the ICD(+)Event group when compared to the HF group or ICD(−) Event group (p<0.05).

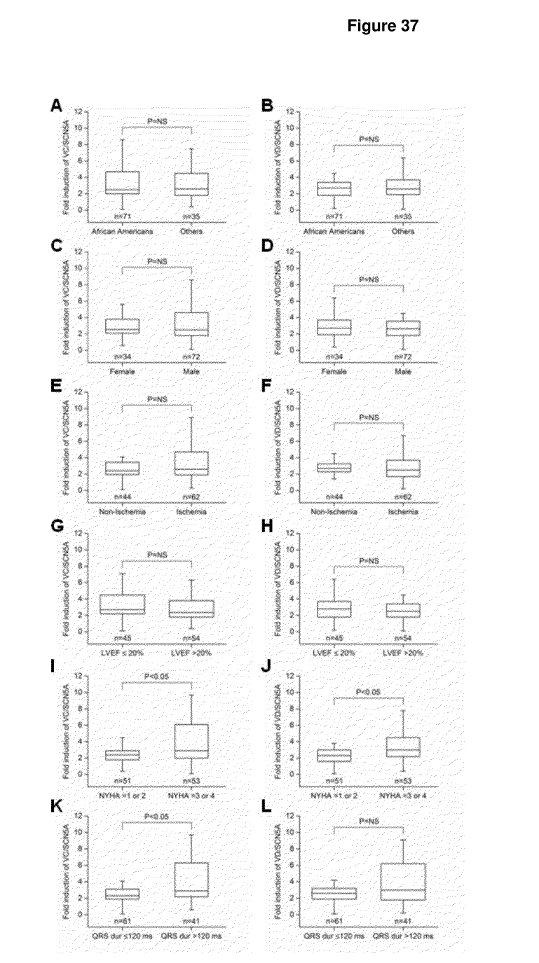

The effect of population characteristics on the expression of the SCN5A variants. There was no difference in the expression of VC and VD between races (P>0.05; FIG. 37A-B), between sexes (P>0.05; FIG. 37C-D), between subjects with an ischemia history and those without an ischemia history (P>0.05; FIG. 37E-F), or between subjects with LVEF<20% and those with LVEF>20% (P>0.05; FIG. 37G-H). Worsening NYHA class was associated with an induction of SCN5A variants, however. The fold induction for NYHA Class I-II versus NYHA Class III-IV was 2.8±1.7 versus 4.1±2.7 and 2.5±1.6 versus 3.8±2.4 for VC and VD, respectively (P<0.05 for each; FIG. 371-J). The fold induction for QRS duration≦120 ms versus>120 ms was 3.0±2.1 versus 4.2±2.6 for VC (P<0.05; FIG. 37K). Although there was no statistically significant difference, a similar fold induction trend was noted for VD (P>0.05; FIG. 37L), where the fold induction for QRS duration<120 ms versus QRS duration>120 ms was 2.9±1.9 versus 3.6±2.4.

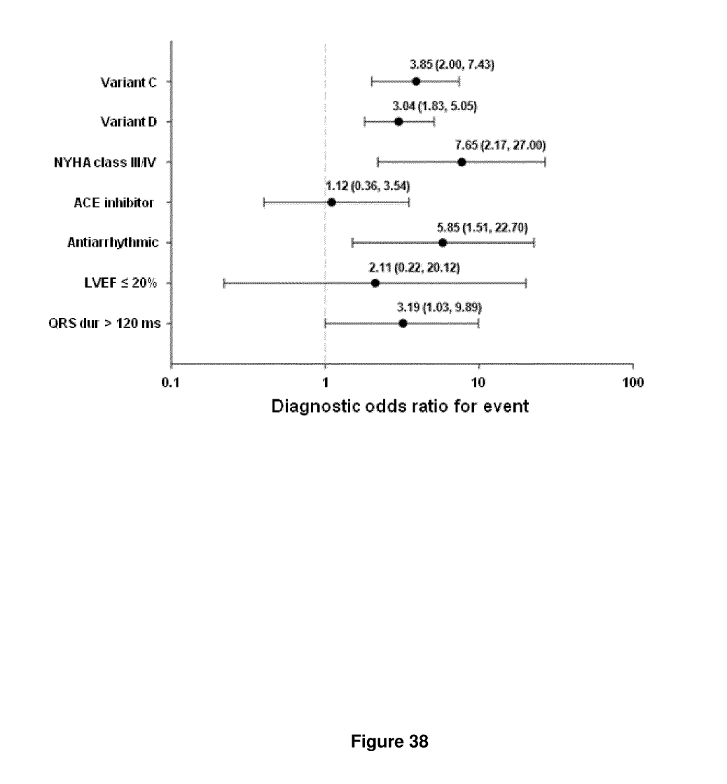

Predictors of ICD events. In FIG. 38, univariate analysis showed that NYHA class III/IV (DOR 7.65; 95% CI 2.17, 27.00), antiarrhythmic drug use (DOR 5.85; 95% CI 1.51, 22.70), VC (DOR 3.85; 95% CI 2.00, 7.43), VD (DOR 3.04; 95% CI 1.83, 5.05), and QRS duration≧120 ms (DOR 3.19; 95% CI 1.03, 9.89) were associated with increased risk of an ICD event. LVEF≦20% (DOR 2.11; 95% CI 0.22, 20.12) and ACE inhibitors (DOR 1.12; 95% CI 0.36, 3.54) were not associated with events.

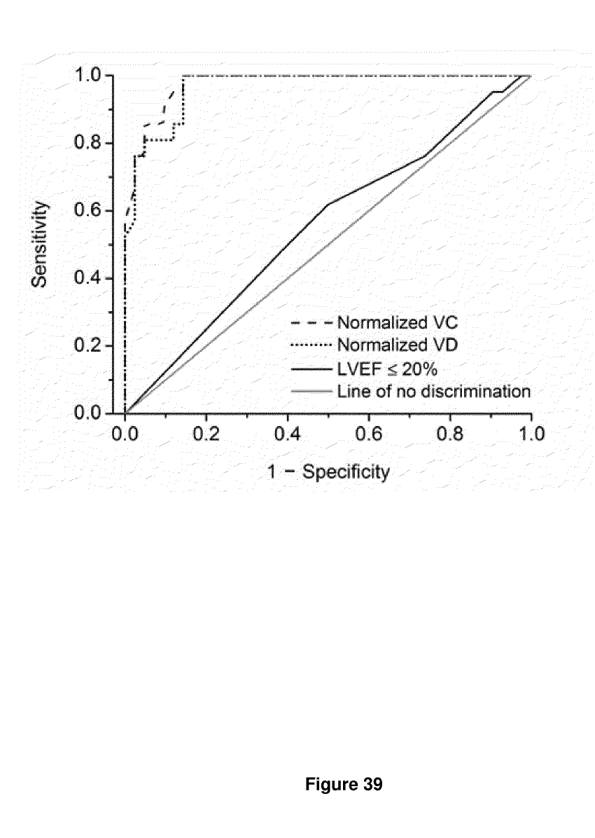

Sensitivity and specificity of SCN5A variants for determination of ICD events. ROC curves were generated to evaluate the performance of the variants and LVEF≦20% in distinguishing between the ICD patients with and without the events. The area under the ROC curve was 0.98 (95% CI 0.95, 1.00), 0.97 (95% CI 0.93, 1.00) and 0.56 (95% CI 0.41, 0.71) for VC, VD and LVEF≦20%, respectively (FIG. 39). The values for the optimal Youden index and cutoff as well as the corresponding maximum sensitivities and specificities are shown in Table 3.

TABLE 3
Optimal discrimination values for VC and VD variants

| | Optimal | Optimal | | |
|---|---|---|---|---|
| | cutoff | Youden | Corresponding | Corresponding |
| Index test | value | index | sensitivity | specificity |
| Normalized VC | 4.2 | 0.9 | 1.0 | 0.9 |
| Normalized VD | 2.9 | 0.9 | 1.0 | 0.9 |
| LVEF ≦20% | 4.5 | 0.1 | 0.6 | 0.5 |

LVEF, Left ventricular ejection fraction;
VC, SCN5A variant C;
VD, SCN5A variant D

The following is a discussion of the results of this example.

Current screening methodologies are too expensive and have insufficient positive predictive power to be used effectively in larger population screening programs. Risk stratification for sudden cardiac death and the need for ICD placement is dependent upon assessment of LVEF. There are no blood tests approved for sudden death risk stratification. Other methods employed for risk stratification are signal averaged electrocardiogram (sensitivity 62.4% and specificity 77.4% at 2 years) (19) and T-wave alternans (sensitivity 74% and specificity 44% at 1 year) (20). Although these methods are sanctioned for risk prediction of sudden cardiac death, such techniques are not widely employed, given equipment and personnel costs to implement them and studies showing low sensitivities and specificities. Invasive electrophysiological testing has been used sparingly for the same reasons (sensitivity 62% and specificity 62% at 1 year) (19). In addition, while risk may change with time, these more demanding techniques, if used at all, are often restricted to a single assessment per patient. Therefore, there is an unmet need for convenient, inexpensive, effective sudden cardiac death risk assessment in the HF population.

It is known that reductions in sodium current, the main current for cardiac conduction can be arrhythmogenic. (15, 21) SCN5A, encoding the α-subunit of the Na+ channel, was cloned by in 1992 and mapped to the chromosomal region 3p21 in 1995 (22-24). Since SCN5A was cloned, hundreds of mutations have been found that cause inherited sudden death syndromes such as Brugada syndrome, the third variant of Long QT syndrome (LTQ3), and sudden infant death (25-27). Alterations in the Na+ current, either up or downregulation, lead to arrhythmias (28). Moreover, we have shown that abnormal SCN5A mRNA splicing results in SCN5A variants that can contribute to arrhythmic risk and that these variants are increased in HF (17, 18).

Using LVAD core samples and concurrently obtained blood samples on the same patient, we have shown that there is a significant degree of correlation between normalized variant levels in the heart and blood. The expression of WBC SCN5A variant abundances were compared in subjects with a graded risk of arrhythmias from controls to HF patients with ICD events. HF patients who had received appropriate ICD intervention had significantly higher levels of SCN5A splice variants compared to controls and to subjects who had not received an intervention. As expected HF subjects with and without an ICD but with no intervention had similar variant levels. The results indicated that SCN5A variants were significantly increased in a graded manner that reflected the increasing risk of sudden death between the groups. Moreover, the separation between groups allowed for sensitive and specific discrimination of patients with and without ICD events. This suggested that the amount of SCN5A variants in the blood might have prospective predictive power to determine HF-associated arrhythmic risk.

The ability of variants to discriminate between groups with and without ICD events was not affected by sex, race, origin of the myopathy, or LVEF value less than 35%. As expect for a process dependent on the severity of HF, SCN5A variants levels showed a significant increase in NYHA class III-IV as compared to less severe HF. Consistent with the expected reduction in cardiac conduction with a reduction in functional sodium channels, variants levels were higher in patients with longer QRS durations. Interestingly, variant levels did not correlate well with LVEF, suggesting that their measure may give added information to risk reflected by left ventricular function. Given the cardiac specific nature of the SCN5A variants and the high degree of sensitivity and specificity of the correlation between SCN5A variants levels and ICD interventions, it might be possible to use WBCs SCN5A variants as a supplement to current methods to improve discrimination of patients most likely to benefit from ICD implantation.

In conclusion, we have shown that cardiac SCN5A mRNA variants are present in myocardium and WBCs and that these levels in these two cell types correlate. Moreover, the SCN5A variants levels increased with risk for SCD, and variants levels were significantly elevated in subjects having received an ICD intervention. The degree of separation of variants levels between HF subjects with and without an ICD intervention suggested variant levels had a strong power to discriminate between these two groups. If true in prospective validation trials, WBC SCN5A variant level determinations may help identify which patients with HF might benefit most from device implantation.

The following references were cited in Example 16:

### REFERENCE LIST

1. Roger V L, et al. Circulation 2011; 123:e18-e209.
2. Kannel W B, et al. Am Heart J 1988; 115:869-75.
3. Hunt S A, et al. ACC/AHA 2005 Circulation 2005; 112:e154-e235.
4. Bardy G H, et al. N Engl J Med 2005; 352:225-37.
5. LaPointe N M, et al. Circ Cardiovasc Qual Outcomes 2011; 4:146-51.
6. Bilchick K C, et al., J Am Coll Cardiol 2012; 60:1647-55.
7. Levy W C, Lee K L, Hellkamp A S, et al. Circulation 2009; 120:835-42.
8. Goldenberg I, Vyas A K, Hall W J, et al. J Am Coll Cardiol 2008; 51:288-96.
9. Huikuri H V, et al. Circulation 2003; 108:110-5.
10. Modrek B, Lee C. Nat Genet. 2002; 30:13-9.
11. Pan Q, et al. Nat Genet. 2008; 40:1413-5.
12. Zhou A, et al. Mol Cell Biol 2008; 28:5924-36.
13. Gao G, et al. Circulation 2011; 124:1124-31.
14. Hu D, et al. Heart Rhythm 2011; 9:760-9.
15. Abriel H, Kass R S. Trends Cardiovasc Med 2005; 15:35-40.
16. Shang L L, et al. Circ Res 2007; 101:1146-54.
17. Shang L L, et al. Circ Res 2007; 101:1146-54.
18. Gao G, et al. Circulation 2011; 124:1124-31.
19. Bailey J J, et al. J Am Coll Cardiol 2001; 38:1902-11.
20. Costantini O, et al. J Am Coll Cardiol 2009; 53:471-9.
21. Shibata E F, et al. J Cardiovasc Electrophysiol 2006; 17 Suppl 1:S34-S42.
22. Gellens M E, et al. Proc Natl Acad Sci USA 1992; 89:554-8.
23. George A L, Jr., et al. Cytogenet Cell Genet. 1995; 68:67-70.
24. Kullmann D M. Brain 2002; 125:1177-95.
25. Antzelevitch C. Pacing Clin Electrophysiol 2006; 29:1130-59.
26. Chung S K, et al. Heart Rhythm 2007; 4:1306-14.
27. Otagiri T, Kijima K, Osawa M, et al. Pediatr Res 2008; 64:482-7.
28. Ruan Y, Liu N, Priori S G. Nat Rev Cardiol 2009; 6:337-48.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed:

1. A method of determining a subject's need for an ICD, comprising the step of determining a ratio, R_{S}, which compares a level of a truncated SCN5A Exon 28 transcript of a biological sample obtained from a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

2. The method of claim 1, wherein the subject needs an ICD, when R_{S} is greater than or equal to a threshold ratio, R_{T}.

3. The method of claim 2, wherein R_{T} is a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as (i) not having an ICD, (ii) not having a cardiac disease, (iii) having normal left ventricular function, (iv) not having diastolic dysfunction, or (v) a combination thereof.

4. The method of claim 2, wherein R_{T}=μ+4.0σ, wherein μ=is the mean value of a Gaussian distribution of a set of data values, wherein each data value of the set represents a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as (i) not having an ICD, (ii) not having a cardiac disease, (iii) having normal left ventricular function, (iv) not having diastolic dysfunction, or (v) a combination thereof, and wherein σ is the standard deviation of the Gaussian distribution of the data values.

5. The method of claim 2, wherein R_{T} is a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as having an ICD that has not given a shock to the control subject.

6. The method of claim 2, wherein R_{T}=μ+Xσ, wherein μ=is the mean value of a Gaussian distribution of a set of data values, wherein each data value of the set represents a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as having an ICD that has not given a shock to the control subject, wherein σ is the standard deviation of the Gaussian distribution of the data values, and X is a number between 0.7 and 4.0.

7. The method of claim 2, wherein R_{T}=μ−Xσ, wherein μ=is the mean value of a Gaussian distribution of a set of data values, wherein each data value of the set represents a ratio wherein the control subject is a subject known as having an ICD that has not given a shock to the control subject, wherein the control subject is a subject known as having an ICD that has given a shock to the control subject, wherein σ is the standard deviation of the Gaussian distribution of the data values, and X is a number between 0.7 and 4.0.

8. The method of claim 7, wherein X is a number between about 0.7 and about 1.0.

9. The method of claim 7, wherein X is a number between about 2.0 and about 4.0.

10. The method of claim 9, wherein X is a number between about 2.326 and about 4.0.

11. The method of any one of the preceding claims, wherein: (A) when R_{S} is determined, (i) the level of the truncated SCN5A Exon 28 transcript of the biological sample or (ii) the level of the full length SCN5A Exon 28 transcript of the biological sample or (iii) the level of all SCN5A Exon 28 transcripts of the biological sample obtained from the subject are normalized to a level of a housekeeping gene of the biological sample obtained from the subject, (B) when R_{T} is determined, (i) the level of the truncated SCN5A Exon 28 transcript of the biological sample or (ii) the level of the full length SCN5A Exon 28 transcript of the biological sample or (iii) the level of all SCN5A Exon 28 transcripts of the biological sample obtained from the subject are normalized to a level of a housekeeping gene of the biological sample obtained from the control subject, or (C) both (A) and (B).

12. The method of claim 11, wherein the housekeeping gene is glyceraldehyde-3-phosphate dehydrogenase (GAPDH) or β-actin.

13. The method of any one of the preceding claims, wherein R_{S} is
$$\frac{Truncated SCN 5 A Exon 28 transcript}{Full length SCN 5 A Exon 28 transcript} = \frac{\begin{matrix}Calibrated abundance of \\ truncated SCN 5 A Exon 28 transcript\end{matrix}}{\begin{matrix}Calibrated abundance of full \\ length SCN 5 A Exon 28 transcript\end{matrix}}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{truncated}=([Ct of truncated SCN5A Exon 28 transcript of subject sample]−[Ct of truncated SCN5A Exon 28 transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts;
or R_{S} is
$$\frac{Truncated SCN 5 A Exon 28 transcript}{All SCN 5 A Exon 28 transcripts} = \frac{\begin{matrix}Calibrated abundance of \\ truncated SCN 5 A Exon 28 transcript\end{matrix}}{\begin{matrix}Calibrated abundance of all \\ SCN 5 A Exon 28 transcripts\end{matrix}}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{truncated}=([Ct of truncated SCN5A Exon 28 transcript of subject sample]−[Ct of truncated SCN5A Exon 28 transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

14. The method of any one of claims 6 and 11-13, wherein, when R_{T}=μ+Xσ, the ratio of the biological sample obtained from the
$$\begin{matrix}control subject = \frac{Truncated SCN 5 A Exon 28 transcipt}{Full length SCN 5 A Exon 28 transcript} \\ = \frac{\begin{matrix}Calibrated abundance of truncated \\ SCN 5 A Exon 28 transcript\end{matrix}}{\begin{matrix}Calibrated abundance of full length \\ SCN 5 A Exon 28 transcirpt\end{matrix}}\end{matrix}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of ICD patient (−) shock]−[Ct of full length SCN5A Exon 28 transcript of control sample])
ΔCt_{truncated}=([Ct of truncated SCN5A Exon 28 transcript of ICD patient (−) shock]−[Ct of truncated SCN5A Exon 28 transcript of control sample])
ΔCt_{housekeeping gene}=([Ct of housekeeping gene of ICD patient (−) shock]−[Ct of housekeeping gene of control sample]).
wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts. or wherein, when R_{T}=μ+Xσ, the ratio of the biological sample obtained from the
$$\begin{matrix}control subject = \frac{Truncated SCN 5 A Exon 28 transcript}{All SCN 5 A Exon 28 transcripts} \\ = \frac{\begin{matrix}Calibrated abundance of truncated \\ SCN 5 A Exon 28 transcript\end{matrix}}{\begin{matrix}Calibrated abundance of all \\ SCN 5 A Exon 28 transcripts\end{matrix}}\end{matrix}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of ICD patient (−) shock]−[Ct of all SCN5A Exon 28 transcripts of control sample])
ΔCt_{truncated}=([Ct of truncated SCN5A Exon 28 transcript of ICD patient (−) shock]−[Ct of truncated SCN5A Exon 28 transcript of control sample])
ΔCt_{housekeeping gene}=([Ct of housekeeping gene of ICD patient (−) shock]−[Ct of housekeeping gene of control sample])
wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

15. The method of any one of claims 7-13, wherein, when R_{T}=μ−Xσ, the ratio of the biological sample obtained from the control subject is
$$\frac{Truncated SCN 5 A Exon 28 transcript}{Full length SCN 5 A Exon 28 transcript} = \frac{\begin{matrix}Calibrated abudance of truncated \\ SCN 5 A Exon 28 transcript\end{matrix}}{\begin{matrix}Calibrated abundance of full \\ length SCN 5 A Exon 28 transcript\end{matrix}}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of ICD patient (+) shock]−[Ct of full length SCN5A Exon 28 transcript of control sample])
ΔCt_{truncated}=([Ct of truncated SCN5A Exon 28 transcript of ICD patient (+) shock]−[Ct of truncated SCN5A Exon 28 transcript of control sample])
ΔCt_{housekeeping gene}=([Ct of housekeeping gene of ICD patient (+) shock]−[Ct of housekeeping gene of control sample]).
wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts; or when R_{T}=μ−Xσ, the ratio of the biological sample obtained from the control subject is
$$\frac{Truncated SCN 5 A Exon 28 transcript}{All SCN 5 A Exon 28 transcripts} = \frac{Calibrated abudance of truncated SCN 5 A Exon 28 transcript}{Calibrated abundance of all SCN 5 A Exon 28 transcripts}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of ICD patient (+) shock]−[Ct of all SCN5A Exon 28 transcripts of control sample])
ΔCt_{truncated}=([Ct of truncated SCN5A Exon 28 transcript of ICD patient (+) shock]−[Ct of truncated SCN5A Exon 28 transcript of control sample])
ΔCt_{housekeeping gene}=([Ct of housekeeping gene of ICD patient (+) shock]−[Ct of housekeeping gene of control sample])
wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

16. The method of any one of claims 13 to 15, wherein each mRNA level is determined via Real Time-Polymerase Chain Reaction (R_{T}-PCR).

17. The method of any one of the preceding claims, wherein the level of the truncated SCN5A Exon 28 transcript is a level of SCN5A Exon 28 Splice Variant C (E28C) or a level of SCN5A Exon 28 Splice Variant D (E28D).

18. The method of claim 17, wherein the level of a truncated SCN5A Exon 28 transcript is a level of E28C and E28D.

19. The method of any one of the preceding claims, wherein the level of the truncated SCN5A Exon 28 transcript is compared to (i) a level of WT SCN5A Exon 28 transcripts, (ii) a level of E28A-S, or (iii) a combination of (i) and (ii).

20. The method of any one of the preceding claims, wherein the level of the truncated SCN5A Exon 28 transcript is compared to a level of all of WT SCN5A Exon 28 transcripts, E28A-S, E28B, E28C, and E28D.

21. The method of any one of the preceding claims, wherein the level of the truncated SCN5A Exon 28 transcript is compared to a level of (A) and (B), wherein:
(A) is one of (i) a level of WT SCN5A Exon 28 transcripts, (ii) a level of E28A-S, or (iii) a combination of (i) and (ii), and
(B) is one or more of E28B, E28C, and E28D.

22. The method of claim 19 or 20, wherein the truncated SCN5A Exon 28 transcript is E28C.

23. The method of claim 22, wherein
$${R}_{S} = {R}_{E 28 C} = \frac{Calibrated abundance of E 28 C transcript}{\begin{matrix}Calibrated abundance of a \\ full length SCN 5 A Exon 28 transcript\end{matrix}}$$
wherein:
calibrated abundance of E28C transcript=2^{-ΔΔCtE28C transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{E28C transcript}=ΔCt_{E28C transcript}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{E28C}=([Ct of E28C transcript of subject sample]−[Ct of E28C transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts;
or wherein
$${R}_{E 28 C} = \frac{Calibrated abundance of E 28 C transcript}{Calibrated abundance of all SCN 5 A Exon 28 transcripts}$$
wherein:
calibrated abundance of E28C transcript=2^{-ΔΔCtE28C transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{E28C transcript}=ΔCt_{E28C transcript}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{E28C}=([Ct of E28C transcript of subject sample]−[Ct of E28C transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

24. The method of claim 19 or 21, wherein truncated SCN5A Exon 28 transcript is E28D.

25. The method of claim 24, wherein
$${R}_{S} = {R}_{E 28 D} = \frac{Calibrated abundance of E 28 D transcript}{\begin{matrix}Calibrated abundance of full \\ length SCN 5 A Exon 28 transcript\end{matrix}}$$
wherein:
calibrated abundance of E28D transcript=2^{-ΔΔCtE28D transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{E28D transcript}=ΔCt_{E28D transcript}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{E28D}=([Ct of E28D transcript of subject sample]−[Ct of E28D transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts,
or wherein
$${R}_{E 28 D} = \frac{Calibrated abundance of E 28 D transcript}{Calibrated abundance of all SCN 5 A Exon 28 transcripts}$$
wherein:
calibrated abundance of E28D transcript=2^{-ΔΔCtE28D transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{E28D transcript}=ΔCt_{E28D transcript}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{E28D}=([Ct of E28D transcript of subject sample]−[Ct of E28D transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

26. The method of any one of claims 22 to 25, comprising determining a first R_{S} and a second R_{S}, wherein the first R_{S} compares a level of E28C to the level of all SCN5A Exon 28 transcripts and the second R_{S} compares a level of E28D to the level of all SCN5A Exon 28 transcripts.

27. The method of claim 26, wherein the first R_{S} is RE28C and the second R_{S} is E28D.

28. A method of determining a subject's need for an implanted cardiac defibrillator (ICD), comprising the steps of:
(A) determining a ratio, Rₛ, which compares a level of a truncated SCN5A Exon 28 transcript of a biological sample of a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, and
(B) comparing R_{S} as determined in (A) to a threshold ratio, R_{T}, wherein the R_{T} is determined by the system of any one of claims 97-108.

29. The method of claim 28, wherein the subject needs an ICD, when Rs is greater than or equal to the R_{T}.

30. A method of determining a subject's risk for sudden cardiac death (SCD), comprising the step of determining a ratio, Rₛ, which compares a level of a truncated SCN5A Exon 28 transcript of a biological sample obtained from a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

31. The method of claim 30, wherein the subject is at risk for SCD, when Rs is greater than or equal to a threshold ratio, R_{T}.

32. A method of determining a subject's need for an anti-arrhythmic therapy, comprising the step of determining a ratio, Rₛ, which compares a level of a truncated SCN5A Exon 28 transcript of a biological sample obtained from a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

33. The method of claim 32, wherein the subject needs an anti-arrhythmic therapy when Rs is greater than or equal to a threshold ratio, R_{T}.

34. The method of claim 32 or 33, wherein the anti-arrhythmic agent is a Singh Vaughan Williams Class IA, IB, IC, or III anti-arrhythmic agent.

35. The method of claim 32 or 33, wherein the Singh Vaughan Williams Class IA anti-arrhythmic agent is Quinidine, Procainamide, or Disopyramide.

36. The method of claim 32 or 33, wherein the Singh Vaughan Williams Class IB anti-arrhythmic agent is Lidocaine, Phenyloin, Mexiletine, or Tocamide.

37. The method of claim 32 or 33, wherein the Singh Vaughan Williams Class IC anti-arrhythmic agent is Flecamide, Propafenone, Moricizine, or Encamide.

38. The method of claim 32 or 33, wherein the Singh Vaughan Williams Class III anti-arrhythmic agent is Dronedarone, Amiodarone, or Ibutilide.

39. The method of claim 32 or 33, wherein the anti-arrhythmic agent is NAD+ or mitoTEMPO.

40. A method of reducing risk of sudden cardiac death (SCD) in a subject, comprising the step of the steps of
(a) determining a ratio, Rs, which compares a level of a truncated SCN5A Exon 28 transcript to (i) a level of a full length SCN5A Exon 28 transcript or (ii) a level of all SCN5A Exon 28 transcripts, of a biological sample obtained from the subject,
(b) implanting in the subject an ICD, when Rs is increased relative to a threshold ratio, R_{T}.

41. The method of any one of claims 31, and 33-40, wherein R_{T} is a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as (i) not having an ICD, (ii) not having a cardiac disease, (iii) having normal left ventricular function, (iv) not having diastolic dysfunction, or (v) a combination thereof.

42. The method of any one of claims 31, and 33-40, wherein R_{T}=μ+4.0σ, wherein μ=is the mean value of a Gaussian distribution of a set of data values, wherein each data value of the set represents a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as (i) not having an ICD, (ii) not having a cardiac disease, (iii) having normal left ventricular function, (iv) not having diastolic dysfunction, or (v) a combination thereof, and wherein a is the standard deviation of the Gaussian distribution of the data values.

43. The method of any one of claims 31, and 33-40, wherein R_{T} is a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as having an ICD that has not given a shock to the control subject.

44. The method of any one of claims 31, and 33-40, wherein R_{T}=μ+Xσ, wherein μ=is the mean value of a Gaussian distribution of a set of data values, wherein each data value of the set represents a ratio which compares a level of a truncated SCN5A Exon 28 transcript of a biological sample obtained from a control subject to (i) a level of a full length SCN5A Exon 28 transcript of the biological sample obtained from the control subject or (ii) a level of all SCN5A Exon 28 transcripts of the biological sample obtained from the control subject or (iii) a level of a full length SCN5A Exon 28 transcript and a level or one or more truncated SCN5A Exon 28 transcripts of the biological sample obtained from the control subject, wherein the control subject is a subject known as having an ICD that has not given a shock to the control subject, wherein σ is the standard deviation of the Gaussian distribution of the data values, and X is a number between 0.7 and 4.0.

45. The method of any one of claims 31, and 33-40, wherein R_{T}=μ−Xσ, wherein μ=is the mean value of a Gaussian distribution of a set of data values, wherein each data value of the set represents a ratio wherein the control subject is a subject known as having an ICD that has not given a shock to the control subject, wherein the control subject is a subject known as having an ICD that has given a shock to the control subject, wherein σ is the standard deviation of the Gaussian distribution of the data values, and X is a number between 0.7 and 4.0.

46. The method of claim 45, wherein X is a number between about 0.7 and about 1.0.

47. The method of claim 45, wherein X is a number between about 2.0 and about 4.0.

48. The method of claim 47, wherein X is a number between about 2.326 and about 4.0.

49. The method of any one of claims 30 to 48, wherein: (A) when R_{S} is determined, (i) the level of the truncated SCN5A Exon 28 transcript of the biological sample or (ii) the level of the full length SCN5A Exon 28 transcript of the biological sample or (iii) the level of all SCN5A Exon 28 transcripts of the biological sample obtained from the subject are normalized to a level of a housekeeping gene of the biological sample obtained from the subject, (B) when R_{T} is determined, (i) the level of the truncated SCN5A Exon 28 transcript of the biological sample or (ii) the level of the full length SCN5A Exon 28 transcript of the biological sample or (iii) the level of all SCN5A Exon 28 transcripts of the biological sample obtained from the subject are normalized to a level of a housekeeping gene of the biological sample obtained from the control subject, or (C) both (A) and (B).

50. The method of claim 49, wherein the housekeeping gene is glyceraldehyde-3-phosphate dehydrogenase (GAPDH) or β-actin.

51. The method of any one of claims 28 to 50, wherein R_{S} is
$$\frac{Truncated SCN 5 A Exon 28 transcript}{Full length SCN 5 A Exon 28 transcript} = \frac{\begin{matrix}Calibrated abundance of \\ truncated SCN 5 A Exon 28 transcript\end{matrix}}{\begin{matrix}Calibrated abundance of \\ full length SCN 5 A Exon 28 transcipt\end{matrix}}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{truncated}=([Ct of truncated SCN5A Exon 28 transcript of subject sample]−[Ct of truncated SCN5A Exon 28 transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts;
or R_{S} is
$$\frac{Truncated SCN 5 A Exon 28 transcript}{All SCN 5 A Exon 28 transcripts} = \frac{Calibrated abundance of truncated SCN 5 A Exon 28 transcript}{Calibrated abundance of all SCN 5 A Exon 28 transcipts}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{truncated}=([Ct of truncated SCN5A Exon 28 transcript of subject sample]−[Ct of truncated SCN5A Exon 28 transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

52. The method of any one of claims 44 and 49-51, wherein, when R_{T}=μ+Xσ, the ratio of the biological sample obtained from the
$$control subject = \frac{Truncated SCN 5 A Exon 28 transcript}{Full length SCN 5 A Exon 28 transcript} = \frac{Calibrated abundance of truncated SCN 5 A Exon 28 transcript}{\begin{matrix}Calibrated abundance of full \\ length SCN 5 A Exon 28 transcript\end{matrix}}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of ICD patient (−) shock]−[Ct of full length SCN5A Exon 28 transcript of control sample])
ΔCt_{truncated}=([Ct of truncated SCN5A Exon 28 transcript of ICD patient (−) shock]−[Ct of truncated SCN5A Exon 28 transcript of control sample])
ΔCt_{housekeeping gene}=([Ct of housekeeping gene of ICD patient (−) shock]−[Ct of housekeeping gene of control sample]).
wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts. or wherein, when R_{T}=μ+Xσ, the ratio of the biological sample obtained from the
$$control subject = \frac{Truncated SCN 5 A Exon 28 transcript}{All Exon 28 transcripts} = \frac{\begin{matrix}Calibrated abundance of \\ truncated SCN 5 A Exon 28 transcript\end{matrix}}{\begin{matrix}Calibrated abundance of all \\ SCN 5 A Exon 28 transcripts\end{matrix}}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of ICD patient (−) shock]−[Ct of all SCN5A Exon 28 transcripts of control sample])
ΔCt_{truncated}=([Ct of truncated SCN5A Exon 28 transcript of ICD patient (−) shock]−[Ct of truncated SCN5A Exon 28 transcript of control sample])
ΔCt_{housekeeping gene}=([Ct of housekeeping gene of ICD patient (−) shock]−[Ct of housekeeping gene of control sample])
wherein “ICD patient (−) shock” is a biological sample obtained from a subject known as having an ICD that has not given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

53. The method of any one of claims 45 to 51, wherein, when R_{T}=μ−Xσ, the ratio of the biological sample obtained from the control subject is
$$\frac{Truncated SCN 5 A Exon 28 transcript}{Full length SCN 5 A Exon 28 transcript} = \frac{\begin{matrix}Calibrated abundance of \\ truncated SCN 5 A Exon 28 transcript\end{matrix}}{\begin{matrix}Calibrated abundance of full \\ length SCN 5 A Exon 28 transcript\end{matrix}}$$
wherein:
calibrated abundance of truncated SCN5A Exon 28 transcript=2^{-ΔΔCttruncated SCN5A Exon 28 transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{truncated SCN5A Exon 28 transcript}=ΔCt_{truncated}−ΔCt_{housekeeping gene}
ΔCt_{full length}=([Ct of full length SCN5A Exon 28 transcript of ICD patient (+) shock]−[Ct of full length SCN5A Exon 28 transcript of control sample])
ΔCt_{truncated}=([Ct of truncated SCN5A Exon 28 transcript of ICD patient (+) shock]−[Ct of truncated SCN5A Exon 28 transcript of control sample])
ΔCt_{housekeeping gene}=([Ct of housekeeping gene of ICD patient (+) shock]−[Ct of housekeeping gene of control sample]).
wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts; or when R_{T}=μ−Xσ, the ratio of the biological sample obtained from the control subject is
$$\frac{Truncated SCN 5 A Exon 28 transcript}{All SCN 5 A Exon 28 transcripts} = \frac{\begin{matrix}Calibrated abundance of \\ truncated SCN 5 A Exon 28 transcript\end{matrix}}{Calibrated abundance of all SCN 5 A Exon 28 transcripts}$$
wherein:
calibrated abundance of truncated SCN5A Exon 28 transcript=2^{-ΔΔCttruncated SCN5A Exon 28 transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{truncated SCN5A Exon 28 transcript}=ΔCt_{truncated}−ΔCt_{housekeeping gene}
ΔCt_{all SCN5A Exon 28 transcripts}=([Ct of all SCN5A Exon 28 transcripts of ICD patient (+) shock]−[Ct of all SCN5A Exon 28 transcripts of control sample])
ΔCt_{truncated}=([Ct of truncated SCN5A Exon 28 transcript of ICD patient (+) shock]−[Ct of truncated SCN5A Exon 28 transcript of control sample])
ΔCt_{housekeeping gene}=([Ct of housekeeping gene of ICD patient (+) shock]−[Ct of housekeeping gene of control sample])
wherein “ICD patient (+) shock” is a biological sample obtained from a subject known as having an ICD that has given a shock and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

54. The method of any one of claims 28 to 53, wherein each level is determined via Real Time-Polymerase Chain Reaction (R_{T}-PCR).

55. The method of any one of claims 28 to 54, wherein the level of the truncated SCN5A Exon 28 transcript is a level of SCN5A Exon 28 Splice Variant C (E28C) or a level of SCN5A Exon 28 Splice Variant D (E28D).

56. The method of claim 55, wherein the level of a truncated SCN5A Exon 28 transcript is a level of E28C and E28D.

57. The method of any one of claims 28 to 56, wherein the level of the truncated SCN5A Exon 28 transcript is compared to (i) a level of WT SCN5A Exon 28 transcripts, (ii) a level of E28A-S, or (iii) a combination of (i) and (ii).

58. The method of any one of claims 28 to 56, wherein the level of the truncated SCN5A Exon 28 transcript is compared to a level of all of WT SCN5A Exon 28 transcripts, E28A-S, E28B, E28C, and E28D.

59. The method of any one of claims 28 to 56, wherein the level of the truncated SCN5A Exon 28 transcript is compared to a level of (A) and (B), wherein:
(A) is one of (i) a level of WT SCN5A Exon 28 transcripts, (ii) a level of E28A-S, or (iii) a combination of (i) and (ii), and
(B) is one or more of E28B, E28C, and E28D.

60. The method of claim 57 or 58, wherein the truncated SCN5A Exon 28 transcript is E28C.

61. The method of claim 60, wherein
$${R}_{S} = {R}_{E 28 C} = \frac{Calibrated abundance of E 28 C transcript}{\begin{matrix}Calibrated abundance of a full \\ length SCN 5 A Exon 28 transcript\end{matrix}}$$
wherein:
calibrated abundance of E28C transcript=2^{-ΔΔCtE28C transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{E28C transcript}=ΔCt_{E28C transcript}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{E28C}=([Ct of E28C transcript of subject sample]−[Ct of E28C transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts; or wherein
$${R}_{E 28 C} = \frac{Calibrated abundance of E 28 C transcript}{Calibrated abundance of all SCN 5 A Exon 28 transcripts}$$
wherein:
calibrated abundance of E28C transcript=2^{-ΔΔCtE28C transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{E28C transcript}=ΔCt_{E28C transcript}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{E28C}=([Ct of E28C transcript of subject sample]−[Ct of E28C transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

62. The method of claim 57 or 58, wherein truncated SCN5A Exon 28 transcript is E28D.

63. The method of claim 62, wherein
$${R}_{S} = {R}_{E 28 D} = \frac{Calibrated abundance of E 28 D transcript}{\begin{matrix}Calibrated abundance of full \\ length SCN 5 A Exon 28 transcript\end{matrix}}$$
wherein:
calibrated abundance of E28D transcript=2^{-ΔΔCtE28D transcript}
calibrated abundance of full length SCN5A Exon 28 transcript=2^{-ΔΔCtfull length SCN5A Exon 28 transcript}
ΔΔCt_{full length SCN5A Exon 28 transcript}=ΔCt_{full length}−ΔCt_{housekeeping gene}
ΔΔCt_{E28D transcript}=ΔCt_{E28D transcript}−ΔCt_{housekeeping gene}
ΔCt_{full length}=([Ct of full length SCN5A Exon 28 transcript of subject sample]−[Ct of full length SCN5A Exon 28 transcript of control sample])
ΔCt_{E28D}=([Ct of E28D transcript of subject sample]−[Ct of E28D transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “full length” refers to WT SCN5A Exon 28 transcripts or E28A-S transcripts or both WT SCN5A Exon 28 transcripts and E28A-S transcripts,
or wherein
$${R}_{E 28 D} = \frac{Calibrated abundance of E 28 D transcript}{Calibrated abundance of all SCN 5 A Exon 28 transcripts}$$
wherein:
calibrated abundance of E28D transcript=2^{-ΔΔCtE28D transcript}
calibrated abundance of all SCN5A Exon 28 transcripts=2^{-ΔΔCtall SCN5A Exon 28 transcripts}
ΔΔCt_{all SCN5A Exon 28 transcripts}=ΔCt_{all SCN5A Exon 28 transcripts}−ΔCt_{housekeeping gene}
ΔΔCt_{E28D transcript}=ΔCt_{E28D transcript}−ΔCt_{housekeeping gene}
ΔCt_{all SCN5A Exon 28 transcripts}=([Ct of all SCN5A Exon 28 transcripts of subject sample]−[Ct of all SCN5A Exon 28 transcripts of control sample])
ΔCt_{E28D}=([Ct of E28D transcript of subject sample]−[Ct of E28D transcript of control sample])
ΔCt_{housekeeping gene}=([Ct of housekeeping gene of subject sample]−[Ct of housekeeping gene of control sample])
wherein “subject sample” is a biological sample obtained from the subject and “control sample” is a biological sample obtained from a control subject known as (i) not having an ICD, (ii) not having a cardiac disease, (iii) having normal left ventricular function, (iv) not having diastolic dysfunction, or (v) a combination thereof, wherein “all SCN5A Exon 28 transcripts” refers to all of WT, E28A-S, E28B, E28C, and E28D.

64. The method of any one of claims 28 to 63, comprising determining a first R_{S} and a second R_{S}, wherein the first R_{S} compares a level of E28C to the level of all SCN5A Exon 28 transcripts and the second R_{S} compares a level of E28D to the level of all SCN5A Exon 28 transcripts.

65. The method of claim 64, wherein the first R_{S} is RE28C and the second R_{S} is E28D.

66. A method of determining a subject's risk for arrhythmias, comprising the step of determining a ratio, Rs, which compares a level of a truncated SCN5A Exon 28 transcript of a biological sample obtained from a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

67. The method of claim 66, wherein the subject being at risk for arrhythmias when Rs is greater than or equal to a threshold ratio, R_{T}.

68. The method of claim 66 or 67, wherein the arrhythmias is atrial arrhythmias, optionally, atrial fibrillation.

69. A method of determining a subject's risk for heart failure comprising the step of determining a ratio, Rs, which compares a level of a truncated SCN5A Exon 28 transcript of a biological sample obtained from a subject to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample.

70. The method of claim 69, wherein the subject is at risk for heart failure when Rₛ is greater than or equal to a threshold ratio, R_{T}.

71. The method of any one of the preceding claims, comprising the step of measuring (i) a level of a full length SCN5A Exon 28 transcript, (ii) a level of truncated SCN5A transcript, or (iii) a level of all SCN5A Exon 28 transcripts, of a biological sample obtained from the subject.

72. The method of claim 71, wherein the step of measuring comprises carrying out a polymerase chain reaction, optionally, a Real-Time PCR.

73. The method of claim 71 or 72, comprising the steps of (A) measuring (i) a level of a full length SCN5A Exon 28 transcript, (ii) a level of truncated SCN5A transcript, or (iii) a level of all SCN5A Exon 28 transcripts, of a biological sample obtained from the subject, and (B) obtaining from a medical record of the subject (i) a level of a full length SCN5A Exon 28 transcript, (ii) a level of a truncated SCN5A transcript, or (iii) a level of all SCN5A Exon 28 transcripts.

74. The method of any one of the preceding claims, comprising providing to the subject an anti-arrhythmic therapy.

75. The method of claim 74, comprising the step of implanting an ICD into the subject determined to have a need therefor.

76. The method of claim 74 or 75, comprising the step of administering to the subject an anti-arrhythmic agent.

77. The method of claim 76, wherein the anti-arrhythmic agent is a sodium channel blocking agent.

78. The method of any one of the preceding claims, comprising the steps of (i) obtaining a biological sample from the subject every 6-12 months and (ii) determining R_{S} of each biological sample obtained.

79. The method of any one of the preceding claims, comprising determining a level of a SCN5A splicing factor or a UPR protein in the sample or a chaperone protein.

80. The method of claim 79, comprising determining a level of hLuc7A, RBM25, or PERK.

81. The method of claim 79, wherein the chaperone protein is CHOP or calnexin.

82. The method of any one of the preceding claims, wherein the biological sample comprises white blood cells, cardiac cells, or muscle cells from the subject.

83. The method of claim 82, wherein the biological sample is blood from the subject.

84. The method of any one of the preceding claims, wherein the subject suffers from chronic cardiac left ventricular ejection fraction of less than or about 50%.

85. The method of claim 84, wherein the subject suffers from chronic cardiac left ventricular ejection fraction of less than or about 40%.

86. The method of claim 85, wherein the subject suffers from chronic cardiac left ventricular ejection fraction of less than or about 35%.

87. The method of any one of the preceding claims, wherein the subject (i) suffers from nonischemic dilated cardiomyopathy or ischemic heart disease at least 40 days post-myocardial infarction, (ii) is classified as an NYHA functional class II or III subject, while receiving chronic medical therapy, or (iii) both (i) and (ii).

88. The method of any one of the preceding claims, wherein the subject has a structural heart disease.

89. The method of any one of claims 30-44, and 46-83, wherein the subject has an ICD.

90. The method of any one of the preceding claims, wherein the subject has been diagnosed with HF.

91. A kit comprising (a) an E28C binding agent and/or an E28D binding agent, (b) a WT SCN5A binding agent and/or an E28A-S binding agent and instructions for use.

92. The kit of claim 91, further comprising a binding agent to all SCN5A Exon 28 transcripts: WT, E28A-S, E28B, E28C, and E28D

93. The kit of claim 91 or 92, wherein the binding agents are nucleic acids.

94. A kit comprising a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having an ICD that has given a shock; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values, or access to any one or more of (I) to (V).

95. A kit comprising a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as having an ICD that has not given a shock; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values, or access to any one or more of (I) to (V).

96. A kit comprising a computer-readable storage medium having stored thereon (I) a plurality of data values, each data value is a ratio determined from a biological sample obtained from a subject of a first population, wherein the ratio compares a level of a truncated SCN5A Exon 28 transcript to (i) a level of a full length SCN5A Exon 28 transcript of the biological sample or (ii) a level of all SCN5A Exon 28 transcripts of the biological sample or (iii) a level of a full length SCN5A Exon 28 transcript and a level or one or more truncated SCN5A Exon 28 transcripts of the biological sample, wherein each subject of the first population is a subject known as (I) not having an ICD, (II) not having a cardiac disease, (III) having normal left ventricular function, (IV) not having diastolic dysfunction, or (V) a combination thereof; (II) a Gaussian distribution of the plurality of data values; (IV) the mean value and the standard deviation of the Gaussian distribution, or (V) a threshold ratio, R_{T}, which is based on the mean value and the standard deviation of the Gaussian distribution of the plurality of data values, or access to any one or more of (I) to (V).

97. A system comprising:
a processor;
a memory device coupled to the processor, the memory device storing machine readable instructions that, when executed by the processor, cause the processor to:
(i) receive a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) fit the plurality of data values to a first Gaussian distribution;
(iii) determine a mean value, μ, and a standard deviation, σ, of the first Gaussian distribution;
(iv) set a first threshold ratio, R_{T}, at μ−Xσ, wherein X is a number between 0.7 and 4.0.

98. The system of claim 97, wherein R_{T}=μ−Xσ, and X is a number between 0.7 and 1.0.

99. The system of claim 97, wherein R_{T}=μ−Xσ, and X is a number between 2.0 and 4.0.

100. The system of claim 99, wherein R_{T}=μ−Xσ, and X is a number between 2.326 and 4.0.

101. The system of any one of claims 97 to 100, comprising machine readable instructions that, when executed by the processor, cause the processor to receive as input an Rs and compare Rs to R_{T}.

102. The system of claim 101, comprising machine readable instructions that, when executed by the processor, cause the processor to provide an output indicating the relationship between Rs and R_{T}.

103. The system of any one of claims 97 to 102, comprising machine readable instructions that, when executed by the processor, cause the processor to:
(i) receive a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fit the second plurality of data values to a second Gaussian distribution;
(iii) determine a mean value, μ, and a standard deviation, σ, of the second Gaussian distribution;
(iv) set a second threshold ratio, R_{T2}, at μ+Xσ, wherein X is a number between 0.7 and 4.0.

104. The system of claim 103, wherein R_{T2}=μ+Xσ, and X is a number between 0.7 and 1.0.

105. The system of claim 103, wherein R_{T2}=μ+Xσ, and X is a number between 2.0 and 4.0.

106. The system of claim 105, wherein R_{T2}=μ+Xσ, and X is a number between 2.326 and 4.0.

107. The system of any one of claims 97 to 106, comprising machine readable instructions that, when executed by the processor, cause the processor to:
(i) receive a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fit the third plurality of data values to a third Gaussian distribution;
(iii) determine a mean value, μ, and a standard deviation, σ, of the third Gaussian distribution;
(iv) set a third threshold ratio, R_{T3}, at μ+4.0σ.

108. The system of any one of claims 103 to 107, comprising machine readable instructions that, when executed by the processor, cause the processor to set a combined threshold ratio, R_{Tcombined}, at a point where the area under the curve of the first Gaussian distribution is maximized and the area under the curve of the second Gaussian distribution is minimized.

109. A system comprising:
a processor;
a memory device coupled to the processor, the memory device storing machine readable instructions that, when executed by the processor, cause the processor to:
(i) receive a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as having an ICD that has not given a shock;
(ii) fit the plurality of data values to a Gaussian distribution;
(iii) determine a mean value, μ, and a standard deviation, σ, of the Gaussian distribution;
(iv) set a threshold ratio, R_{T2}, at μ+Xσ, wherein X is a number between 0.7 and 4.0.

110. The system of claim 109, wherein R_{T2}=μ+Xσ, and X is a number between 0.7 and 1.0.

111. The system of claim 109, wherein R_{T2}=μ+Xσ, and X is a number between 2.0 and 4.0.

112. The system of claim 111, wherein R_{T2}=μ+Xσ, and X is a number between 2.326 and 4.0.

113. A system comprising machine readable instructions that, when executed by the processor, cause the processor to:
(i) receive a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as (I) not having an ICD, (II) not having a cardiac disease, (III) having normal left ventricular function, (IV) not having diastolic dysfunction, or (V) a combination thereof;
(ii) fit the plurality of data values to a Gaussian distribution;
(iii) determine a mean value, μ, and a standard deviation, σ, of the Gaussian distribution;
(iv) set a threshold ratio, R_{T3}, at μ+4.0σ.

114. A computer-readable storage medium having stored thereon machine-readable instructions executable by a processor, comprising:
(i) instructions for causing the processor to receive a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) instructions for causing the processor to fit the plurality of data values to a first Gaussian distribution;
(ii) instructions for causing the processor to determine a mean value, μ, and a standard deviation, σ, of the first Gaussian distribution;
(iii) instructions for causing the processor to set a first threshold ratio, R_{T}, at μ−Xσ, wherein X is a number between 0.7 and 4.0.

115. The computer-readable storage medium of claim 114, wherein R_{T}=μ−Xσ, and X is a number between 0.7 and 1.0.

116. The computer-readable storage medium of claim 114, wherein R_{T}=μ−Xσ, and X is a number between 2.0 and 4.0.

117. The computer-readable storage medium of claim 116, wherein R_{T}=μ−Xσ, and X is a number between 2.326 and 4.0.

118. The computer-readable storage medium of any one of claims 114 to 117, comprising instructions for causing the processor to receive as input an Rₛ and compare Rₛ to R_{T}.

119. The computer-readable storage medium of claim 118, comprising instructions for causing the processor to provide an output indicating the relationship between Rₛ and R_{T}.

120. The computer-readable storage medium of any one of claims 114 to 119, comprising instructions for causing the processor to:
(i) receive a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fit the second plurality of data values to a second Gaussian distribution;
(iii) determine a mean value, μ, and a standard deviation, σ, of the second Gaussian distribution;
(iv) set a second threshold ratio, R_{T2}, at μ+Xσ, wherein X is a number between 0.7 and 4.0.

121. The computer-readable storage medium of claim 120, wherein R_{T2}=μ+Xσ, and X is a number between 0.7 and 1.0.

122. The computer-readable storage medium of claim 120, wherein R_{T2}=μ+Xσ, and X is a number between 2.0 and 4.0.

123. The computer-readable storage medium of claim 122, wherein R_{T2}=μ+Xσ, and X is a number between 2.326 and 4.0.

124. The computer-readable storage medium of any one of claims 114 to 123, comprising instructions for causing the processor to:
(i) receive a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fit the third plurality of data values to a third Gaussian distribution;
(iii) determine a mean value, μ, and a standard deviation, σ, of the third Gaussian distribution;
(iv) set a third threshold ratio, R_{T3}, at μ+4.0σ.

125. The computer-readable storage medium of any one of claims 120 to 124, comprising instructions for causing the processor to set a combined threshold ratio, R_{Tcombined}, at a point where the area under the curve of the first Gaussian distribution is maximized and the area under the curve of the second Gaussian distribution is minimized.

126. A computer-readable storage medium having stored thereon machine-readable instructions executable by a processor, comprising:
(i) instructions for causing the processor to receive a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as having an ICD that has not given a shock;
(ii) instructions for causing the processor to fit the plurality of data values to a Gaussian distribution;
(iii) instructions for causing the processor to determine a mean value, μ, and a standard deviation, σ, of the Gaussian distribution;
(iv) instructions for causing the processor to set a threshold ratio, R_{T2}, at μ+Xσ, wherein X is a number between 0.7 and 4.0.

127. The computer-readable storage medium of claim 126, wherein R_{T2}=μ+Xσ, and X is a number between 0.7 and 1.0.

128. The computer-readable storage medium of claim 126, wherein R_{T2}=μ+Xσ, and X is a number between 2.0 and 4.0.

129. The computer-readable storage medium of claim 128, wherein R_{T2}=μ+Xσ, and X is a number between 2.326 and 4.0.

130. A computer-readable storage medium having stored thereon machine-readable instructions executable by a processor, comprising:
(i) instructions for causing the processor to receive a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as (I) not having an ICD, (II) not having a cardiac disease, (III) having normal left ventricular function, (IV) not having diastolic dysfunction, or (V) a combination thereof;
(ii) instructions for causing the processor to fit the plurality of data values to a Gaussian distribution;
(iii) instructions for causing the processor to determine a mean value, μ, and a standard deviation, σ, of the Gaussian distribution;
(iv) instructions for causing the processor to set a threshold ratio, R_{T3}, at μ+4.0σ.

131. A method implemented by a processor in a computer, the method comprising the steps of:
(i) receiving a plurality of data values, each data value is a ratio determined from a biological sample obtained from a subject of a first population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the first population is a subject known as having an ICD that has given a shock;
(ii) fitting the plurality of data values to a first Gaussian distribution;
(ii) determining a mean value, μ, and a standard deviation, σ, of the first Gaussian distribution;
(iii) setting a first threshold ratio, R_{T}, at μ−Xσ, wherein X is a number between 0.7 and 4.0.

132. The method of claim 131, wherein R_{T}=μ−Xσ, and X is a number between 0.7 and 1.0.

133. The method of claim 131, wherein R_{T}=μ−Xσ, and X is a number between 2.0 and 4.0.

134. The method of claim 133, wherein R_{T}=μ−Xσ, and X is a number between 2.326 and 4.0.

135. The method of any one of claims 131 to 134, comprising the step of receiving as input an Rₛ and compare Rₛ to R_{T}.

136. The method of claim 135, comprising the step of providing an output indicating the relationship between Rₛ and R_{T}.

137. The method of any one of claims 131 to 136, comprising the steps of:
(i) receiving a second plurality of data values, each data value of the second plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the second population is a subject known as having an ICD that has not given a shock;
(ii) fitting the second plurality of data values to a second Gaussian distribution;
(iii) determining a mean value, μ, and a standard deviation, σ, of the second Gaussian distribution;
(iv) setting a second threshold ratio, R_{T2}, at μ+Xσ, wherein X is a number between 0.7 and 4.0.

138. The method of claim 137, wherein R_{T2}=μ+Xσ, and X is a number between 0.7 and 1.0.

139. The method of claim 137, wherein R_{T2}=μ+Xσ, and X is a number between 2.0 and 4.0.

140. The method of claim 139, wherein R_{T2}=μ+Xσ, and X is a number between 2.326 and 4.0.

141. The method of any one of claims 131 to 140, comprising the steps of:
(i) receiving a third plurality of data values, each data value of the third plurality is a ratio determined from a biological sample obtained from a subject of a second population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the third population is a subject known as (I) not having an ICD, (II) not having a cardiac disease, (III) having normal left ventricular function, (IV) not having diastolic dysfunction, or (V) a combination thereof;
(ii) fitting the third plurality of data values to a third Gaussian distribution;
(iii) determining a mean value, μ, and a standard deviation, σ, of the third Gaussian distribution;
(iv) setting a third threshold ratio, R_{T3}, at μ+4.0σ.

142. The method of any one of claims 137 to 141, comprising the step of setting a combined threshold ratio, R_{Tcombined}, at a point where the area under the curve of the first Gaussian distribution is maximized and the area under the curve of the second Gaussian distribution is minimized.

143. A method implemented by a processor in a computer, the method comprising the steps of:
(i) receiving a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as having an ICD that has not given a shock;
(ii) fitting the plurality of data values to a Gaussian distribution;
(iii) determining a mean value, μ, and a standard deviation, σ, of the Gaussian distribution;
(iv) setting a threshold ratio, R_{T2}, at μ+Xσ, wherein X is a number between 0.7 and 4.0.

144. The method of claim 134, wherein R_{T2}=μ+Xσ, and X is a number between 0.7 and 1.0.

145. The method of claim 134, wherein R_{T2}=μ+Xσ, and X is a number between 2.0 and 4.0.

146. The method of claim 145, wherein R_{T2}=μ+Xσ, and X is a number between 2.326 and 4.0.

147. A method implemented by a processor in a computer, the method comprising the steps of:
(i) receiving a plurality of data values, each data value of the plurality is a ratio determined from a biological sample obtained from a subject of a population, wherein the ratio compares a level of a truncated SCN5A Exon 28 transcript of a biological sample obtained from a subject to (A) a level of a full length SCN5A Exon 28 transcript of the biological sample or (B) a level of all SCN5A Exon 28 transcripts of the biological sample or (C) a level of a full length SCN5A Exon 28 transcript and a level or one or more truncated SCN5A Exon 28 transcripts of the biological sample, wherein each subject of the population is a subject known as (I) not having an ICD, (II) not having a cardiac disease, (III) having normal left ventricular function, (IV) not having diastolic dysfunction, or (V) a combination thereof;
(ii) fitting the plurality of data values to a Gaussian distribution;
(iii) determining a mean value, μ, and a standard deviation, σ, of the Gaussian distribution;
(iv) setting a threshold ratio, R_{T3}, at μ+4.0σ.
